(19) United States
(12) Patent Application Publication
(10) Pub. No.: US 2026/0337646 A1
(43) Pub. Date: Oct. 29, 2026

(54) FLAVONOID BIOPRODUCTION USING MICROORGANISM HOSTS

(71) Applicant: DEBUT BIOTECHNOLOGY, INC., San Diego, CA (US)

(72) Inventors: Nicholas Brideau, San Diego, CA (US); Joshua Britton, San Diego, CA (US); Jingyi LI, San Diego, CA (US)

(21) Appl. No.: 19/104,124

(22) PCT Filed: Aug. 18, 2023
(86) PCT No.: PCT/US2023/072449

§ 371 Date: Feb. 14, 2025

Related U.S. Application Data
(60) Provisional application No. 63/399,005, filed on Aug. 18, 2022.

(51) Int. Cl. C12P 17/06; C12N 1/20; C12N 9/02; C12N 15/52; C12R 1/19

(52) U.S. Cl. CPC C12P 17/06; C12N 1/20; C12N 9/0071; C12N 15/52; C12Y 114/11009; C12Y 114/11023; C12Y 114/14; C12R 2001/19

(57) ABSTRACT

The invention is directed to methods involved in the production of flavonoids, anthocyanins and other organic compounds. The invention provides cells engineered for the production of flavonoids, anthocyanins and other organic compounds, where the engineered cells include one or more genetic modifications that increase flavonoid production by increasing metabolic flux to flavonoid precursors and/or reducing carbon losses resulting from the production of byproducts.

### I. FIELD OF THE INVENTION

The invention related to materials (including engineered cells and cell lines) and methods involved in the production of flavonoids, anthocyanins and other organic compounds.

### II. BACKGROUND OF THE INVENTION

Flavonoids and anthocyanins are natural products produced in plants that find a variety of roles such as antioxidants, ultraviolet (UV) defense mechanisms, and colors. Over the past several years, the health benefits of flavonoids and anthocyanins have been widely demonstrated. These compounds are capable of scavenging radicals and can act as enzyme inhibitors and anti-inflammatory agents. With these recognized health and color benefits, much research has gone into understanding how these compounds are made in nature.

Myricetin is a common plant-derived flavonoid and is well recognized for its nutraceuticals value. It is one of the key ingredients of various foods and beverages. The compound exhibits a wide range of activities that include strong antioxidant, anticancer, antidiabetic and anti-inflammatory activities. Kaempferol is a polyphenol antioxidant found in fruits and vegetables. Many studies have described the beneficial effects of dietary kaempferol in reducing the risk of chronic diseases, especially cancer. Quercetin, a plant pigment is a potent antioxidant flavonoid, found mostly in onions, grapes, berries, cherries, broccoli, and citrus fruits. It is a versatile antioxidant known to possess protective abilities against tissue injury induced by various drug toxicities.

Flavonoids and anthocyanins are synthesized from phenylpropanoid starter units and malonyl-Cofactor-A (malonyl-CoA) extender units that then undergo modifications to create many polyphenol compounds such as taxifolin, naringenin, and (+)-catechin. However, in most cases, these compounds are extracted or chemically manufactured.

### III. SUMMARY OF THE INVENTION

To move away from agriculture and chemically derived products, we have created engineered cells for the bioproduction of flavonoids and anthocyanins. This approach provides a feasible route for the rapid, safe, economical, and sustainable production of a wide variety of important flavonoids, including myricetin, kaempeferol, and quercetin.

Herein, a range of flavonoids and anthocyanins including naringenin, eriodictyol, taxifolin, dihydrokaempferol, (+)-catechin, cyanidin, cyaninidin-3-glucoside, myricetin, kaempeferol, and quercetin are biomanufactured using a modified microbial host. Herein, the engineered cells include one or more genetic modifications that increase(s) flavonoid and anthocyanin bioproduction by increasing metabolic flux to flavonoid precursors and/or reducing carbon losses resulting from the production of byproducts.

In the first aspect, the invention provides engineered host cells, wherein the engineered host cells comprise one or more genetic modifications to increase the production of flavonols through transformation by one or more enzymes of a carbon source by the engineered host cell. In one embodiment, the flavonol is selected from a group consisting of kaempferol, myricetin, and quercetin. In another embodiment, the carbon source is selected from a group consisting of naringenin, dihydrokaempferol (DHK), dihydromyricetin (DHM), dihydroquercetin (DHQ), and eriodictyol (EDL). In another embodiment, one or more genetic modifications are at least one genetic modification selected from the group consisting of: (i) one or more modifications for over-expressing one or more endogenous genes in the engineered host cells; (ii) one or more modifications for under-expressing one or more endogenous genes in the engineered host cells; (iii) one or more genetic modification is expressing one or more non-native genes in the engineered host cells; and (iv) a combination thereof. In another embodiment, the engineered host cell comprises at least one or more nucleic acid sequences selected from the group consisting of: (i) nucleic acid sequences encoding native or modified flavanone-3-hydroxylase (F3H) or a homolog thereof, (ii) nucleic acid sequences encoding native or modified flavanone-3′-hydroxylase (F3′H) or a homolog thereof, (iii) nucleic acid sequences encoding native or modified flavonoid 3′,5′-hydroxylase (F3′5′H) or a homolog thereof, (iv) nucleic acid sequences encoding native or modified flavonol synthase (FLS) or a homolog thereof, and (v) any combinations thereof. In another embodiment, the engineered host cell is E. coli. In another embodiment, the production of flavonols through enzymatic transformation of a carbon source includes one or more chemical intermediates. In another embodiment, the flavonol is myricetin, the carbon source is dihydromyricetin (DHM), and the one or more enzyme is flavonol synthase (FLS). In another embodiment, the flavonol is myricetin, the carbon source is dihydrokaempferol (DHK), and the one or more enzymes are selected from a group consisting of (i) flavonoid 3′,5′-hydroxylase (F3′5′H), (ii) flavonol synthase (FLS), and (iii) any combination thereof. In another embodiment, flavonoid 3′,5′-hydroxylase (F3′5′H) has an amino acid sequence at least 80% identical to the polypeptides selected from a group consisting of: (i) SEQ ID NO. 10, (ii) SEQ ID NO. 56, and (iii) SEQ ID NO. 57. In another embodiment, the flavonol is kaempferol, the carbon source is dihydrokaempferol (DHK), and the one or more enzyme is flavonol synthase (FLS). In another embodiment, flavonol synthase (FLS) has an amino acid sequence at least 80% identical to any one of the polypeptides listed in SEQ ID NOS. 99-122. In another embodiment, the flavonol is quercetin, the carbon source is eriodictoyl (EDL), and the one or more enzyme is selected from a group consisting of (i) flavanone-3-hydroxylase (F3H), (ii) flavonol synthase (FLS), and (iii) any combination thereof. In another embodiment, the flavanone-3-hydroxylase (F3H) has an amino acid sequence at least 80% identical to the polypeptides selected from a group consisting of: (i) SEQ ID NO. 7, (ii) SEQ ID NO. 45, (iii) SEQ ID NO. 46, (iv) SEQ ID NO. 47, and (v) SEQ ID NO. 48. In another embodiment, production of quercetin leads to formation of dihydroquercetin (DHQ) as an intermediate. In another embodiment, the flavonol is quercetin, the carbon source is dihydroquercetin (DHQ) and the one or more enzyme is flavonol synthase (FLS). In another embodiment, the flavonol is quercetin, the carbon source is kaempferol, and the enzyme is flavanone-3′-hydroxylase (F3′H). In another embodiment, flavanone-3′-hydroxylase (F3′H) has an amino acid sequence at least 80% identical to the polypeptide set forth in (i) SEQ ID NO: 8 (ii) SEQ ID NO. 49, (iii) SEQ ID NO. 50, (iv) SEQ ID NO. 51, and (v) SEQ ID NO. 52. In another embodiment, the flavonol is kaempferol, the carbon source is dihydrokaempferol (DHK), naringenin, or a combination thereof, and the one or more enzyme is selected from a group consisting of (i) flavanone-3-hydroxylase (F3H), (ii) flavonol synthase (FLS), or (iii) any combination thereof. In another embodiment, the flavonol is quercetin, the carbon source is dihydroquerctin (DHQ), dihydrokaempferol (DHK), eriodictyol (EDL), naringenin, kaempferol, or any combination thereof, and the one or more enzyme is selected from a group consisting of: (i) flavonol synthase (FLS), (ii) flavanone-3′-hydroxylase (F3′H), (iii) flavanone-3-hydroxylase (F3H), or (iv) any combination thereof. In another embodiment, the flavonol is myricetin, the carbon source is dihydromyricetin (DHM), dihydroquerctin (DHQ), dihydrokaempferol (DHK), eriodictyol (EDL), naringenin, quercetin, kaempferol, or any combination thereof, and the one or more enzyme is selected from a group consisting of: (i) flavonol synthase (FLS), (ii) flavanone-3′-hydroxylase (F3′H), (iii) flavanone-3-hydroxylase (F3H), (iv) flavonoid-3′-5′-hydroxylase (F3′5′H), or (v) any combination thereof.

In another aspect, the invention provides a method of increasing the production of flavonols, comprising engineered host cells, wherein the engineered host cells comprise one or more genetic modifications to increase the production of flavonols through transformation by one or more enzymes of a carbon source by the engineered host cell. In one embodiment, the flavonol is selected from a group consisting of kaempferol, myricetin, and quercetin. In another embodiment, the carbon source is selected from a group consisting of naringenin, dihydrokaempferol (DHK), dihydromyricetin (DHM), dihydroquercetin (DHQ), and eriodictyol (EDL). In another embodiment, one or more genetic modifications are at least one genetic modification selected from the group consisting of: (i) one or more modifications for over-expressing one or more endogenous genes in the engineered host cells; (ii) one or more modifications for under-expressing one or more endogenous genes in the engineered host cells; (iii) one or more genetic modification is expressing one or more non-native genes in the engineered host cells; and (iv) a combination thereof. In another embodiment, the engineered host cell comprises at least one or more nucleic acid sequences selected from the group consisting of: (i) nucleic acid sequences encoding native or modified flavanone-3-hydroxylase (F3H) or a homolog thereof, (ii) nucleic acid sequences encoding native or modified flavanone-3′-hydroxylase (F3′H) or a homolog thereof, (iii) nucleic acid sequences encoding native or modified flavonoid 3′,5′-hydroxylase (F3′5′H) or a homolog thereof, (iv) nucleic acid sequences encoding native or modified flavonol synthase (FLS) or a homolog thereof, and (v) any combinations thereof. In another embodiment, the engineered host cell is E. coli. In another embodiment, the production of flavonols through enzymatic transformation of a carbon source includes one or more chemical intermediates. In another embodiment, the flavonol is myricetin, the carbon source is dihydromyricetin (DHM), and the one or more enzyme is flavonol synthase (FLS). In another embodiment, the flavonol is myricetin, the carbon source is dihydrokaempferol (DHK), and the one or more enzymes are selected from a group consisting of (i) flavonoid 3′,5′-hydroxylase (F3′5′H), (ii) flavonol synthase (FLS), and (iii) any combination thereof. In another embodiment, flavonoid 3′,5′-hydroxylase (F3′5′H) has an amino acid sequence at least 80% identical to the polypeptides selected from a group consisting of: (i) SEQ ID NO. 10, (ii) SEQ ID NO. 56, and (iii) SEQ ID NO. 57. In another embodiment, the flavonol is kaempferol, the carbon source is dihydrokaempferol (DHK), and the one or more enzyme is flavonol synthase (FLS). In another embodiment, flavonol synthase (FLS) has an amino acid sequence at least 80% identical to any one of the polypeptides listed in SEQ ID NOS. 99-122. In another embodiment, the flavonol is quercetin, the carbon source is eriodictoyl (EDL), and the one or more enzyme is selected from a group consisting of (i) flavanone-3-hydroxylase (F3H), (ii) flavonol synthase (FLS), and (iii) any combination thereof. In another embodiment, the flavanone-3-hydroxylase (F3H) has an amino acid sequence at least 80% identical to the polypeptides selected from a group consisting of: (i) SEQ ID NO. 7, (ii) SEQ ID NO. 45, (iii) SEQ ID NO. 46, (iv) SEQ ID NO. 47, and (v) SEQ ID NO. 48. In another embodiment, production of quercetin leads to formation of dihydroquercetin (DHQ) as an intermediate. In another embodiment, the flavonol is quercetin, the carbon source is dihydroquercetin (DHQ) and the one or more enzyme is flavonol synthase (FLS). In another embodiment, the flavonol is quercetin, the carbon source is kaempferol, and the enzyme is flavanone-3′-hydroxylase (F3′H). In another embodiment, flavanone-3′-hydroxylase (F3′H) has an amino acid sequence at least 80% identical to the polypeptide set forth in (i) SEQ ID NO: 8 (ii) SEQ ID NO. 49, (iii) SEQ ID NO. 50, (iv) SEQ ID NO. 51, and (v) SEQ ID NO. 52. In another embodiment, the flavonol is kaempferol, the carbon source is dihydrokaempferol (DHK), naringenin, or a combination thereof, and the one or more enzyme is selected from a group consisting of (i) flavanone-3-hydroxylase (F3H), (ii) flavonol synthase (FLS), or (iii) any combination thereof. In another embodiment, the flavonol is quercetin, the carbon source is dihydroquerctin (DHQ), dihydrokaempferol (DHK), eriodictyol (EDL), naringenin, kaempferol, or any combination thereof, and the one or more enzyme is selected from a group consisting of: (i) flavonol synthase (FLS), (ii) flavanone-3′-hydroxylase (F3′H), (iii) flavanone-3-hydroxylase (F3H), or (iv) any combination thereof. In another embodiment, the flavonol is myricetin, the carbon source is dihydromyricetin (DHM), dihydroquerctin (DHQ), dihydrokaempferol (DHK), eriodictyol (EDL), naringenin, quercetin, kaempferol, or any combination thereof, and the one or more enzyme is selected from a group consisting of: (i) flavonol synthase (FLS), (ii) flavanone-3′-hydroxylase (F3′H), (iii) flavanone-3-hydroxylase (F3H), (iv) flavonoid-3′-5′-hydroxylase (F3′5′H), or (v) any combination thereof.

### IV. BRIEF DESCRIPTION OF THE SEVERAL VIEWS OF THE DRAWING(S)

FIG. 1 shows the metabolic pathway of flavonoid and anthocyanin bioproduction in engineered cells and methods of preparing anthocyanins described herein.

FIG. 2 shows structures of the flavonoid and anthocyanin molecules that may be produced using engineered cells and methods of preparing anthocyanins described herein.

FIG. 3 shows HPLC spectra showing peaks corresponding to the molecules prepared using engineered cells and methods of preparing anthocyanins described herein.

FIG. 4 shows the pathway of flavonoid and anthocyanin bioproduction in engineered cells and methods of preparing anthocyanins described herein.

FIG. 5 shows the pathway of production of flavonols, including myricetin, kaempferol, and quercetin.

FIG. 6 shows the specific carbon source, intermediate, and the enzyme combinations to generate flavonols, including kaempferol, quercetin, and myricetin.

FIG. 7 provides the data for bioproduction of quercetin using the methods of the invention.

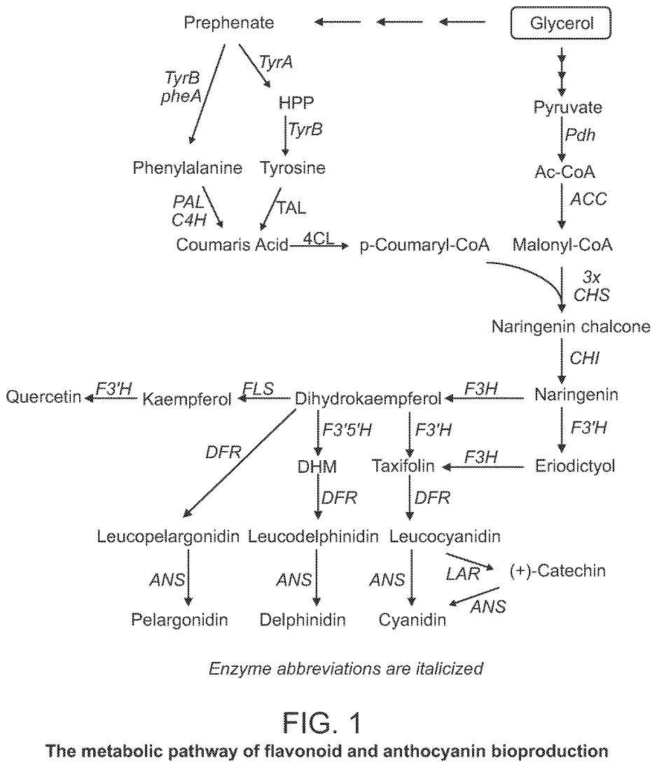

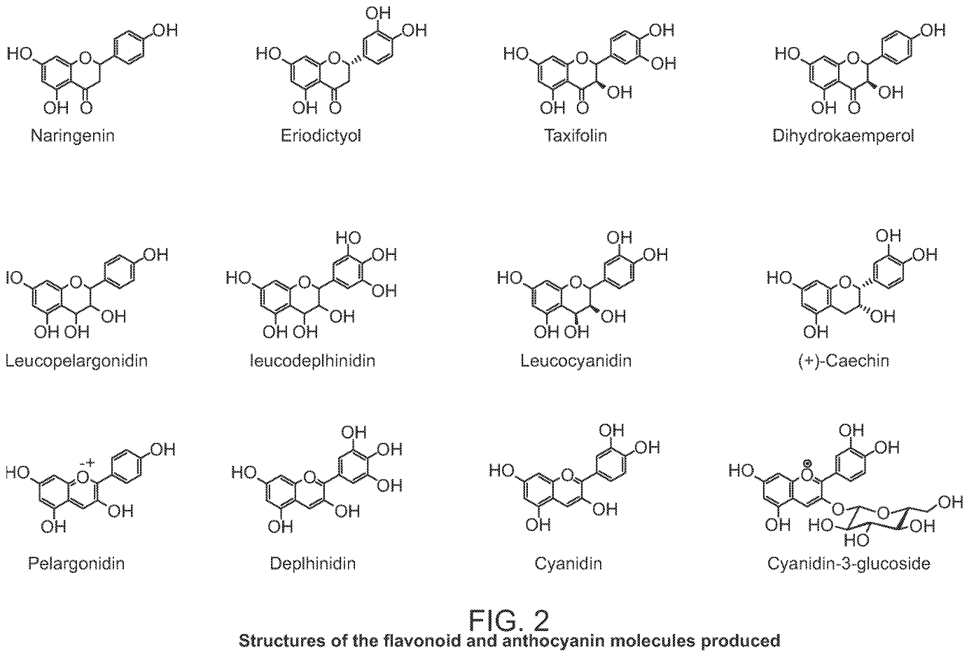

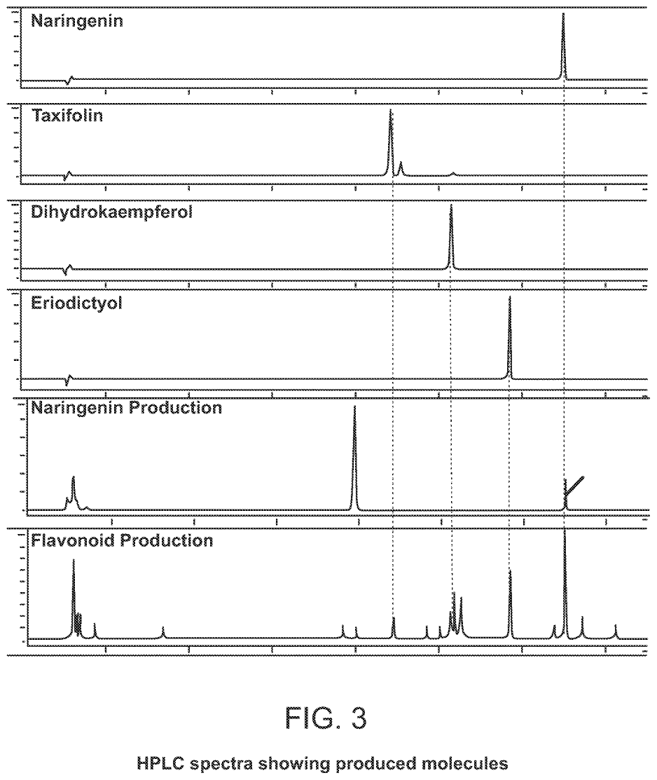

### V. DETAILED DESCRIPTION OF THE INVENTION

The present application provides engineered cells for producing one or more flavonoids, cultures that include the engineered cells, and methods of producing one or more flavonoids, or at least one anthocyanin. The terms “flavonoid”, “flavonoid product”, or “flavonoid compound” are used herein to refer to a member of a diverse group of phytonutrients found in almost all fruits and vegetables. As used herein, the terms “flavonoid”, “flavonoid product”, or “flavonoid compound” are used interchangeably to refer a molecule containing the general structure of a 15-carbon skeleton, which consists of two phenyl rings (A and B) and a heterocyclic ring. Flavonoids may include, but are not limited to, isoflavone type (e.g., genistein), flavone type (e.g., apigenin), flavonol type (e.g., kaempferol), flavanone type (e.g., naringenin), chalcone type (e.g., phloretin), anthocyanidin type (e.g., cyanidin), catechins, flavanones, and flavanonols. Flavonoid compounds of interest include, without limitation, naringenin, naringenin chalcone, eriodictyol, taxifolin, dihydrokaempferol, dihydroquercetin, dihydromyricetin, leucocyanidin, leucopelargonidin, leucodelphindin, pentahydroxyflavone, cyanidin, catechin, delphinidin, pelargonidin, and kaempferol. Anthocyanins are in the forms of anthocyanidin glycosides and acylated anthocyanins. Anthocyanin compounds of interest include, without limitation, cyanidin glycoside, delphinidin glycoside, pelargonidin glycoside, peonidin glycoside, and petunidin glycoside.

The terms ‘precursor’ or ‘flavonoid precursor’ as used herein may refer to any intermediate present in the biosynthetic pathway that leads to the production of catechins or anthocyanins. flavonoid precursors may include, but are not limited to tyrosine, phenylalanine, coumaric acid, p-coumaroyl-CoA, malonyl-CoA, pyruvate, acetyl-CoA, and naringenin.

Cells engineered for the production of a flavonoid or an anthocyanin can have one or multiple modifications, including, without limitation, the downregulation, disruption, or deletion of endogenous genes, the upregulation of an endogenous gene, and the introduction of exogenous genes.

The term “non-naturally occurring”, when used in reference to an enzyme is intended to mean that nucleic acids or polypeptides include at least one genetic alteration not normally found in a naturally occurring polypeptide or nucleic acid sequence. Naturally occurring nucleic acids, and polypeptides can be referred to as “wild-type” or “original”. A host cell, organism, or microorganism that includes at least one genetic modification generated by human intervention can also be referred to as “non-naturally occurring”, “engineered”, “genetically engineered,” or “recombinant”.

A host cell, organism, or microorganism engineered to express or overexpress a gene or nucleic acid sequence, or to overexpress an enzyme or polypeptide has been genetically engineered through recombinant DNA technology to include a gene or nucleic acid sequence that does not naturally encode the enzyme or polypeptide or to express an endogenous gene at a level that exceeds its level of expression in a non-altered cell. As nonlimiting examples, a host cell, organism, or microorganism engineered to express or overexpress a gene or a nucleic acid sequence, or to overexpress an enzyme or polypeptide can have any modifications that affect a coding sequence of a gene, the position of a gene on a chromosome or regulatory elements associated with a gene. Overexpression of a gene can also be by increasing the copy number of a gene in the cell or organism. Similarly, a host cell, organism, or microorganism engineered to under-express or to have reduced expression of a gene, nucleic acid sequence, or to under-express an enzyme or polypeptide can have any modifications that affect a coding sequence of a gene, the position of a gene on a chromosome or regulatory elements associated with a gene. Specifically included are gene disruptions, which include any insertions, deletions, or sequence mutations into or of the gene or a portion of the gene that affect its expression or the activity of the encoded polypeptide. Gene disruptions include “knockout” mutations that eliminate expression of the gene. Modifications to under-express a gene also include modifications to regulatory regions of the gene that can reduce its expression.

The term “exogenous” or “heterologous” is intended to mean that the referenced molecule or the referenced activity is introduced into the host microbial organism. The molecule can be introduced, for example, by introduction of an encoding nucleic acid into the host genetic material such as by integration into a host chromosome or as non-chromosomal genetic material that may be introduced on a vehicle such as a plasmid. Therefore, the term “endogenous” refers to a referenced molecule or activity that is naturally present in the host.

Genes or nucleic acid sequences can be introduced stably or transiently into a host cell using techniques well known in the art including, but not limited to, conjugation, electroporation, chemical transformation, transduction, and transfection. Optionally, for exogenous expression in E. coli or other prokaryotic cells, some nucleic acid sequences in the genes or cDNAs of eukaryotic nucleic acids can encode targeting signals such as an N-terminal mitochondrial or other targeting signal, which can be removed before transformation into prokaryotic host cells, if desired. Furthermore, genes can be subjected to codon optimization with techniques well known in the art to achieve optimized expression of the proteins.

The percent identity (% identity) between two sequences is determined when sequences are aligned for maximum homology. Algorithms well known to those skilled in the art, such as Align, BLAST, Clustal Omega, and others compare and determine a raw sequence similarity or identity, and also determine the presence or significance of gaps in the sequence which can be assigned a weight or score. Such algorithms also are known in the art and are similarly applicable for determining nucleotide or amino acid sequence similarity or identity and can be useful in identifying orthologs of genes of interest. Additional sequences added to a polypeptide sequence, such as but not limited to immunodetection tags, purification tags, localization sequences (presence or absence), etc., do not affect the % identity.

A homolog is a gene or genes that have the same or identical functions in different organisms. Genes that are orthologous can encode proteins with sequence similarity of about 45% to 100% amino acid sequence identity, and more preferably about 60% to 100% amino acid sequence identity. Genes can also be considered orthologs if they share three-dimensional structure but not necessarily sequence similarity, of a sufficient amount to indicate that they have evolved from a common ancestor to the extent that the primary sequence similarity is not identifiable. Paralogs are genes related by duplication within a genome, and can evolve new functions, even if these are related to the original one.

Provided herein are cells engineered for the production of flavonoids, anthocyanins and other organic compounds, where the engineered cells include one or more genetic modifications that increase flavonoid production by increasing metabolic flux to flavonoid precursors and/or reducing carbon losses resulting from the production of byproducts. As nonlimiting examples, a genetic modification can be a modification for over-expressing or under-expressing one or more endogenous genes in the engineered host cell or can be a modification for expressing one or more non-native genes in the engineered host cell. Engineered cells as provided herein can include multiple genetic modifications.

Also provided are cell cultures for producing one or more flavonoids or anthocyanins. The cell cultures include engineered cells as disclosed herein in a culture medium that includes a carbon source that can also be an energy source, such as glycerol, sugar, or an organic acid. In various embodiments, the culture medium can include at least one feed molecule such as but not limited to one or more organic acids or amino acids that can be converted into a flavonoid precursor (such as tyrosine, p-coumaroyl-CoA or malonyl-CoA). Examples of feed molecules include, but are not limited to, acetate, malonate, tyrosine, phenylalanine, pantothenate, coumarate, etc. In some embodiments, the feed molecules may be of reduced or low purity. For example, glycerol as a feed molecule may be crude glycerol, including a biomass comprising glycerol, for example, glycerol obtained as a byproduct of biodiesel processing. Alternatively, or in addition, the culture medium can include a supplemental compound that can be a cofactor or a precursor of a cofactor used by an enzyme that functions in a flavonoid pathway, such as, for examples, bicarbonate, biotin, thiamine, pantothenate, alpha-ketoglutarate, ascorbate, or 5-aminolevulinic acid.

Further provided are methods for producing flavonoids and anthocyanins that include culturing a cell engineered for the production of flavonoids or anthocyanins as provided herein under conditions in which the cell produces flavonoids or anthocyanins. In some examples, the methods include culturing the engineered cells in a culture medium that includes at least one feed molecule or supplement such as but not limited to: tyrosine, phenylalanine, malonate, p-coumarate, bicarbonate, acetate, pantothenate, biotin, thiamine, alpha-ketoglutarate, ascorbate, and 5-aminolevulinic acid. The methods can further include recovering at least one of the flavonoids from culture medium, whole culture, or the cells.

In a first aspect, provided herein are cells engineered to produce one or more flavonoids or anthocyanins, wherein the cells include, in addition to nucleic acid sequences encoding either tyrosine ammonia lyase activity and/or phenylalanine ammonia lyase activity and cinnamate-4-hydroxylase activity, 4-coumarate-CoA ligase activity, chalcone synthase activity, chalcone isomerase activity, flavanone-3-hydroxylase activity, flavonoid 3′-hydroxylase activity or flavonoid 3′5′-hydroxylase activity, cytochrome P450 reductase activity, leucoanthocyanidin reductase activity, and dihydroflavonol-4-reductase activity, one or more genetic modifications for improving production of the flavonoids or anthocyanins. As set forth herein, a cell that is engineered to produce one or more of the flavonoids is engineered to include an exogenous nucleic acid sequence encoding tyrosine ammonia lyase activity that can form 4-coumaric acid using tyrosine as substrate (e.g., tyrosine ammonia lyase TAL, EC: 4.3.1.25) or, alternatively or in addition, an exogenous nucleic acid sequence encoding phenylalanine ammonia lyase activity that can convert phenylalanine to trans-cinnamic acid and an exogenous nucleic acid sequence encoding cinnamate-4-hydroxylase activity that forms 4-coumaric acid from trans-cinnamic acid, an exogenous nucleic acid sequence encoding CoA ligase activity that forms p-coumaroyl-CoA from coumaric acid (e.g., 4-coumarate—CoA ligase, 4CL, EC:6.2.1.12), an exogenous nucleic acid sequence encoding polyketide synthase activity that forms naringenin chalcone using malonyl-CoA and p-coumaroyl-CoA as substrates (e.g., chalcone synthase, CHS, EC:2.3.1.74), an exogenous nucleic acid sequence encoding chalcone isomerase activity that forms naringenin from naringenin chalcone via its cyclase activity (e.g., chalcone-flavanone isomerase, CHI, EC:5.5.1.6), an exogenous nucleic acid sequence encoding flavanone-3-hydroxylase activity that forms dihydrokaempferol from naringenin or forms taxifolin from eriodictyol (e.g., naringenin 3-dioxygenase, F3H, EC: 1.14.11.9), an exogenous nucleic acid sequence encoding flavonoid 3′-hydroxylase or flavonoid 3′5′-hydroxylase activity coupled with an exogenous nucleic acid sequence encoding cytochrome P450 reductase activity to form taxifolin or dihydromyricetin from dihydrokaempferol or to form eriodictyol or pentahydroxyflavone from naringenin (e.g., flavonoid 3-monooxygenase, F3′H, EC: 1.14.13.21, EC: 1.14.14.82; cytochrome P450/NADPH—P450 reductase, EC:1.14.14.1; F3′5′H, EC:1.14.14.81), an exogenous nucleic acid sequence encoding dihydroflavonol-4-reductase activity that forms leucocyanidin from taxifolin, leucodelphinidin from dihydromyricetin, or leucopelargonidin from dihydrokaempferol (e.g., dihydroflavonol 4-reductase, EC:1.1.1), and an exogenous nucleic acid sequence encoding leucoanthocyanidin reductase activity that forms catechin from leucocyanidin (e.g., leucoanthocyanidin reductase, LAR, EC:1.17.1.3). Optionally, a cell that is engineered to produce anthocyanins is further engineered to include an exogenous nucleic acid sequence encoding anthocyanin synthase activity that forms cyanidin from catechin or leucocyanidin, forms delphinidin from leucodelphinidin, or forms pelargonidin from leucopelargonidin (e.g., anthocyanin synthase, ANS, EC:1.14.20.4) and to include an exogenous nucleic acid sequence encoding glucosyltransferase activity that forms cyanidin-3-O-beta-D-glucoside from cyanidin, delphinidin-3-O-beta-D-glucoside from delphinidin, or pelagonidin-3-O-beta-D-glucoside from pelagonidin (e.g., anthocyanidin 3-O-glucosyltransferase, 3GT, EC:2.4.1.115). The cells provided herein that are engineered to produce flavonoids or anthocyanins are further engineered to increase the production of flavonoids or anthocyanins product, for example by increasing metabolic flux to a flavonoid or anthocyanin pathway, or by decreasing byproduct formation.

A cell engineered to produce a flavonoid is further engineered to increase the supply of precursor malonyl-CoA. One strategy for increasing malonyl-CoA includes increasing acetyl-CoA carboxylase (ACC) activity. In various embodiments, the ACC enzyme, which in most eukaryotes, including fungi, is a large single chain polypeptide, and in plant and bacteria such as E. coli is a multi-subunit enzyme, is overexpressed in the host strain. Examples of acetyl-CoA carboxylase that can be expressed in a host cell engineered to produce a flavonoid or anthocyanin include, without limitation, the ACC genes of Mucor circinelloides, Rhodotorula toruloides, Lipomyces starkeyi, Ustilago maydis, and orthologs of these ACCs in other species having at least 50% amino acid identity to these ACCs.

Additional strategies for increasing malonyl-CoA include increasing acetyl-CoA, which is converted to malonyl-CoA by acetyl-CoA carboxylase (ACC). In some embodiments, acetyl-CoA synthase (ACS) that converts acetate and CoA to acetyl-CoA is over-expressed in the host cells. Cultures of engineered host cells that include overexpressed nucleic acid sequence encoding ACS can optionally include acetate in the culture medium. Examples of acetyl-CoA synthase that can be expressed in a host cell engineered to produce a flavonoid or anthocyanin include, without limitation, the ACS gene of E. coli, the ACS of Salmonella typhimurium, orthologs of these ACSs in other species having at least 50% amino acid identity to these ACSs.

Also considered, in further embodiments, is an engineered host cell that overexpresses a gene encoding pyruvate dehydrogenase (PDH), which converts pyruvate to acetyl-CoA. Further, in E. coli, a variant of the Lpd subunit of PDH can be expressed that includes a mutation (E354K) that reduces inhibition of PDH by NADH.

Alternatively, or in addition to strategies for increasing ACC activity and strategies for increasing acetyl-CoA, strategies for increasing malonyl-CoA by mechanisms that do not rely on the activity of an ACC can be employed. In some embodiments, a cell engineered to produce a flavonoid, or an anthocyanin, is further engineered to increase the cell's supply of malonyl-CoA includes an exogenous nucleic acid sequence encoding a malonyl-CoA synthetase that generates malonyl-CoA from malonate. Examples of malonyl-CoA synthetases include the malonyl-CoA synthetases of Streptomyces coelicolor, Rhodopseudomonas palustris, or a malonyl-CoA synthetase having at least 50% identity to any of these or other naturally occurring malonyl-CoA synthetases. Malonate can optionally be added to the culture medium of a culture that includes a cell engineered to express a malonyl-CoA synthetase. An engineered cell that includes an exogenous gene encoding a malonyl-CoA synthetase can also include an exogenous nucleic acid sequence encoding a malonate transporter, such as a malonate transporter encoded by a matC gene, for example, of Streptomyces coelicolor, or a malonate transporter encoded by DctPQM of Sinorhizobium medicae.

In additional embodiments, a cell engineered to produce a flavonoid or an anthocyanin is further engineered to include an exogenous nucleic acid sequence encoding malonate CoA-transferase that makes malonyl-CoA by direct transfer of the CoA from acetyl-CoA. Examples of malonate CoA-transferase that can be expressed in an engineered cell as provided herein include, without limitation, the alpha subunit (mdcA) of malonate decarboxylase from Acinetobacter calcoaceticus, Geobacillus sp, or a transferase having at least 50% identity to any of these or other naturally occurring malonate CoA-transferases.

In some embodiments, a cell engineered to produce flavonoids or anthocyanins is further engineered to increase the supply of coenzyme A (CoA) to increase its availability for producing acetyl-CoA, malonyl-CoA, and/or p-coumaroyl-CoA. Strategies for increasing CoA supply include upregulating endogenous pantothenate kinase (PanK) (EC:2.7.1.33) that produces CoA from pantothenate. Alternatively, or in addition, a host cell can be engineered to include a nucleic acid sequence encoding type III pantothenate kinase that is not feedback inhibited by coenzyme A, such as CoaX gene of Pseudomonas aeruginosa (EC:2.7.1.33). Cultures of cells engineered for the production of flavonoids or anthocyanins can in some embodiments include a medium that includes pantothenate, a precursor of CoA biosynthesis, and can optionally also include cysteine, used in the CoA biosynthesis.

Additional strategies to increase malonyl-CoA flux to the flavonoid pathway include mutation or downregulation of one or more genes that function in fatty acid biosynthesis. Without limiting the embodiments to any particular mechanism, limiting fatty acid biosynthesis can increase the malonyl-CoA supply available for flavonoid biosynthesis. In some embodiments, the gene beta-ketoacyl-ACP synthase II (E. coli fabF) can be disrupted to reduce fatty acid biosynthesis. Another example of a fatty acid biosynthesis gene of a host cell that may be mutated or downregulated is a gene encoding malonyl-CoA-ACP transacylase (E. coli fabD). Other fatty acid biosynthesis genes of the engineered host cell that can be downregulated include a beta-ketoacyl-ACP synthase I enzyme (E. coli fabB) and acyl carrier protein (E. coli acpP).

Additional genetic modifications that may be present in a host cell engineered to produce flavonoids or anthocyanins include downregulation, disruption, or deletion of genes encoding alcohol dehydrogenase, lactate dehydrogenase, pyruvate oxidase, acetyl phosphate transferase and acetate kinase. In an E. coli host cell, genes that are downregulated, disrupted, or deleted can include aldehyde-alcohol dehydrogenase (adhE), lactate dehydrogenase (ldhA), pyruvate oxidase (poxB), and enzyme acetate kinase phosphate acetyltransferase (ackA-pta).

Further, a cell engineered for the production of flavonoids or anthocyanins can have one or more genes encoding thioesterases downregulated, disrupted, or deleted to prevent hydrolysis of precursors malonyl-CoA, actetyl-CoA, and/or p-coumaryol-CoA. For example, in an E. coli host one or more of the thioesterase genes tesA, tesB, yciA, and ybgC can be downregulated, disrupted, or deleted.

Alternatively, or in addition, genes encoding enzymes of the tricarboxylic acid cycle (TCA), such as succinate dehydrogenase, can be disrupted or downregulated to increase alpha-ketoglutarate supply which serves as a cofactor for one or more of the flavonoid and anthocyanin pathway enzymes. Other TCA enzymes that can be downregulated include citrate synthase that converts acetyl-CoA to citrate.

Also considered, in further embodiments, is an engineered host cell for the production of flavonoids or anthocyanins to upregulate the endogenous biosynthesis of amino acid tyrosine. Tyrosine is one of the precursors for the flavonoid biosynthesis and its conversion to coumaric acid is the first committed step of the pathway. L-tyrosine is one of the three aromatic amino acids derived from the shikimate pathway. The initial step of the shikimate pathway is catalyzed by DAHP synthase isozymes and regulated through feedback-inhibition. Strategies to increase tyrosine production can include, without limitation, transcriptional deregulation, removing feedback inhibition, overexpression of rate-limiting enzymes, and/or deletion of the L-phenylalanine branch of the aromatic acid biosynthetic pathway. For example, in an E. coli host the tyrR gene can be disrupted, feedback-inhibition-resistant versions of the DAHP synthase (aroG) and chorismate mutase (tyrA) can be introduced, and/or rate-limiting enzymes, shikimate kinase (aroK or aroL) and quinate (QUIN)/shikimate dehydrogenase (ydiB) can be overexpressed. Further, the Phosphoenolpyruvate synthase (ppsA) and transketolase (tktA) can be exogenously introduced to enhance tyrosine production.

Also considered, in further embodiments, is an engineered host cell for the production of flavonoids or anthocyanins further engineered to upregulate the endogenous biosynthesis of cofactor heme. Cytochrome P450 (CYPs), one of the exogenous genes in the engineered cells provided herein, contain heme as a cofactor. Improving heme supply can be an effective strategy to increase flavonoid biosynthesis. 5-aminolevulinic acid (ALA) is the first committed precursor to the heme pathway. Strategies to increase heme supply include overexpression of the genes that synthesize the precursor ALA. In an E. coli host, ALA is formed from the 5-carbon skeleton of glutamate (the C5 pathway). The three enzymes involved in ALA biosynthesis are glutamyl-tRNA synthetase (gltX), glutamyl-tRNA reductase (hemA), and glutamate-1-semialdehyde aminotransferase (hemL). In an E. coli host, the engineered cells provided herein can be further engineered to express or overexpress hemA or its variants, and/or hemL to increase the heme precursor ALA production. The nonlimiting examples of hemA gene that can be overexpressed include a mutated hemA (inserting two lysine residuals between Thr-2 and Leu-3 at N terminus of hemA gene from Salmonella typhimurium (EC:1.1.1.70). Alternatively, or in addition, a heterologous ALAS gene can be introduced to produce ALA via the C4 pathway (ALS is synthesized by the condensation of glycine and succinyl-CoA). Nonlimiting examples of heterologous ALAS that can be expressed in E. coli include ALAS of Bradyrhizobium japonicum (EC: 2.3.1.37), ALAS of Rhodobacter capsulatus, or an ALAS having at least 50% sequence identity to a naturally occurring ALAS. Further, one or more of the downstream genes (e.g., in E. coli hemB, hemC, hemD, hemE, hemF, hemG, hemI, or hemH) that catalyze the synthesis of heme from ALA can be overexpressed to drive the flux from ALA to heme production. Cultures of cells engineered for the production of flavonoids or anthocyanins can in some embodiments include a medium that includes succinate and/or glycine, precursors of heme biosynthesis via the C4 pathway.

In another aspect, provided herein are cell cultures that include engineered cells as provided herein in a culture medium, where the culture medium includes a carbon source that is also an energy source for the cells, where the carbon source can be, for example, glycerol, a sugar, or an organic acid, as nonlimiting examples. The culture medium can further include a feed molecule that is used to produce flavonoids or anthocyanins. A feed molecule can be, for example, acetate, malonate, tyrosine, pantothenate, coumarate, biotin, alpha-ketoglutarate, ascorbate, 5-aminolevulinic acid, succinate, or glycine. In some embodiments, the culture comprises a culture medium that includes a carbon source and at least one supplement that is a cofactor of an enzyme or is a precursor of an enzyme cofactor.

In yet another aspect, methods for producing flavonoids and anthocyanins that include incubating a culture of engineered host cell as provided herein to produce flavonoids or anthocyanins. The methods can further include recovering at least one of the flavonoids from the cells, the culture medium, or the whole culture.

In yet another aspect, the invention provides an engineered host cell that comprises one or more genetic modifications resulting in production of flavonoid or anthocyanin from a carbon source that can also be an energy source, through multiple chemical intermediates, by the engineered host cell. In certain embodiments, the production of flavonoid or anthocyanin from glycerol occurs through enzymatic transformation. In certain embodiments, the production of flavonoid or anthocyanin from a carbon source that can also be an energy source occurs through enzymatic transformation. In certain embodiments, the carbon source is selected from a group consisting of: (i) glycerol, (ii) a sugar, (iii) an organic acid, (iv) an amino acid, (v) a biomass comprising glycerol; and (vi) any combination thereof. In certain embodiments, the engineered host cell is cultured in a medium comprising molecules selected from a group consisting of: (i) glycerol, (ii) a sugar, (iii) an organic acid, (iv) an amino acid, (v) a biomass comprising glycerol; and (vi) any combination thereof. In certain embodiments, one or more genetic modifications lead to increase metabolic flux to flavonoid precursors or cofactors. In certain embodiments, one or more genetic modifications cause reduction of formation of byproducts. In certain embodiments, one or more genetic modifications are selected from: (i) one or more modifications for over-expressing one or more endogenous genes in the engineered host cells; (ii) one or more modifications for under-expressing one or more endogenous genes in the engineered host cells; (iii) one or more genetic modification is expressing one or more non-native genes in the engineered host cells; and (iv) a combination thereof. In certain embodiments, the engineered host cell is cultured in a medium comprising molecules selected from: tyrosine, phenylalanine, malonate, p-coumarate, bicarbonate, acetate, pantothenate, biotin, thiamine, alpha-ketoglutarate, ascorbate, and 5-aminolevulinic acid, wherein one or more of the selected molecules are the chemical intermediates, including molecules in the biosynthesis pathway or cofactors. In certain embodiments, the engineered host cell comprises at least one or more nucleic acid sequences selected from: (i) nucleic acid sequences encoding tyrosine ammonia lyase activity; (ii) nucleic acid sequences encoding phenylalanine ammonia lyase activity; (iii) nucleic acid sequences encoding cinnamate 4-hydroxylase activity; (iv) nucleic acid sequences encoding 4-courmarate-CoA ligase (4CL) activity; and (v) any combination thereof. In certain embodiments, the engineered host cell comprises at least one or more peptides selected from: (i) chalcone isomerase; (ii) chalcone synthase; (iii) a fusion protein comprises a chalcone synthase and a chalcone isomerase; and (iv) any combination thereof. In certain embodiments, the engineered cell is E. coli. In certain embodiments, one or more genetic modifications decreases fatty acid biosynthesis. In certain embodiments, the engineered host cell comprises an exogenous nucleic acid sequence selected from: (i) nucleic acid sequence encoding tyrosine ammonia lyase, wherein the encoded tyrosine ammonia lyase forms 4-coumaric acid using tyrosine as a substrate; (ii) nucleic acid sequence encoding phenylalanine ammonia lyase, wherein the encoded phenylalanine ammonia lyase converts phenylalanine to trans-cinnamic acid; (iii) nucleic acid sequence encoding cinnamate-4-hydroxylase, wherein the cinnamate-4-hydroxylase produces 4-coumaric acid from trans-cinnamic acid; (iv) nucleic acid sequence encoding flavanone-3-hydroxylase, wherein flavanone-3-hydroxylase forms dihydrokaempferol from naringenin; and (v) any combinations thereof. In certain embodiments, the engineered host cell comprises an exogenous nucleic acid sequence selected from the group consisting of: (i) nucleic acid sequences encoding tyrosine ammonia lyase, wherein the encoded tyrosine ammonia lyase forms 4-coumaric acid using tyrosine as a substrate; (ii) nucleic acid sequence encoding phenylalanine ammonia lyase, wherein the encoded phenylalanine ammonia lyase converts phenylalanine to trans-cinnamic acid; (iii) nucleic acid sequence encoding cinnamate-4-hydroxylase, wherein the cinnamate-4-hydroxylase produces 4-coumaric acid from trans-cinnamic acid; (iv) nucleic acid sequence encoding 4-courmarate-CoA ligase activity, wherein 4-courmarate-CoA ligase forms p-coumaroyl-CoA from coumaric acid (v) nucleic acid sequence encoding chalcone synthase activity, wherein chalcone synthase forms naringenin chalcone from malonyl-CoA and p-coumaroyl-CoA; (vi) nucleic acid sequence encoding chalcone isomerase activity, wherein chalcone isomerase forms naringenin from naringenin chalcone; (vii) nucleic acid sequence encoding flavanone-3-hydroxylase, wherein flavanone-3-hydroxylase forms dihydrokaempferol from naringenin; and (viii) any combinations thereof. In certain embodiments, the flavonoid is catechin.

In yet another aspect, the invention provides a method of increasing the production of flavonoids or anthocyanins, the method comprising: providing an engineered host cell that comprises one or more genetic modifications resulting in production of flavonoid or anthocyanin from a carbon source that can also be an energy source, through multiple chemical intermediates, by the engineered host cell. In certain embodiments, the production of flavonoid or anthocyanin from a carbon source that can also be an energy source occurs through enzymatic transformation. In certain embodiments, the carbon source is selected from a group consisting of: (i) glycerol, (ii) a sugar, (iii) an organic acid, (iv) an amino acid, (v) a biomass comprising glycerol; and (vi) any combination thereof. In certain embodiments, the engineered host cell is cultured in a medium comprising molecules selected from a group consisting of: (i) glycerol, (ii) a sugar, (iii) an organic acid, (iv) an amino acid, (v) a biomass comprising glycerol; and (vi) any combination thereof. In certain embodiments, one or more genetic modifications lead to increase metabolic flux to flavonoid precursors or cofactors. In certain embodiments, one or more genetic modifications cause increased metabolic flux to flavonoid precursors. In certain embodiments, one or more genetic modifications cause reduction in the formation of byproducts. In certain embodiments, one or more genetic modifications are selected from: (i) one or more modifications for over-expressing one or more endogenous genes in the engineered host cells; (ii) one or more modifications for under-expressing one or more endogenous genes in the engineered host cells; (iii) one or more genetic modification is expressing one or more non-native genes in the engineered host cells; and (iv) a combination thereof. In certain embodiments, the engineered host cell is cultured in a medium comprising molecules selected from: tyrosine, phenylalanine, malonate, p-coumarate, bicarbonate, acetate, pantothenate, biotin, thiamine, alpha-ketoglutarate, ascorbate, and 5-aminolevulinic acid, wherein one or more of the selected molecules are the chemical intermediates, including molecules in the biosynthesis pathway or cofactors. In certain embodiments, the engineered host cell comprises at least one or more nucleic acid sequences selected from: (i) a nucleic acid sequences encoding tyrosine ammonia lyase activity; (ii) a nucleic acid sequences encoding phenylalanine ammonia lyase activity; (iii) cinnamate 4-hydroxylase; and (iv) any combination thereof. In certain embodiments, the engineered host cell comprises at least one or more peptides selected from: (i) chalcone isomerase; (ii) chalcone synthase; (iii) a fusion protein comprises a chalcone synthase and a chalcone isomerase; and (iv) any combination thereof. In certain embodiments, the engineered cell is E. Coli. In certain embodiments, one or more genetic modifications decreases fatty acid biosynthesis. In certain embodiments, the engineered host cell comprises an exogenous nucleic acid sequence selected from: (i) nucleic acid sequences encoding tyrosine ammonia lyase, wherein the encoded tyrosine ammonia lyase forms 4-coumaric acid using tyrosine as a substrate; (ii) nucleic acid sequence encoding phenylalanine ammonia lyase, wherein the encoded phenylalanine ammonia lyase converts phenylalanine to trans-cinnamic acid; (iii) nucleic acid sequence encoding cinnamate-4-hydroxylase, wherein the cinnamate-4-hydroxylase produces 4-coumaric acid from trans-cinnamic acid; (iv) nucleic acid sequence encoding flavanone-3-hydroxylase, wherein flavanone-3-hydroxylase forms dihydrokaempferol from naringenin; and (v) any combinations thereof. In certain embodiments, the engineered host cell comprises an exogenous nucleic acid sequence selected from the group consisting of: (i) nucleic acid sequence encoding tyrosine ammonia lyase, wherein the encoded tyrosine ammonia lyase forms 4-coumaric acid using tyrosine as a substrate; (ii) nucleic acid sequence encoding phenylalanine ammonia lyase, wherein the encoded phenylalanine ammonia lyase converts phenylalanine to trans-cinnamic acid; (iii) nucleic acid sequence encoding cinnamate-4-hydroxylase, wherein the cinnamate-4-hydroxylase produces 4-coumaric acid from trans-cinnamic acid; (iv) nucleic acid sequence encoding 4-courmarate-CoA ligase activity, wherein 4-courmarate-CoA ligase forms p-coumaroyl-CoA from coumaric acid (v) nucleic acid sequence encoding chalcone synthase activity, wherein chalcone synthase forms naringenin chalcone from malonyl-CoA and p-coumaroyl-CoA; (vi) nucleic acid sequence encoding chalcone isomerase activity, wherein chalcone isomerase forms naringenin from naringenin chalcone; (vii) nucleic acid sequence encoding flavanone-3-hydroxylase, wherein flavanone-3-hydroxylase forms dihydrokaempferol from naringenin; and (viii) any combinations thereof. In certain embodiments, the flavonoid is catechin.

In yet another aspect, the invention provides a plurality of engineered host cells, wherein each of the plurality of the engineered host cells comprises one or more genetic modifications resulting in production of flavonoid or anthocyanin from a carbon source that can also be an energy source, through multiple chemical intermediates. In certain embodiments, the production of flavonoid or anthocyanin from a carbon source that can also be an energy source occurs through enzymatic transformation. In certain embodiments, the carbon source is selected from a group consisting of: (i) glycerol, (ii) a sugar, (iii) an organic acid, (iv) an amino acid, (v) a biomass comprising glycerol; and (vi) any combination thereof. In certain embodiments, the engineered host cell is cultured in a medium comprising molecules selected from a group consisting of: (i) glycerol, (ii) a sugar, (iii) an organic acid, (iv) an amino acid, (v) a biomass comprising glycerol; and (vi) any combination thereof. In certain embodiments, one or more genetic modifications lead to increase metabolic flux to flavonoid precursors or cofactors. In certain embodiments, one or more genetic modifications lead to increase metabolic flux to flavonoid precursors or cofactors. In certain embodiments, one or more genetic modifications cause reduction of formation of byproducts. In certain embodiments, one or more genetic modifications are selected from: (i) one or more modifications for over-expressing one or more endogenous genes in the engineered host cells; (ii) one or more modifications for under-expressing one or more endogenous genes in the engineered host cells; (iii) one or more genetic modification is expressing one or more non-native genes in the engineered host cells; and (iv) a combination thereof. In certain embodiments, at least one of the engineered cells from the plurality of the engineered host cells is cultured in a medium comprising molecules selected from: tyrosine, phenylalanine, malonate, p-coumarate, bicarbonate, acetate, pantothenate, biotin, thiamine, alpha-ketoglutarate, ascorbate, and 5-aminolevulinic acid, wherein one or more of the selected molecules are the chemical intermediates, including molecules in biosynthesis pathway or cofactors. In certain embodiments, at least one of the engineered cells from the plurality of the engineered host cells comprise at least one or more nucleic acid sequences selected from: (i) nucleic acid sequences encoding tyrosine ammonia lyase activity; (ii) nucleic acid sequences encoding phenylalanine ammonia lyase activity; (iii) nucleic acid sequences encoding cinnamate 4-hydroxylase activity; (iv) nucleic acid sequences encoding 4-courmarate-CoA ligase (4CL) activity; and (v) any combination thereof. In certain embodiments, at least one of the engineered host cell from the plurality of engineered host cells comprise at least one or more peptides selected from: (i) chalcone isomerase; (ii) chalcone synthase; (iii) a fusion protein comprises a chalcone synthase and a chalcone isomerase; and (iv) any combination thereof. In certain embodiments, at least one the engineered host cell is E. coli. In certain embodiments, one or more genetic modifications decreases fatty acid biosynthesis. In certain embodiments, at least one of the engineered host cell from the plurality of the engineered host cells comprises an exogenous nucleic acid sequence selected from: (i) nucleic acid sequence encoding tyrosine ammonia lyase, wherein the encoded tyrosine ammonia lyase forms 4-coumaric acid using tyrosine as a substrate; (ii) nucleic acid sequence encoding phenylalanine ammonia lyase, wherein the encoded phenylalanine ammonia lyase converts phenylalanine to trans-cinnamic acid; (iii) nucleic acid sequence encoding cinnamate-4-hydroxylase, wherein the cinnamate-4-hydroxylase produces 4-coumaric acid from trans-cinnamic acid; (iv) nucleic acid sequence encoding flavanone-3-hydroxylase, wherein flavanone-3-hydroxylase forms dihydrokaempferol from naringenin; and (v) any combinations thereof. In certain embodiments, the engineered host cell comprises an exogenous nucleic acid sequence selected from the group consisting of: (i) nucleic acid sequence encoding tyrosine ammonia lyase, wherein the encoded tyrosine ammonia lyase forms 4-coumaric acid using tyrosine as a substrate; (ii) nucleic acid sequence encoding phenylalanine ammonia lyase, wherein the encoded phenylalanine ammonia lyase converts phenylalanine to trans-cinnamic acid; (iii) nucleic acid sequence encoding cinnamate-4-hydroxylase, wherein the cinnamate-4-hydroxylase produces 4-coumaric acid from trans-cinnamic acid; (iv) nucleic acid sequence encoding 4-courmarate-CoA ligase activity, wherein 4-courmarate-CoA ligase forms p-coumaroyl-CoA from coumaric acid (v) nucleic acid sequence encoding chalcone synthase activity, wherein chalcone synthase forms naringenin chalcone from malonyl-CoA and p-coumaroyl-CoA; (vi) nucleic acid sequence encoding chalcone isomerase activity, wherein chalcone isomerase forms naringenin from naringenin chalcone; (vii) nucleic acid sequence encoding flavanone-3-hydroxylase, wherein flavanone-3-hydroxylase forms dihydrokaempferol from naringenin; and (viii) any combinations thereof. In certain embodiments, the flavonoid is catechin.

In yet another aspect, the invention provides a method of increasing the production of flavonoids or anthocyanins, the method comprising: providing a plurality of engineered host cells, wherein each of the plurality of the engineered host cell comprises one or more genetic modifications resulting production of flavonoid or anthocyanin from a carbon source that can also be an energy source, through multiple chemical intermediates, by the engineered host cell. In certain embodiments, the production of flavonoid or anthocyanin from a carbon source that can also be an energy source occurs through enzymatic transformation. In certain embodiments, the carbon source is selected from a group consisting of: (i) glycerol, (ii) a sugar, (iii) an organic acid, (iv) an amino acid, (v) a biomass comprising glycerol; and (vi) any combination thereof. In certain embodiments, the engineered host cell is cultured in a medium comprising molecules selected from a group consisting of: (i) glycerol, (ii) a sugar, (iii) an organic acid, (iv) an amino acid, (v) a biomass comprising glycerol; and (vi) any combination thereof. In certain embodiments, one or more genetic modifications lead to increase metabolic flux to flavonoid precursors or cofactors. In certain embodiments, one or more genetic modifications lead to increase metabolic flux to flavonoid precursors or cofactors. In certain embodiments, one or more genetic modifications cause reduction of formation of byproducts. In certain embodiments, one or more genetic modifications are selected from: (i) one or more modifications for over-expressing one or more endogenous genes in the engineered host cells; (ii) one or more modifications for under-expressing one or more endogenous genes in the engineered host cells; (iii) one or more genetic modification is expressing one or more non-native genes in the engineered host cells; and (iv) a combination thereof. In certain embodiments, at least one of the engineered cells from the plurality of the engineered host cells is cultured in a medium comprising molecules selected from: tyrosine, phenylalanine, malonate, p-coumarate, bicarbonate, acetate, pantothenate, biotin, thiamine, alpha-ketoglutarate, ascorbate, and 5-aminolevulinic acid, wherein one or more of the selected molecules are the chemical intermediates, including molecules in biosynthesis pathway or cofactors. In certain embodiments, at least one of the engineered cells from the plurality of the engineered host cells comprise at least one or more nucleic acid sequences selected from: (i) nucleic acid sequences encoding tyrosine ammonia lyase activity; (ii) nucleic acid sequences encoding phenylalanine ammonia lyase activity; (iii) nucleic acid sequences encoding cinnamate 4-hydroxylase activity; (iv) nucleic acid sequences encoding 4-courmarate-CoA ligase (4CL) activity; and (v) any combination thereof. In certain embodiments, at least one of the engineered host cell from the plurality of engineered host cells comprise at least one or more peptides selected from: (i) chalcone isomerase; (ii) chalcone synthase; (iii) a fusion protein comprises a chalcone synthase and a chalcone isomerase; and (iv) any combination thereof. In certain embodiments, at least one the engineered host cell is E. coli. In certain embodiments, one or more genetic modifications decreases fatty acid biosynthesis. In certain embodiments, at least one of the engineered host cell from the plurality of the engineered host cells comprises an exogenous nucleic acid sequence selected from: (i) nucleic acid sequence encoding tyrosine ammonia lyase, wherein the encoded tyrosine ammonia lyase forms 4-coumaric acid using tyrosine as a substrate; (ii) nucleic acid sequence encoding phenylalanine ammonia lyase, wherein the encoded phenylalanine ammonia lyase converts phenylalanine to trans-cinnamic acid; (iii) nucleic acid sequence encoding cinnamate-4-hydroxylase, wherein the cinnamate-4-hydroxylase produces 4-coumaric acid from trans-cinnamic acid; (iv) nucleic acid sequence encoding flavanone-3-hydroxylase, wherein flavanone-3-hydroxylase forms dihydrokaempferol from naringenin; and (v) any combinations thereof. In certain embodiments, the engineered host cell comprises an exogenous nucleic acid sequence selected from the group consisting of: (i) nucleic acid sequence encoding tyrosine ammonia lyase, wherein the encoded tyrosine ammonia lyase forms 4-coumaric acid using tyrosine as a substrate; (ii) nucleic acid sequence encoding phenylalanine ammonia lyase, wherein the encoded phenylalanine ammonia lyase converts phenylalanine to trans-cinnamic acid; (iii) nucleic acid sequence encoding cinnamate-4-hydroxylase, wherein the cinnamate-4-hydroxylase produces 4-coumaric acid from trans-cinnamic acid; (iv) nucleic acid sequence encoding 4-courmarate-CoA ligase activity, wherein 4-courmarate-CoA ligase forms p-coumaroyl-CoA from coumaric acid (v) nucleic acid sequence encoding chalcone synthase activity, wherein chalcone synthase forms naringenin chalcone from malonyl-CoA and p-coumaroyl-CoA; (vi) nucleic acid sequence encoding chalcone isomerase activity, wherein chalcone isomerase forms naringenin from naringenin chalcone; (vii) nucleic acid sequence encoding flavanone-3-hydroxylase, wherein flavanone-3-hydroxylase forms dihydrokaempferol from naringenin; and (viii) any combinations thereof. In certain embodiments, the flavonoid is catechin.

In yet another aspect, the engineered host cell comprises one or more genetic modifications to increase the production and/or availability of malonyl-CoA. In certain embodiments, the production and/or availability of malonyl-CoA is increased by transformation of acetyl-CoA to malonyl-CoA. In certain embodiments, the engineered host cell comprises one or more genetic modifications selected from: (i) expression of acetyl-CoA carboxylase (ACC); and (ii) overexpression of acetyl-CoA carboxylase. In another embodiment, the engineered host cell is an E. coli. In certain embodiments, the E. coli cell further comprises genes from fungi. In certain embodiments, the acetyl-CoA carboxylase is from: Mucor circinelloides, Rhodotorula toruloides, Lipomyces starkeyi, and Ustilago maydis, and orthologs of acetyl-CoA carboxylase having at least 50% amino acid identity to the acetyl-CoA carboxylase of these aforementioned species. In certain embodiments, one or more genetic modification is deletion or attenuation of one or more fatty biosynthetic genes resulting in decrease in fatty acid biosynthesis. In certain embodiments, one or more genetic modification is overexpression of acetyl-CoA synthase (ACS). In certain embodiments, the acetyl-CoA synthase is selected from: acetyl-CoA synthase gene of E. coli, acetyl-CoA synthase gene of Salmonella typhimurium, and orthologs of acetyl-CoA synthase gene in any other species having at least 50% amino acid identity to the acetyl-CoA synthase gene of E. coli and Salmonella typhimurium. In certain embodiments, one or more genetic modification is selected from a group consisting of: (i) overexpression a gene encoding pyruvate dehydrogenase (PDH), wherein the PDH may include E354K mutation; (ii) exogenous nucleic acid sequence encoding a malonyl-CoA synthetase; (iii) upregulation of endogenous pantothenate kinase (PanK), wherein PanK is not feedback inhibited by coenzyme A; (iv) exogenous nucleic acid sequence encoding a malonate transporter; and (v) any combinations thereof. In certain embodiments, the malonyl-CoA synthetase is selected from of malonyl-CoA synthetases of Streptomyces coelicolor, Rhodopseudomonas palustris, or a malonyl-CoA synthetase having at least 50% identity to any of these or other naturally occurring malonyl-CoA synthetases. In certain embodiments, one or more genetic modifications to decrease fatty acid biosynthesis is selected from: (i) mutation or downregulation of a gene encoding malonyl-CoA-ACP transacylase (E. coli fabD); (ii) modifications to the gene beta-ketoacyl-ACP synthase II (E. coli fabF); (iii) downregulation of beta-ketoacyl-ACP synthase I enzyme (E. coli fabB); (iv) downregulation of acyl carrier protein (E. coli acpP); and (v) any combinations thereof. In certain embodiments, the engineered host cell comprises peptides selected from: (i) acetyl-CoA carboxylase (ACC) having an amino acid sequence at least 80% identical to the polypeptide set forth in SEQ ID NO: 15 or SEQ ID NO: 16; (ii) malonate CoA-transferase having an amino acid sequence at least 80% identical to the polypeptide set forth in SEQ ID NO: 19; (iii) acetyl-CoA synthase (ACS) having an amino acid sequence at least 80% identical to the polypeptide set forth in SEQ ID NO: 16; (iv) malonyl-CoA synthase having an amino acid sequence at least 80% identical SEQ ID NO: 77, SEQ ID NO: 78, or SEQ ID NO: 79; (v) malonate transporter having an amino acid sequence at least 80% identical to SEQ ID NO: 80, SEQ ID NO: 81, SEQ ID NO: 82, SEQ ID NO: 83, SEQ ID NO: 84, SEQ ID NO: 85, SEQ ID NO: 86, or SEQ ID NO: 87; (vi) pantothenate kinase having an amino acid sequence at least 80% identical to SEQ ID NO: 88, SEQ ID NO: 89, or SEQ ID NO: 90; and (vii) any combinations thereof.

In another aspect, the invention provides a method of increasing the production of flavonoids comprising an engineered host cell, wherein the one or more engineered host cells comprise one or more genetic modifications to increase the production and/or availability of malonyl-CoA. In certain embodiments, the production and/or availability of malonyl-CoA is increased by transformation of acetyl-CoA to malonyl-CoA. In certain embodiments, the engineered host cell comprises one or more genetic modifications selected from: (i) expression of acetyl-CoA carboxylase (ACC); and (ii) overexpression of acetyl-CoA carboxylase. In another embodiment, the engineered host cell is an E. coli. In certain embodiments, the E. coli cell further comprises genes from fungi. In certain embodiments, the acetyl-CoA carboxylase is from: Mucor circinelloides, Rhodotorula toruloides, Lipomyces starkeyi, and Ustilago maydis, and orthologs of acetyl-CoA carboxylase having at least 50% amino acid identity to the acetyl-CoA carboxylase of these aforementioned species. In certain embodiments, one or more genetic modification is deletion or attenuation of one or more fatty biosynthetic genes resulting in decrease in fatty acid biosynthesis. In certain embodiments, one or more genetic modification is overexpression of acetyl-CoA synthase (ACS). In certain embodiments, the acetyl-CoA synthase is selected from: acetyl-CoA synthase gene of E. coli, acetyl-CoA synthase gene of Salmonella typhimurium, and orthologs of acetyl-CoA synthase gene in any other species having at least 50% amino acid identity to the acetyl-CoA synthase gene of E. coli and Salmonella typhimurium. In certain embodiments, one or more genetic modification is selected from a group consisting of: (i) overexpression a gene encoding pyruvate dehydrogenase (PDH), wherein the PDH may include E354K mutation; (ii) exogenous nucleic acid sequence encoding a malonyl-CoA synthetase; (iii) upregulation of endogenous pantothenate kinase (PanK), wherein PanK is not feedback inhibited by coenzyme A; (iv) exogenous nucleic acid sequence encoding a malonate transporter; and (v) any combinations thereof. In certain embodiments, the malonyl-CoA synthetase is selected from of malonyl-CoA synthetases of Streptomyces coelicolor, Rhodopseudomonas palustris, or a malonyl-CoA synthetase having at least 50% identity to any of these or other naturally occurring malonyl-CoA synthetases. In certain embodiments, one or more genetic modifications to decrease fatty acid biosynthesis is selected from: (i) mutation or downregulation of a gene encoding malonyl-CoA-ACP transacylase (E. coli fabD); (ii) modifications to the gene beta-ketoacyl-ACP synthase II (E. coli fabF); (iii) downregulation of beta-ketoacyl-ACP synthase I enzyme (E. coli fabB); (iv) downregulation of acyl carrier protein (E. coli acpP); and (v) any combinations thereof. In certain embodiments, the engineered host cell comprises peptides selected from: (i) acetyl-CoA carboxylase (ACC) having an amino acid sequence at least 80% identical to the polypeptide set forth in SEQ ID NO: 15 or SEQ ID NO: 16; (ii) malonate CoA-transferase having an amino acid sequence at least 80% identical to the polypeptide set forth in SEQ ID NO: 19; (iii) acetyl-CoA synthase (ACS) having an amino acid sequence at least 80% identical to the polypeptide set forth in SEQ ID NO: 16; (iv) malonyl-CoA synthase having an amino acid sequence at least 80% identical SEQ ID NO: 77, SEQ ID NO: 78, or SEQ ID NO: 79; (v) malonate transporter having an amino acid sequence at least 80% identical to SEQ ID NO: 80, SEQ ID NO: 81, SEQ ID NO: 82, SEQ ID NO: 83, SEQ ID NO: 84, SEQ ID NO: 85, SEQ ID NO: 86, or SEQ ID NO: 87; (vi) pantothenate kinase having an amino acid sequence at least 80% identical to SEQ ID NO: 88, SEQ ID NO: 89, or SEQ ID NO: 90; and (vii) any combinations thereof.

In another aspect, the invention provides an engineered host cell, wherein the engineered host cell comprises one or more genetic modifications to increase endogenous biosynthesis of tyrosine. In certain embodiments, one or more genetic modifications comprises upregulation of 3-deoxy-D-arabino-heptulosonate synthase. In certain embodiments, one or more genetic modifications are selected from: (i) upregulation of chorismate mutase; (ii) upregulation of prephenate dehydrogenase; (iii) overexpression of shikimate kinase; (iv) overexpression of shikimate dehydrogenase; and (v) any combinations thereof. In certain embodiments, one or more genetic modifications comprises downregulation of L-phenylalanine biosynthetic pathway. In certain embodiments, one or more genetic modifications comprises expression of exogenous phosphoenolpyruvate synthase (ppsA). In certain embodiments, one or more genetic modifications comprises expression of exogenous transketolase (tktA). In certain embodiments, wherein the one or more genetic modifications comprises disruption of tyrR gene. In certain embodiments, one or more genetic modifications are selected from a group consisting of: (i) expression or overexpression of (D146N) variant of phospho-2-dehydro-3-deoxyheptonate aldolase; (ii) expression or overexpression of variant of 3-dehydroquinate synthase (aroB); (iii) overexpression of transketolase tktA; (iv) deletion of shikimate kinase (aroK); (v) deletion of tyrR; (vi) expression or overexpression of A354V variant of chorismate mutase (tyrA); (vi) and any combination thereof.

In another aspect, the invention provides a method of increasing endogenous biosynthesis of tyrosine comprising an engineered cell, wherein the engineered host cell comprises one or more genetic modifications to increase endogenous biosynthesis of tyrosine. In certain embodiments, one or more genetic modifications comprises upregulation of 3-deoxy-D-arabino-heptulosonate synthase. In certain embodiments, one or more genetic modifications are selected from: (i) upregulation of chorismate mutase; (ii) upregulation of prephenate dehydrogenase; (iii) overexpression of shikimate kinase; (iv) overexpression of shikimate dehydrogenase; and (v) any combinations thereof. In certain embodiments, one or more genetic modifications comprises downregulation of L-phenylalanine biosynthetic pathway. In certain embodiments, one or more genetic modifications comprises expression of exogenous phosphoenolpyruvate synthase (ppsA). In certain embodiments, one or more genetic modifications comprises expression of exogenous transketolase (tktA). In certain embodiments, wherein the one or more genetic modifications comprises disruption of tyrR gene. In certain embodiments, one or more genetic modifications are selected from a group consisting of: (i) expression or overexpression of (D146N) variant of phospho-2-dehydro-3-deoxyheptonate aldolase; (ii) expression or overexpression of variant of 3-dehydroquinate synthase (aroB); (iii) overexpression of transketolase tktA; (iv) deletion of shikimate kinase (aroK); (v) deletion of tyrR; (vi) expression or overexpression of A354V variant of chorismate mutase (tyrA); (vi) and any combination thereof.

In another aspect, the invention provides an engineered host cell, wherein the engineered host cell comprises one or more genetic modifications to increase transformation of leucocyanidin or catechin to cyanidin-3-glucoside (Cy3G). In certain embodiments, one or more genetic modifications comprises overexpression of anthocyanin synthase. In certain embodiments, the anthocyanin synthase is selected from: (i) anthocyanin synthase of Carica papaya (SEQ. TD NO:13); (ii) the anthocyanin synthase has an amino acid sequence at least 80% identical to SEQ. ID NO: 66, SEQ. ID NO: 67, SEQ. ID NO: 68, or SEQ. ID NO: 69; (iii) the anthocyanin synthase has an amino acid sequence at least 80% identical to SEQ. ID NO: 13; and (iv) any combinations thereof. In certain embodiments, one or more engineered host cells comprises flavonoid-3-glucosyl transferase (3GT). In certain embodiments, flavonoid-3-glucosyl transferase is selected from: (i) flavonoid-3-glucosyl transferase in Vitis labrusca (SEQ. ID NO:14); (ii) the flavonoid-3-glucosyl transferase has an amino acid sequence at least 80% identical to SEQ. ID NO: 70, SEQ. ID NO: 71, SEQ. ID NO: 72, or SEQ. ID NO: 73; and (iii) any combinations thereof. In certain embodiments, one or more genetic modifications comprises overexpression of anthocyanin synthase and flavonoid-3-glucosyl transferase (3GT). In certain embodiments, the one or more genetic modifications comprises overexpression of anthocyanin synthase and flavonoid-3-glucosyl transferase (3GT). In certain embodiments, the one or more genetic modifications are selected from a group consisting of: (i) anthocyanin synthase, (ii) flavonoid-3-glucosyl transferase (3GT), and (iii) a combination thereof.

In another aspect, the invention provides a method for increasing the production of flavonoids comprising an engineered host cell, wherein the engineered host cell comprises one or more genetic modifications to increase transformation of leucocyanidin or catechin to cyanidin-3-glucoside (Cy3G). In certain embodiments, one or more genetic modifications comprises overexpression of anthocyanin synthase. In certain embodiments, the anthocyanin synthase is selected from: (i) anthocyanin synthase of Carica papaya (SEQ. ID NO:13); (ii) the anthocyanin synthase has an amino acid sequence at least 80% identical to SEQ. ID NO: 66, SEQ. ID NO: 67, SEQ. ID NO: 68, or SEQ. ID NO: 69; (iii) the anthocyanin synthase has an amino acid sequence at least 80% identical to SEQ. ID NO: 13; and (iv) any combinations thereof. In certain embodiments, one or more engineered host cells comprises flavonoid-3-glucosyl transferase (3GT). In certain embodiments, flavonoid-3-glucosyl transferase is selected from: (i) flavonoid-3-glucosyl transferase in Vitis labrusca (SEQ. ID NO:14); (ii) the flavonoid-3-glucosyl transferase has an amino acid sequence at least 80% identical to SEQ. ID NO: 70, SEQ. ID NO: 71, SEQ. ID NO: 72, or SEQ. ID NO: 73; and (iii) any combinations thereof. In certain embodiments, one or more genetic modifications comprises overexpression of anthocyanin synthase and flavonoid-3-glucosyl transferase (3GT). In certain embodiments, the one or more genetic modifications comprises overexpression of anthocyanin synthase and flavonoid-3-glucosyl transferase (3GT). In certain embodiments, the one or more genetic modifications are selected from a group consisting of: (i) anthocyanin synthase, (ii) flavonoid-3-glucosyl transferase (3GT), and (iii) a combination thereof.

In another aspect, the invention provides a method of increasing the transformation of leucocyanidin or catechin to cyanidin-3-glucoside (Cy3G), delphinidin or gallocatechin to delphindin-3-glucoside (De3G), or afzelechin or pelargonidin to pelargonidin-3-glucoside (Pe3G) comprising anthocyanin synthase, wherein the anthocyanin synthase is selected from: (i) anthocyanin synthase of Carica papaya (SEQ. ID NO:13); (ii) the anthocyanin synthase has an amino acid sequence at least 80% identical to SEQ. ID NO: 66, SEQ. ID NO: 67, SEQ. ID NO: 68, or SEQ. ID NO: 69; (iii) the anthocyanin synthase has an amino acid sequence at least 80% identical to SEQ. ID NO: 13; and (iv) any combinations thereof. In certain embodiments, one or more genetic modifications comprises overexpression of anthocyanin synthase and flavonoid-3-glucosyl transferase (3GT). In certain embodiments, the one or more genetic modifications are selected from a group consisting of: (i) anthocyanin synthase, (ii) flavonoid-3-glucosyl transferase (3GT), and (iii) a combination thereof.

In another aspect, the invention provides a method of increasing the transformation of cyanidin to cyanidin-3-glucoside (Cy3G), delphindin to delphindin-3-glucoside (De3G), or pelargonidin to pelagonidin-3-glucoside (Pe3G), comprising flavonoid-3-glucosyl transferase (3GT), wherein the flavonoid-3-glucosyl transferase is selected from: (i) flavonoid-3-glucosyl transferase in Vitis labrusca (SEQ. ID NO: 14); (ii) the flavonoid-3-glucosyl transferase has an amino acid sequence at least 80% identical to SEQ. ID NO: 70, SEQ. ID NO: 71, SEQ. ID NO: 72, or SEQ. ID NO: 73; and (iii) any combinations thereof.

In another aspect, the invention provides an engineered host cell comprises one or more genetic modifications to increase the production of dihydroquercetin (DHQ), dihydromyricein (DHM), eriodictoyl (EDL), and/or pentahydroxyflayaone (PHF), wherein the engineered host cell comprises cytochrome P450 reductase (CPR) and at least one of flavanone-3-hydroxylase (F3H), flavanone-3′-hydroxylase (F3′H), or flavonoid 3′,5′-hydroxylase (F3′5′H). In certain embodiments, the precursor for increase in production of dihydroquercetin (DHQ), dihydromyricein (DHM), eriodictoyl (EDL), and/or pentahydroxyflayanone (PHF) is naringenin and/or dihydrokaempferol (DHK). In certain embodiments, the engineered host cell further comprises peptides selected from a group consisting of: (i) flavonoid 3′-hydroxylase (F3′H); (ii) cytochrome P450 reductase (CPR); and (iii) any combination thereof. In certain embodiments, the engineered host cell produces eriodictyol or taxifolin. In certain embodiments, the engineered host cell further comprises flavonoid 3′,5′-hydroxylase (F3′5′H). In certain embodiments, the engineered host cell produces pentahydroxyflavone or dihydromyricetin. In certain embodiments, flavonoid 3′-hydroxylase (F3′H) is truncated to remove the N-terminal leader sequence. In certain embodiments, cytochrome P450 reductase (CPR) is truncated to remove the N-terminal leader sequence. In certain embodiments, flavonoid 3′-hydroxylase (F3′H) is fused with cytochrome P450 reductase (CPR). In certain embodiments, flavonoid 3′,5′-hydroxylase (F3′5′H) is fused with cytochrome P450 reductase (CPR). In certain embodiments, flavanone-3-hydroxylase (F3H) has an amino acid sequence at least 80% identical to the polypeptide set forth in SEQ ID NO. 7. In certain embodiments, flavanone-3′-hydroxylase (F3′H) has an amino acid sequence at least 80% identical to the polypeptide set forth in SEQ ID NO. 8. In certain embodiments, cytochrome P450 reductase (CPR) has an amino acid sequence at least 80% identical to the polypeptide set forth in SEQ ID NO. 9. In certain embodiments, flavonoid 3′,5′-hydroxylase (F3′5′H) has an amino acid sequence at least 80% identical to the polypeptides selected from a group consisting of: (i) SEQ ID NO. 10, (ii) SEQ ID NO. 56, and (iii) SEQ ID NO. 57. In certain embodiments, the engineered host cell further comprises cytochrome b₅. In certain embodiments, cytochrome b₅ has an amino acid sequence at least 80% identical to the polypeptide set forth in SEQ ID NO. 98. In certain embodiments, wherein the flavanone-3-hydroxylase (F3H) has an amino acid sequence at least 80% identical to the polypeptides selected from a group consisting of: (i) SEQ ID NO. 7, (ii) SEQ ID NO. 45, (iii) SEQ ID NO. 46, (iv) SEQ ID NO. 47, and (v) SEQ ID NO. 48. In another aspect, the invention provides method of increasing the production of dihydroquercetin (DHQ), dihydromyricein (DHM), eriodictoyl (EDL), and/or pentahydroxyflayaone (PHF) comprising an engineered host cell, wherein the engineered host cell comprises cytochrome P450 reductase (CPR) and at least one of flavanone-3-hydroxylase (F3H), flavanone-3′-hydroxylase (F3′H), or flavonoid 3′,5′-hydroxylase (F3′5′H). In certain embodiments, the precursor for increase in production of dihydroquercetin (DHQ), dihydromyricein (DHM), eriodictoyl (EDL), and/or pentahydroxyflayanone (PHF) is naringenin and/or dihydrokaempferol (DHK). In certain embodiments, the engineered host cell further comprises peptides selected from a group consisting of: (i) flavonoid 3′-hydroxylase (F3′H); (ii) cytochrome P450 reductase (CPR); and (iii) any combination thereof. In certain embodiments, the engineered host cell produces eriodictyol or taxifolin. In certain embodiments, the engineered host cell further comprises flavonoid 3′,5′-hydroxylase (F3′5′H). In certain embodiments, the engineered host cell produces pentahydroxyflavone or dihydromyricetin. In certain embodiments, flavonoid 3′-hydroxylase (F3′H) is truncated to remove the N-terminal leader sequence. In certain embodiments, cytochrome P450 reductase (CPR) is truncated to remove the N-terminal leader sequence. In certain embodiments, flavonoid 3′-hydroxylase (F3′H) is fused with cytochrome P450 reductase (CPR). In certain embodiments, flavonoid 3′,5′-hydroxylase (F3′5′H) is fused with cytochrome P450 reductase (CPR). In certain embodiments, flavanone-3-hydroxylase (F3H) has an amino acid sequence at least 80% identical to the polypeptide set forth in SEQ ID NO. 7. In certain embodiments, flavanone-3′-hydroxylase (F3′H) has an amino acid sequence at least 80% identical to the polypeptide set forth in SEQ ID NO. 8. In certain embodiments, cytochrome P450 reductase (CPR) has an amino acid sequence at least 80% identical to the polypeptide set forth in SEQ ID NO. 9. In certain embodiments, flavonoid 3′,5′-hydroxylase (F3′5′H) has an amino acid sequence at least 80% identical to the polypeptides selected from a group consisting of: (i) SEQ ID NO. 10, (ii) SEQ ID NO. 56, and (iii) SEQ ID NO. 57. In certain embodiments, the engineered host cell further comprises cytochrome bs. In certain embodiments, cytochrome b₅ has an amino acid sequence at least 80% identical to the polypeptide set forth in SEQ ID NO. 98. In certain embodiments, wherein the flavanone-3-hydroxylase (F3H) has an amino acid sequence at least 80% identical to the polypeptides selected from a group consisting of: (i) SEQ ID NO. 7, (ii) SEQ ID NO. 45, (iii) SEQ ID NO. 46, (iv) SEQ ID NO. 47, and (v) SEQ ID NO. 48. An engineered cell for producing flavonoids include an exogenous nucleic acid sequence encoding tyrosine ammonia lyase (TAL) activity (alternatively or in addition, an exogenous nucleic acid encoding phenylalanine ammonia-lyase (PAL) activity and an exogenous nucleic acid encoding cinnamate-4-hydroxylase (C4H) activity), an exogenous nucleic acid sequence encoding 4-coumarate-CoA ligase (4CL) activity, an exogenous nucleic acid sequence encoding chalcone synthase (CHS) activity, and an exogenous nucleic acid sequence encoding chalcone isomerase (CHI) activity. Optionally, the engineered cell can further include an exogenous nucleic acid sequence encoding an exogenous nucleic acid sequence encoding flavanone-3-hydroxylase (F3H) activity, an exogenous nucleic acid sequence encoding flavonoid 3′-hydroxlase (F3′H) activity or flavonoid 3′,5′-hydroxylase (F3′5′H), an exogenous nucleic acid sequence encoding cytochrome P450 reductase (CPR) activity, an exogenous nucleic acid sequence encoding dihydroflavonol-4-reductase (DFR) activity, and/or an exogenous nucleic acid sequence encoding leucoanthocyanidin reductase (LAR) activity.

Tyrosine ammonia-lyase (TAL) can be, for example, a member of the aromatic amino acid deaminase family that catalyzes the elimination of ammonia from L-tyrosine to yield p-coumaric acid. An exemplary tyrosine ammonia lyase is the Saccharothrix espanaensis tyrosine ammonia lyase (TAL; SEQ ID NO: 1). Also considered for use in the engineered cells provided herein are TALs with SEQ ID NOS: 23-26, TALs listed in Table 1, TAL homologs and variants having at least 50%, at least 55%, at least 60%, at least 65%, at least 70%, at least 75%, at least 80%, at least 85%, at least 90%, at least 95%, at least 96%, at least 97%, at least 98%, or at least 99% identity to SEQ ID: 1 that have the activity of a tyrosine ammonia lyase that produces p-coumaric acid from tyrosine.

TABLE 1
Tyrosine ammonia-lyase

| Organism | GenBank Accession Number |
|---|---|
| Rhodotorula glutini | AGZ04575.1 |
| Flavobacterium johnsoniae | WP_012023194.1 |
| Herpetosiphon aurantiacus | ABX02653.1 |
| Rhodobacter capsulatus | ADE83766.1 |
| Saccharothrix espanaensis | AKE50820.1 |
| Trichosporon cutaneum | AKE50834.1 |

Similar to tyrosine ammonia-lyase, phenylalanine ammonia-lyase (PAL) can be a member of the aromatic amino acid deaminase family that catalyzes the non-oxidative deamination of L-phenylalanine to form trans-cinnamic acid. An exemplary phenylalanine ammonia-lyase is the Brevibacillus laterosporus phenylalanine ammonia-lyase (PAL; SEQ ID NO:2). Also considered for use in the engineered cells provided herein are PALs with SEQ ID NOS: 27-29, PAL homologs and variants having at least 50%, at least 55%, at least 60%, at least 65%, at least 70%, at least 75%, at least 80%, at least 85%, at least 90%, at least 95%, at least 96%, at least 97%, at least 98%, or at least 99% identity to SEQ ID NO: 2 that have the activity of a phenylalanine ammonia lyase that produces trans-cinnamic acid from phenylalanine.

Cinnamate-4-hydroxylase (C4H) belongs to the cytochrome P450-dependent monooxygenase family and catalyzes the formation of p-coumaric acid from trans-cinnamic acid. Considered for use in the engineered cells provided herein are C4H of Helianthus annuus L. (C4H; SEQ ID NO: 3), C4Hs with SEQ ID NOS: 30-32, and C4H homologs of other species, as well as variants of naturally occurring C4Hs having at least 50%, at least 55%, at least 60%, at least 65%, at least 70%, at least 75%, at least 80%, at least 85%, at least 90%, at least 95%, at least 96%, at least 97%, at least 98%, or at least 99% amino acid identity to the SEQ ID NO: 3 (C4H, Helianthus annuus L.) that have the activity of a C4H. 4-coumarate-CoA ligase (4CL) catalyzes the activation of 4-coumarate to its CoA ester. Considered for use in the engineered cells provided herein are 4CLs of Petroselinum crispum (SEQ ID NO: 4), 4CLs in Table 2, 4CLs with SEQ ID NOS: 33-36, and 4CL homologs of other species, as well as variants of naturally occurring 4CLs having at least 50%, at least 55%, at least 60%, at least 65%, at least 70%, at least 75%, at least 80%, at least 85%, at least 90%, at least 95%, at least 96%, at least 97%, at least 98%, or at least 99% amino acid identity to SEQ ID No: 4 (4CL, Petroselinum crispum) that have the activity of a 4CL.

TABLE 2
4-coumarate-CoA ligases

| Organism | GenBank Accession Number |
|---|---|
| Petroselinum crispum | CAA31697.1 |
| Camellia sinensis | ASU87409.1 |
| Capsicum annuum | KAF3620173.1 |
| Castanea mollissima | KAF3954751.1 |
| Daucus carota | AIT52344.1 |
| Gynura bicolor | BAJ17664.1 |
| Ipomoea purpurea | AHJ60263.1 |
| Lonicera japonica | AGE10594.1 |
| Lycium chinense | QDL52638.1 |
| Nelumbo nucifera | XP_010265453.1 |
| Nyssa sinensis | KAA8540582.1 |
| Solanum lycopersicum | NP_001333770.1 |
| Striga asiatica | GER48539.1 |

The chalcone synthase (CHS) can be, for example, a type III polyketide synthase that sequentially condenses three molecules of malonyl-CoA with one molecule of p-coumaryol-CoA to produce the naringenin precursor naringenin chalcone or naringenin. An exemplary chalcone synthase is the chalcone synthase of Petunia x hybrida (CHS, SEQ ID NO: 5). Also considered for use in the engineered cells provided herein are the genes listed in Table 3, CHSs with SEQ ID: 37-40, and CHS homologs and variants having at least 50%, at least 55%, at least 60%, at least 65%, at least 70%, at least 75%, at least 80%, at least 85%, at least 90%, at least 95%, at least 96%, at least 97%, at least 98%, or at least 99% amino acid identity to SEQ ID NO: 5 (CHS, Petunia x hybrida) that have the activity of a chalcone synthase.

TABLE 3
Chalcone synthases

| Organism | GenBank Accession Number |
|---|---|
| Petunia hybrida | AAF60297.1 |
| Acer palmatum | AWN08245.1 |
| Callistephus chinensis | CAA91930.1 |
| Camellia japonica | BAI66465.1 |
| Capsicum annuum | XP_016566084.1 |
| Coffea arabica | XP_027118978.1 |
| Curcuma alismatifolia | ADP08987.1 |
| Dendrobium catenatum | ALE71934.1 |
| Garcinia mangostana | ACM62742.1 |
| Iochroma calycinum | AIY22758.1 |
| Iris germanica | BAE53636.1 |
| Lilium speciosum | BAE79201.1 |
| Lonicera caerulea | ALU09326.1 |
| Lycium ruthenicum | ATB56297.1 |
| Magnolia liliiflora | AHJ60259.1 |
| Matthiola incana | BBM96372.1 |
| Morus alba var. multicaulis | AHL83549.1 |
| Nelumbo nucifera | NP_001305084.1 |
| Nyssa sinensis | KAA8548459.1 |
| Paeonia lactiflora | AEK70334.1 |
| Panax notoginseng | QKV26463.1 |
| Ranunculus asiaticus | AYV99476.1 |
| Rosa chinensis | AEC13058.1 |
| Theobroma cacao | XP_007032052.2 |

Chalcone isomerase (CHI, also referred to as chalcone flavanone isomerase) catalyzes the stereospecific and intramolecular isomerization of naringenin chalcone into its corresponding (2S)-flavanones. Considered for use in the engineered cells provided herein are CHI of Medicago sativa (SEQ ID NO: 6), CHI of Table 4, CHIs with SEQ ID NOS: 41-44, and CHI homologs of other species, as well as variants of naturally occurring CHI having at least 50%, at least 55%, at least 60%, at least 65%, at least 70%, at least 75%, at least 80%, at least 85%, at least 90%, at least 95%, at least 96%, at least 97%, at least 98%, or at least 99% amino acid identity to SEQ ID NO: 6 (CHI, Medicago sativa) that have the activity of a chalcone isomerase.

TABLE 4
Chalcone Isomerases

| Organism | GenBank Accession Number |
|---|---|
| Medicago sativa | AGZ04578.1 |
| Dendrobium hybrid cultivar | AGY46120.1 |
| Abrus precatorius | XP_027366189.1 |
| Antirrhinum majus | BAO32070.1 |
| Arachis duranensis | XP_015942246.1 |
| Astragalus membranaceus | ATY39974.1 |
| Camellia sinensis | XP_028119616.1 |
| Castanea mollissima | KAF3958409.1 |
| Cephalotus follicularis | GAV77263.1 |
| Clarkia gracilis subsp. | QPF47150.1 |
| sonomensis | |
| Dianthus caryophyllus | CAA91931.1 |
| Glycyrrhiza uralensis | AXO59749.1 |
| Handroanthus impetiginosus | PIN05040.1 |
| Lotus japonicus | CAD69022.1 |
| Morus alba | AFM29131.1 |
| Phaseolus vulgaris | XP_007142690.1 |
| Punica granatum | ANB66204.1 |
| Rhodamnia argentea | XP_030524476.1 |
| Spatholobus suberectus | TKY50621.1 |
| Trifolium subterraneum | GAU12132.1 |

A nucleic acid sequence encoding a CHI can in some embodiments be fused to a nucleic acid sequence encoding a CHS in an engineered cell as provided herein, such that the CHI activity is fused to the chalcone synthase activity, i.e., a fusion protein is produced in the engineered cell that has both condensing and cyclization activities.

Flavanone 3-hydroxylase (F3H) catalyzes the stereospecific hydroxylation of (2S)-naringenin to form (2R,3R)-dihydrokaempferol. Other substrates include (2S)-eriodictyol, (2S)-dihydrotricetin and (2S)-pinocembrin. Some F3H enzymes are bifunctional and also catalyzes as flavonol synthase (EC: 1.14.20.6). Considered for use in the engineered cells provided herein are F3H of Rubus occidentalis (SEQ ID NO: 7), F3Hs with SEQ ID NOS: 45-48, F3Hs listed in Table 5, and other F3H homologs and variants having at least 50%, at least 55%, at least 60%, at least 65%, at least 70%, at least 75%, at least 80%, at least 85%, at least 90%, at least 95%, at least 96%, at least 97%, at least 98%, or at least 99% amino acid identity to SEQ ID NO:7 (F3H, Rubus occidentalis) that have the activity of a F3H.

TABLE 5
Flavanone 3-hydroxylases

| Organism | GenBank Accession Number |
|---|---|
| Rubus occidentalis | ACM17897.1 |
| Abrus precatorius | XP_027347564.1 |
| Nyssa sinensis | KAA8547483.1 |
| Camellia sinensis | AAT68774.1 |
| Morella rubra | KAB1219056.1 |
| Rosa chinensis | PRQ47414.1 |
| Malus domestica | AAD26206.1 |
| Vitis amurensis | ALB75302.1 |
| Iochroma ellipticum | AMQ48669.1 |
| Hibiscus sabdariffa | ALB35017 |
| Cephalotus follicularis | GAV71832 |

Flavonoid 3′-hydroxylases (F3′H) belongs to the cytochrome P450 family with systematic name of flavonoid, NADPH:oxygen oxidoreductase (3′-hydroxylating). In the flavonoid biosynthetic pathway, F3′H converts dihydrokaempferol to dihydroquercetin (taxifolin) or naringenin to eriodictyol. In the cells engineered for enhancing production of quercetin, flavonoid 3′-hydroxylases (F3′H) may be used to convert kaempferol to quercetin. Considered for use in the engineered cells provided herein are F3′H of Brassica napus (F3′H; SEQ ID NO: 8), F3′H with SEQ ID NOS: 49-52, those listed in Table 6, and homologs and variants having at least 50%, at least 55%, at least 60%, at least 65%, at least 70%, at least 75%, at least 80%, at least 85%, at least 90%, at least 95%, at least 96%, at least 97%, at least 98%, or at least 99% amino acid identity to these F3′H. F3′H is a cytochrome P450 enzyme that requires a cytochrome P450 reductase (CPR) to function. Cytochrome P450 reductases are diflavin oxidoreductases that supply electrons to F3′Hs. The P450 reductase can be from the same species as F3′H or different species from F3′H. Considered for use in the engineered cells provided herein are CPR of Catharanthus roseus (SEQ ID NO: 9), additional CPRs listed in Table 7, CPRs with SEQ ID NOS: 53-55, CPR homologs of other species, and variants of naturally occurring CPRs having at least 50%, at least 55%, at least 60%, at least 65%, at least 70%, at least 75%, at least 80%, at least 85%, at least 90%, at least 95%, at least 96%, at least 97%, at least 98%, or at least 99% amino acid identity to these CPRs that have the activity of a CPR. In various embodiments, the N-terminal nucleic acid sequences in the genes of F3′H and/or CPR originated from eukaryotic cells can encode targeting leader peptides, which can be removed before introduction into prokaryotic host cells, if desired. In some embodiments, the hydroxylase complex HpaBC from E. coli was used to hydroxylate naringenin to eriodictyol or dihydrokaempferol to dihydroquercetin (taxifolin).

TABLE 6
Flavonoid 3′-hydroxylases

| Organism | GenBank Accession Number |
|---|---|
| Brassica napus | ABC58722.1 |
| Gerbera hybrid cultivar D1 | ABA64468.1 |
| Cephalotus follicularis | GAV84063.1 |
| Theobroma cacao | XP_007037548.1 |
| Phoenix dactylifera | XP_008791304.2 |

TABLE 7
Cytochrome P450 reductases

| Organism | GenBank Accession Number |
|---|---|
| Catharanthus roseus | CAA49446.1 |
| Brassica napus | XP_013706600.1 |
| Cephalotus follicularis | GAV59576.1 |
| Camellia sinensis | XP_028084858.1 |

A nucleic acid sequence encoding a F3′H can in some embodiments be fused to a nucleic acid sequence encoding a CPR in an engineered cell as provided herein, such that the F3′H activity is fused to the CPR activity.

In the cells engineered to produce dihydromyricetin, flavonoid 3′, 5′-hydroxylase (F3′5′H) can be used to convert dihydrokaempferol to dihydromyricetin or naringenin to pentahydroxyflavone, which is further converted to dihydromyricetin by a F3H. F3′5′H has the systematic name flavanone, NADPH:oxygen oxidoreductase and catalyzes the formation of 3′,5′-dihydroxyflavanone from flavanone.

In cells engineered to enhance production of myricetin, flavonoid 3′, 5′-hydroxylase (F3′5′H) may be used to convert kaempferol and quercetin to myricetin. An exemplary F3′5′H is the Delphinium grandiflorum F3′5′H (SEQ ID NO: 10), Also considered for use in the engineered cells provided herein include F3′5′H with SEQ ID NOS:56-57, F3′5′H homologs of other species, and variants of naturally occurring F3′5′H having at least 50%, at least 55%, at least 60%, at least 65%, at least 70%, at least 75%, at least 80%, at least 85%, at least 90%, at least 95%, at least 96%, at least 97%, at least 98%, or at least 99% amino acid identity to SEQ ID NOS:10 that have the activity of a F3′5′H.

Flavonol synthase (FLS) catalyzes the oxidation of dihydroflavonol to produce flavonol. In cells engineered to enhance production of myricetin, kampferol, and/or quercetin, flavonol synthase (FLS) may be used for catalyzing the conversion of dihydromyricetin (DHM) to myricetin; conversion of dihydrokaempferol (DHK) to kampferol; and conversion of dihydroquercetin (DHQ) to quercetin. An exemplary flavonol synthase (FLS) is FLS from Petroselinum crispum (EC:1.14.11.23). Considered for use in the engineered cells provided herein are FLS of Petroselinum crispum (SEQ ID NO: 99), and other FLS homologs and variants having at least 50%, at least 55%, at least 60%, at least 65%, at least 70%, at least 75%, at least 80%, at least 85%, at least 90%, at least 95%, at least 96%, at least 97%, at least 98%, or at least 99% amino acid identity to SEQ ID NOS: 99-122 that have the activity of FLS.

Dihydroflavonol 4-reductase (DFR) acts on (+)-dihydrokaempferol (DHK), (+)-dihydroquercetin (Taxifolin, DHQ), or dihydromyricein (DHM) to reduce those compounds to the corresponding cis-flavan-3,4-diol (DHK to leucopelargonidin; Taxifolin to leucocyanidin; DHM to leucodelphinidin). An exemplary DFR is the Anthurium andraeanum DFR (SEQ ID NO: 11). Also considered for use in the engineered cells provided herein include DFRs in Table 8, DFRs with SEQ ID NOS: 58-61, and DFR homologs of other species, as well as variants of naturally occurring DFR having at least 50%, at least 55%, at least 60%, at least 65%, at least 70%, at least 75%, at least 80%, at least 85%, at least 90%, at least 95%, at least 96%, at least 97%, at least 98%, or at least 99% amino acid identity to SEQ ID NO: 11.

TABLE 8
Dihydroflavonol 4-reductases

| Organism | GenBank Accession Number |
|---|---|
| Eustoma grandiflorum | BAD34461.1 |
| Anthurium andraeanum | AAP20866.1 |
| Camellia sinensis | AAT66505.1 |
| Morella rubra | KAB1203810.1 |
| Dendrobium moniliforme | AEB96144.1 |
| Fragaria × ananassa | AHL46451.1 |
| Rosa chinensis | XP_024167119.1 |
| Acer palmatum | AWN08247.1 |
| Nyssa sinensis | KAA8531902.1 |
| Vitis amurensis | I82380.1 |
| Abrus precatorius | XP_027329642.1 |
| Angelonia angustifolia | AHM27144.1 |
| Pyrus pyrifolia | Q84KP0.1 |
| Theobroma cacao | XP_017985307 |
| Theobroma cacao | XP_007051597.2 |
| Brassica oleracea var. capitata | QKO29328.1 |
| Rubus idaeus | AXK92786.1 |
| Citrus sinensis | AAY87035.1 |
| Gerbera hybrida | P51105.1 |
| Cephalotus follicularis | GAV76940.1 |
| Ginkgo biloba | AGR34043.1 |
| Dryopteris erythrosora | QFQ61498.1 |
| Dryopteris erythrosora | QFQ61499.1 |
| Cephalotus follicularis | GAV76942.1 |

Leucoanthocyanidin reductase (LAR) catalyzes the synthesis of catechin from 3,4-cis-leucocyanidin. LAR also synthesizes afzelechin and gallocatechin. Considered for use in the engineered cells provided herein are LAR of Desmodium uncinatum (SEQ ID NO: 12), LARs with SEQ ID NOS: 62-65, and LAR homologs of other species, as well as variants of naturally occurring LAR having at least 50%, at least 55%, at least 60%, at least 65%, at least 70%, at least 75%, at least 80%, at least 85%, at least 90%, at least 95%, at least 96%, at least 97%, at least 98%, or at least 99% amino acid identity to SEQ ID NO: 12 (LAR, Desmodium uncinatum) that have the activity of a LAR.

Optionally, the cells are further engineered to include an anthocyanin synthase (ANS) which catalyzes the conversion of leucoanthocyanidin or catechin to anthocyanidin, leucopelargonidin to pelargonidin, or leucodelphinidin to delphinidin. Considered for use in the engineered cells provided herein are ANS of Carica papaya (SEQ ID NO: 13), ANS with SEQ ID NOS: 66-69, and ANS homologs of other species, as well as variants of naturally occurring ANS having at least 50%, at least 55%, at least 60%, at least 65%, at least 70%, at least 75%, at least 80%, at least 85%, at least 90%, at least 95%, at least 96%, at least 97%, at least 98%, or at least 99% amino acid identity to SEQ ID NO:13 (ANS, Carica papaya) that have the activity of a ANS.

Optionally, the cells are further engineered to include a flavonoid-3-glucosyl transferase (3GT) to generate anthocyanins by transfer of a sugar moiety such as, without limitation, UDP-a-D-glucose to anthocyanidins to form glycosylated anthocyanins. Considered for use in the engineered cells provided herein are 3GT of Vitis labrusca (SEQ ID NO: 14), 3GT with SEQ ID NOS: 70-73, and 3GT homologs of other species, as well as variants of naturally occurring 3GT having at least 50%, at least 55%, at least 60%, at least 65%, at least 70%, at least 75%, at least 80%, at least 85%, at least 90%, at least 95%, at least 96%, at least 97%, at least 98%, or at least 99% amino acid identity to SEQ ID NO: 14 (3GT, Vitis labrusca) that have the activity of a 3GT.

In various aspects, host cells may be engineered for enhanced production of flavonoids or anthocyanins by introducing additional exogenous pathways and/or modifying endogenous metabolic pathways to remove or downregulate competitive pathways to reduce carbon loss, increase precursor supply, improve cofactor availability, reduce byproduct formation, or improve cell fitness. Enhancing or improving production of flavonoids or anthocyanins can be increasing yield, titer, or rate of production.

Thus, a host cell engineered for the production of a flavonoid or anthocyanin can be engineered to include any or any combination of: overexpression of an acetyl-CoA carboxylase (ACC) or an ACC variant; expression or overexpression of at least one enzyme for increasing cell's malonyl-CoA supply that does not rely on the ACC step; expression or overexpression of at least one enzyme to increase tyrosine supply; expression or overexpression of at least one enzyme to increase CoA availability for synthesizing precursors malonyl-CoA or p-coumaryol-CoA; expression or overexpression at least one enzyme to increase heme biosynthesis; deletion or downregulation of at least one fatty acid synthesis enzyme; at least one alcohol dehydrogenase, lactate dehydrogenase, pyruvate oxidase, phosphate acetyl transferase, or acetate kinase; at least one enzyme of a fatty acid degradation pathway, at least one thioesterase, or at least one TCA gene. The foregoing list of modifications is nonlimiting.

Malonyl-CoA is the direct precursor for chalcone synthase to perform sequential condensations with p-coumaryol-CoA. Malonyl-CoA supply can be increased by one or more modifications. Malonyl-CoA is synthesized by acetyl-CoA carboxylase (ACC) via the ATP-dependent carboxylation of acetyl-CoA in a multistep reaction. First, the biotin carboxylase domain catalyzes the ATP-dependent carboxylation of biotin using bicarbonate as a C02 donor. In the second reaction, the carboxyl-group is transferred from biotin to acetyl-CoA to form malonyl-CoA. In most eukaryotes, including fungi, both reactions are catalyzed by a large single chain protein, but in E. coli and other bacteria, the activity is catalyzed by a multi-subunit enzyme. Host cells can be engineered for example to express an exogenous acetyl-CoA carboxylase or a variant ACC to increase malonyl-CoA synthesis from acetyl-CoA. For example, Mucor circinelloides (SEQ ID NO: 15) acetyl-CoA carboxylase can be introduced into the host cells. Additional examples of ACC genes that may be used in the engineered cells provided herein include, without limitation, the genes listed in Table 9, genes with SEQ ID NOS: 74-76, naturally occurring orthologs of these ACCs, or variants having at least 50%, at least 55%, at least 60%, at least 65%, at least 70%, at least 75%, at least 80%, at least 85%, at least 90%, at least 95%, at least 96%, at least 97%, at least 98%, or at least 99% amino acid identity to referenced genes. Further, naturally occurring acetyl-CoA carboxylase genes can be further engineered to introduce single or multiple amino acid mutations to increase catalytic activity and/or remove feedback inhibition.

TABLE 9
Acetyl-CoA carboxylases

| Organism | GenBank Accession Number |
|---|---|
| Lipomyces starkeyi | AJT60321.1 |
| Rhodotorula toruloides | GEM08739.1 |
| Ustilago maydis | XP_011390921.1 |
| Mucor circinelloides | EPB82652.1 |
| Kalaharituber pfeilii | KAF8466702.1 |
| Aspergillus fumigatus | KEY77072.1 |
| Rhodotorula diobovata | TNY18634.1 |
| Leucosporidium creatinivorum | ORY74050.1 |
| Microbotryum intermedium | SCV70467.1 |
| Mixia osmundae | GAA98306.1 |
| Puccinia graminis | KAA1079218.1 |
| Suillus occidentalis | KAG1764021.1 |
| Gymnopilus junonius | KAF8909366.1 |

Additional strategies for increasing malonyl-CoA include increasing acetyl-CoA, which is converted to malonyl-CoA by acetyl-CoA carboxylase (ACC). Acetyl-CoA can be synthesized from acetate by an acyl-CoA ligase in an ATP-dependent reaction. Acetyl-CoA synthetase (ACS) or acetate-CoA ligase (EC 6.2.1.1.) catalyzes the formation of a new chemical bond between acetate and CoA coenzyme A (CoA). ACSs with native activity on acetate will provide the function of increasing acetyl-CoA supply when cells are either supplied with acetate as a co-feed, or where acetate is produced as a by-product. Other acyl-CoA ligases, having their main activity on other acid substrates, may also have substantial activity on acetate, and are viable candidates for providing acetate-CoA ligase activity in the engineered cells provided herein. The ACSs expressed in the host cells can be prokaryotic or eukaryotic. Cultures of engineered host cells that overexpress a nucleic acid sequence encoding ACS can optionally include acetate in the culture medium. Examples of acetyl-CoA synthase that can be expressed in a host cell engineered to produce a flavonoid or anthocyanin include, without limitation, the ACS gene of E. coli, the ACS of Salmonella typhimurium (SEQ ID NO:16), and orthologs of these ACSs in other species having at least 50%, at least 55%, at least 60%, at least 65%, at least 70%, at least 75%, at least 80%, at least 85%, at least 90%, at least 95%, at least 96%, at least 97%, at least 98%, or at least 99% amino acid identity to these ACSs.

Alternatively, or in addition, an engineered host cell can overexpress a gene encoding pyruvate dehydrogenase (PDH), which converts pyruvate to acetyl-CoA, to increase acetyl-CoA supply. PDH catalyzes an irreversible metabolic step, and the control of its activity is complex and involves control by its substrates and products. Nicotinamide adenine dinucleotide hydrogen (NADH), a product of the PDH reaction, is a competitive inhibitor of the PDH complex. The NADH sensitivity of the PDH complex has been demonstrated to reside in LPD, the enzyme that interacts with NAD+ as a substrate. Thus, a variant of the Lpd subunit of PDH can be expressed that includes one or more mutations that reduces inhibition of PDH by NADH. Such an example is a LPD variant in E. coli that contains E354K mutation, and the mutated enzyme was less sensitive to NADH inhibition than the native LPD.

Alternatively, or in addition to strategies for increasing ACC activity and strategies for increasing acetyl-CoA, strategies for increasing malonyl-CoA by mechanisms that do not rely on the activity of an ACC can be employed. For example, a cell engineered to produce a flavonoid or an anthocyanin as provided herein can include an exogenous nucleic acid sequence encoding a malonyl-CoA synthetase (EC 6.2.1.14) that generates malonyl-CoA from malonate. Acyl-CoA synthetase catalyzes the conversion of a carboxylic acid to its acyl-CoA thioester through an ATP-dependent two-step reaction. In the first step, the free fatty acid is converted to an acyl-AMP intermediate with the release of pyrophosphate. In the second step, the activated acyl group is coupled to the thiol group of CoA, releasing AMP and the acyl-CoA product. Nonlimiting examples of malonyl-CoA synthetases include the malonyl-CoA synthetases of Streptomyces coelicolor (SEQ ID NO:17), matB of Rhodopseudomonas palustris (SEQ ID NO: 77), matB of Rhizobium sp, BUS003 (SEQ ID NO: 78), matB of Ochrobactrum sp. (SEQ ID NO: 79), or other homologs having at least 50%, at least 55%, at least 60%, at least 65%, at least 70%, at least 75%, at least 80%, at least 85%, at least 90%, at least 95%, at least 96%, at least 97%, at least 98%, or at least 99% identity to the referenced sequences. Malonate can optionally be added to the culture medium of a culture that includes a cell engineered to express a malonyl-CoA synthetase. In Rhizobium trifolii, the matB gene is part of the matABC operon, with matA encoding a malonyl-CoA decarboxylase and matC encoding a putative dicarboxylate carrier protein or malonate transporter. An engineered cell that includes an exogenous gene encoding a malonyl-CoA synthetase can also include an exogenous nucleic acid sequence encoding a malonate transporter, such as a malonate transporter encoded by a matC gene, for example of Streptomyces coelicolor (SEQ ID NO:18), of Rhizobiales bacterium (SEQ ID NO:80), of Rhizobium leguminosarum (SEQ ID NO:81), of Agrobacterium vitis (SEQ ID NO: 82), of Neorhizobium sp. (SEQ ID NO: 83), or a malonate transporter encoded by DctPQM of Sinorhizobium medicae, or encoding a malonyl-CoA transporter having at least 50%, at least 55%, at least 60%, at least 65%, at least 70%, at least 75%, at least 80%, at least 85%, at least 90%, at least 95%, at least 96%, at least 97%, at least 98%, or at least 99% identity to a naturally-occurring malonate transporter. Cell cultures of a host cell engineered to express a malonyl-CoA synthetase and a malonate transporter can include a culture medium that includes malonate.

In additional embodiments, a cell engineered to produce a flavonoid or an anthocyanin is further engineered to include an exogenous nucleic acid sequence encoding malonate CoA-transferase (EC:2.8.3.3; also referred to as the alpha subunit of malonate decarboxylase) that makes malonyl-CoA by direct transfer of the CoA from acetyl-CoA. For example, the alpha subunit of malonate decarboxylase from the mdcACDE gene cluster in Acinetobacter calcoaceticus has the malonate CoA-transferase activity. The mdcA gene product, the a subunit, is malonate CoA-transferase, and mdcD gene product, the β subunit, is a malonyl-CoA decarboxylase. The mdcE gene product, the γ subunit, may play a role in subunit interaction to form a stable complex or as a codecarboxylase. The mdcC gene product, the δ subunit, was an acyl-carrier protein, which has a unique CoA-like prosthetic group. When the a subunit is removed from the complex and incubated with malonate and acetyl-CoA, the acetyl-CoA moiety of the prosthetic group binds on an a subunit to exchange the acetyl group for a malonyl group. As the thioester transfer should be thermodynamically favorable, the engineered cells can include a nucleic acid encoding a malonate CoA-transferase to increase malonyl-CoA supply. Examples of mdcAs that can be expressed in an engineered cell as provided herein include, without limitation, mdcA of Acinetobacter calcoaceticus (SEQ ID NO: 19), mdcAs of Table 10, mdcAs with SEQ ID NOS: 84-87, or a transferase having at least 50%, at least 55%, at least 60%, at least 65%, at least 70%, at least 75%, at least 80%, at least 85%, at least 90%, at least 95%, at least 96%, at least 97%, at least 98%, or at least 99% identity to any of these or other naturally occurring malonate CoA-transferases.

TABLE 10
Malonate CoA-transferases (malonate decarboxylase subunit alpha)

| Organism | GenBank Accession Number |
|---|---|
| Acinetobacter calcoaceticus | AAB97627.1 |
| Geobacillus sp. | QNU36929.1 |
| Acinetobacter johnsonii | WP_087014029.1 |
| Acinetobacter marinus | WP_092618543.1 |
| Acinetobacter rudis | WP_016655668.1 |
| Psychrobacter sp. G | WP_020444454.1 |
| Moraxella catarrhalis | WP_064617969.1 |
| Zoogloea sp. | MBL0283742.1 |
| Dechloromonas sp. | KAB2923906.1 |
| Stenotrophomonas rhizophila | WP_123729366.1 |
| Xanthomonas cucurbitae | WP_159407614.1 |

In some embodiments, a cell engineered to produce flavonoids or anthocyanins is further engineered to increase the supply of coenzyme A (CoA) to increase its availability for producing acetyl-CoA, malonyl-CoA, and/or p-coumaroyl-CoA. Strategies for increasing CoA supply include expressing or overexpressing at least one enzyme of a CoA biosynthesis pathway. Pantothenate kinase (EC 2.7.1.33, PanK; CoaA) is the first enzyme in the coenzyme CoA biosynthetic pathway. It phosphorylates pantothenate (vitamin B5) to form 4′-phosphopantothenate at the expense of a molecule of adenosine triphosphate (ATP). It is the rate-limiting step in the biosynthesis of CoA. Three distinct types of PanK have been identified—PanK-I (found in bacteria), PanK-II (mainly found in eukaryotes, but also in the Staphylococci) and PanK-III, also known as CoaX (found in bacteria). In E. coli, pantothenate kinase is competitively inhibited by CoA itself, as well as by some CoA esters. The type III enzymes CoaX are not subject to feedback inhibition by CoA. In some embodiments, a host cell can be engineered to include a nucleic acid sequence encoding type III pantothenate kinase that is not feedback inhibited by coenzyme A, such as, without limitation, CoaX gene of Pseudomonas aeruginosa (EC:2.7.1.33, SEQ ID NO: 20), CoaX of Streptomyces sp. CL12509 (SEQ ID NO: 88), CoaX of Streptomyces cinereus (SEQ ID: 89), or CoaX of Kitasatospora kifunensis (SEQ ID NO: 90) Cultures of cells engineered for the production of flavonoids or anthocyanins can in some embodiments include a medium that includes pantothenate, a precursor of CoA biosynthesis, and can optionally also include cysteine, used in the CoA biosynthesis.

Additional strategies to increase malonyl-CoA flux to the flavonoid pathway include mutation or downregulation of one or more genes that function in fatty acid biosynthesis. Fatty acid biosynthesis directly competes with flavonoid biosynthesis for the precursor malonyl-CoA and thus limits flavonoid formation. Without limiting the embodiments to any particular mechanism, limiting fatty acid biosynthesis can increase the malonyl-CoA supply available for flavonoid biosynthesis. In some embodiments, the gene beta-ketoacyl-ACP synthase II (E. coli fabF) can be disrupted, attenuated or deleted to reduce fatty acid biosynthesis. Another example of a fatty acid biosynthesis gene of a host cell that may be mutated or downregulated is a gene encoding malonyl-CoA-ACP transacylase (E. coli fabD). Other fatty acid biosynthesis genes of the engineered host cell that can be downregulated include a beta-ketoacyl-ACP synthase I enzyme (E. coli fabB) and/or acyl carrier protein (E. coli acpP).

Additional genetic modifications that may be present in a host cell engineered to produce flavonoids or anthocyanins include downregulation, disruption, or deletion of the gene targets that divert carbon flux to form byproducts such as ethanol, acetate, and lactate. They include genes encoding alcohol dehydrogenase, lactate dehydrogenase, pyruvate oxidase, acetyl phosphate transferase and acetate kinase. In an E. coli host cell, genes that are downregulated, disrupted, or deleted can include adhE, ldhA, poxB, and ackA-pta.

Further, a cell engineered for the production of flavonoids or anthocyanins can have one or more genes encoding thioesterases downregulated, disrupted, or deleted to prevent hydrolysis of precursors malonyl-CoA, acetyl-CoA, and/or p-coumaryol-CoA. Acyl-CoA thioesterase enzymes (ACOTs) catalyze the hydrolysis of acyl-CoAs (short-, medium-, long- and very long-chain), bile acid-CoAs, and methyl branched-CoAs, to the free fatty acid and coenzyme A. For example, in an E. coli host one or more of the thioesterase genes tesA, tesB, yciA, and/or ybgC can be downregulated, disrupted, or deleted.

In further embodiments, a cell engineered for the production of flavonoids or anthocyanins can have one or more of fatty acid degradation genes downregulated, disrupted, or deleted to improve precursor supply to the flavonoid pathway. In E. coli, for example, the acyl-coenzyme A dehydrogenase (fade) gene encoding acyl-CoA dehydrogenase, adhesion A (fadA) gene encoding 3-ketoacyl-CoA thiolase, and/or gene encoding fatty acid oxidation complex subunit alpha (fadB) can be downregulated, disrupted, or deleted.

Alternatively, or in addition, genes encoding enzymes of the tricarboxylic acid cycle (TCA), such as succinate dehydrogenase, can be disrupted or downregulated to increase alpha-ketoglutarate supply which serves as a cofactor for the flavonoid and anthocyanin pathway enzymes. Other TCA enzymes that can be downregulated include citrate synthase that converts acetyl-CoA to citrate.

Also considered, in further embodiments, is an engineered host cell for the production of flavonoids or anthocyanins to upregulate the endogenous biosynthesis of amino acid tyrosine. Tyrosine is one of the precursors for the flavonoid biosynthesis and its conversion to 4-coumaric acid is the first committed step of the pathway. Efficient biosynthesis of L-tyrosine from feedstock such as glucose or glycerol is necessary to make biological production economically viable. L-tyrosine is one of the three aromatic amino acids derived from the shikimate pathway. The shikimate pathway is the central metabolic route leading to formation of tryptophan (TRP), tyrosine (TYR), and phenylalanine (PHE), this pathway exclusively exists in plants and microorganisms. It starts with the condensation of intermediates of glycolysis and pentosephosphate-pathway, phosphoenolpyruvate (PEP), and erythrose-4-phosphate (E4P), respectively, which enter the pathway through a series of condensation and redox reactions via 3-deoxy-d-arabino-heptulosonate-7-phosphate (DAHP), 3-dehydroquinate (DHQ), 3-dehydroshikimate (DHS) to shikimate. From there the central branch point metabolite chorismate is obtained via shikimate-3-phosphate under ATP hydrolysis and introduction of a second PEP. The initial step of the shikimate pathway is catalyzed by DAHP synthase isozymes and regulated through feedback-inhibition. In E. coli three DAHP synthase isozymes exist (aroF, aroG, aroH), which are each feedback inhibited by one of the three aromatic amino acids (TYR, PHE, TRP), in contrast the two DAHP synthases of plants are not subject to feedback-inhibition. In plants and bacteria, the subsequent five steps are catalyzed by single enzymes. From the central intermediate chorismate the pathway branches off to anthranilate and prephenate leading to aromatic amino acid, para-hydroxybenzoic acid (pHBA) and para-aminobenzoic acid (pABA) synthesis, the latter being a precursor for folate metabolism. Strategies to increase L-tyrosine production can include, without limitation, transcriptional deregulation, removing feedback inhibition, overexpression of rate-limiting enzymes, and/or deletion of the L-phenylalanine branch of the aromatic acid biosynthetic pathway. For example, in an E. coli host the tyrR gene can be disrupted, feedback-inhibition-resistant versions of the DAHP synthase (aroG) and chorismate mutase (tyrA) can be introduced, and/or rate-limiting enzymes, shikimate kinase (aroK or aroL) and quinate (QUIN)/shikimate dehydrogenase (ydiB) can be overexpressed. Further, the ppsA, aroG, and/or transketolase (tktA) can be overexpressed or exogenously introduced to enhance tyrosine production.

Also considered, in further embodiments, is an engineered host cell for the production of flavonoids or anthocyanins further engineered to upregulate the endogenous biosynthesis of cofactor heme. Cytochrome P450 (CYPs), one of the exogenous genes in the engineered cells provided herein, contain heme as a cofactor. Improving heme supply can be an effective strategy to increase flavonoid biosynthesis. 5-aminolevulinic acid (ALA) is the first committed precursor to the heme pathway. There exist two known alternate routes by which this committed intermediate is generated. One route is the C4 pathway (Shemin pathway), which involves the condensation of succinyl-CoA and glycine to D-aminolevulinic acid by ALA synthase (ALAS). The C4 pathway is restricted to mammals, fungi and purple nonsulfur bacteria. The second route is the C5 pathway, which involves three enzymatic reactions resulting in the biosynthesis of ALA from the five-carbon skeleton of glutamate. The C5 pathway is active in most bacteria, all archaea and plants. Seven additional reactions, including assembly of eight ALA molecules into a cyclic tetrapyrrole, modification of the side chains, and incorporation of reduced iron into the molecule, are required to convert ALA to heme. In an E. coli host, the three enzymes involved in ALA biosynthesis are glutamyl-tRNA synthetase (GltX), glutamyl-tRNA reductase (hemA), and glutamate-1-semialdehyde aminotransferase (hemL). In an E. coli host, the engineered cells provided herein can be further engineered to express or overexpress hemA or its variants, and/or hemL to increase the heme precursor ALA production. The nonlimiting examples of hemA gene that can be overexpressed include, without limitation, a mutated hemA gene from Salmonella typhimurium (EC:1.1.1.70, SEQ ID NO: 21) and hemA with SEQ ID NOS: 91-93. Alternatively, or in addition, a heterologous ALAS gene can be introduced to produce ALA via the C4 pathway. Nonlimiting examples of heterologous ALAS that can be expressed in E. coli include ALAS of Rhodobacter capsulatus (SEQ ID: 22), ALAS with SEQ ID NOS: 94-97, or an ALAS having at least 50%, at least 55%, at least 60%, at least 65%, at least 70%, at least 75%, at least 80%, at least 85%, at least 90%, at least 95%, at least 96%, at least 97%, at least 98%, or at least 99% identity to any of these or other naturally-occurring ALAS. Further, one or more of the downstream genes (E. coli hemB, hemC, hemD, hemE, hemF, hemG, hemL, or hemH) that catalyze the synthesis of heme from ALA can be overexpressed to drive the flux from ALA to heme production. Cultures of cells engineered for the production of flavonoids or anthocyanins can in some embodiments include a medium that includes succinate and/or glycine, precursors of heme biosynthesis via the C4 pathway.

Engineered cells that produce a flavonoid can be engineered to include multiple pathways to enhance flavonoid production. Those skilled in the art will recognize that the embodiments described herein can be combined in multiple ways. Examples of engineered cells having multiple genetic modifications are exemplary only and do not limit the scope of the invention.

Enzymes to be expressed or overexpressed in engineered cells according to the invention are set forth in Table 11.

### Host Cells

A host cell as provided herein can be a prokaryotic cell or a eukaryotic cell. Eukaryotic cells may be microbial eukaryotic cells, such as, for example, fungal cells or yeast cells. Prokaryotic cells that can be engineered as provided herein include bacterial cells and cyanobacterial cells.

Host can be selected based on their ability to take up and utilize particular carbon sources, nitrogen sources, or precursor molecules or may be engineered to take up and utilize molecules that may be added to the culture medium.

Nonlimiting examples of suitable microbial hosts for the bio-production of a flavonoid include, but are not limited to, any gram-negative organisms, more particularly a member of the family Enterobacteriaceae, such as E. coli, any gram-positive microorganism, for example Bacillus subtilis, Lactobacillus sp. or Lactococcus sp.; a yeast, for example Saccharomyces cerevisiae, Pichia pastoris or Pichia stipitis; and other groups or microbial species. More particularly, suitable microbial hosts for the bio-production of a flavonoid generally include, but are not limited to, members of the genera Clostridium, Zymomonas, Escherichia, Salmonella, Rhodococcus, Pseudomonas, Bacillus, Lactobacillus, Enterococcus, Alcaligenes, Klebsiella, Paenibacillus, Arthrobacter, Corynebacterium, Brevibacterium, Pichia, Candida, Hansenula, and Saccharomyces.

### Culture Medium

In yet another aspect, methods for producing a flavonoid or an anthocyanin that include incubating a culture of an engineered host cell as provided herein to produce a flavonoid or an anthocyanin. The methods can further include recovering the flavonoid or anthocyanin from the culture medium, whole culture, or cells.

The culture comprises cells engineered for the production of flavonoids or anthocyanins in a culture medium. In various embodiments the engineered cells can be prokaryotic or eukaryotic cells. The culture medium includes at least one carbon source that is also an energy source. Exemplary carbon sources include glucose, glycerol, sucrose, fructose, and xylose. Such carbon sources may be purified or crude, including a biomass comprising glycerol, for example, crude glycerol produced as a byproduct of biodiesel production from corn waste. In addition, the culture medium can include one or more other carbon sources or compounds to increase precursor generation or cofactor supply such as, without limitation, tyrosine, phenylalanine, coumaric acid, acetate, malonate, succinate, glycine, bicarbonate, biotin, naringenin, 5-aminolevulinic acid, thiamine, pantothenate, alpha-ketoglutarate, and ascorbate. In some embodiments, tyrosine and coumaric acid are provided in the culture medium. In some embodiments, tyrosine, alpha-ketoglutarate, 5-aminolevulinic acid, and ascorbate are provided in the culture medium. Culture conditions can include aerobic, microaerobic or any combination alternating aerobic/microaerobic growth conditions. Further, culture conditions can include shake flasks, fermentation, and other large scale culture procedures. An exemplary growth condition for achieving a flavonoid product include aerobic or microaerobic fermentation conditions. The culture conditions can be scaled up and grown continuously for manufacturing flavonoid product. Exemplary growth procedures include, for example, fed-batch fermentation and batch separation. In an exemplary batch fermentation protocol, the cells are grown in a bioreactor that is well controlled for growth temperature, oxygen, pH, carbon sources, and other compounds. The desired temperature can be from, for example, 20-37 °C, depending on the growth characteristics of the production cells and desired conditions for the fermented products. The pH of the bioreactor can be controlled to range from 5-8 or left uncontrolled in some cases. The batch fermentation period can last in the range of several hours to several days, for examples, 8 to 96 hours. Upon completion of the cultivation period, the fermenter contents can be passed through a cell separation unit to remove cells and cell debris. The cells can be lysed or disrupted enzymatically or chemically prior to or after separation of cells from the fermentation broth, as desired, in order to release additional product. To purify the flavonoids and/or anthocyanins to homogeneity the solution containing the flavonoids and/or anthocyanins was concentrated and the product purified via ion exchange or silica-based chromatography. The resulting solution was either lyophilized to yield the products in a solid form or was concentrated into a liquid solution.

In some embodiments, a method of producing a flavonoid or an anthocyanin comprises culturing an engineered cell disclosed herein in a culture medium to produce a flavonoid or an anthocyanin. In some embodiments, glycerol is used as a carbon feedstock. In some embodiments, the glycerol is crude glycerol. In some embodiments, the method comprises isolating naringenin, dihydrokaempferol, taxifolin, eriodictyol, leucocyanidin, leucodelphinidin, leucopelargonidin, (+)-catechin, cyanidin, delphinidin, pelargonidin, cyanidin glucoside, delphinidin glucoside or pelargonidin glucoside. In some embodiments the naringenin, dihydrokaempferol, taxifolin, eriodictyol, leucocyanidin, leucodelphinidin, leucopelargonidin, (+)-catechin, cyanidin, delphinidin, pelargonidin, cyanidin glucoside, delphinidin glucoside or pelargonidin glucoside may be isolated at a purity of greater than 50%, greater than 55%, greater than 60%, greater than 65%, greater than 70%, greater than 75%, greater than 80%, greater than 85%, greater than 90%, or greater than 95%. In some embodiments, the naringenin, dihydrokaempferol, taxifolin, eriodictyol, leucocyanidin, leucodelphinidin, leucopelargonidin, (+)-catechin, cyanidin, delphinidin, pelargonidin, cyanidin glucoside, delphinidin glucoside or pelargonidin glucoside may be isolated at a purity of from about 50% to about 99%, e.g., from about 50% to about 95% (for example from: about 50%, 55%, 60%, 65%, 70%, 75%, 80% to about: 85%, 90%, 95%, 97.5%, 99% or 99.9%). In some embodiments, the naringenin, dihydrokaempferol, taxifolin, eriodictyol, leucocyanidin, leucodelphinidin, leucopelargonidin, (+)-catechin, cyanidin, delphinidin, pelargonidin, cyanidin glucoside, delphinidin glucoside or pelargonidin glucoside may be isolated at a purity of from about 50% to: about 55%, about 60%, about 65%, about 70%, about 75%, about 80%, about 85%, about 90%, about 95%, or about 99%. In some embodiments, the naringenin, dihydrokaempferol, taxifolin, eriodictyol, leucocyanidin, leucodelphinidin, leucopelargonidin, (+)-catechin, cyanidin, delphinidin, pelargonidin, cyanidin glucoside, delphinidin glucoside or pelargonidin glucoside may be isolated at a purity of from about 55% to: about 60%, about 65%, about 70%, about 75%, about 80%, about 85%, about 90%, about 95%, or about 99%. In some embodiments, the naringenin, dihydrokaempferol, taxifolin, eriodictyol, leucocyanidin, leucodelphinidin, leucopelargonidin, (+)-catechin, cyanidin, delphinidin, pelargonidin, cyanidin glucoside, delphinidin glucoside or pelargonidin glucoside may be isolated at a purity of from about 60% to: about 65%, about 70%, about 75%, about 80%, about 85%, about 90%, about 95%, or about 99%. In some embodiments, the naringenin, dihydrokaempferol, taxifolin, eriodictyol, leucocyanidin, leucodelphinidin, leucopelargonidin, (+)-catechin, cyanidin, delphinidin, pelargonidin, cyanidin glucoside, delphinidin glucoside or pelargonidin glucoside may be isolated at a purity of from about 65% to: about 70%, about 75%, about 80%, about 85%, about 90%, about 95%, or about 99%. In some embodiments, the naringenin, dihydrokaempferol, taxifolin, eriodictyol, leucocyanidin, leucodelphinidin, leucopelargonidin, (+)-catechin, cyanidin, delphinidin, pelargonidin, cyanidin glucoside, delphinidin glucoside or pelargonidin glucoside may be isolated at a purity of from about 70% to: about 75%, about 80%, about 85%, about 90%, about 95%, or about 99%. In some embodiments, the naringenin, dihydrokaempferol, taxifolin, eriodictyol, leucocyanidin, leucodelphinidin, leucopelargonidin, (+)-catechin, cyanidin, delphinidin, pelargonidin, cyanidin glucoside, delphinidin glucoside or pelargonidin glucoside may be isolated at a purity of from about 75% to: about 80%, about 85%, about 90%, about 95%, or about 99%, from about 80% to about 85%, about 90%, about 95%, or about 99%. In some embodiments, the naringenin, dihydrokaempferol, taxifolin, eriodictyol, leucocyanidin, leucodelphinidin, leucopelargonidin, (+)-catechin, cyanidin, delphinidin, pelargonidin, cyanidin glucoside, delphinidin glucoside or pelargonidin glucoside may be isolated at a purity of from about 85% to: about 90%, about 95%, or about 99%. In some embodiments, the naringenin, dihydrokaempferol, taxifolin, eriodictyol, leucocyanidin, leucodelphinidin, leucopelargonidin, (+)-catechin, cyanidin, delphinidin, pelargonidin, cyanidin glucoside, delphinidin glucoside or pelargonidin glucoside may be isolated at a purity of from about 90% to about 95%, or about 99%, or from about 95% to about 99% or greater.

### VI. EXAMPLES

### Using the Modified Cell to Create Products

### Example 1—Production of Naringenin in E. coli

An E. coli cell derived from MG1655 was engineered to overexpress ACC (SEQ ID NO: 15), TAL (SEQ ID NO: 1), 4CL (SEQ ID NO: 4), CHS (SEQ ID NO: 5), and CHI (SEQ ID NO: 6) to produce naringenin when substrates tyrosine and coumaric acid were supplied in culture medium. ACC was expressed on a medium-copy plasmid (15-20 copies) while TAL, 4CL, CHS, and CHI were expressed on the chromosome. Cells of an OD 2.5 were cultured in a 48-well plate at 30 degree for 24 hours with a shaking speed of 600 RPM in minimal medium supplied with trace element, vitamins, 1 mM tyrosine, 1 mM coumaric acid, and 2% glycerol. Cell cultures were extracted with DMSO at 1:1 ratio and centrifuged for 15 mins. The supernatant was analyzed for naringenin with HPLC. The cells produced 232 μM naringenin.

Variants of the foregoing host cell may be prepared using one or more of ACC (SEQ ID NO: 15), TAL (SEQ ID NO: 1), 4CL (SEQ ID NO: 4), CHS (SEQ ID NO: 5), and CHI (SEQ ID NO: 6) with one or more homologs of ACC (SEQ ID NO: 15), TAL (SEQ ID NO: 1), 4CL (SEQ ID NO: 4), CHS (SEQ ID NO: 5), or CHI (SEQ ID NO: 6), or combinations of two or more thereof, wherein the homologous enzymes have at least 50%, at least 55%, at least 60%, at least 65%, at least 70%, at least 75%, at least 80%, at least 85%, at least 90%, at least 95%, at least 96%, at least 97%, at least 98%, or at least 99% identity to the referenced enzymes.

### Example 2—Production of Dihydrokaempferol in E. coli

An E. coli cell derived from MG1655 was engineered to overexpress F3H (SEQ ID NO: 7) on the chromosome to produce dihydrokaempferol when substrate naringenin was supplied in culture medium. Cells of an OD 0.5-0.7 were cultured in a 24-well plate at 30 degree for 18 hours with a shaking speed of 200 RPM in minimal medium supplied with 2% glycerol, trace elements, 0.8 mM naringenin, 65 mg/L 5-aminoleuvinic acid, 0.1 mM ferrous sulfate, 0.1 mM 2-oxoglutarate, and 2.5 mM ascorbic acid. Cell cultures were extracted with DMSO and centrifuged for 15 minutes. The supernatant was analyzed for dihydrokaempferol with HPLC. The cells produced 315 μM dihydrokaempferol.

Variants of the foregoing host cell may be prepared using a homolog of F3H (SEQ ID NO: 7), wherein the homologous enzyme has at least 50%, at least 55%, at least 60%, at least 65%, at least 70%, at least 75%, at least 80%, at least 85%, at least 90%, at least 95%, at least 96%, at least 97%, at least 98%, or at least 99% identity to the referenced enzyme.

### Example 3—Production of Taxifolin in E. coli

An E. coli strain derived from MG1655 was engineered to overexpress F3H (SEQ ID NO: 7), F3′H (SEQ ID NO: 8), and CPR (SEQ ID NO: 9) to produce taxifolin when the substrate naringenin was supplied in culture medium. F3H was overexpressed on the chromosome while F3′H and CPR were overexpressed on a medium-copy plasmid. Cells of an OD 0.5-0.7 were cultured in a 24-well plate at 30 degree for 18 hours with a shaking speed of 200 RPM in minimal medium supplied with 2% glucose, 0.8 mM naringenin, 65 mg/L 5-aminoleuvinic acid, 0.1 mM ferrous sulfate, 0.1 mM 2-oxoglutarate, and 2.5 mM ascorbic acid. Cell cultures were extracted with 50% DMSO and centrifuged for 15 minutes. The supernatant was analyzed for taxifolin with HPLC. The cells produced 500 μM taxifolin.

Variants of the foregoing host cell may be prepared using one or more of F3H (SEQ ID NO: 7), F3′H (SEQ ID NO: 8), and CPR (SEQ ID NO: 9) along with one or more homologs of F3H (SEQ ID NO: 7), F3′H (SEQ ID NO: 8), and CPR (SEQ ID NO: 9), or combinations of two or more thereof, wherein the homologous enzymes have at least 50%, at least 55%, at least 60%, at least 65%, at least 70%, at least 75%, at least 80%, at least 85%, at least 90%, at least 95%, at least 96%, at least 97%, at least 98%, or at least 99% identity to the referenced enzymes.

### Example 4—Production of Anthocyanidins and Anthocyanins

An E. coli strain derived from MG1655 was engineered to overexpress ANS (SEQ ID NO: 13) and 3GT (SEQ ID NO: 14) to produce cyanidin-3-O-glucoside when the substrate (+)-catechin was supplied in culture medium. ANS and 3GT were overexpressed on the chromosome. Cells of an OD 0.5-0.7 were cultured in a 24-well plate at 30 °C for 18 hours with a shaking speed of 200 RPM in minimal medium supplied with 1.0% glucose, 2.0 mM (+)-catechin, 0.1 mM 2-oxoglutarate, and 2.5 mM ascorbic acid. Cell cultures were acidified with 2M HCL and extracted with 100% Ethanol. The supernatant was analyzed for cyanidin-3-O-glucoside by HPLC. The cells produced 50 mg/L cyanidin-3-O-glucoside.

Variants of the foregoing host cell may be prepared using one or both of ANS (SEQ ID NO: 13) and 3GT (SEQ ID NO: 14) along with a homolog of ANS (SEQ ID NO: 13), 3GT (SEQ ID NO: 14), or both, wherein the homologous enzymes have at least 50%, at least 55%, at least 60%, at least 65%, at least 70%, at least 75%, at least 80%, at least 85%, at least 90%, at least 95%, at least 96%, at least 97%, at least 98%, or at least 99% identity to the referenced enzymes.

### Example 5—Production of Quercetin

An E. coli cell derived from MG1655 was engineered to overexpress ACC (SEQ ID NO:15), TAL (SEQ ID NO: 1), 4CL (SEQ ID NO: 4), CHS (SEQ ID NO: 5), CHI (SEQ ID NO: 6), F3H (SEQ ID NO: 7), F3′H (SEQ ID NO: 8), CPR (SEQ ID NO: 9), and FLS (SEQ ID NO: 99) to produce quercetin when the substrate glycerol was supplied in culture medium. ACC, TAL, 4CL, CHS, CHI, F3H, F3′H, CPR, and FLS were expressed on the chromosome. Cells of an OD 2.0 were cultured in a 48-well plate at 30 °C for 24 hours with a shaking speed of 600 RPM in minimal medium supplied with trace elements, vitamins, 2% glycerol, 2.0 mM 5-aminoleuvinic acid, 0.1 mM ferrous sulfate, 0.1 mM 2-oxoglutarate, and 2.5 mM ascorbic acid. Cell cultures were extracted with methanol at 5:1 ratio (MeOH:cells) and centrifuged for 15 minutes. The supernatant was analyzed for quercetin by HPLC. The cells produced 256 μM quercetin.

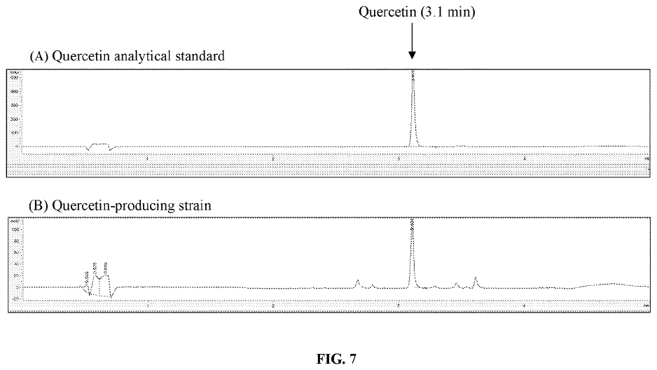

FIG. 7 provides the data demonstrating the production of quercetin by the method described herein. Panel (A) provides the trace of quercetin analytical standard, and panel (B) provides the trace from the quercetin producing strain. As evident from the data provided in FIG. 7, the method provided herein demonstrates production of quercetin in engineered cells.

Variants of the foregoing host cell may be prepared using one or more of ACC (SEQ ID NO:15), TAL (SEQ ID NO: 1), 4CL (SEQ ID NO: 4), CHS (SEQ ID NO: 5), CHI (SEQ ID NO: 6) F3H (SEQ ID NO: 7), F3′H (SEQ ID NO: 8), CPR (SEQ ID NO: 9), and FLS (SEQ ID NO: 99) with one or more homologs of ACC (SEQ ID NO: 15), TAL (SEQ ID NO: 1), 4CL (SEQ ID NO: 4), CHS (SEQ ID NO: 5), CHI (SEQ ID NO: 6), F3H (SEQ ID NO: 7), F3′H (SEQ ID NO: 8), CPR (SEQ ID NO: 9), and FLS (SEQ ID NO: 99) or combinations of two or more thereof, wherein the homologous enzymes have at least 50%, at least 55%, at least 60%, at least 65%, at 15 least 70%, at least 75%, at least 80%, at least 85%, at least 90%, at least 95%, at least 96%, at least 97%, at least 98%, or at least 99% identity to the referenced enzymes.

### Analytical Methods

### Example 6—Flavonoid Precursors and Flavonoids

For sampling naringenin, eriodictyol, dihydrokaempferol and taxifolin, extraction of total flavonoids from E. coli were performed on whole cell broth. 500 μL of whole cell broth was vortexed for 30 seconds with 500 μL of DMSO (dimethyl sulfoxide) and centrifuged for 15 minutes. For HPLC analysis, 50 μL of supernatant was transferred to an HPLC vial.

The HPLC method was as follows: An Agilent 1200 HPLC was fitted with an Ascentis C18 Column 150 mm×4.6 mm, 3 μm, equipped with a R-18 (3 μm) guard column. The column was heated to 30 °C with the sample block being maintained at 25 °C. For each sample, 5 μL was injected and the product was eluted at a flow rate of 1.5 mL/min using 0.1% phosphoric acid in water (solvent A), acetonitrile (solvent B), and methanol (solvent C) with the following gradient:

| | A | B | C |
|---|---|---|---|
| Time | (%) | (%) | (%) |
| 0 | 85 | 10 | 5 |
| 2.5 | 85 | 10 | 5 |
| 7.5 | 70 | 25 | 5 |
| 12.5 | 50 | 45 | 5 |
| 15 | 85 | 10 | 5 |

The run time was a total of 15 minutes with naringenin, eriodictyol, dihydrokaempferol and taxifolin eluting at 12.50, 11.56, 10.20, and 8.85 minutes respectively. A diode array detector (DAD) was used for the detection of the molecule of interest at 288 nm.

### Example 7—Anthocyanidins and Anthocyanins

For sampling (+)-catechin, cyanidin, and cyanidin-3-glucoside the reaction fluid was acidified with 13 M HCl (1:40 v/v), and extracted with 100% ethanol followed by mixing, centrifugation and filtration through a 0.45 m filter. The HPLC method was as follows: An Agilent 1200 HPLC was fitted with a LiChrospher RP-8 Column 250 mm×4.6 mm, 5 μm, equipped with a LiChrospher 100 RP-8 (5 m) LiChroCART 4-4 guard column. The column was heated to 25 °C with the sample block being maintained at 25 °C. For each sample, 10 μL was injected and the product was eluted at a flow rate of 1.0 ml/min using 0.1% phosphoric acid in water (solvent A) and acetonitrile (solvent B) with the following gradient: 90% A to 10% A for 12 min, 90% A for 0.5 min, and 90% A for 3.5 min for column equilibration. The run time was a total of 16 minutes with cyanidin-3-glycoside eluting at 6.95 mins and cyanidin eluting at 8.9 minutes. A diode array detector (DAD) was used for the detection of the molecule of interest at either 280 nm or 530 nm.

### Example 8—Flavonoid Production

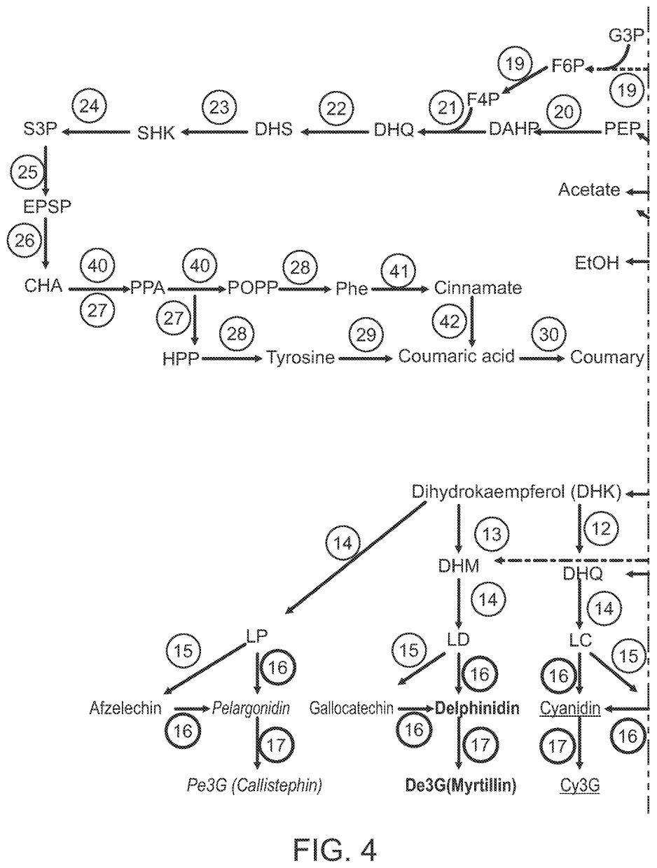

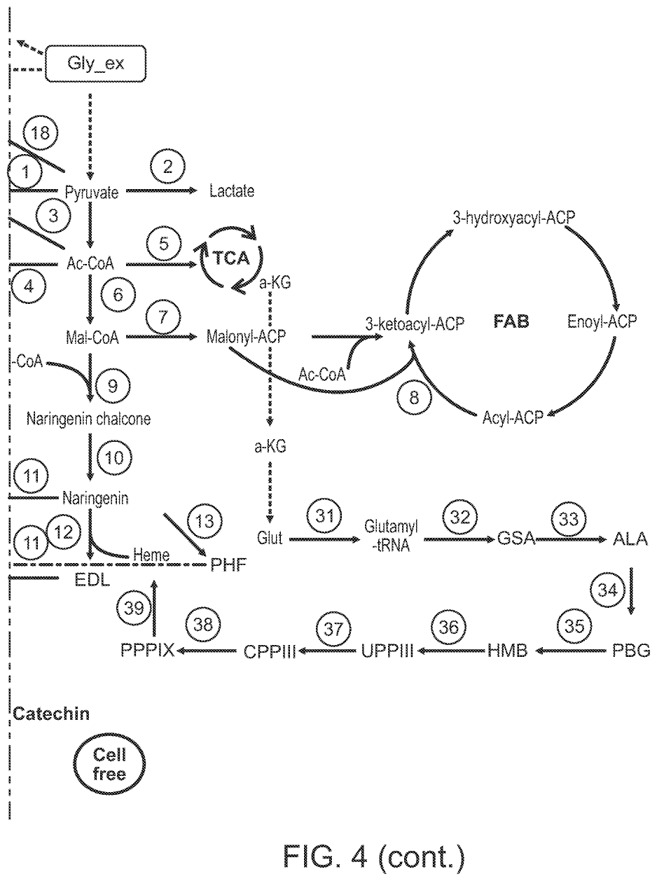

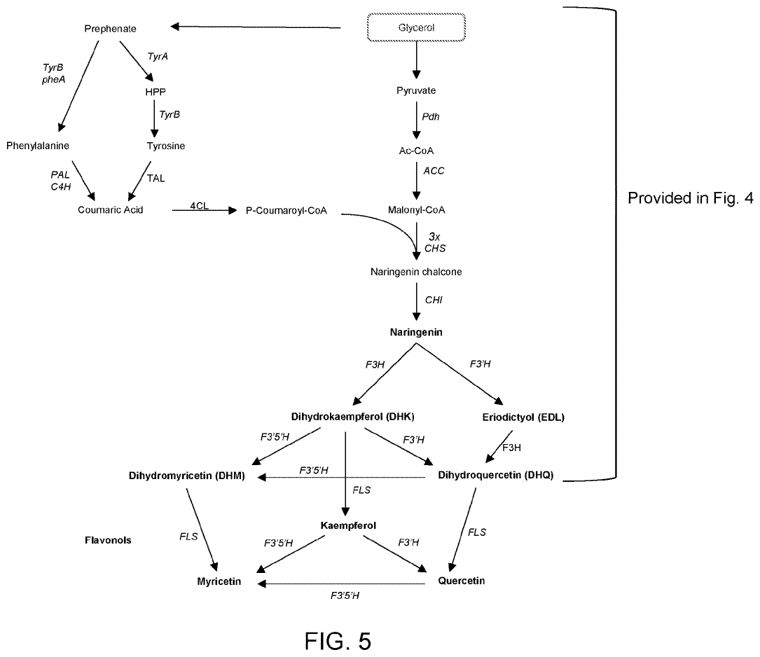

The example provides a combination of modifications to the E. coli host genome including deletions and overexpression of enzymes from other organisms to recapitulate the bioproduction pathway described in FIG. 4. Accordingly, the invention provides an engineered host cell that comprises one or more genetic modifications (as shown in FIG. 4 and described in this Example 8 and herein above in this application) that result in production of flavonoid or anthocyanin from a carbon source that can also be an energy source, through multiple chemical intermediates, by the engineered host cell. In certain embodiments, the production of flavonoid or anthocyanin from a carbon source that can also be an energy source occurs through enzymatic transformation. In certain embodiments, the carbon source is selected from a group consisting of: (i) glycerol, (ii) a sugar, (iii) an organic acid, (iv) an amino acid, and (v) any combination thereof. In certain embodiments, the engineered host cell is cultured in a medium comprising molecules selected from a group consisting of: (i) glycerol, (ii) a sugar, (iii) an organic acid, (iv) an amino acid, and (v) any combination thereof. As shown in FIG. 4, in certain embodiments, one or more genetic modifications lead to increase in metabolic flux to flavonoid precursors or cofactors. As shown in FIG. 4, in certain embodiments, one or more of the genetic modifications cause reduction of formation of byproducts. As shown in FIG. 4, in certain embodiments, one or more genetic modifications are selected from: (i) one or more modifications for over-expressing one or more endogenous genes in the engineered host cells; (ii) one or more modifications for under-expressing one or more endogenous genes in the engineered host cells; (iii) one or more genetic modification is expressing one or more non-native genes in the engineered host cells; and (iv) a combination thereof.

As shown in FIG. 4, in certain embodiments, the engineered host cell is cultured in a medium comprising molecules selected from: tyrosine, phenylalanine, malonate, p-coumarate, bicarbonate, acetate, pantothenate, biotin, thiamine, alpha-ketoglutarate, ascorbate, and 5-aminolevulinic acid.

As shown in FIG. 4, in certain embodiments, the engineered host cell comprises at least one or more nucleic acid sequences selected from: (i) a nucleic acid sequences encoding tyrosine ammonia lyase activity; (ii) a nucleic acid sequences encoding phenylalanine ammonia lyase activity; (iii) cinnamate 4-hydroxylase; and (iv) any combination thereof. As shown in FIG. 4, in certain embodiments, the engineered host cell comprises at least one or more peptides selected from: (i) chalcone isomerase; (ii) chalcone synthase; (iii) a fusion protein comprises a chalcone synthase and a chalcone isomerase; and (iv) any combination thereof.

As shown in FIG. 4, in certain embodiments, one or more genetic modifications decreases fatty acid biosynthesis. As shown in FIG. 4, in certain embodiments, the engineered host cell comprises an exogenous nucleic acid sequence selected from: (i) nucleic acid sequence encoding tyrosine ammonia lyase, wherein the encoded tyrosine ammonia lyase forms 4-coumaric acid using tyrosine as a substrate; (ii) nucleic acid sequence encoding phenylalanine ammonia lyase, wherein the encoded phenylalanine ammonia lyase converts phenylalanine to trans-cinnamic acid; (iii) nucleic acid sequence encoding cinnamate-4-hydroxylase, wherein the cinnamate-4-hydroxylase produces 4-coumaric acid from trans-cinnamic acid; (iv) nucleic acid sequence encoding flavanone-3-hydroxylase, wherein flavanone-3-hydroxylase forms dihydrokaempferol from naringenin; and (v) any combinations thereof.

As shown in FIG. 4, in certain embodiments, the engineered host cell comprises at least one or more nucleic acid sequences selected from: (i) nucleic acid sequences encoding tyrosine ammonia lyase activity; (ii) nucleic acid sequences encoding phenylalanine ammonia lyase activity; (iii) nucleic acid sequences encoding cinnamate 4-hydroxylase activity; (iv) nucleic acid sequences encoding 4-courmarate-CoA ligase (4CL) activity; and (v) any combination thereof. As shown in FIG. 4, in certain embodiments, the engineered host cell comprises an exogenous nucleic acid sequence selected from the group consisting of: (i) nucleic acid sequence encoding tyrosine ammonia lyase, wherein the encoded tyrosine ammonia lyase forms 4-coumaric acid using tyrosine as a substrate; (ii) nucleic acid sequence encoding phenylalanine ammonia lyase, wherein the encoded phenylalanine ammonia lyase converts phenylalanine to trans-cinnamic acid; (iii) nucleic acid sequence encoding cinnamate-4-hydroxylase, wherein the cinnamate-4-hydroxylase produces 4-coumaric acid from trans-cinnamic acid; (iv) nucleic acid sequence encoding 4-courmarate-CoA ligase activity, wherein 4-courmarate-CoA ligase forms p-coumaroyl-CoA from coumaric acid (v) nucleic acid sequence encoding chalcone synthase activity, wherein chalcone synthase forms naringenin chalcone from malonyl-CoA and p-coumaroyl-CoA; (vi) nucleic acid sequence encoding chalcone isomerase activity, wherein chalcone isomerase forms naringenin from naringenin chalcone; (vii) nucleic acid sequence encoding flavanone-3-hydroxylase, wherein flavanone-3-hydroxylase forms dihydrokaempferol from naringenin; and (viii) any combinations thereof.

The compositions as described above, can be used in methods described herein for increasing the production of flavonoids or anthocyanins. Such methods involve providing any of the compositions described above to result in enzymatic transformation by the engineered host cell of glycerol through multiple chemical intermediates into a flavonoid or anthocyanin (such as shown in part or in whole in FIG. 4).

In yet another aspect, it is envisioned that the pathway illustrated in FIG. 4 can be carried out using a plurality of engineered host cells, as opposed to a single host cell as described above. In such embodiments, the plurality of the engineered host cells have one or more genetic modifications that result in enzymatic transformation by the engineered host cell of glycerol through multiple chemical intermediates into a flavonoid or anthocyanin (as shown in FIG. 4).

Aspects of the invention are now described with reference herein to FIG. 4.

Step 1: conversion of pyruvate to acetate. poxB is deleted to reduce carbon loss and eliminate the byproducts.

Step 2: conversion of pyruvate to lactate. ldhA is deleted to reduce carbon loss and eliminate the byproducts.

Step 3: conversion of Acetyl-CoA to acetate. ackA-pta is deleted to reduce carbon loss and eliminate the byproducts.

Step 4: conversion of Acetyl-CoA to ethanol (EtOH). adhE is deleted to reduce carbon loss and eliminate the byproducts.

Step 5: conversion of acetyl-CoA to a substrate for the tricarboxylic acid cycle (TCA).

Step 6: conversion of acetyl-CoA to mal-CoA. Heterologous ACC is expressed to increase the concentration of available mal-CoA. Heterologous ACC may be obtained from fungal species. Accordingly, embodiments of the invention provide an engineered host cell that comprises one or more genetic modifications to increase the production and/or availability of malonyl-CoA. In certain embodiments, the engineered host cell comprises one or more genetic modifications selected from: (i) expression of acetyl-CoA carboxylase (ACC); and (ii) overexpression of acetyl-CoA carboxylase. In another embodiment, the engineered host cell is an E. coli. In certain embodiments, the acetyl-CoA carboxylase is from: Mucor circinelloides, Rhodotorula toruloides, Lipomyces starkeyi, and Ustilago maydis, and orthologs of acetyl-CoA carboxylase having at least 50% amino acid identity to the acetyl-CoA carboxylase of these aforementioned species. In certain embodiments, one or more genetic modification is deletion or attenuation of one or more fatty biosynthetic genes resulting in decrease in fatty acid biosynthesis. In certain embodiments, one or more genetic modification is overexpression of acetyl-CoA synthase (ACS). In certain embodiments, the acetyl-CoA synthase is selected from: acetyl-CoA synthase gene of E. coli, acetyl-CoA synthase gene of Salmonella typhimurium, and orthologs of acetyl-CoA synthase gene in any other species having at least 50% amino acid identity to the acetyl-CoA synthase gene of E. coli and Salmonella typhimurium. In certain embodiments, one or more genetic modification is selected from a group consisting of: (i) overexpression a gene encoding pyruvate dehydrogenase (PDH), wherein the PDH may include E354K mutation; (ii) exogenous nucleic acid sequence encoding a malonyl-CoA synthetase; (iii) upregulation of endogenous pantothenate kinase (PanK), wherein PanK is not feedback inhibited by coenzyme A; (iv) exogenous nucleic acid sequence encoding a malonate transporter; and (v) any combinations thereof. In certain embodiments, the malonyl-CoA synthetase is selected from of malonyl-CoA synthetases of Streptomyces coelicolor, Rhodopseudomonas palustris, or a malonyl-CoA synthetase having at least 50% identity to any of these or other naturally occurring malonyl-CoA synthetases. In certain embodiments, one or more genetic modifications to decrease fatty acid biosynthesis is selected from: (i) mutation or downregulation of a gene encoding malonyl-CoA-ACP transacylase (E. coli fabD); (ii) modifications to the gene beta-ketoacyl-ACP synthase II (E. coli fabF); (iii) downregulation of beta-ketoacyl-ACP synthase I enzyme (E. coli fabB); (iv) downregulation of acyl carrier protein (E. coli acpP); and (v) any combinations thereof. In certain embodiments, the engineered host cell comprises peptides selected from: (i) acetyl-CoA carboxylase (ACC) having an amino acid sequence at least 80% identical to the polypeptide set forth in SEQ ID NO: 15 or SEQ ID NO: 16; (ii) malonate CoA-transferase having an amino acid sequence at least 80% identical to the polypeptide set forth in SEQ ID NO: 19; (iii) acetyl-CoA synthase (ACS) having an amino acid sequence at least 80% identical to the polypeptide set forth in SEQ ID NO: 16; (iv) malonyl-CoA synthase having an amino acid sequence at least 80% identical SEQ ID NO: 77, SEQ ID NO: 78, or SEQ ID NO: 79; (v) malonate transporter having an amino acid sequence at least 80% identical to SEQ ID NO: 80, SEQ ID NO: 81, SEQ ID NO: 82, SEQ ID NO: 83, SEQ ID NO: 84, SEQ ID NO: 85, SEQ ID NO: 86, or SEQ ID NO: 87; (vi) pantothenate kinase having an amino acid sequence at least 80% identical to SEQ ID NO: 88, SEQ ID NO: 89, or SEQ ID NO: 90; and (vii) any combinations thereof.

In another aspect, the invention provides a method of increasing the production of flavonoids comprising an engineered host cell, wherein the one or more engineered host cells comprise one or more genetic modifications to increase production and/or availability of malonyl-CoA. In certain embodiments, the engineered host cell comprises one or more genetic modifications selected from: (i) expression of acetyl-CoA carboxylase (ACC); and (ii) overexpression of acetyl-CoA carboxylase. In another embodiment, the engineered host cell is an E. coli. In certain embodiments, the acetyl-CoA carboxylase is from: Mucor circinelloides, Rhodotorula toruloides, Lipomyces starkeyi, and Ustilago maydis, and orthologs of acetyl-CoA carboxylase having at least 50% amino acid identity to the acetyl-CoA carboxylase of these aforementioned species. In certain embodiments, one or more genetic modification is deletion or attenuation of one or more fatty biosynthetic genes resulting in decrease in fatty acid biosynthesis. In certain embodiments, one or more genetic modification is overexpression of acetyl-CoA synthase (ACS). In certain embodiments, the acetyl-CoA synthase is selected from: acetyl-CoA synthase gene of E. coli, acetyl-CoA synthase gene of Salmonella typhimurium, and orthologs of acetyl-CoA synthase gene in any other species having at least 50% amino acid identity to the acetyl-CoA synthase gene of E. coli and Salmonella typhimurium. In certain embodiments, one or more genetic modification is selected from a group consisting of: (i) overexpression a gene encoding pyruvate dehydrogenase (PDH), wherein the PDH may include E354K mutation; (ii) exogenous nucleic acid sequence encoding a malonyl-CoA synthetase; (iii) upregulation of endogenous pantothenate kinase (PanK), wherein PanK is not feedback inhibited by coenzyme A; (iv) exogenous nucleic acid sequence encoding a malonate transporter; and (v) any combinations thereof. In certain embodiments, the malonyl-CoA synthetase is selected from of malonyl-CoA synthetases of Streptomyces coelicolor, Rhodopseudomonas palustris, or a malonyl-CoA synthetase having at least 50% identity to any of these or other naturally occurring malonyl-CoA synthetases. In certain embodiments, one or more genetic modifications to decrease fatty acid biosynthesis is selected from: (i) mutation or downregulation of a gene encoding malonyl-CoA-ACP transacylase (E. coli fabD); (ii) modifications to the gene beta-ketoacyl-ACP synthase II (E. coli fabF); (iii) downregulation of beta-ketoacyl-ACP synthase I enzyme (E. coli fabB); (iv) downregulation of acyl carrier protein (E. coli acpP); and (v) any combinations thereof. In certain embodiments, the engineered host cell comprises peptides selected from: (i) acetyl-CoA carboxylase (ACC) having an amino acid sequence at least 80% identical to the polypeptide set forth in SEQ ID NO: 15 or SEQ ID NO: 16; (ii) malonate CoA-transferase having an amino acid sequence at least 80% identical to the polypeptide set forth in SEQ ID NO: 19; (iii) acetyl-CoA synthase (ACS) having an amino acid sequence at least 80% identical to the polypeptide set forth in SEQ ID NO: 16; (iv) malonyl-CoA synthase having an amino acid sequence at least 80% identical SEQ ID NO: 77, SEQ ID NO: 78, or SEQ ID NO: 79; (v) malonate transporter having an amino acid sequence at least 80% identical to SEQ ID NO: 80, SEQ ID NO: 81, SEQ ID NO: 82, SEQ ID NO: 83, SEQ ID NO: 84, SEQ ID NO: 85, SEQ ID NO: 86, or SEQ ID NO: 87; (vi) pantothenate kinase having an amino acid sequence at least 80% identical to SEQ ID NO: 88, SEQ ID NO: 89, or SEQ ID NO: 90; and (vii) any combinations thereof.

Step 7: conversion of mal-CoA to malonyl-ACP (acyl carrier protein). malonyl-coA-ACP transacylase (fabD) is downregulated to increase carbon flux.

Step 8: conversion of malonyl-ACP to 3-ketyoacyl-ACP. beta-ketoacyl-ACP synthase II (fabF) is downregulated to increase carbon flux.

Step 9: conversion to mal-CoA to naringenin chalcone; conversion of coumaryl-CoA to naringenin chalcone. A heterologous CHS is overexpressed.

Step 10: conversion to naringenin chalcone to naringenin. A heterologous CHI is overexpressed.

Steps 11, 12, and 13: conversion of naringenin to dihydrokaempferol (DHK); conversion of naringenin to eriodictyol (EDL); conversion of eriodictyol (EDL) to dihydroquercetin (DHQ); conversion of (DHK) to dihydroquercetin (DHQ); conversion of dihydrokaempferol (DHK) to dihydromyricetin (DHM); conversion of pentahydroxyflayaone (PHF) to dihydromyricein (DHM). Heterologous F3′5′H, F3H, F3H, and/or CPR are overexpressed. Accordingly, as shown in FIG. 4, in another aspect, the invention provides method of increasing the production of dihydroquercetin (DHQ), dihydromyricein (DHM), eriodictyol (EDL), and/or pentahydroxyflayaone (PHF) comprising an engineered host cell, wherein the engineered host cell comprises cytochrome P450 reductase (CPR) and at least one of flavanone-3′-hydroxylase (F3′H) or flavonoid 3′,5′-hydroxylase (F3′5′H). In certain embodiments, the precursor for increase in production of dihydroquercetin (DHQ), dihydromyricein (DHM), eriodictyol (EDL), and/or pentahydroxyflavone (PTF) is naringenin and/or dihydrokaempferol (DHK). In certain embodiments, the engineered host cell further comprises peptides selected from a group consisting of: (i) flavonoid 3′-hydroxylase (F3′H); (ii) cytochrome P450 reductase (CPR); and (iii) any combination thereof. In certain embodiments, the engineered host cell produces eriodictyol or taxifolin. In certain embodiments, the engineered host cell further comprises flavonoid 3′,5′-hydroxylase (F3′5′H). In certain embodiments, the engineered host cell produces pentahydroxyflavone or dihydromyricetin. In certain embodiments, flavonoid 3′-hydroxylase (F3′H) is truncated to remove the N-terminal leader sequence. In certain embodiments, cytochrome P450 reductase (CPR) is truncated to remove the N-terminal leader sequence. In certain embodiments, flavonoid 3′-hydroxylase (F3′H) is fused with cytochrome P450 reductase (CPR). In certain embodiments, flavonoid 3′,5′-hydroxylase (F3′5′H) is fused with cytochrome P450 reductase (CPR). In certain embodiments, flavanone-3-hydroxylase (F3H) has an amino acid sequence at least 80% identical to the polypeptide set forth in SEQ ID NO. 7. In certain embodiments, flavanone-3′-hydroxylase (F3′H) has an amino acid sequence at least 80% identical to the polypeptide set forth in SEQ ID NO. 8. In certain embodiments, cytochrome P450 reductase (CPR) has an amino acid sequence at least 80% identical to the polypeptide set forth in SEQ ID NO. 9. In certain embodiments, flavonoid 3′,5′-hydroxylase (F3′5′H) has an amino acid sequence at least 80% identical to the polypeptides selected from a group consisting of: (i) SEQ ID NO. 10, (ii) SEQ ID NO. 56, and (iii) SEQ ID NO. 57. In certain embodiments, the engineered host cell further comprises cytochrome b₅. In certain embodiments, cytochrome b₅ has an amino acid sequence at least 80% identical to the polypeptide set forth in SEQ ID NO. 98.

As shown in FIG. 4, in another aspect, the invention provides method of increasing the production of dihydroquercetin (DHQ), dihydromyricein (DHM), eriodictyol (EDL), and/or pentahydroxyflayaone (PHF) comprising an engineered host cell, wherein the engineered host cell comprises cytochrome P450 reductase (CPR) and at least one of flavanone-3′-hydroxylase (F3′H) or flavonoid 3′,5′-hydroxylase (F3′5′H). In certain embodiments, the precursor for increase in production of dihydroquercetin (DHQ), dihydromyricein (DHM), eriodictyol (EDL), and/or pentahydroxyflavone (PHF) is naringenin and/or dihydrokaempferol (DHK). In certain embodiments, the engineered host cell further comprises peptides selected from a group consisting of: (i) flavonoid 3′-hydroxylase (F3′H); (ii) cytochrome P450 reductase (CPR); and (iii) any combination thereof. In certain embodiments, the engineered host cell produces eriodictyol or taxifolin. In certain embodiments, the engineered host cell further comprises flavonoid 3′,5′-hydroxylase (F3′5′H). In certain embodiments, the engineered host cell produces pentahydroxyflavone or dihydromyricetin. In certain embodiments, flavonoid 3′-hydroxylase (F3′H) is truncated to remove the N-terminal leader sequence. In certain embodiments, cytochrome P450 reductase (CPR) is truncated to remove the N-terminal leader sequence. In certain embodiments, flavonoid 3′-hydroxylase (F3′H) is fused with cytochrome P450 reductase (CPR). In certain embodiments, flavonoid 3′,5′-hydroxylase (F3′5′H) is fused with cytochrome P450 reductase (CPR). In certain embodiments, flavanone-3-hydroxylase (F3H) has an amino acid sequence at least 80% identical to the polypeptide set forth in SEQ ID NO. 7. In certain embodiments, flavanone-3′-hydroxylase (F3′H) has an amino acid sequence at least 80% identical to the polypeptide set forth in SEQ ID NO. 8. In certain embodiments, cytochrome P450 reductase (CPR) has an amino acid sequence at least 80% identical to the polypeptide set forth in SEQ ID NO. 9. In certain embodiments, flavonoid 3′,5′-hydroxylase (F3′5′H) has an amino acid sequence at least 80% identical to the polypeptides selected from a group consisting of: (i) SEQ ID NO. 10, (ii) SEQ ID NO. 56, and (iii) SEQ ID NO. 57. In certain embodiments, the engineered host cell further comprises cytochrome b₅. In certain embodiments, cytochrome b₅ has an amino acid sequence at least 80% identical to the polypeptide set forth in SEQ ID NO. 98.

Step 14: conversion of dihydroquercetin (DHQ) to leucocyanidin (LC); conversion of dihydrokaempferol (DHK) to leucopelargonidin (LP); and conversion of dihydromyricetin (DHM) to leucodelphinidin (LD). Heterologous DFR is overexpressed.

Step 15: conversion of leucocyanidin (LC) to catechin; conversion of leucodelphinidin (LD) to gallocatechin; and conversion of leucopelargonidin (LP) to afzelechin. Heterologous LAR is overexpressed.

Step 16: conversion of catechin to cyanidin; conversion of leucocyanidin (LC) to catechin; conversion to leucodelphinidin (LD) to delphinidin; conversion of gallocatechin to delphinidin; conversion of leucopelargonidin (LP) to pelargonidin; or conversion of afzelechin to pelargonidin. Heterologous ANS is overexpressed. Step 16 could be carried in vivo or in a cell-free medium. Accordingly, as shown in FIG. 4, in another aspect, the invention provides an engineered host cell, wherein the engineered host cell comprises one or more genetic modifications to increase transformation of leucocyanidin or catechin to cyanidin-3-glucoside (Cy3G). In certain embodiments, one or more genetic modifications comprises overexpression of anthocyanin synthase. In certain embodiments, the anthocyanin synthase is selected from: (i) anthocyanin synthase of Carica papaya (SEQ. ID NO:13); (ii) the anthocyanin synthase has an amino acid sequence at least 80% identical to SEQ. ID NO: 66, SEQ. ID NO: 67, SEQ. ID NO: 68, or SEQ. ID NO: 69; (iii) the anthocyanin synthase has an amino acid sequence at least 80% identical to SEQ. ID NO: 13; and (iv) any combinations thereof. In certain embodiments, one or more engineered host cells comprises flavonoid-3-glucosyl transferase (3GT). In certain embodiments, flavonoid-3-glucosyl transferase is selected from: (i) flavonoid-3-glucosyl transferase in Vitis labrusca (SEQ. ID NO:14); (ii) the flavonoid-3-glucosyl transferase has an amino acid sequence at least 80% identical to SEQ. ID NO: 70, SEQ. ID NO: 71, SEQ. ID NO: 72, or SEQ. ID NO: 73; and (iii) any combinations thereof. In certain embodiments, one or more genetic modifications comprises overexpression of anthocyanin synthase and flavonoid-3-glucosyl transferase (3GT). In certain embodiments, one or more genetic modifications comprises overexpression of anthocyanin synthase and flavonoid-3-glucosyl transferase (3GT). In certain embodiments, the one or more genetic modifications comprises overexpression of anthocyanin synthase and flavonoid-3-glucosyl transferase (3GT). In certain embodiments, the one or more genetic modifications are selected from a group consisting of: (i) anthocyanin synthase, (ii) flavonoid-3-glucosyl transferase (3GT), and (iii) a combination thereof.

In another aspect, the invention provides a method for increasing the production of flavonoids comprising an engineered host cell, wherein the engineered host cell comprises one or more genetic modifications to increase transformation of leucocyanidin or catechin to cyanidin-3-glucoside (Cy3G). In certain embodiments, one or more genetic modifications comprises overexpression of anthocyanin synthase. In certain embodiments, the anthocyanin synthase is selected from: (i) anthocyanin synthase of Carica papaya (SEQ. ID NO:13); (ii) the anthocyanin synthase has an amino acid sequence at least 80% identical to SEQ. ID NO: 66, SEQ. ID NO: 67, SEQ. ID NO: 68, or SEQ. ID NO: 69; (iii) the anthocyanin synthase has an amino acid sequence at least 80% identical to SEQ. ID NO: 13; and (iv) any combinations thereof. In certain embodiments, one or more engineered host cells comprises flavonoid-3-glucosyl transferase (3GT). In certain embodiments, flavonoid-3-glucosyl transferase is selected from: (i) flavonoid-3-glucosyl transferase in Vitis labrusca (SEQ. ID NO:14); (ii) the flavonoid-3-glucosyl transferase has an amino acid sequence at least 80% identical to SEQ. ID NO: 70, SEQ. ID NO: 71, SEQ. ID NO: 72, or SEQ. ID NO: 73; and (iii) any combinations thereof. In certain embodiments, one or more genetic modifications comprises overexpression of anthocyanin synthase and flavonoid-3-glucosyl transferase (3GT). In certain embodiments, one or more genetic modifications comprises overexpression of anthocyanin synthase and flavonoid-3-glucosyl transferase (3GT). In certain embodiments, the one or more genetic modifications comprises overexpression of anthocyanin synthase and flavonoid-3-glucosyl transferase (3GT). In certain embodiments, the one or more genetic modifications are selected from a group consisting of: (i) anthocyanin synthase, (ii) flavonoid-3-glucosyl transferase (3GT), and (iii) a combination thereof.

In another aspect, the invention provides a method of increasing the transformation of leucocyanidin or catechin to cyanidin-3-glucoside (Cy3G) comprising anthocyanin synthase, wherein the anthocyanin synthase is selected from: (i) anthocyanin synthase of Carica papaya (SEQ. ID NO:13); (ii) the anthocyanin synthase has an amino acid sequence at least 80% identical to SEQ. ID NO: 66, SEQ. ID NO: 67, SEQ. ID NO: 68, or SEQ. ID NO: 69; (iii) the anthocyanin synthase has an amino acid sequence at least 80% identical to SEQ. ID NO: 13; and (iv) any combinations thereof.

In another aspect, the invention provides a method of increasing the transformation of leucocyanidin or catechin to cyanidin-3-glucoside (Cy3G) comprising flavonoid-3-glucosyl transferase (3GT), wherein the flavonoid-3-glucosyl transferase is selected from: (i) flavonoid-3-glucosyl transferase in Vitis labrusca (SEQ. ID NO:14); (ii) the flavonoid-3-glucosyl transferase has an amino acid sequence at least 80% identical to SEQ. ID NO: 70, SEQ. ID NO: 71, SEQ. ID NO: 72, or SEQ. ID NO: 73; and (iii) any combinations thereof.

Step 17: conversion of pelargonidin to callistephin; conversion of delphinidin to myrtillin (De3G); conversion of cyanidin to Cy3G. Heterologous 3GT was overexpressed in E. coli. Step 17 could be carried in vivo or as a cell-free reaction.

Step 18: conversion of pyruvate to phosphoenolpyruvate (PEP). ppsA is overexpressed to upregulate tyrosine.

Step 19: conversion of fructose-6-phosphate (F6P) to erythrose-4-phosphate (E4P). tktA is overexpressed to upregulate tyrosine.

Step 20: conversion of phosphoenolpyruvate (PEP) to deoxy-d-arabino-heptulosonate-7-phosphate (DAHP). aroG variant is overexpressed to upregulate tyrosine.

Step 21: conversion of deoxy-d-arabino-heptulosonate-7-phosphate (DAHP) to dehydroquinate (DHQ); conversion of erythrose-4-phosphate (E4P) to dehydroquinate (DHQ).

Step 22: conversion of dehydroquinate (DHQ) to 3-dehydroshikimate (DHS).

Step 23: conversion of 3-dehydroshikimate (DHS) to shikimic acid (SHK). aroE is overexpressed to upregulate tyrosine.

Step 24: conversion of shikimic acid (SHK) to shikimate-3-phosphate (S3P).

Step 25: conversion of shikimate-3-phosphate (S3P) to 5-enolpyruvylshikimate-3-phosphate (EPSP).

Step 26: conversion of 5-enolpyruvylshikimate-3-phosphate (EPSP) to chorismic acid (CHA).

Step 27: conversion of chorismic acid (CHA) to prephenate (PPA); conversion of prephenate (PPA) to 4-hydroxy-phenylpyruvate (HPP). tryA variant is overexpressed.

Step 28: conversion of 4-hydroxy-phenylpyruvate (HPP) to tyrosine; conversion of phenylpyruvate (POPP) to phenylalanine (Phe). Accordingly, as shown in FIG. 4, embodiments of the invention provide an engineered host cell, wherein the engineered host cell comprises one or more genetic modifications to increase endogenous biosynthesis of tyrosine. In certain embodiments, one or more genetic modifications comprises upregulation of 3-deoxy-D-arabino-heptulosonate synthase. In certain embodiments, one or more genetic modifications are selected from: (i) upregulation of chorismate mutase; (ii) upregulation of prephenate dehydrogenase; (iii) overexpression of shikimate kinase; (iv) overexpression of shikimate dehydrogenase; and (v) any combinations thereof. In certain embodiments, one or more genetic modifications comprises downregulation of L-phenylalanine biosynthetic pathway. In certain embodiments, one or more genetic modifications comprises expression of exogenous phosphoenolpyruvate synthase (ppsA). In certain embodiments, one or more genetic modifications comprises expression of exogenous transketolase (tktA). In certain embodiments, wherein the one or more genetic modifications comprises disruption of tyrR gene.

As shown in FIG. 4, in another aspect, the invention provides a method of increasing endogenous biosynthesis of tyrosine comprising an engineered cell, wherein the engineered host cell comprises one or more genetic modifications to increase endogenous biosynthesis of tyrosine. In certain embodiments, one or more genetic modifications comprises upregulation of 3-deoxy-D-arabino-heptulosonate synthase. In certain embodiments, one or more genetic modifications are selected from: (i) upregulation of chorismate mutase; (ii) upregulation of prephenate dehydrogenase; (iii) overexpression of shikimate kinase; (iv) overexpression of shikimate dehydrogenase; and (v) any combinations thereof. In certain embodiments, one or more genetic modifications comprises downregulation of L-phenylalanine biosynthetic pathway. In certain embodiments, one or more genetic modifications comprises expression of exogenous phosphoenolpyruvate synthase (ppsA). In certain embodiments, one or more genetic modifications comprises expression of exogenous transketolase (tktA). In certain embodiments, wherein the one or more genetic modifications comprises disruption of tyrR gene.

Step 29: conversion of tyrosine to coumaric acid. A heterologous TAL is overexpressed.

Step 30: conversion of courmaric acid to coumaryl-CoA. A heterologous 4CL is overexpressed.

Step 31: conversion of glutamate (Glut) to glutamyl-tRNA.

Step 32: conversion of glutamyl-tRNA to glutamate semialdehyde (GSA). hemA is overexpressed to upregulate ALA.

Step 33: conversion of glutamate semialdehyde (GSA) to 6 amino levulinic acid (ALA). hemL is overexpressed to upregulate ALA.

Step 34: conversion of 6 amino levulinic acid (ALA) to porphobilinogen (PBG).

Step 35: conversion of porphobilinogen (PBG) to hydroxymethylbilane (HMB).

Step 36: conversion of hydroxymethylbilane (HMB) to uroporphyrinogen III (UPPIII).

Step 37: conversion of uroporphyrinogen III (UPPIII) to coproporphyrinogen III (CPPIII).

Step 38: conversion of coproporphyrinogen III (CPPIII) to protoporphyrinogen IX (PPPIX).

Step 39: conversion of protoporphyrinogen IX (PPPIX) to protoporphyrin IX, which is subsequently covered to heme.

Step 40: conversion of prephenate (PPA) to phenylpyruvate (POPP).

Step 41: conversion of phenylalanine (Phe) to cinnamate. Heterologous PAL and/or TAL are overexpressed.

Step 42: conversion of cinnamate to coumaric acid. Heterologous C4H/CPR are overexpressed.

As shown in FIG. 5, in another aspect, the invention provides engineered cells for increasing production of kaempferol, myricetin, and quercetin through transformation by one or more enzymes of a carbon source by the engineered host cell. As evident from FIG. 5, the enzymatic transformations described in FIG. 5 provide additional transformations to those provided in FIG. 4. The enzymatic transformations in FIG. 4 are also discussed in U.S. application Ser. No. 17/720,020, which is hereby incorporated by reference in its entirety. In one embodiment, the carbon source is selected from a group consisting of naringenin, dihydrokaempferol (DHK), dihydromyricetin (DHM), dihydroquercetin (DHQ), and eriodictyol (EDL). In another embodiment, one or more genetic modifications are at least one genetic modification selected from the group consisting of: (i) one or more modifications for over-expressing one or more endogenous genes in the engineered host cells; (ii) one or more modifications for under-expressing one or more endogenous genes in the engineered host cells; (iii) one or more genetic modification is expressing one or more non-native genes in the engineered host cells; and (iv) a combination thereof. In another embodiment, the engineered host cell comprises at least one or more nucleic acid sequences selected from the group consisting of: (i) nucleic acid sequences encoding native or modified flavanone-3-hydroxylase (F3H) or a homolog thereof, (ii) nucleic acid sequences encoding native or modified flavanone-3′-hydroxylase (F3′H) or a homolog thereof, (iii) nucleic acid sequences encoding native or modified flavonoid 3′,5′-hydroxylase (F3′5′H) or a homolog thereof, (iv) nucleic acid sequences encoding native or modified flavonol synthase (FLS) or a homolog thereof, and (v) any combinations thereof. In another embodiment, the engineered host cell is E. coli. In another embodiment, the production of flavonols through enzymatic transformation of a carbon source includes one or more chemical intermediates. In another embodiment, the flavonol is myricetin, the carbon source is dihydromyricetin (DHM), and the one or more enzyme is flavonol synthase (FLS). In another embodiment, the flavonol is myricetin, the carbon source is dihydrokaempferol (DHK), and the one or more enzymes are selected from a group consisting of (i) flavonoid 3′,5′-hydroxylase (F3′5′H), (ii) flavonol synthase (FLS), and (iii) any combination thereof. In another embodiment, flavonoid 3′,5′-hydroxylase (F3′5′H) has an amino acid sequence at least 80% identical to the polypeptides selected from a group consisting of: (i) SEQ ID NO. 10, (ii) SEQ ID NO. 56, and (iii) SEQ ID NO. 57. In another embodiment, the flavonol is kaempferol, the carbon source is dihydrokaempferol (DHK), and the one or more enzyme is flavonol synthase (FLS). In another embodiment, flavonol synthase (FLS) has an amino acid sequence at least 80% identical to any one of the polypeptides listed in SEQ ID NOS. 99-122. In another embodiment, the flavonol is quercetin, the carbon source is eriodictoyl (EDL), and the one or more enzyme is selected from a group consisting of (i) flavanone-3-hydroxylase (F3H), (ii) flavonol synthase (FLS), and (iii) any combination thereof. In another embodiment, the flavanone-3-hydroxylase (F3H) has an amino acid sequence at least 80% identical to the polypeptides selected from a group consisting of: (i) SEQ ID NO. 7, (ii) SEQ ID NO. 45, (iii) SEQ ID NO. 46, (iv) SEQ ID NO. 47, and (v) SEQ ID NO. 48. In another embodiment, production of quercetin leads to formation of dihydroquercetin (DHQ) as an intermediate. In another embodiment, the flavonol is quercetin, the carbon source is dihydroquercetin (DHQ) and the one or more enzyme is flavonol synthase (FLS). In another embodiment, the flavonol is quercetin, the carbon source is kaempferol, and the enzyme is flavanone-3′-hydroxylase (F3′H). In another embodiment, flavanone-3′-hydroxylase (F3′H) has an amino acid sequence at least 80% identical to the polypeptide set forth in SEQ ID NO: 8. In another embodiment, the flavonol is kaempferol, the carbon source is dihydrokaempferol (DHK), naringenin, or any combination thereof, and the one or more enzyme is selected from a group consisting of: (i) flavanone-3′-hydroxylase (F3′H), (ii) flavonol synthase (FLS), and (iii) any combination thereof. In another embodiment, the flavonol is quercetin, the carbon source is dihydroquerctin (DHQ), dihydrokaempferol, eriodictyol (EDL), naringenin, kaempferol, or any combination thereof, and the one or more enzyme is selected from a group consisting of: (i) flavonol synthase (FLS), (ii) flavanone-3′-hydroxylase (F3′H), (iii) flavanone-3-hydroxylase (F3H), and (iv) any combination thereof. In another embodiment, the flavonol is myricetin, the carbon source is dihydromyricetin (DHM), dihydroquerctin (DHQ), dihydrokaempferol, eriodictyol (EDL), naringenin, quercetin, kaempferol, or any combination thereof, and the one or more enzyme is selected from a group consisting of (i) flavonol synthase (FLS), (ii) flavanone-3′-hydroxylase (F3′H), (iii) flavanone-3-hydroxylase (F3H), (iv) flavonoid-3′-5′-hydroxylase (F3′5′H), and (v) any combination thereof.

In another aspect, the invention provides a method of increasing the production of flavonols, comprising engineered host cells, wherein the engineered host cells comprise one or more genetic modifications to increase the production of flavonols through transformation by one or more enzymes of a carbon source by the engineered host cell. In one embodiment, the flavonol is selected from a group consisting of kaempferol, myricetin, and quercetin. In another embodiment, the carbon source is selected from a group consisting of naringenin, dihydrokaempferol (DHK), dihydromyricetin (DHM), dihydroquercetin (DHQ), and eriodictyol (EDL). In another embodiment, one or more genetic modifications are at least one genetic modification selected from the group consisting of: (i) one or more modifications for over-expressing one or more endogenous genes in the engineered host cells; (ii) one or more modifications for under-expressing one or more endogenous genes in the engineered host cells; (iii) one or more genetic modification is expressing one or more non-native genes in the engineered host cells; and (iv) a combination thereof. In another embodiment, the engineered host cell comprises at least one or more nucleic acid sequences selected from the group consisting of: (i) nucleic acid sequences encoding native or modified flavanone-3-hydroxylase (F3H) or a homolog thereof, (ii) nucleic acid sequences encoding native or modified flavanone-3′-hydroxylase (F3′H) or a homolog thereof, (iii) nucleic acid sequences encoding native or modified flavonoid 3′,5′-hydroxylase (F3′5′H) or a homolog thereof, (iv) nucleic acid sequences encoding native or modified flavonol synthase (FLS) or a homolog thereof, and (v) any combinations thereof. In another embodiment, the engineered host cell is E. coli. In another embodiment, the production of flavonols through enzymatic transformation of a carbon source includes one or more chemical intermediates. In another embodiment, the flavonol is myricetin, the carbon source is dihydromyricetin (DHM), and the one or more enzyme is flavonol synthase (FLS). In another embodiment, the flavonol is myricetin, the carbon source is dihydrokaempferol (DHK), and the one or more enzymes are selected from a group consisting of (i) flavonoid 3′,5′-hydroxylase (F3′5′H), (ii) flavonol synthase (FLS), and (iii) any combination thereof. In another embodiment, flavonoid 3′,5′-hydroxylase (F3′5′H) has an amino acid sequence at least 80% identical to the polypeptides selected from a group consisting of: (i) SEQ ID NO. 10, (ii) SEQ ID NO. 56, and (iii) SEQ ID NO. 57. In another embodiment, the flavonol is kaempferol, the carbon source is dihydrokaempferol (DHK), and the one or more enzyme is flavonol synthase (FLS). In another embodiment, flavonoid synthase (FLS) has an amino acid sequence at least 80% identical to any one of the polypeptides selected from a group consisting of: SEQ ID NOS: 99-122. In another embodiment, the flavonol is quercetin, the carbon source is eriodictoyl (EDL), and the one or more enzyme is selected from a group consisting of (i) flavanone-3-hydroxylase (F3H), (ii) flavonol synthase (FLS), and (iii) any combination thereof. In another embodiment, the flavanone-3-hydroxylase (F3H) has an amino acid sequence at least 80% identical to the polypeptides selected from a group consisting of: (i) SEQ ID NO. 7, (ii) SEQ ID NO. 45, (iii) SEQ ID NO. 46, (iv) SEQ ID NO. 47, and (v) SEQ ID NO. 48. In another embodiment, production of quercetin leads to formation of dihydroquercetin (DHQ) as an intermediate. In another embodiment, the flavonol is quercetin, the carbon source is dihydroquercetin (DHQ) and the one or more enzyme is flavonol synthase (FLS). In another embodiment, the flavonol is quercetin, the carbon source is kaempferol, and the enzyme is flavanone-3′-hydroxylase (F3′H). In another embodiment, flavanone-3′-hydroxylase (F3′H) has an amino acid sequence at least 80% identical to the polypeptide set forth in SEQ ID NO: 8. In another embodiment, the flavonol is kaempferol, the carbon source is dihydrokaempferol (DHK), naringenin, or any combination thereof, and the one or more enzyme is selected from a group consisting of: (i) flavanone-3′-hydroxylase (F3′H), (ii) flavonol synthase (FLS), and (iii) any combination thereof. In another embodiment, the flavonol is quercetin, the carbon source is dihydroquerctin (DHQ), dihydrokaempferol, eriodictyol (EDL), naringenin, kaempferol, or any combination thereof, and the one or more enzyme is selected from a group consisting of: (i) flavonol synthase (FLS), (ii) flavanone-3′-hydroxylase (F3′H), (iii) flavanone-3-hydroxylase (F3H), and (iv) any combination thereof. In another embodiment, the flavonol is myricetin, the carbon source is dihydromyricetin (DHM), dihydroquerctin (DHQ), dihydrokaempferol, eriodictyol (EDL), naringenin, quercetin, kaempferol, or any combination thereof, and the one or more enzyme is selected from a group consisting of (i) flavonol synthase (FLS), (ii) flavanone-3′-hydroxylase (F3′H), (iii) flavanone-3-hydroxylase (F3H), (iv) flavonoid-3′-5′-hydroxylase (F3′5′H), and (v) any combination thereof.

FIG. 5 shows the enzymatic conversion of dihydromyricetin (DHM) to myricetin through flavonol synthase (FLS). FIG. 5 also shows the enzymatic conversion of kaempferol and quercetin to myricetin through flavonoid 3′,5′-hydroxylase (F3′5′H). FIG. 5 further shows the enzymatic conversion of dihydrokaempferol (DHK) to kaempferol through flavonol synthase (FLS). Finally, FIG. 5 shows the enzymatic conversion of kaempferol to quercetin through flavanone-3′-hydroxylase (F3′H) and enzymatic conversion of dihydroquercetin (DHQ) to quercetin through flavonol synthase (FLS).

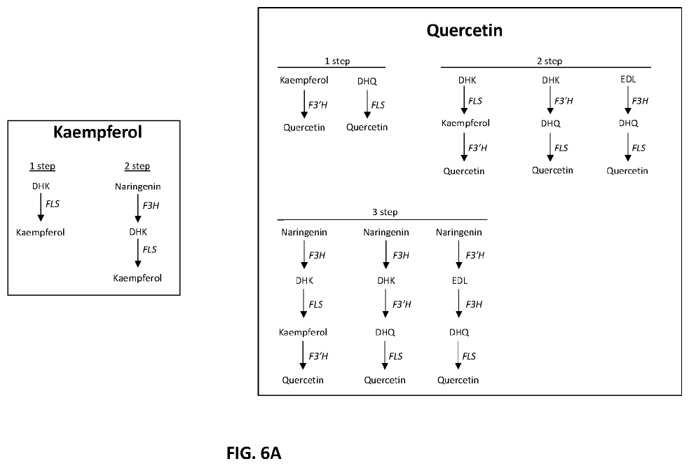

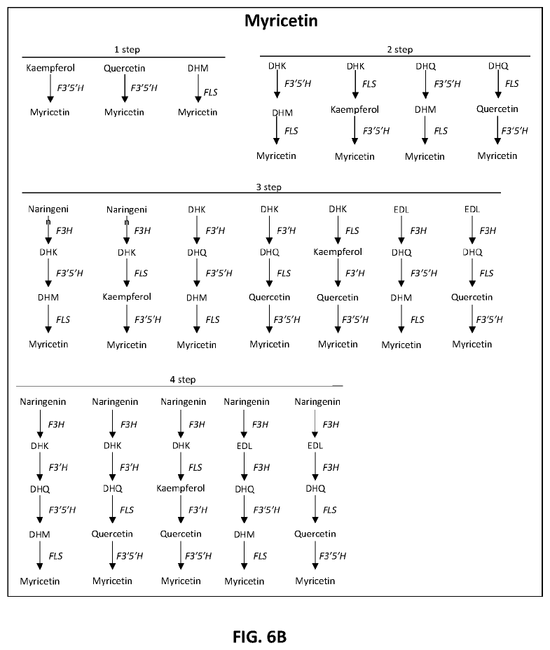

FIGS. 6A and 6B show several enzymatic pathways for bioproduction of kaempferol, quercetin, and myricetin. As evident from FIGS. 6A and 6B, the invention provides several pathways involving transformation of various precursors to kaempferol, quercetin, and myricetin.

TABLE 11
Enzyme Sequences:

| Enzyme: | Sequence: | SEQ ID: |
|---|---|---|
| Tyrosine ammonia- | MTQVVERQADRLSSREYLARVVRSAGWDAGLTSCTD | 1 |
| lyase (TAL) | EEIVRMGASARTIEEYLKSDKPIYGLTQGFGPLVLFDA | |
| Saccharothrix | DSELEQGGSLISHLGTGQGAPLAPEVSRLILWLRIQNM | |
| espanaensis | RKGYSAVSPVFWQKLADLWNKGFTPAIPRHGTVSAS | |
| Accession: | GDLQPLAHAALAFTGVGEAWTRDADGRWSTVPAVD | |
| ABC88669.1 | ALAALGAEPFDWPVREALAFVNGTGASLAVAVLNHR | |
| | SALRLVRACAVLSARLATLLGANPEHYDVGHGVARG | |
| | QVGQLTAAEWIRQGLPRGMVRDGSRPLQEPYSLRCA | |
| | PQVLGAVLDQLDGAGDVLAREVDGCQDNPITYEGEL | |
| | LHGGNFHAMPVGFASDQIGLAMHMAAYLAERQLGL | |
| | LVSPVTNGDLPPMLTPRAGRGAGLAGVQISATSFVSRI | |
| | RQLVFPASLTTLPTNGWNQDHVPMALNGANSVFEAL | |
| | ELGWLTVGSLAVGVAQLAAMTGHAAEGVWAELAGI | |
| | CPPLDADRPLGAEVRAARDLLSAHADQLLVDEADGK | |
| | DFG | |
| Phenylalanine | MSQVALFEQELMLHGKHTLLLNGNDLTITDVAQMAK | 2 |
| ammonia-lyase | GTFEAFTFHISEEANKRIEECNELKHEIMNQHNPIYGV | |
| (PAL) | TTGFGDSVHRQISGEKAWDLQRNLIRFLSCGVGPVAD | |
| Brevibacillus | EAVARATMLIRTNCLVKGNSAVRLEVIHQLIAYMERG | |
| laterosporus LMG | ITPIIPERGSVGASGDLVPLSYLASILVGEGKVLYKGEE | |
| 15441 | REVAEALGAEGLEPLTLEAKEGLALVNGTSFMSAFAC | |
| Accession: | LAYADAEEIAFIADICTAMASEALLGNRGHFYSFIHEQ | |
| WP_003337219.1 | KPHLGQMASAKNIYTLLEGSQLSKEYSQIVGNNEKLD | |
| | SKAYLELTQSIQDRYSIRCAPHVTGVLYDTLDWVKK | |
| | WLEVEINSTNDNPIFDVETRDVYNGGNFYGGHVVQA | |
| | MDSLKVAVANIADLLDRQLQLVVDEKFNKDLTPNLIP | |
| | RFNNDNYEIGLHHGFKGMQIASSALTAEALKMSGPVS | |
| | VFSRSTEAHNQDKVSMGTISSRDARTIVELTQHVAAIH | |
| | LIALCQALDLRDSKKMSPQTTKIYNMIRKQVPFVERD | |
| | RALDGDIEKVVQLIRSGNLKKEIHDQNVND | |
| Cinnamate-4- | MDLLLIEKTLLALFAAIIGAIVISKLRGKRFKLPPGPLP | 3 |
| hydroxylase (C4H) | VPIFGNWLQVGDDLNHRNLTDLAKKFGEIFLLRMGQ | |
| Helianthus annuus | RNLVVVSSPDLAKEVLHTQGVEFGSRTRNVVFDIFTG | |
| L. | KGQDMVFTVYGEHWRKMRRIMTVPFFTNKVVQQYR | |
| Accession: | YGWEAEAAAVVEDVKKNPAAATEGVVIRRRLQLMM | |
| QJC72299.1 | YNNMFRIMFDRRFESEDDPLFVKLKALNGERSRLAQS | |
| | FEYNYGDFIPILRP | |
| | FLKGYLKLCKEVKEKRFQLFKDYFVDERKKLESTKSV | |
| | DNNQLKCAIDHILDAKEKGEINEDNVLYIVENINVAAI | |
| | ETTLWSIEWGIAELVNHPEIQAKLRNELDTKLGPGVQ | |
| | VTEPDLHKLPYLQAVIKETLRLRMAIPLLVPHMNLHD | |
| | AKLGGYDIPAESKILVNAWWLANNPEQWKKPEEFRP | |
| | ERFFEEESKVEANGNDFRYLPFGVGRRSCPGIILALPIL | |
| | GITIGRLVQNFELLPPPGQSKVDTTEKGGQFSLHILKHS | |
| | TIVAKPRAL | |
| 4-coumarate-CoA | MGDCVAPKEDLIFRSKLPDIYIPKHLPLHTYCFENISKV | 4 |
| ligase (4CL) | GDKSCLINGATGETFTYSQVELLSRKVASGLNKLGIQ | |
| Petroselinum | QGDTIMLLLPNSPEYFFAFLGASYRGAISTMANPFFTS | |
| crispum | AEVIKQLKASQAKLIITQACYVDKVKDYAAEKNIQIIC | |
| Accession: | IDDAPQDCLHFSKLMEADESEMPEVVINSDDVVALPY | |
| P14912.1 | SSGTTGLPKGVMLTHKGLVTSVAQQVDGDNPNLYM | |
| | HSEDVMICILPLFHIYSLNAVLCCGLRAGVTILIMQKF | |
| | DIVPFLELIQKYKVTIGPFVPPIVLAIAKSPVVDKYDLS | |
| | SVRTVMSGAAPLGKELEDAVRAKFPNAKLGQGYGM | |
| | TEAGPVLAMCLAFAKEPYEIKSGACGTVVRNAEMKIV | |
| | DPETNASLPRNQRGEICIRGDQIMKGYLNDPESTRTTI | |
| | DEEGWLHTGDIGFIDDDDELFIVDRLKEIIKYKGFQVA | |
| | PAELEALLLTHPTISDAAVVPMIDEKAGEVPVAFVVRT | |
| | NGFTTTEEEIKQFVSKQVVFYKRIFRVFFVDAIPKSPSG | |
| | KILRKDLRARIASGDLPK | |
| Chalcone synthase | MVTVEEYRKAQRAEGPATVMAIGTATPTNCVDQSTY | 5 |
| (CHS) | PDYYFRITNSEHKTDLKEKFKRMCEKSMIKKRYMHLT | |
| Petunia x hybrida | EEILKENPSMCEYMAPSLDARQDIVVVEVPKLGKEAA | |
| Accession: | QKAIKEWGQPKSKITHLVFCTTSGVDMPGCDYQLTKL | |
| AAF60297.1 | LGLRPSVKRLMMYQQGCFAGGTVLRLAKDLAENNK | |
| | GARVLVVCSEITAVTFRGPNDTHLDSLVGQALFGDGA | |
| | GAIIIGSDPIPGVERPLFELVSAAQTLLPDSHGAIDGHL | |
| | REVGLTFHLLKDVPGLISKNIEKSLEEAFRPLSISDWNS | |
| | LFWIAHPGGPAILDQVEIKLGLKPEKLKATRNVLSNY | |
| | GNMSSACVLFILDEMRKASAKEGLGTTGEGLEWGVL | |
| | FGFGPGLTVETVVLHSVAT | |
| Chalcone isomerase | MAASITAITVENLEYPAVVTSPVTGKSYFLGGAGERG | 6 |
| (CHI) | LTIEGNFIKFTAIGVYLEDIAVASLAAKWKGKSSEELL | |
| Medicago sativa | ETLDFYRDIISGPFEKLIRGSKIRELSGPEYSRKVMENC | |
| Accession: | VAHLKSVGTYGDAEAEAMQKFAEAFKPVNFPPGASV | |
| P28012.1 | FYRQSPDGILGLSFSPDTSIPEKEAALIENKAVSSAVLE | |
| | TMIGEHAVSPDLKRCLAARLPALLNEGAFKIGN | |
| Flavanone 3- | MAPTPTTLTAIAGEKTLQQSFVRDEDERPKVAYNQFS | 7 |
| hydroxylase (F3H) | NEIPIISLSGIDEVEGRRAEICNKIVEACEDWGVFQIVD | |
| Rubus occidentalis | HGVDAKLISEMTRLARDFFALPPEEKLRFDMSGGKKG | |
| Accession: | GFIVSSHLQGEAVQDWREIVTYFSYPVRHRDYSRWPD | |
| ACM17897.1 | KPEGWRAVTQQYSDELMGLACKLLEVLSEAMGLEKE | |
| | ALTKACVDMDQKVVVNFYPKCPQPDLTLGLKRHTDP | |
| | GTITLLLQDQVGGLQATRDGGKTWITVQPVEGAFVV | |
| | NLGDHGHFLSNGRFKNADHQAVVNSNHSRLSIATFQ | |
| | NPAQEAIVYPLKVREGEKPILEEPITYTEMYKKKMSK | |
| | DLELARLKKLAKEQQPEDSEKAKLEVKQVDDIFA | |
| Flavonoid 3′ | MTNLYLTILLPTFIFLIVLVLSRRRNNRLPPGPNPWPIIG | 8 |
| hydroxylase (F3′H) | NLPHMGPKPHQTLAAMVTTYGPILHLRLGFADVVVA | |
| Brassica napus | ASKSVAEQFLKVHDANFASRPPNSGAKHMAYNYQDL | |
| Accession: | VFAPYGQRWRMLRKISSVHLFSAKALEDFKHVRQEE | |
| ABC58723.1 | VGTLMRELARANTKPVNLGQLVNMCVLNALGREMI | |
| | GRRLFGADADHKAEEFRSMVTEMMALAGVFNIGDFV | |
| | PALDCLDLQGVAGKMKRLHKRFDAFLSSILEEHEAM | |
| | KNGQDQKHTDMLSTLISLKGTDFDGEGGTLTDTEIKA | |
| | LLLNMFTAGTDTSASTVDWAIAELIRHPEIMRKAQEE | |
| | LDSVVGRGRPINESDLSQLPYLQAVIKENFRLHPPTPLS | |
| | LPHIASESCEINGYHIPKGSTLLTNIWAIARDPDQWSDP | |
| | LTFRPERFLPGGEKAGVDVKGNDFELIPFGAGRRICAG | |
| | LSLGLRTIQLLTATLVHGFEWELAGGVTPEKLNMEET | |
| | YGITLQRAVPLVVHPKLRLDMSAYGLGSA | |
| Cytochrome P450 | MDSSSEKLSPFELMSAILKGAKLDGSNSSDSGVAVSPA | 9 |
| reductase (CPR) | VMAMLLENKELVMILTTSVAVLIGCVVVLIWRRSSGS | |
| Catharanthus | GKKVVEPPKLIVPKSVVEPEEIDEGKKKFTIFFGTQTGT | |
| roseus | AEGFAKALAEEAKARYEKAVIKVIDIDDYAADDEEYE | |
| Accession: | EKFRKETLAFFILATYGDGEPTDNAARFYKWFVEGND | |
| Q05001 | RGDWLKNLQYGVFGLGNRQYEHFNKIAKVVDEKVA | |
| | EQGGKRIVPLVLGDDDQCIEDDFAAWRENVWPELDN | |
| | LLRDEDDTTVSTTYTAAIPEYRVVFPDKSDSLISEANG | |
| | HANGYANGNTVYDAQHPCRSNVAVRKELHTPASDRS | |
| | CTHLDFDIAGTGLSYGTGDHVGVYCDNLSETVEEAER | |
| | LLNLPPETYFSLHADKEDGTPLAGSSLPPPFPPCTLRTA | |
| | LTRYADLLNTPKKSALLALAAYASDPNEADRLKYLAS | |
| | PAGKDEYAQSLVANQRSLLEVMAEFPSAKPPLGVFFA | |
| | AIAPRLQPRFYSISSSPRMAPSRIHVTCALVYEKTPGGR | |
| | IHKGVCSTWMKNAIPLEESRDCSWAPIFVRQSNFKLP | |
| | ADPKVPVIMIGPGTGLAPFRGFLQERLALKEEGAELGT | |
| | AVFFFGCRNRKMDYIYEDELNHFLEIGALSELLVAFSR | |
| | EGPTKQYVQHKMAEKASDIWRMISDGAYVYVCGDA | |
| | KGMARDVHRTLHTIAQEQGSMDSTQAEGFVKNLQM | |
| | TGRYLRDVW | |
| Flavonoid 3′,5′- | MSTSLLLAAAAILFFITHLFLRFLLSPRRTRKLPPGPKG | 10 |
| hydroxylase | WPVVGALPMLGNMPHAALADLSRRYGPIVYLKLGSR | |
| (F3′5′H) | GMVVASTPDSARAFLKTQDLNFSNRPTDAGATHIAYN | |
| Delphinium | SQDMVFADYGPRWKLLRKLSSLHMLGGKAVEDWAV | |
| grandiflorum | VRRDEVGYMVKAIYESSCAGEAVHVPDMLVFAMAN | |
| Accession: | MLGQVILSRRVFVTKGVESNEFKEMVIELMTSAGLFN | |
| BAO66642 | VGDFIPSIAWMDLQGIVRGMKRLHKKFDALLDKILRE | |
| | HTATRRERKEKPDLVDVLMDNRDNKSEQERLTDTNI | |
| | KALLLNLFSAGTDTSSSTIEWALTEMIKNPSIFGRAHA | |
| | EMDQVIGRNRRLEESDIPKLPYLQAICKETFRKHPSTP | |
| | LNLPRVAIEPCEVEGYHIPKGTRLSVNIWAIGRDPNVW | |
| | ENPLEFNPDRFLTGKMAKIDPRGNNFELIPFGAGRRIC | |
| | AGTRMGIVLVEYILGSLVHAFEWKLRDGETLNMEETF | |
| | GIALQKAVPLAAVVTPRLPPSAYVV | |
| Dihydroflavonol 4- | MMHKGTVCVTGAAGFVGSWLIMRLLEQGYSVKATV | 11 |
| reductase (DFR) | RDPSNMKKVKHLLDLPGAANRLTLWKADLVDEGSFD | |
| Anthurium | EPIQGCTGVFHVATPMDFESKDPESEMIKPTIEGMLNV | |
| andraeanum | LRSCARASSTVRRVVFTSSAGTVSIHEGRRHLYDETS | |
| Accession: | WSDVDFCRAKKMTGWMYFVSKTLAEKAAWDFAEK | |
| AAP20866.1 | NNIDFISIIPTLVNGPFVMPTMPPSMLSALALITRNEPH | |
| | YSILNPVQFVHLDDLCNAHIFLFECPDAKGRYICSSHD | |
| | VTIAGLAQILRQRYPEFDVPTEFGEMEVFDIISYSSKKL | |
| | TDLGFEFKYSLEDMFDGAIQSCREKGLLPPATKEPSYA | |
| | TEQLIATGQDNGH | |
| Leucoanthocyanidin | MTVSGAIPSMTKNRTLVVGGTGFIGQFITKASLGFGYP | 12 |
| reductase (LAR) | TFLLVRPGPVSPSKAVIIKTFQDKGAKVIYGVINDKEC | |
| Desmodium | MEKILKEYEIDVVISLVGGARLLDQLTLLEAIKSVKTIK | |
| uncinatum | RFLPSEFGHDVDRTDPVEPGLTMYKEKRLVRRAVEEY | |
| Accession: | GIPFTNICCNSIASWPYYDNCHPSQVPPPMDQFQIYGD | |
| Q84V83.1 | GNTKAYFIDGNDIGKFTMKTIDDIRTLNKNVHFRPSSN | |
| | CYSINELASLWEKKIGRTLPRFTVTADKLLAHAAENII | |
| | PESIVSSFTHDIFINGCQVNFSIDEHSDVEIDTLYPDEKF | |
| | RSLDDCYEDFVPMVHDKIHAGKSGEIKIKDGKPLVQT | |
| | GTIEEINKDIKTLVETQPNEEIKKDMKALVEAVPISAM | |
| | G | |
| Anthocyanin | MFSSVAVPRVEILASSGIESIPKEYVRPQEELTTIGNIFD | 13 |
| dioxygenase (ANS) | EEKKDEGPQVPTIDLRDIDSDDQQVRQRCRDELKKAA | |
| Carica papaya | VDWGVMHLVNHGIPDHLIDRVKKAGQAFFELPVEVK | |
| Accession: | EKYANDQASGNIQGYGSKLANNASGQLEWEDYYFHL | |
| XP_021901846.1 | IFPEEKRDLAIWPNNPADYIEVTSEYARQLRRLVSKIL | |
| | GVLSLGLGLEEGRLEKEVGGLDELLLQMKINYYPTCP | |
| | QPELALGVEAHTDISALTFILHNMVPGLQLFYEGKWV | |
| | TAKCVPNSIVMHVGDTIEILSNGKYKSILHRGLVNKEK | |
| | VRISWAVFCEPPKEKIILKPLPETVSENEPPLFPPRTFAQ | |
| | HIQHKLFRKNQENLEAK | |
| Anthocyanidin-3- | MSQTTTNPHVAVLAFPFSTHAAPLLAVVRRLAVAAPH | 14 |
| O-glycotransferase | AVFSFFSTSESNASIFHDSMHTMQCNIKSYDVSDGVPE | |
| (3GT) | GYVFTGRPQEGIDLFMRAAPESFRQGMVMAVAETGR | |
| Vitis labrusca | PVSCLVADAFIWFAADMAAEMGVAWLPFWTAGPNS | |
| Accession: | LSTHVYIDEIREKIGVSGIQGREDELLNFIPGMSKVRFR | |
| ABR24135 | DLQEGIVFGNLNSLFSRLLHRMGQVLPKATAVFINSFE | |
| | ELDDSLTNDLKSKLKTYLNIGPFNLITPPPVVPNTTGCL | |
| | QWLKERKPTSVVYISFGTVTTPPPAELVALAEALEASR | |
| | VPFIWSLRDKARMHLPEGFLEKTRGHGMVVPWAPQA | |
| | EVLAHEAVGAFVTHCGWNSLWESVAGGVPLICRPFF | |
| | GDQRLNGRMVEDVLEIGVRIEGGVFTKSGLMSCFDQI | |
| | LSQEKGKKLRENLRALRETADRAVGPKGSSTENFKTL | |
| | VDLVSKPKDV | |
| Acetyl-CoA | MVEHRSLPGHFLGGNSLESAPQGPVKDFVQAHEGHT | 15 |
| carboxylase (ACC) | VISKVLIANNGMAAMKEIRSVRKWAYETFGNERAIEF | |
| Mucor | TVMATPEDLKANAEYIRMADNFVEVPGGSNNNNYAN | |
| circinelloides | VELIVDVAERTAVHAVWAGWGHASENPRLPEMLAKS | |
| 1006PhL | KHKCLFIGPPASAMRSLGDKISSTIVAQSAQVPTMGW | |
| Accession: | SGDGITETEFDAAGHVIVPDNAYNEACVKTAEQGLKA | |
| EPB82652.1 | AEKIGFPVMIKASEGGGGKGIRMVKDGSNFAQLFAQV | |
| | QGEIPGSPIFIMKLAGNARHLEVQLLADQYGNAISLFG | |
| | RDCSVQRRHQKIIEEAPVTIAKPDVFEQMEKAAVRLG | |
| | KLVGYVSAGTVEYLYSHHDDQFYFLELNPRLQVEHPT | |
| | TEMVSGVNLPAAQLQIAMGIPLHRIRDIRVLYGVQPNS | |
| | ASEIDFGFEHPTSLTSHRRPTPKGHVIACRITAENPDAG | |
| | FKPSSGIMQELNFRSSTNVWGYFSVVSAGGLHEYADS | |
| | QFGHIFAYGENRQQARKNMVIALKELSIRADFRSTVE | |
| | YIIRLLETPDFEENTINTGWLDMLISKKLTAERPDTML | |
| | AVFCGAVTKAHMASLDCFQQYKQSLEKGQVPSKGSL | |
| | KTVFTVDFIYEEVRYNFTVTQSAPGIYTLYLNGTKTQV | |
| | GIRDLSDGGLLISIDGKSHTTYSRDEVQATRMMVDGK | |
| | TCLLEKESDPTQLRSPSPGKLVNLLVENGDHLNAGDA | |
| | YAEIEVMKMYMPLIATEDGHVQFIKQAGATLEAGDII | |
| | GILSLDDPSRVKHALPFNGTVPAFGAPHITGDKPVQRF | |
| | NATKLTLQHILQGYDNQALVQTVVKDFADILNNPDLP | |
| | YSELNSVLSALSGRIPQRLEASIHKLADESKAANQEFP | |
| | AAQFEKLVEDFAREHITLQSEATAYKNSVAPLSSIFAR | |
| | YRNGLTEHAYSNYVELMEAYYDVEILFNQQREEEVIL | |
| | SLRDQHKDDLDKVLAVTLSHAKVNIKNNVILMLLDLI | |
| | NPVSTGSALDKYFTPILKRLSEIESRATQKVTLKAREL | |
| | LILCQLPSYEERQAQMYQILKNSVTESVYGGGSEYRTP | |
| | SYDAFKDLIDTKFNVFDVLPHFFYHADPYIALAAIEVY | |
| | CRRSYHAYKILDVAYNLEHKPYVVAWKFLLQTAANG | |
| | IDSNKRIASYSDLTFLLNKTEEEPIRTGAMTACNSLAD | |
| | LQAELPRILTAFEEEPLPPMLQRNAAPKEERMENILNI | |
| | AVRADEDMDDTAFRTKICEMITANADVFRQAHLRRL | |
| | SVVVCRDNQWPDYYTFRERENYQEDETIRHIEPAMA | |
| | YQLELARLSNFDIKPCFIENRQMHVYYAVAKENPSDC | |
| | RFFIRALVRPGRVKSSMRTADYLISESDRLLTDILDTLE | |
| | IVSHEYKNSDCNHLFINFIPTFAIEADDVEHALKDFVD | |
| | RHGKRLWKLRVTGAEIRFNVQSKKPDAPIIPMRFTVD | |
| | NVSGFILKVEVYQEVKTEKSGWILKSVNKIPGAMHM | |
| | QPLSTPYPTKEWLQPRRYKAHLMGTTYVYDFPELFRQ | |
| | SVQNQWTQAIKRNPLLKQPSHLVEAKELVLDEDDVL | |
| | QEIDRAPGTNTVGMVAWIMTIRTPEYPSGRRIIAIANDI | |
| | TFKIGSFGVAEDQVFYKASELARALGIPRIYLSANSGA | |
| | RIGLADELISQFRAAWKDASNPTAGFKYLYLTPAEYD | |
| | VLAQQGDAKSVLVEEIQDEGETRLRITDVIGHTDGLG | |
| | VENLKGSGLIAGATSRAYDDIFTITLVTCRSVGIGAYL | |
| | VRLGQRTIQNEGQPIILTGAPALNKVLGREVYTSNLQL | |
| | GGTQIMYKNGVSHLTAENDLEGIAKIVQWLSFVPDVR | |
| | NAPVSMRLGADPIDRDIEYTPPKGPSDPRFFLAGKSEN | |
| | GKWLSGFFDQDSFVETLSGWARTVVVGRARLGGIPM | |
| | GVVSVETRTVENIVPADPANSDSTEQVFMEAGGVWFP | |
| | NSAYKTAQAINDFNKGEQLPLMIFANWRGFSGGQRD | |
| | MYNEVLKYGAQIVDALSNYKQPVFVYIIPNGELRGGA | |
| | WVVVDPTINKDMMEMYADNNARGGVLEPEGIVEIKY | |
| | RKPALLATMERLDATYASLKKQLAEEGKTDEEKAAL | |
| | KVQVEAREQELLPVYQQISIQFADLHDRAGRMKAKG | |
| | VIRKALDWRRARHYFYWRVRRRLCEEYTFRKIVTATS | |
| | AAPMPREQMLDLVKQWFTNDNETVNFEDADELVSE | |
| | WFEKRASVIDQRISKLKSDATKEQIVSLGNADQEAVIE | |
| | GFSQLIENLSEDARAEILRKLNSRF | |
| Acetyl-CoA | MSQTHKHAIPANIADRCLINPEQYETKYKQSINDPDTF | 16 |
| synthase (ACS) | WGEQGKILDWITPYQKVKNTSFAPGNVSIKWYEDGT | |
| Salmonella | LNLAANCLDRHLQENGDRTAIIWEGDDTSQSKHISYR | |
| typhimurium | ELHRDVCRFANTLLDLGIKKGDVVAIYMPMVPEAAV | |
| Accession: | AMLACARIGAVHSVIFGGFSPEAVAGRIIDSSSRLVITA | |
| NP_463140.1 | DEGVRAGRSIPLKKNVDDALKNPNVTSVEHVIVLKRT | |
| | GSDIDWQEGRDLWWRDLIEKASPEHQPEAMNAEDPL | |
| | FILYTSGSTGKPKGVLHTTGGYLVYAATTFKYVFDYH | |
| | PGDIYWCTADVGWVTGHSYLLYGPLACGATTLMFEG | |
| | VPNWPTPARMCQVVDKHQVNILYTAPTAIRALMAEG | |
| | DKAIEGTDRSSLRILGSVGEPINPEAWEWYWKKIGKE | |
| | KCPVVDTWWQTETGGFMITPLPGAIELKAGSATRPFF | |
| | GVQPALVDNEGHPQEGATEGNLVITDSWPGQARTLF | |
| | GDHERFEQTYFSTFKNMYFSGDGARRDEDGYYWITG | |
| | RVDDVLNVSGHRLGTAEIESALVAHPKIAEAAVVGIP | |
| | HAIKGQAIYAYVTLNHGEEPSPELYAEVRNWVRKEIG | |
| | PLATPDVLHWTDSLPKTRSGKIMRRILRKIAAGDTSNL | |
| | GDTSTLADPGVVEKLLEEKQAIAMPS | |
| Malonyl-CoA | MSSLFPALSPAPTGAPADRPALRFGERSLTYAELAAA | 17 |
| synthase (matB) | AGATAGRIGGAGRVAVWATPAMETGVAVVAALLAG | |
| Streptomyces | VAAVPLNPKSGDKELAHILSDSAPSLVLAPPDAELPPA | |
| coelicolor | LGALERVDVDVRARGAVPEDGADDGDPALVVYTSGT | |
| Accession: | TGPPKGAVIPRRALATTLDALADAWQWTGEDVLVQG | |
| WP_011028356 | LPLFHVHGLVLGILGPLRRGGSVRHLGRFSTEGAAREL | |
| | NDGATMLFGVPTMYHRIAETLPADPELAKALAGARL | |
| | LVSGSAALPVHDHERIAAATGRRVIERYGMTETLMNT | |
| | SVRADGEPRAGTVGVPLPGVELRLVEEDGTPIAALDG | |
| | ESVGEIQVRGPNLFTEYLNRPDATAAAFTEDGFFRTG | |
| | DMAVRDPDGYVRIVGRKATDLIKSGGYKIGAGEIENA | |
| | LLEHPEVREAAVTGEPDPDLGERIVAWIVPADPAAPP | |
| | ALGTLADHVAARLAPHKRPRVVRYLDAVPRNDMGKI | |
| | MKRALNRD | |
| Malonate | MSPELISILVLVVVFVIATTRSVNMGALAFAAAFGVGT | 18 |
| transporter (matC) | LVADLDADGIFAGFPGDLFVVLVGVTYLFAIARANGT | |
| Streptomyces | TDWLVHAAVRLVRGRVALIPWVMFALTGALTAIGAV | |
| coelicolor | SPAAVAIVAPVALSFATRYSISPLLMGTMVVHGAQAG | |
| Accession: | GFSPISIYGSIVNGIVEREKLPGSEIGLFLASLVANLLIA | |
| NP_626686.1 | AVLFAVLGGRKLWARGAVTPEGDGAPGKAGTGTTGS | |
| | GSDTGTGTGTGTGTSAGTGGTAPTAVAVRSDRETGG | |
| | AEGTGVRLTPARVATLVALVALVVAVLGFDLDAGLT | |
| | AVTLAVVLSTAWPDDSRRAVGEIAWSTVLLICGVLTY | |
| | VGVLEEMGTITWAGEGVGGIGVPLLAAVLLCYIGAIV | |
| | SAFASSVGIMGALIPLAVPFLAQGEIGAVGMVAALAV | |
| | SATVVDVSPFSTNGALVLAAAPDVDRDRFFRQLMVY | |
| | GGIVVAAVPALAWLVLVVPGFG | |
| Malonate CoA- | MVKKRLWDKQRTRRQEKLNLAQQKGFAKQVEHARA | 19 |
| transferase (MdcA) | IELLETVIASGDRVCLEGNNQKQADFLSKCLSQCNPD | |
| Acinetobacter | AVNDLHIVQSVLALPSHIDVFEKGIASKVDFSFAGPQS | |
| calcoaceticus | LRLAQLVQQQKISIGSIHTYLELYGRYFIDLTPNICLITA | |
| Accession: | HAADREGNLYTGPNTEDTPAIVEATAFKSGIVIAQVNE | |
| AAB97627.1 | IVDKLPRVDVPADWVDFYIESPKHNYIEPLFTRDPAQI | |
| | TEVQILMAMMVIKGIYAPYQVQRLNHGIGFDTAAIEL | |
| | LLPTYAASLGLKGQICTNWALNPHPTLIPAIESGFVDS | |
| | VHSFGSEVGMEDYIKERPDVFFTGSDGSMRSNRAFSQ | |
| | TAGLYACDSFIGSTLQIELQGNSSTATVDRISGFGGAP | |
| | NMGSDPHGRRHASYAYTKAGREATDGKLIKGRKLVV | |
| | QTVETYREHMHPVFVEELDAWQLQDKMDSELPPIMI | |
| | YGEDVTHIVTEEGIANLLLCRTDEEREQAIRGVAGYTP | |
| | VGLKRDAAKVEELRQRGIIQRPEDLGIDPTQVSRDLLA | |
| | AKSVKDLVKWSGGLYSPPSRFRNW | |
| Pantothenate | MILELDCGNSLIKWRVIEGAARSVAGGLAESDDALVE | 20 |
| kinase (CoaX) | QLTSQQALPVRACRLVSVRSEQETSQLVARLEQLFPV | |
| Pseudomonas | SALVASSGKQLAGVRNGYLDYQRLGLDRWLALVAA | |
| aeruginosa | HHLAKKACLVIDLGTAVTSDLVAADGVHLGGYICPG | |
| Accession: | MTLMRSQLRTHTRRIRYDDAEARRALASLQPGQATA | |
| Q9HWC1.1 | EAVERGCLLMLRGFVREQYAMACELLGPDCEIFLTGG | |
| | DAELVRDELAGARIMPDLVFVGLALACPIE | |
| glutamyl-tRNA | MTKKLLALGINHKTAPVSLRERVTFSPDTLDQALDSL | 21 |
| reductase (hemA^{m}) | LAQPMVQGGVVLSTCNRTELYLSVEEQDNLQEALIR | |
| Salmonella | WLCDYHNLNEDDLRNSLYWHQDNDAVSHLMRVASG | |
| typhimurium | LDSLVLGEPQILGQVKKAFADSQKGHLNASALRRMF | |
| Accession: | QKSFSVAKRVRTETDIGASAVSVAFAACTLARQIFESL | |
| AAA88610.1 | STVTVLLVGAGETIELVARHLREHKVQKMIIANRTRE | |
| | RAQALADEVGAEVISLSDIDARLQDADIIISSTASPLPII | |
| | GKGMVERALKSRRNQPMLLVDIAVPRDVEPEVGKLA | |
| | NAYLYSVDDLQSIISHNLAQRQAAAVEAETIVEQEASE | |
| | FMAWLRAQGASETIREYRSQSEQIRDELTTKALSALQ | |
| | QGGDAQAILQDLAWKLINRLIHAPTKSLQQAARDGD | |
| | DERLNILRDSLGLE | |
| 5-aminolevulinic | MDYNLALDKAIQKLHDEGRYRTFIDIEREKGAFPKAQ | 22 |
| acid synthase | WNRPDGGKQDITVWCGNDYLGMGQHPVVLAAMHE | |
| (ALAS) | ALEAVGAGSGGTRNISGTTAYHRRLEAEIADLHGKEA | |
| Rhodobacter | ALVFSSAYIANDATLSTLRLLFPGLIIYSDSLNHASMIE | |
| capsulatus | GIKRNAGPKRIFRHNDVAHLRELIAADDPAAPKLIAFE | |
| Accession: | SVYSMDGDFGPIKEICDIADEFGALTYIDEVHAVGMY | |
| CAA37857 | GPRGAGVAERDGLMHRIDIFNGTLAKAYGVFGGYIA | |
| | ASAKMVDAVRSYAPGFIFSTSLPPAIAAGAQASIAFLK | |
| | TAEGQKLRDAQQMHAKVLKMRLKALGMPIIDHGSHI | |
| | VPVVIGDPVHTKAVSDMLLSDYGVYVQPINFPTVPRG | |
| | TERLRFTPSPVHDLKQIDGLVHAMDLLWARCA | |
| Tyrosine ammonia- | MTLQSQTAKDCLALDGALTLVQCEAIATHRSRISVTP | 23 |
| lyase (TAL) | ALRERCARAHARLEHAIAEQRHIYGITTGFGPLANRLI | |
| Rhodobacter | GADQGAELQQNLIYHLATGVGPKLSWAEARALMLAR | |
| capsulatus SB 1003 | LNSILQGASGASPETIDRIVAVLNAGFAPEVPAQGTVG | |
| Accession: | ASGDLTPLAHMVLALQGRGRMIDPSGRVQEAGAVM | |
| ADE84832.1 | DRLCGGPLTLAARDGLALVNGTSAMTAIAALTGVEA | |
| | ARAIDAALRHSAVLMEVLSGHAEAWHPAFAELRPHP | |
| | GQLRATERLAQALDGAGRVCRTLTAARRLTAADLRP | |
| | EDHPAQDAYSLRVVPQLVGAVWDTLDWHDRVVTCE | |
| | LNSVTDNPIFPEGCAVPALHGGNFMGVHVALASDAL | |
| | NAALVTLAGLVERQIARLTDEKLNKGLPAFLHGGQA | |
| | GLQSGFMGAQVTATALLAEMRANATPVSVQSLSTNG | |
| | ANQDVVSMGTIAARRARAQLLPLSQIQAILALALAQA | |
| | MDLLDDPEGQAGWSLTARDLRDRIRAVSPGLRADRP | |
| | LAGHIEAVAQGLRHPSAAADPPA | |
| Tyrosine ammonia- | MITETNVAKPASTKVMNGDAAKAAPVEPFATYAHSQ | 24 |
| lyase (TAL) | ATKTVVIDGHNMKVGDVVAVARHGAKVELAASVAG | |
| Trichosporon | PVQASVDFKESKKHTSIYGVTTGFGGSADTRTSDTEA | |
| cutaneum | LQISLLEHQLCGYLPTDPTYEGMLLAAMPIPIVRGAM | |
| Accession: | AVRVNSCVRGHSGVRLEVLQSFADFINIGLVPCVPLR | |
| XP_018276715 | GTISASGDLSPLSYIAGAICGHPDVKVFDTAASPPTVLT | |
| | APEAIAKYKLKTVRLASKEGLGLVNGTAVSAAAGAL | |
| | ALYDAECLAMMSQTNTALTVEALDGHVGSFAPFIQEI | |
| | RPHVGQIEAAKNIRHMLSNSKLAVHEEPELLADQDAG | |
| | ILRQDRYALRTSAQWIGPQLEMLGLARQQIETELNSTT | |
| | DNPLIDVEGGMFHHGGNFQAMAVTSAMDSTRIVLQN | |
| | LGKLSFAQVTELINCEMNHGLPSNLAGSEPSTNYHCK | |
| | GLDIHCGAYCAELGFLANPMSNHVQSTEMHNQSVNS | |
| | MAFASARKTMEANEVLSLLLGSQMYCATQALDLRV | |
| | MEVKFKMAIVKLLNDTLTKHFSTFLTPEQLAKLNTTA | |
| | AITLYKRLNQTPSWDSAPRFEDAAKHLVGCIMDALM | |
| | VNDDITDLTNLPKWKKEFAKDAGDLYRSILTATTADG | |
| | RNDLEPAEYLGQTRAVYEAIRSDLGVKVRRGDVAEG | |
| | KSGKSIGSNVARIVEAMRDGRLMGAVSKMFF | |
| Tyrosine ammonia- | MNTINEYLSLEEFEAIIFGNQKVTISDVVVNRVNESFNF | 25 |
| lyase (TAL) | LKEFSGNKVIYGVNTGFGPMAQYRIKESDQIQLQYNLI | |
| Flavobacterium | RSHSSGTGKPLSPVCAKAAILARLNTLSLGNSGVHPSV | |
| johnsoniae | INLMSELINKDITPLIFEHGGVGASGDLVQLSHLALVLI | |
| Accession: | GEGEVFYKGERRPTPEVFEIEGLKPIQVEIREGLALING | |
| WP_012023194 | TSVMTGIGVVNVYHAKKLLDWSLKSSCAINELVQAY | |
| | DDHFSAELNQTKRHKGQQEIALKMRQNLSDSTLIRKR | |
| | EDHLYSGENTEEIFKEKVQEYYSLRCVPQILGPVLETI | |
| | NNVASILEDEFNSANDNPIIDVKNQHVYHGGNFHGDY | |
| | ISLEMDKLKIVITKLTMLAERQLNYLLNSKINELLPPFV | |
| | NLGTLGFNFGMQGVQFTATSTTAESQMLSNPMYVHSI | |
| | PNNNDNQDIVSMGTNSAVITSKVIENAFEVLAIEMITIV | |
| | QAIDYLGQKDKISSVSKKWYDEIRNIIPTFKEDQVMYP | |
| | FVQKVKDHLINN | |
| Tyrosine ammonia- | MSTTLILTGEGLGIDDVVRVARHQDRVELTTDPAILA | 26 |
| lyase (TAL) | QIEASCAYINQAVKEHQPVYGVTTGFGGMANVIISPEE | |
| Herpetosiphon | AAELQNNAIWYHKTGAGKLLPFTDVRAAMLLRANSH | |
| aurantiacus DSM | MRGASGIRLEIIQRMVTFLNANVTPHVREFGSIGASGD | |
| 785 | LVPLISITGALLGTDQAFMVDFNGETLDCISALERLGL | |
| Accession: | PRLRLQPKEGLAMMNGTSVMTGIAANCVHDARILLA | |
| ABX04526.1 | LALEAHALMIQGLQGTNQSFHPFIHRHKPHTGQVWA | |
| | ADHMLELLQGSQLSRNELDGSHDYRDGDLIQDRYSL | |
| | RCLPQFLGPIIDGMAFISHHLRVEINSANDNPLIDTASA | |
| | ASYHGGNFLGQYIGVGMDQLRYYMGLMAKHLDVQI | |
| | ALLVSPQFNNGLPASLVGNIQRKVNMGLKGLQLTANS | |
| | IMPILTFLGNSLADRFPTHAEQFNQNINSQGFGSANLA | |
| | RQTIQTLQQYIAITLMFGVQAVDLRTHKLAGHYNAAE | |
| | LLSPLTAKIYHAVRSIVKHPPSPERPYIWNDDEQVLEA | |
| | HISALAHDIANDGSLVSAVEQTLSGLRSIILFR | |
| Phenylalanine | MHDDNTSPYCIGQLGNGAVHGADPLNWAKTAKAME | 27 |
| ammonia-lyase | CSHLEEIKRMVDTYQNATQVMIEGATLTVPQVAAIAR | |
| (PAL) | RPEVHVVLDAANARSRVDESSNWVLDRIMGGGDIYG | |
| Physcomitrella | VTTGFGATSHRRTQQGVELQRELIRFLNAGVLSKGNS | |
| patens | LPSETARAAMLVRTNTLMQGYSGIRWEILHAMEKLL | |
| Accession: | NAHVTPKLPLRGTITASGDLVPLSYIAGLLTGRPNSKA | |
| XP_001758374.1 | VTEDGREVSALEALRIAGVEKPFELAPKEGLALVNGT | |
| | AVGSALASTVCYDANIMVLLAEVLSALFCEVMQGKP | |
| | EFADPLTHKLKHHPGQMEAAAVMEWVLDGSSFMKA | |
| | AAKFNETDPLRKPKQDRYALRTSPQWLGPQVEVIRNA | |
| | THAIEREINSVNDNPIIDAARGIALHGGNFQGTPIGVSM | |
| | DNMRLSLAAIAKLMFAQFSELVNDYYNNGLPSNLSG | |
| | GPNPSLDYGMKGAEIAMASYLSEINYLANPVTTHVQS | |
| | AEQHNQDVNSLGLVSARKTEEAMEILKLMSATFLVG | |
| | LCQAIDLRHVEETMQSAVKQVVTQVAKKTLFMGSDG | |
| | SLLPSRFCEKELLMVVDRQPVFSYIDDSTSDSYPLMEK | |
| | LRGVLVSRALKSADKETSNAVFRQIPVFEAELKLQLSR | |
| | VVPAVREAYDTKGLSLVPNRIQDCRTYPLYKLVRGDL | |
| | KTQLLSGQRTVSPGQEIEKVFNAISAGQLVAPLLECVQ | |
| | GWTGTPGPFSARASC | |
| Phenylalanine | MIETNHKDNFLIDGENKNLEINDIISISKGEKNIIFTNEL | 28 |
| ammonia-lyase | LEFLQKGRDQLENKLKENVAIYGINTGFGGNGDLIIPF | |
| (PAL) | DKLDYHQSNLLDFLTCGTGDFFNDQYVRGIQFIIIIALS | |
| Dictyostelium | RGWSGVRPMVIQTLAKHLNKGIIPQVPMHGSVGASG | |
| discoideum AX4 | DLVPLSYIANVLCGKGMVKYNEKLMNASDALKITSIE | |
| Accession: | PLVLKSKEGLALVNGTRVMSSVSCISINKFETIFKAAIG | |
| XP_644510.1 | SIALAVEGLLASKDHYDMRIHNLKNHPGQILIAQILNK | |
| | YFNTSDNNTKSSNITFNQSENVQKLDKSVQEVYSLRC | |
| | APQILGIISENISNAKIVIKREILSVNDNPLIDPYYGDVL | |
| | SGGNFMGNHIARIMDGIKLDISLVANHLHSLVALMMH | |
| | SEFSKGLPNSLSPNPGIYQGYKGMQISQTSLVVWLRQE | |
| | AAPACIHSLTTEQFNQDIVSLGLHSANGAASMLIKLCD | |
| | IVSMTLIIAFQAISLRMKSIENFKLPNKVQKLYSSIIKIIPI | |
| | LENDRRTDIDVREITNAILQDKLDFINLNL | |
| Phenylalanine | MSQVALFEQELMLHGKHTLLLNGNDLTITDVAQMAK | 29 |
| ammonia-lyase | GTFEAFTFHISEEANKRIEECNELKHEIMNQHNPIYGV | |
| (PAL) | TTGFGDSVHRQISGEKAWDLQRNLIRFLSCGVGPVAD | |
| Brevibacillus | EAVARATMLIRTNCLVKGNSAVRLEVIHQLIAYMERG | |
| laterosporus LMG | ITPIIPERGSVGASGDLVPLSYLASILVGEGKVLYKGEE | |
| 15441 | REVAEALGAEGLEPLTLEAKEGLALVNGTSFMSAFAC | |
| Accession: | LAYADAEEIAFIADICTAMASEALLGNRGHFYSFIHEQ | |
| WP_003337219.1 | KPHLGQMASAKNIYTLLEGSQLSKEYSQIVGNNEKLD | |
| | SKAYLELTQSIQDRYSIRCAPHVTGVLYDTLDWVKK | |
| | WLEVEINSTNDNPIFDVETRDVYNGGNFYGGHVVQA | |
| | MDSLKVAVANIADLLDRQLQLVVDEKFNKDLTPNLIP | |
| | RFNNDNYEIGLHHGFKGMQIASSALTAEALKMSGPVS | |
| | VFSRSTEAHNQDKVSMGTISSRDARTIVELTQHVAAIH | |
| | LIALCQALDLRDSKKMSPQTTKIYNMIRKQVPFVERD | |
| | RALDGDIEKVVQLIRSGNLKKEIHDQNVND | |
| Cinnamate-4- | MDLLLMEKTLLGLFVAVVVAITVSKLRGKKFKLPPGP | 30 |
| hydroxylase (C4H) | IPVPVFGNWLQVGDDLNHRNLTEMAKKFGEVFMLR | |
| Rubus sp. SSL-2007 | MGQRNLVVVSSPDLAKEVLHTQGVEFGSRTRNVVFDI | |
| Accession: | FTGKGQDMVFTVYGEHWRKMRRIMTVPFFTNKVVQ | |
| ABX74781.1 | QYRYGWESEAAAVVEDVKKHPEAATNGMVLRRRLQ | |
| | LMMYNNMYRIMFDRRFESEDDPLFVKLKGLNGERSR | |
| | LAQSFEYNYGDFIPVLRPFLRGYLKICKEVKEKRIQLF | |
| | KDYFVDERKKLSSTQATTNEGLKCAIDHILDAQQKGE | |
| | INEDNVLYIVENINVAAIETTLWSIEWGIAELVNHPEIQ | |
| | KKLRDELDTVLGRGVQITEPEIQKLPYLQAVVKETLR | |
| | LRMAIPLLVPHMNLHDAKLGGFDIPAESKILVNAWWL | |
| | ANNPAHWKKPEEFRPERFLEEESKVEANGNDFRYLPF | |
| | GVGRRSCPGIILALPILGITLGRLVQNFELLPPPGQTQL | |
| | DTTEKGGQFSLHILKHSPIVMKPRT | |
| Cinnamate-4- | MDLLLLEKTLIGLFIAIVVAIIVSKLRGKKFKLPPGPIPV | 31 |
| hydroxylase (C4H) | PVFGNWLQVGDDLNHRNLTDMAKKFGDVFMLRMG | |
| Fragaria vesca | QRNLVVVSSPDLAKEVLHTQGVEFGSRTRNVVFDIFT | |
| Accession: | GKGQDMVFTVYGEHWRKMRRIMTVPFFTNKVVQQY | |
| XP_004294725.1 | RHGWEAEAAAVVEDVKKHPEAATSGMVLRRRLQLM | |
| | MYNNMYRIMFDRRFESEEDPLFVKLKGLNGERSRLA | |
| | QSFEYNYGDFIPVLRPFLRGYLKICKEVKEKRIQLFKD | |
| | YFVDERKKLASTQVTTNEGLKCAIDHILDAQQKGEIN | |
| | EDNVLYIVENINVAAIETTLWSIEWGIAELVNHPEIQK | |
| | KLRDELDTVLGHGVQVTEPELHKLPYLQAVVKETLR | |
| | LRMAIPLLVPHMNLHDAKLGGFDIPAESKILVNAWWL | |
| | ANNPAHWKKPEEFRPERFLEEESKVEANGNDFRYLPF | |
| | GVGRRSCPGIILALPILGVTLGRLVQNFEMLPPPGQTQ | |
| | LDTTEKGGQFSLHILKHSTIVMKPRA | |
| Cinnamate-4- | MDLLLLEKTLIGLFFAILIAIIVSKLRSKRFKLPPGPIPVP | 32 |
| hydroxylase (C4H) | VFGNWLQVGDDLNHRNLTEYAKKFGDVFLLRMGQR | |
| Solanum tuberosum | NLVVVSSPELAKEVLHTQGVEFGSRTRNVVFDIFTGK | |
| Accession: | GQDMVFTVYGEHWRKMRRIMTVPFFTNKVVQQYRG | |
| ABC69046.1 | GWESEAASVVEDVKKNPESATNGIVLRKRLQLMMYN | |
| | NMFRIMFDRRFESEDDPLFVKLRALNGERSRLAQSFE | |
| | YNYGDFIPILRPFLRGYLKICKEVKEKRLKLFKDYFVD | |
| | ERKKLANTKSMDSNALKCAIDHILEAQQKGEINEDNV | |
| | LYIVENINVAAIETTLWSIEWGIAELVNHPHIQKKLRD | |
| | EIDTVLGPGMQVTEPDMPKLPYLQAVIKETLRLRMAI | |
| | PLLVPHMNLHDAKLAGYDIPAESKILVNAWWLANNP | |
| | AHWKKPEEFRPERFFEEEKHVEANGNDFRFLPFGVGR | |
| | RSCPGIILALPILGITLGRLVQNFEMLPPPGQSKLDTSE | |
| | KGGQFSLHILKHSTIVMKPRSF | |
| 4-coumarate-CoA | MGDCAAPKQEIIFRSKLPDIYIPKHLPLHSYCFENISKV | 33 |
| ligase (4CL) | SDRACLINGATGETFSYAQVELISRRVASGLNKLGIHQ | |
| Daucus carota | GDTMMILLPNTPEYFFAFLGASYRGAVSTMANPFFTS | |
| Accession: | PEVIKQLKASQAKLIITQACYVEKVKEYAAENNITVVC | |
| AIT52344.1 | IDEAPRDCLHFTTLMEADEAEMPEVAIDSDDVVALPY | |
| | SSGTTGLPKGVMLTHKGLVTSVAQRVDGENPNLYIHS | |
| | EDVMICILPLFHIYSLNAVLCCGLRAGATILIMQKFDIV | |
| | PFLELIQKYKVTIGPFVPPIVLAIAKSPVVDNYDLSSVR | |
| | TVMSGAAPLGKELEDAVRAKFPNAKLGQGYGMTEA | |
| | GPVLAMCLAFAKEPYEIKSGACGTVVRNAEMKIVDPE | |
| | THASLPRNQSGEICIRGDQIMKGYLNDPESTKTTIDEE | |
| | GWLHTGDIGFIDEDDELFIVDRLKEIIKYKGFQVAPAEI | |
| | EALLLTHPTISDAAVVPMIDEKAGEVPVAFVVRLNGS | |
| | TTTEEEIKQFVSKQVVFYKRVFRVFFVDAIPKSPSGKIL | |
| | RKELRARIASGDLPK | |
| 4-coumarate-CoA | MEPTTKSKDIIFRSKLPDIYIPKHLPLHTYCFENISRFGS | 34 |
| ligase (4CL) | RPCLINGSTGEILTYDQVELASRRVGSGLHRLGIRQGD | |
| Striga asiatica | TIMLLLPNSPEFVLAFLGASHIGAVSTMANPFFTPAEV | |
| Accession: | VKQAAASRAKLIVTQACHVDKVRDYAAEHGVKVVC | |
| GER48539.1 | VDGAPPEECLPFSEVASGDEAELPAVKISPDDVVALPY | |
| | SSGTTGLPKGVMLTHKGLVTSVAQQVDGENPNLYIHS | |
| | DDVIMCVLPLFHIYSLNSIMLCGLRVGAAILIMQKFEIV | |
| | PFLELIQRYRVTIGPFVPPIVLAIEKSPVVEKYDLSSVRT | |
| | VMSGAAPLGRELEDAVRLKFPNAKLGQGYGMTEAGP | |
| | VLAMCLAFAKEPFEIKSGACGTVVRNAEMKIVDTETG | |
| | ASLGRNQPGEICIRGDQIMKGYLNDPESTERTIDKEGW | |
| | LHTGDIGFIDDDDELFIVDRLKEIIKYKGFQVAPAELEA | |
| | LLLNHPNISDAAVVSMKDEQAGEVPVAYVVKSNGSTI | |
| | TEDEIKQFVSKQVIFYKRINRVFFIDAIPKSPSGKILRKD | |
| | LRARLAAGVPN | |
| 4-coumarate-CoA | MPMENEAKQGDIIFRSKLPDIYIPNHLSLHSYCFENISE | 35 |
| ligase (4CL) | FSSRPCLINGANNQIYTYADVELNSRKVAAGLHKQFGI | |
| Capsicum annuum | QQKDTIMILLPNSPEFVFAFLGASYLGAISTMANPLFTP | |
| Accession: | AEVVKQVKASNAEIIVTQACHVNKVKDYALENDVKI | |
| KAF3620179.1 | VCIDSAPEGCVHFSELIQADEHDIPEVQIKPDDVVALP | |
| | YSSGTTGLPKGVMLTHKGLVTSVAQQVDGENPNLYI | |
| | HSEDVMLCVLPLFHIYSLNSVLLCGLRVGAAILIMQKF | |
| | DIVPFLELIQNYKVTIGPFVPPIVLAIAKSPMVDNYDLS | |
| | SVRTVMSGAAPLGKELEDTVRAKFPNAKLGQGYGMT | |
| | EAGPVLAMCLAFAKEPFEIKSGACGTVVRNAEMKIVD | |
| | PDTGNSLHRNQSGEICIRGDQIMKGYLNDPEATAGTID | |
| | KEGWLHTGDIGYIDNDDELFIVDRLKELIKYKGFQVA | |
| | PAELEALLLNHPNISDAAVVPMKDEQAGEVPVAFVVR | |
| | SNGSTITEDEVKEFISKQVIFYKRIKRVFFVDAVPKSPS | |
| | GKILRKDLRAKLAAGFPN | |
| 4-coumarate-CoA | MDTKTTQQEIIFRSKLPDIYIPKQLPLHSYCFENISQFSS | 36 |
| ligase (4CL) | KPCLINGSTGKVYTYSDVELTSRKVAAGFHNLGIQQR | |
| Camellia sinensis | DTIMLLLPNCPEFVFAFLGASYLGAIITMANPFFTPAET | |
| Accession: | IKQAKASNSKLIITQSSYTSKVLDYSSENNVKIICIDSPP | |
| ASU87409.1 | DGCLHFSELIQSNETQLPEVEIDSNEVVALPYSSGTTGL | |
| | PKGVMLTHKGLVTSVAQQVDGENPNLYIHSEDMMM | |
| | CVLPLFHIYSLNSVLLCGLRVGAAILIMQKFEIGSFLKL | |
| | IQRYKVTIGPFVPPIVLAIAKSEVVDDYDLSTIRTMMS | |
| | GAAPLGKELEDAVRAKFPHAKLGQGYGMTEAGPVLA | |
| | MCLAFAKKPFEIKSGACGTVVRNAEMKIVDPDAGFSL | |
| | PRNQPGEICIRGDQIMKGYLNDPEATERTIDKQGWLH | |
| | TGDIGYIDDDDELFIVDRLKELIKYKGFQVAPAELEAL | |
| | LLNHPTISDAAVVPMKDESAGEVPVAFVVRTNGFEVT | |
| | ENEIKKYISEQVVFYKKINRVYFVDAIPKAPSGKILRK | |
| | DLRARLAAGIPS | |
| Chalcone synthase | MVTVEEYRKAQRAEGPATVMAIGTATPSNCVDQSTY | 37 |
| (CHS) | PDYYFRITNSEHKTELKEKFKRMCEKSMIKTRYMHLT | |
| Capsicum annuum | EEILKENPNMCAYMAPSLDARQDIVVVEVPKLGKEA | |
| Accession: | AQKAIKEWGQPKSKITHLVFCTTSGVDMPGCDYQLA | |
| XP_016566084.1 | KLLGLRPSVKRLMMYQQGCFAGGTVLRLAKDLAEN | |
| | NKGARVLVVCSEITAVTFRGPSESHLDSLVGQALFGD | |
| | GAAAIIMGSDPIPGVERPLFQLVSAAQTLLPDSEGAID | |
| | GHLREVGLTFHLLKDVPGLISKNIEKSLVEAFQPLGISD | |
| | WNSLFWIAHPGGPAILDQVELKLGLKPEKLKATREVL | |
| | SNYGNMSSACVLFILDEMRKASTKEGLGTSGEGLEW | |
| | GVLFGFGPGLTVETVVLHSVAI | |
| Chalcone synthase | MVTVEEVRKAQRAEGPATVLAIGTATPPNCIDQSTYP | 38 |
| (CHS) | DYYFRITKSEHKAELKEKFQRMCDKSMIKKRYMYLT | |
| Rosa chinensis | EEILKENPSMCEYMAPSLDARQDMVVVEIPKLGKEAA | |
| Accession: | TKAIKEWGQPKSKITHLVFCTTSGVDMPGADYQLTKL | |
| AEC13058.1 | LGLRPSVKRLMMYQQGCFAGGTVLRLAKDLAENNK | |
| | GARVLVVCSEITAVTFRGPSDTHLDSLVGQALFGDGA | |
| | AAIIVGSDPLPEVEKPLFELVSAAQTILPDSDGAIDGHL | |
| | REVGLTFHLLKDVPGLISKNIEKSLNEAFKPLNITDWN | |
| | SLFWIAHPGGPAILDQVEAKLGLKPEKLEATRHILSEY | |
| | GNMSSACVLFILDEVRRKSAANGHKTTGEGLEWGVL | |
| | FGFGPGLTVETVVLHSVAA | |
| Chalcone synthase | MSMTPSVHEIRKAQRSEGPATVLSIGTATPTNFVPQAD | 39 |
| (CHS) | YPDYYFRITNSDHMTDLKDKFKRMCEKSMITKRHMY | |
| Morus alba var. | LTEEILKENPKMCEYMAPSLDARQDIVVVEVPKLGKE | |
| multicaulis | AAAKAIKEWGQPKSKITHLIFCTTSGVDMPGADYQLT | |
| Accession: | KLLGLRPSVKRFMMYQQGCFAGGTVLRLAKDLAENN | |
| AHL83549.1 | KGARVLVVCSEITAVTFRGPSHTHLDSLVGQALFGDG | |
| | AAAVILGADPDTSVERPIFELVSAAQTILPDSEGAIDGH | |
| | LREVGLTFHLLKDVPGLISKNIEKSLVEAFTPIGISDWN | |
| | SIFWIAHPGGPAILDQVEAKLGLKQEKLSATRHVLSEY | |
| | GNMSSACVLFILDEVRKKSVEEGKATTGEGLEWGVLF | |
| | GFGPGLTVETIVLHSLPAV | |
| Chalcone synthase | MAPPAMEEIRRAQRAEGPATVLAIGASTPPNALYQAD | 40 |
| (CHS) | YPDYYFRITKSEHLTELKEKFKQMCDKSMIRKRYMYL | |
| Dendrobium | TEEILKENPNICAFMAPSLDARQDIVVTEVPKLAKEAS | |
| catenatum | ARAIKEWGQPKSRITHLIFCTTSGVDMPGADYQLTRL | |
| Accession: | LGLRPSVNRIMLYQQGCFAGGTVLRLAKDLAENNAG | |
| ALE71934.1 | ARVLVVCSEITAVTFRGPSESHLDSLVGQALFGDGAA | |
| | AIIVGSDPDLTTERPLFQLVSASQTILPESEGAIDGHLRE | |
| | MGLTFHLLKDVPGLISKNIQKSLVETFKPLGIHDWNSI | |
| | FWIAHPGGPAILDQVEIKLGLKEEKLASSRNVLAEYG | |
| | NMSSACVLFILDEMRRRSAEAGQATTGEGLEWGVLF | |
| | GFGPGLTVETVVLRSVPIAGAV | |
| Chalcone isomerase | MSAITAIHVENIEFPAVITSPVTGKSYFLGGAGERGLTI | 41 |
| (CHI) | EGNFIKFTAIGVYLEDVAVASLATKWKGKSSEELLET | |
| Trifolium pratense | LDFYRDIISGPFEKLIRGSKIRELSGPEYSRKVTENCVA | |
| Accession: | HLKSVGTYGDAEVEAMEKFVEAFKPINFPPGASVFYR | |
| PNX83855.1 | QSPDGILGVSISIHFFP | |
| Chalcone isomerase | MAAASLTAVQVENLEFPAVVTSPATGKTYFLGGAGV | 42 |
| (CHI) | RGLTIEGNFIKFTGIGVYLEDQAVASLATKWKGKSSEE | |
| Abrus precatorius | LVESLDFFRDIISGPFEKLIRGSKIRQLSGPEYSKKVME | |
| Accession: | NCVAHMKSVGTYGDAEAAGIEEFAQAFKPVNFPPGA | |
| XP_027366189.1 | SVFYRQSPDGVLGLSFSQDATIPEEEAAVIKNKPVSAA | |
| | VLETMIGEHAVSPDLKRSLAARLPAVLSHGVFKIGN | |
| Chalcone isomerase | MAAEPSITAIQFENLVFPAVVTPPGSSKSYFLAGAGER | 43 |
| (CHI) | GLTIDGKFIKFTGIGVYLEDKAVPSLAGKWKDKSSQQ | |
| Arachis duranensis | LLQTLHFYRDIISGPFEKLIRGSKILALSGVEYSRKVME | |
| Accession: | NCVAHMKSVGTYGDAEAEAIQQFAEAFKNVNFKPGA | |
| XP_015942246.1 | SVFYRQSPLGHLGLSFSQDGNIPEKEAAVIENKPLSSA | |
| | VLETMIGEHAVSPDLKCSLAARLPAVLQQGIIVTPPQH | |
| | N | |
| Chalcone isomerase | MGPSPSVTELQVENVTFPPSVKPPGSTKTLFLGGAGER | 44 |
| (CHI) | GLEIQGKFIKFTAIGVYLEGDAVASLAVKWKGKSKEE | |
| Cephalotus | LTDSVEFFRDIVTGPFEKFTQVTTILPLTGQQYSEKVSE | |
| follicularis | NCVAFWKSVGIYTDAEAKAIEKFIEVFKEETFPPGSSIL | |
| Accession: | FTQSPNGALTIAFSKDGVIPEVGKAVIENKLLAEGLLE | |
| GAV77263.1 | SIIGKHGVSPVAKQCLATRLSELL | |
| Flavanone 3- | MGSASETVCVTGAAGFIGSWLVMRLIQNGYKVRATV | 45 |
| hydroxylase (F3H) | RDPANMKKVKHLLELPNAKTNLSLWKADLAEEGSFD | |
| Abrus precatorius | EAIKGCTGVFHVATPMDFESKDPENEVIKPTINGLIDI | |
| Accession: | MKACMKAKTVRRLVFTSSAGTVDVTEHPKPLFDESC | |
| XP_027329642.1 | WSDVQFCRRVRMTGWMYFVSKTLAEQEAWKFAKEN | |
| | NIDFISVIPPLVVGPFLVPTMPPSLITALSLITGNESHYAI | |
| | IKQGQFVHLDDLCLAHIFLFQHPKAQGRYICCSHEATI | |
| | HDIASLLNQKYPEFNVPTKFKNIPDQLEIIRFSSKKITDL | |
| | GFKFKYSLEDMFTGAVETCKEKRLLSETAEISGTTQK | |
| Flavanone 3- | MKDSVASATASAPGTVCVTGAAGFIGSWLVMRLLER | 46 |
| hydroxylase (F3H) | GYIVRATVRDPANLKKVKHLLDLPKADTNLTLWKAD | |
| Camellia sinensis | LNEEGSFDEAIEGCSGVFHVATPMDFESKDPENEVIKP | |
| Accession: | TINGVLSIIRSCTKAKTVKRLVFTSSAGTVNVQEHQQP | |
| AAT66505.1 | VFDENNWSDLHFINKKKMTGWMYFVSKTLAEKAAW | |
| | EAAKENNIDFISIIPTLVGGPFIMPTFPPSLITALSPITRN | |
| | EGHYSIIKQGQFVHLDDLCESHIFLYERPQAEGRYICSS | |
| | HDATIHDLAKLMREKWPEYNVPTEFKGIDKDLPVVSF | |
| | SSKKLIGMGFEFKYSLEDMFRGAIDTCREKGLLPHSFA | |
| | ENPVNGNKV | |
| Flavanone 3- | MVDMKDDDSPATVCVTGAAGFIGSWLIMRLLQQGYI | 47 |
| hydroxylase (F3H) | VRATVRDPANMKKVKHLQELEKADKNLTLWKADLT | |
| Nyssa sinensis | EEGSFDEAIKGCSGVFHVATPMDFESKDPENEVIKPTI | |
| Accession: | NGVLSIVRSCVKAKTVKRLVFTSSAGTVNLQEHQQLV | |
| KAA8531902.1 | YDENNWSDLDLIYAKKMTGWMYFVSKILAEKAAWE | |
| | ATKENNIDFISIIPTLVVGPFITPTFPPSLITALSLITGNEA | |
| | HYSIIKQGQFVHLDDLCEAHIFLYEQPKAEGRYICSSH | |
| | DATIYDLAKMIREKWPEYNVPTELKGIEKDLQTVSFSS | |
| | KKLIGMGFEFKYSLEDMYKGAIDTCREKGLLPYSTHE | |
| | TPANANANANANVKKNQNENTEI | |
| Flavanone 3- | MASESESVCVTGASGFVGSWLVMRLLDRGYTVRATV | 48 |
| hydroxylase (F3H) | RDPANKKKVKHLLDLPKAATHLTLWKADLAEEGSFD | |
| Rosa chinensis | EAIKGCTGVFHVATPMDFESKDPENEVIKPTINGVLDI | |
| Accession: | MKACLKAKTVRRLVFTASAGSVNVEETQKPVYDESN | |
| XP_024167119.1 | WSDVEFCRRVKMTGWMYFASKTLAEQEAWKFAKEN | |
| | NIDFITIIPTLVIGPFLMPAMPPSLITGLSPLTGNESHYSII | |
| | KQGQFIHLDDLCQSHIYLYEHPKAEGRYICSSHDATIH | |
| | EIAKLLREKYPEYNVPTTFKGIEENLPKVHFSSKKLLE | |
| | TGFEFKYSLEDMFVGAVDACKAKGLLPPPTERVEKQE | |
| | VDESSVVGVKVTG | |
| Flavonoid 3′ | MSPLILYSIALAIFLYCLRTLLKRHPHRLPPGPRPWPIIG | 49 |
| hydroxylase (F3′H) | NLPHMGQMPHHSLAAMARTYGPLMHLRLGFVDVIV | |
| Cephalotus | AASASVASQLLKTHDANFSSRPHNSGAKYIAYNYQDL | |
| follicularis | VFAPYGPRWRMLRKISSVHLFSGKALDDYRHVRQEE | |
| Accession: | VAVLIRALARAESKQAVNLGQLLNVCTANALGRVML | |
| GAV84063.1 | GRRVFGDGSGVSDPMAEEFKSMVVEVMALAGVFNIG | |
| | DFIPALDWLDLQGVAAKMKNLHKRFDTFLTGLLEEH | |
| | KKMLVGDGGSEKHKDLLSTLISLKDSADDEGLKLTDT | |
| | EIKALLLNMFTAGTDTSSSTVEWAIAELIRHPKILAQV | |
| | LKELDTVVGRDRLVTDLDLPQLTYLQAVIKETFRLHP | |
| | STPLSLPRVAAESCEIMGYHIPKGSTLLVNVWAIARDP | |
| | KEWAEPLEFRPERFLPGGEKPNVDIKGNDFEVIPFGAG | |
| | RRICAGMSLGLRMVQLLTATLVHAFDWDLTSGLMPE | |
| | DLSMEEAYGLTLQRAEPLMVHPRPRLSPNVY | |
| Flavonoid 3′ | MASFLLYSILSAVFLYFIFATLRKRHRLPLPPGPKPWPII | 50 |
| hydroxylase (F3′H) | GNLPHMGPVPHHSLAALAKVYGPLMHLRLGFVDVV | |
| Theobroma cacao | VAASASVAAQFLKVHDANFSSRPPNSGAKYVAYNYQ | |
| Accession: | DLVFAPYGPRWRMLRKISSVHLFSGKALDDFRHVRQ | |
| EOY22049.1 | DEVGVLVRALADAKTKVNLGQLLNVCTVNALGRVM | |
| | LGKRVFGDGSGKADPEADEFKSMVVELMVLAGVVNI | |
| | GDFIPALEWLDLQGVQAKMKKLHKRFDRFLSAILEEH | |
| | KIKARDGSGQHKDLLSTFISLEDADGEGGKLTDTEIKA | |
| | LLLNMFTAGTDTSSSTVEWAIAELIRHPKILAQVRKEL | |
| | DSVVGRDRLVSDLDLPNLTYFQAVIKETFRLHPSTPLS | |
| | LPRMASESCEINGYHIPKGATLLVNVWAIARDPDEWK | |
| | DPLEFRPERFLPGGERPNADVRGNDFEVIPFGAGRRIC | |
| | AGMSLGLRMVQLLAATLVHAFDWELADGLMPEKLN | |
| | MEEAFGLTLQRAAPLMVHPRPRLSPRAY | |
| Flavonoid 3′ | MTPLTLLIGTCVTGLFLYVLLNRCTRNPNRLPPGPTPW | 51 |
| hydroxylase (F3′H) | PVVGNLPHLGTIPHHSLAAMAKKYGPLMHLRLGFVD | |
| Gerbera hybrida | VVVAASASVAAQFLKTHDANFADRPPNSGAKHIAYN | |
| Accession: | YQDLVFAPYGPRWRMLRKICSVHLFSTKALDDFRHV | |
| ABA64468.1 | RQEEVAILARALVGAGKSPVKLGQLLNVCTTNALAR | |
| | VMLGRRVFDSGDAQADEFKDMVVELMVLAGEFNIG | |
| | DFIPVLDWLDLQGVTKKMKKLHAKFDSFLNTILEEHK | |
| | TGAGDGVASGKVDLLSTLISLKDDADGEGGKLSDIEI | |
| | KALLLNLFTAGTDTSSSTIEWAIAELIRNPQLLNQARK | |
| | EMDTIVGQDRLVTESDLGQLTFLQAIIKETFRLHPSTPL | |
| | SLPRMALESCEVGGYYIPKGSTLLVNVWAISRDPKIW | |
| | ADPLEFQPTRFLPGGEKPNTDIKGNDFEVIPFGAGRRIC | |
| | VGMSLGLRMVQLLTATLIHAFDWELADGLNPKKLNM | |
| | EEAYGLTLQRAAPLVVHPRPRLAPHVYETTKV | |
| Flavonoid 3′ | MAPLLLLFFTLLLSYLLYYYFFSKERTKGSRAPLPPGP | 52 |
| hydroxylase (F3′H) | RGWPVLGNLPQLGPKPHHTLHALSRAHGPLFRLRLGS | |
| Phoenix dactylifera | VDVVVAASAAVAAQFLRAHDANFSNRPPNSGAEHIA | |
| Accession: | YNYQDLVFAPYGPGWRARRKLLNVHLFSGKALEDLR | |
| XP_008791304.2 | PVREGELALLVRALRDRAGANELVDLGRAANKCATN | |
| | ALARAMVGRRVFQEEEDEKAAEFENMVVELMRLAG | |
| | VFNVGDFVPGIGWLDLQGVVRRMKELHRRYDGFLDG | |
| | LIAAHRRAAEGGGGGGKDLLSVLLGLKDEDLDFDGE | |
| | GAKLTDTDIKALLLNLFTAGTDTTSSTVEWALSELVK | |
| | HPDILRKAQLELDSVVGGDRLVSESDLPNLPFMQAIIK | |
| | ETFRLHPSTPLSLPRMAAEECEVAGYCIPKGATLLVNV | |
| | WAIARDPAVWRDPLEFRPARFLPDGGCEGMDVKGND | |
| | FGIIPFGAGRRICAGMSLGIRMVQFMTATLAHAFHWD | |
| | LPEGQMPEKLDMEEAYGLTLQRATPLMVHPVPRLAP | |
| | TAYQS | |
| Cytochrome P450 | MASNSNLIRAIESALGVSFGSELVSDTAIVVVTTSVAVI | 53 |
| reductase (CPR) | IGLLFFLLKRSSDRSKESKPVVISKPLLVEEEEEEDEVE | |
| Camellia sinensis | AGSGKTKVTMFYGTQTGTAEGFAKSLAKEIKARYEK | |
| Accession: | AIVKVVDLDDYAADDDQYEQKLKKETLVFFMLATYG | |
| XP_028084858 | DGEPTDDAARFYKWFTEENERGAWLQQLTYGVFSLG | |
| | NRQYEHFNKIGKVVDEQLSKQGAKRLIPVGLGDDDQ | |
| | CIEDDFAAWRETLWPELDQLLRDEDDANTVSTPYAA | |
| | AIPEYRVVIHDPLSGRGEAPSFSIDSHLTICEIWSTSREG | |
| | SNQQISEYFWTSNSLKTMASNSNLIRSIESALGVSFGSE | |
| | SVSDTAIVVVTTSVAVIIGLLFFLLKRSSDRSKESKPVV | |
| | ISKPLLVEEEEDEVEAGSGKTKVTLFYGTQTGTAEGFA | |
| | KSLAEEIKARYEKAIVKVVDLDDYAADDDQYEQKLK | |
| | KETLVFFMLATYGDGEPTDNAARFYKWFTEENERGA | |
| | WLQQLTYGVFSLGNRQYEHFNKIGKVVDEQLSKQGA | |
| | KRLIPVGLGDDDQCIEDDFAAWRETLWPELDQLLRDE | |
| | DDANTVSTPYTAAIPEYRVVIHDPTTTSYEDKNLNMA | |
| | NGNASYDIHHPCRVNVAVQRELHKPESDRSCIHLEFDI | |
| | SGTGIIYETGDHVGVYADNFDEVVEEAANLLGQPLEL | |
| | LFSVHADKDDGTSLGGSLPPPFPGPCTLRDALAHYAD | |
| | LLNPPRKAALSALAAHAVEPSEAERLKFLSSPQGKED | |
| | YSQWVVASQRSLLEIMAEFPSAKPPLGVFFAAVAPRL | |
| | QPRYYSISSSPRFVPNRVHVTCALVYGPSPTGRIHKGV | |
| | CSTWMKNAVPLEKSHDCSSAPIFTRTSNFKLPTDPSIPI | |
| | IMVGPGTGLAPFRGFLQERLALKEDGVQLGHAMLFFG | |
| | CRNRRMDFIYEDELNNFVDQGAVSELVVAFSREGPEK | |
| | EYVQHKLNAKAAQVWGLISQGGYLYVCGDAKGMAR | |
| | DVHRMLHTIVEQQENVDSRKAEVIVKKLQMEGRYLR | |
| | DVW | |
| Cytochrome P450 | MASNSNLIRAIESALGVSFGSELVSDTAIVVVTTSVAVI | 54 |
| reductase (CPR) | IGLLFFLLKRSSDRSKESKPVVISKPLLVEEEEEEDEVE | |
| Cephalotus | AGSGKTKVTMFYGTQTGTAEGFAKSLAKEIKARYEK | |
| follicularis | AIVKVVDLDDYAADDDQYEQKLKKETLVFFMLATYG | |
| Accession: | DGEPTDDAARFYKWFTEENERGAWLQQLTYGVFSLG | |
| GAV59576.1 | NRQYEHFNKIGKVVDEQLSKQGAKRLIPVGLGDDDQ | |
| | CIEDDFAAWRETLWPELDQLLRDEDDANTVSTPYAA | |
| | AIPEYRVVIHDPLSGRGEAPSFSIDSHLTICEIWSTSREG | |
| | SNQQISEYFWTSNSLKTMASNSNLIRSIESALGVSFGSE | |
| | SVSDTAIVVVTTSVAVIIGLLFFLLKRSSDRSKESKPVV | |
| | ISKPLLVEEEEDEVEAGSGKTKVTLFYGTQTGTAEGFA | |
| | KSLAEEIKARYEKAIVKVVDLDDYAADDDQYEQKLK | |
| | KETLVFFMLATYGDGEPTDNAARFYKWFTEENERGA | |
| | WLQQLTYGVFSLGNRQYEHFNKIGKVVDEQLSKQGA | |
| | KRLIPVGLGDDDQCIEDDFAAWRETLWPELDQLLRDE | |
| | DDANTVSTPYTAAIPEYRVVIHDPTTTSYEDKNLNMA | |
| | NGNASYDIHHPCRVNVAVQRELHKPESDRSCIHLEFDI | |
| | SGTGIIYETGDHVGVYADNFDEVVEEAANLLGQPLEL | |
| | LFSVHADKDDGTSLGGSLPPPFPGPCTLRDALAHYAD | |
| | LLNPPRKAALSALAAHAVEPSEAERLKFLSSPQGKED | |
| | YSQWVVASQRSLLEIMAEFPSAKPPLGVFFAAVAPRL | |
| | QPRYYSISSSPRFVPNRVHVTCALVYGPSPTGRIHKGV | |
| | CSTWMKNAVPLEKSHDCSSAPIFTRTSNFKLPTDPSIPI | |
| | IMVGPGTGLAPFRGFLQERLALKEDGVQLGHAMLFFG | |
| | CRNRRMDFIYEDELNNFVDQGAVSELVVAFSREGPEK | |
| | EYVQHKLNAKAAQVWGLISQGGYLYVCGDAKGMAR | |
| | DVHRMLHTIVEQQENVDSRKAEVIVKKLQMEGRYLR | |
| | DVW | |
| Cytochrome P450 | MSSSSSSPFDLMSAIIKGEPVVVSDPANASAYESVAAE | 55 |
| reductase (CPR) | LSSMLIENRQFAMIISTSIAVLIGCIVMLLWRRSGGSGS | |
| Brassica napus | SKRAETLKPLVLKPPREDEVDDGRKKVTIFFGTQTGT | |
| Accession: | AEGFAKALGEEARARYEKTRFKIVDLDDYAADDDEY | |
| XP_013706600.1 | EEKLKKEDVAFFFLATYGDGEPTDNAARFYKWFTEG | |
| | DDRGEWLKNLKYGVFGLGNRQYEHFNKVAKVVDDI | |
| | LVEQGAQRLVHVGLGDDDQCIEDDFTAWREALWPEL | |
| | DTILREEGDTAVTPYTAAVLEYRVSIHNSADALNEKN | |
| | LANGNGHAVFDAQHPYRANVAVRRELHTPESDRSCT | |
| | HLEFDIAGSGLTYETGDHVGVLSDNLNETVEEALRLL | |
| | DMSPDTYFSLHSDKEDGTPISSSLPPTFPPCSLRTALTR | |
| | YACLLSSPKKSALLALAAHASDPTEAERLKHLASPAG | |
| | KDEYSKWVVESQRSLLEVMAEFPSAKPPLGVFFAAV | |
| | APRLQPRFYSISSSPKIAETRIHVTCALVYEKMPTGRIH | |
| | KGVCSTWMKSAVPYEKSENCCSAPIFVRQSNFKLPSD | |
| | SKVPIIMIGPGTGLAPFRGFLQERLALVESGVELGPSVL | |
| | FFGCRNRRMDFIYEEELQRFLESGALSELSVAFSREGP | |
| | TKEYVQHKMMDKASDIWNMISQGAYVYVCGDAKG | |
| | MARDVHRSLHTIAQEQGSMDSTKAESFVKNLQMSGR | |
| | YLRDVW | |
| Flavonoid 3′,5′- | MALDTFLLRELAAAAVLFLISHYLIHSLLKKSTPPLPPG | 56 |
| hydroxylase | PKGWPFVGALPLLGTMPHVALAQMAKKYGPVMYLK | |
| (F3′5′H) | MGTCGMVVASTPDAARAFLKTLDLNFSNRPPNAGAT | |
| Cephalotus | HLAYNAQDMVFADYGPRWKLLRKLSNLHMLGGKAL | |
| follicularis | EDWTQVRTVELGHMIQAMCEASRAKEPVVVPEMLTY | |
| Accession: | AMANMIGKVILGHRVFVTQGSESNEFKDMVVELMTS | |
| GAV62131 | AGYFNIGDFIPSIAWMDLQGIERGMKKLHKRFDALLT | |
| | KMFEEHMATAHERKGNPDLLDIVMANRDNSEGERLT | |
| | TTNIKALLLNLFSAGTDTSSSIIEWSLAEMLKNPSILKR | |
| | AHEEMDQVIGRNRRLEESDIKKLPYLQAICKESFRKHP | |
| | STPLNLPRVSSQACQVNGYYIPKDTRLSVNIWAIGRDP | |
| | EVWENPLDFTPERFLSGKNAKIDPRGNDFELIPFGAGR | |
| | RICAGTRMGIVLVEYILGTLVHSFDWSLPHGVKLNMD | |
| | EAFGLALQKAVPLAAIVSPRLAPTAYVV | |
| Flavonoid 3′,5′- | MSIFLITSLLLCLSLHLLLRRRHISRLPLPPGPPNLPIIGA | 57 |
| hydroxylase | LPFIGPMPHSGLALLARRYGPIMFLKMGIRRVVVASSS | |
| (F3′5′H) | TAARTFLKTFDSHFSDRPSGVISKEISYNGQNMVFADY | |
| Dendrobium | GPKWKLLRKVSSLHLLGSKAMSRWAGVRRDEALSMI | |
| moniliforme | QFLKKHSDSEKPVLLPNLLVCAMANVIGRIAMSKRVF | |
| Accession: | HEDGEEAKEFKEMIKELLVGQGASNMEDLVPAIGWL | |
| AEB96145 | DPMGVRKKMLGLNRRFDRMVSKLLVEHAETAGERQ | |
| | GNPDLLDLVVASEVKGEDGEGLCEDNIKGFISDLFVA | |
| | GTDTSAIVIEWAMAEMLKNPSILRRAQEETDRVIGRH | |
| | RLLDESDIPNLPYLQAICKEALRKHPPTPLSIPHYASEP | |
| | CEVEGYHIPGETWLLVNIWAIGRDPDVWENPLVFDPE | |
| | RFLQGEMARIDPMGNDFELIPFGAGRRICAGKLAGMV | |
| | MVQYYLGTLVHAFDWSLPEGVGELDMEEGPGLVLPK | |
| | AVPLAVMATPRLPAAAYGLL | |
| Dihydroflavonol 4- | MGSEAETVCVTGASGFIGSWLIMRLLERGYTVRATVR | 58 |
| reductase (DFR) | DPDNEKKVKHLVELPKAKTHLTLWKADLSDEGSFDE | |
| Acer palmatum | AIHGCTGVFHVATPMDFESKDPENEVIKPTINGVLGIM | |
| Accession: | KACKKAKTVKRLVFTSSAGTVDVEEHKKPVYDENSW | |
| AWN08247.1 | SDLDFVQSVKMTGWMYFVSKTLAEKAAWKFAEENSI | |
| | DFISVIPPLVVGPFLMPSMPPSLITALSPITRNEGHYAII | |
| | KQGNYVHLDDLCMGHIFLYEHAESKGRYFCSSHSATI | |
| | LELSKFLRERYPEYDLPTEYKGVDDSLENVVFCSKKIL | |
| | DLGFQFKYSLEDMFTGAVETCREKGLIPLTNIDKKHV | |
| | AAKGLIPNNSDEIHVAAAEKTTATA | |
| Dihydroflavonol 4- | MGSASETVCVTGAAGFIGSWLVMRLIQNGYKVRATV | 59 |
| reductase (DFR) | RDPANMKKVKHLLELPNAKTNLSLWKADLAEEGSFD | |
| Abrus precatorius | EAIKGCTGVFHVATPMDFESKDPENEVIKPTINGLIDI | |
| Accession: | MKACMKAKTVRRLVFTSSAGTVDVTEHPKPLFDESC | |
| XP_027329642.1 | WSDVQFCRRVRMTGWMYFVSKTLAEQEAWKFAKEN | |
| | NIDFISVIPPLVVGPFLVPTMPPSLITALSLITGNESHYAI | |
| | IKQGQFVHLDDLCLAHIFLFQHPKAQGRYICCSHEATI | |
| | HDIASLLNQKYPEFNVPTKFKNIPDQLEIIRFSSKKITDL | |
| | GFKFKYSLEDMFTGAVETCKEKRLLSETAEISGTTQK | |
| Dihydroflavonol 4- | MENEKKGPVVVTGASGYVGSWLVMKLLQKGYEVRA | 60 |
| reductase (DFR) | TVRDPTNLKKVKPLLDLPRSNELLSIWKADLDGIEGSF | |
| Dendrobium | DEVIRGSIGVFHVATPMNFQSKDPENEVIQPAINGLLGI | |
| moniliforme | LRSCKNAGSVQRVIFTSSAGTVNVEEHQAAAYDETC | |
| Accession: | WSDLDFVNRVKMTGWMYFLSKTLAEKAAWEFVKD | |
| AEB96144.1 | NHIHLITIIPTLVVGSFITSEMPPSMITALSLITGNDAHY | |
| | SILKQIQFVHLDDLCDAHIFLFEHPKANGRYICSSYDST | |
| | IYGLAEMLKNRYPTYAIPHKFKEIDPDIKCVSFSSKKL | |
| | MELGFKYKYTMEEMFDDAIKTCREKKLIPLNTEEIVL | |
| | AAEKFEEVKEQIAVK | |
| Dihydroflavonol 4- | MASESESVCVTGASGFVGSWLVMRLLDRGYTVRATV | 61 |
| reductase (DFR) | RDPANKKKVKHLLDLPKAATHLTLWKADLAEEGSFD | |
| Rosa chinensis | EAIKGCTGVFHVATPMDFESKDPENEVIKPTINGVLDI | |
| Accession: | MKACLKAKTVRRLVFTASAGSVNVEETQKPVYDESN | |
| XP_024167119.1 | WSDVEFCRRVKMTGWMYFASKTLAEQEAWKFAKEN | |
| | NIDFITIIPTLVIGPFLMPAMPPSLITGLSPLTGNESHYSII | |
| | KQGQFIHLDDLCQSHIYLYEHPKAEGRYICSSHDATIH | |
| | EIAKLLREKYPEYNVPTTFKGIEENLPKVHFSSKKLLE | |
| | TGFEFKYSLEDMFVGAVDACKAKGLLPPPTERVEKQE | |
| | VDESSVVGVKVTG | |
| Leucoanthocyanidin | MTVSSPCVGEGQGRVLIIGASGFIGEFIAQASLDSGRTT | 62 |
| reductase (LAR) | FLLVRSLDKGAIPSKSKTINSLHDKGAILIHGVIEDQEF | |
| Camellia sinensis | VEGILKDHKIDIVISAVGGANILNQLTIVKAIKAVGTIK | |
| Accession: | RFLPSEFGHDVDRANPVEPGLAMYKEKRMVRRLIEES | |
| XP_028127206.1 | GVPYTYICCNSIASWPYYDNTHPSEVIPPLDRFQIYGD | |
| | GTVKAYFVDGSDIGKFTMKVVDDIRTLNKSVHFRPSC | |
| | NFLNMNELSSLWEKKIGYMLPRLTVTEDDLLAAAAE | |
| | NIIPQSIVASFTHDIFIKGCQVNFSIDGPNEVEVSNLYPD | |
| | ETFRTMDECFDDFVMKMDRWN | |
| Leucoanthocyanidin | MTRSPSPNGQAEKGSRILIIGATGFIGHFIAQASLASGK | 63 |
| reductase (LAR) | STYILSRAAARCPSKARAIKALEDQGAISIHGSVNDQE | |
| Coffea arabica | FMEKTLKEHEIDIVISAVGGGNLLEQVILIRAMKAVGT | |
| Accession: | IKRFLPSEFGHDVDRAEPVEPGLTMYNEKRRVRRLIEE | |
| XP_027097479.1 | SGVPYTYICCNSIASWPYYDNTHPSEVSPPLDQFQIYG | |
| | DGSVKAYFVAGADIGKFTVKATEDVRTLNKIVHFRPS | |
| | CNFLNINELATLWEKKIGRTLPRVVVSEDDLLAAAEE | |
| | NIIPQSVVASFTHDIFIKGCQVNFPVDGPNEIEVSSLYP | |
| | DEPFQTMDECFNEFAGKIEEDKKHVVGTKGKNIAHRL | |
| | VDVLTAPKLCA | |
| Leucoanthocyanidin | MKSTNMNGSSPNVSEETGRTLVVGSGGFMGRFVTEA | 64 |
| reductase (LAR) | SLDSGRPTYILARSSSNSPSKASTIKFLQDRGATVIYGSI | |
| Theobroma cacao | TDKEFMEKVLKEHKIEVVISAVGGGSILDQFNLIEAIR | |
| Accession: | NVDTVKRFLPSEFGHDTDRADPVEPGLTMYEQKRQIR | |
| ADD51357.1 | RQIEKSGIPYTYICCNSIAAWPYHDNTHPADVLPPLDR | |
| | FKIYGDGTVKAYFVAGTDIGKFTIMSIEDDRTLNKTVH | |
| | FQPPSNLLNINEMASLWEEKIGRTLPRVTITEEDLLQM | |
| | AKEMRIPQSVVAALTHDIFINGCQINFSLDKPTDVEVC | |
| | SLYPDTPFRTINECFEDFAKKIIDNAKAVSKPAASNNAI | |
| | FVPTAKPGALPITAICT | |
| Leucoanthocyanidin | MTVSPSIASAAKSGRVLIIGATGFIGKFVAEASLDSGLP | 65 |
| reductase (LAR) | TYVLVRPGPSRPSKSDTIKSLKDRGAIILHGVMSDKPL | |
| Fragaria x | MEKLLKEHEIEIVISAVGGATILDQITLVEAITSVGTVK | |
| ananassa | RFLPSEFGHDVDRADPVEPGLTMYLEKRKVRRAIEKS | |
| Accession: | GVPYTYICCNSIASWPYYDNKHPSEVVPPLDQFQIYGD | |
| ABH07785.2 | GTVKAYFVDGPDIGKFTMKTVDDIRTMNKNVHFRPSS | |
| | NLYDINGLASLWEKKIGRTLPKVTITENDLLTMAAEN | |
| | RIPESIVASFTHDIFIKGCQTNFPIEGPNDVDIGTLYPEE | |
| | SFRTLDECFNDFLVKVGGKLETDKLAAKNKAAVGVE | |
| | PMAITATCA | |
| Anthocyanin | MTQNKEPVNQGKSEHDEQRVESLASSGIESIPKEYVRL | 66 |
| dioxygenase (ANS) | NEELTSMGNVFEEEKKEEGSQVPTIDIKDIASEDPEVR | |
| Chenopodium | GKAIQELKRAAMEWGVMHLVNHGISDELIDRVKVAG | |
| quinoa | QTFFELPVEEKEKYANDQASGNVQGYGSKLANSASG | |
| Accession: | RLEWEDYYFHLSYPEDKRDLSIWPETPADYIPAVSEYS | |
| XP_021735950.1 | KELRYLATKILSALSLALGLEEGRLEKEVGGLEELLLQ | |
| | FKINYYPKCPQPELALGVEAHTDVSALTFILHNMVPG | |
| | LQLFYEGKWVTAKCVPNSIIMHIGDTIEILSNGKYKSIL | |
| | HRGLVNKEKVRISWAVFCEPPKEKIILKPLPDLVSDEE | |
| | PARYPPRTFAQHVQYKLFRKTQGPQTTITKN | |
| Anthocyanin | MASSKVMPAPARVESLASSGLASIPTEYVRPEWERDD | 67 |
| dioxygenase (ANS) | SLGDALEEIKKTEEGPQIPIVDLRGFDSGDEKERLHCM | |
| Iris sanguinea | EEVKEAAVEWGVMHIVNHGIAPELIERVRAAGKGFFD | |
| Accession: | LPVEAKERYANNQSEGKIQGYGSKLANNASGQLEWE | |
| QCI56004.1 | DYFFHLIFPSDKVDLSIWPKEPADYTEVMMEFAKQLR | |
| | VVVTKMLSILSLGLGFEEEKLEKKLGGMEELLMQMKI | |
| | NYYPKCPQPELALGVEAHTDVSSLSFILHNGVPGLQV | |
| | FHGGRWVNARLVPGSLVVHVGDTLEILSNGRYKSVL | |
| | HRGLVNKEKVRISWAVFCEPPKEKIVLEPLAELVDKR | |
| | SPAKYPPRTFAQHIQHKLFKKAQEQLAGGVHIPEAIQN | |
| Anthocyanin | MATQVASIPRVEMLASAGIQAIPTEYVRPEAERNSIGD | 68 |
| dioxygenase (ANS) | VFEEEKKLEGPQIPVVDLMGLEWENEEVFKKVEEDM | |
| Magnolia sprengeri | KKAASEWGVMHIINHGISMELMDRVRIAGKAFFDLPI | |
| Accession: | EEKEMYANDQASGKIAGYGSKLANNASGQLEWEDYF | |
| AHU88620.1 | FHLIFPEDKRDMSIWPKQPSDYVEATEEFAKQLRGLV | |
| | TKVLVLLSRGLGVEEDRLEKEFGGMEELLLQMKINYY | |
| | PKCPQPDLALGVEAHTDVSALTFILHNMVPGLQVFFD | |
| | DKWVTAKCIPGALVVHIGDSLEILSNGKYRSILHRGLV | |
| | NKEKVRISWAIFCEPPKEKVVLQPLPELVSEAEPARFT | |
| | PRTFSQHVRQKLFKKQQDALENLKSE | |
| Anthocyanin | MVSSAAVVATRVERLATSGIKSIPKEYVRPQEELTNIG | 69 |
| dioxygenase (ANS) | NVFEEEKKEGPEVPTIDLTEIESEDEVVRARCHETLKK | |
| Prosopis alba | AAQEWGVMNLVNHGIPEELLNQLRKAGETFFSLPIEE | |
| Accession: | KEKYANDQASGKIQGYGSKLANNASGQLEWEDYFFH | |
| XP_028787846.1 | LVFPEDKCDLSIWPRTPSDYIEVTSEYARQLRGLATKI | |
| | LGALSLGLGLEKGRLEEEVGGMEELLLQMKINYYPIC | |
| | PQPELALGVEAHTDVSSLTFLLHNMVPGLQLFYNGQ | |
| | WITAKCVPNSIFMHIGDTVEILSNGRYKSILHRGLVNK | |
| | EKVRISWAVFCEPPKEKIILKPLPELVTDDEPARFPPRT | |
| | FAQHIQHKLFRKCQEGLSK | |
| Anthocyanidin-3- | MPQFTTNEPHVAVLAFPFGTHAAPLITIIHRLAVASPN | 70 |
| O-glycotransferase | THFSFLNTSQSNNSIFSSDVYNRQPNLKAHNVWDGVP | |
| (3GT) | EGYVFVGKPQESIELFVKAAPETFRKGVEAAVAETGR | |
| Cephalotus | KVSCLVTDAFFWFAAEIAGELGVPWVPFWTAGPCSLS | |
| follicularis | THVYTDLIRKTIGVGGIEGREDESLEFIPGMSQVVIRDL | |
| Accession: | QEGIVFGNLESVFSDMVHRMGIVLPQAAAIFINSFEEL | |
| GAV66155.1 | DLTITNDLKSKFKQFLSIGPLNLASPPPRVPDTNGCLP | |
| | WLDQQKVASVAYISFGTVMAPSPPELVALAEALEASK | |
| | IPFIWSLGEKLKVHLPKGFLDKTRTHGIVVPWAPQSDV | |
| | LENGAVGVFITHCGWNSLLESIAGGVPMICRPFFGDQ | |
| | RLNGRMVQDVWEIGVTATGGPFTTEGVMGDLDLILS | |
| | QARGKKMKDNISVLKTLAQTAVGPEGSSAKNYEALL | |
| | NLVRLSI | |
| Anthocyanidin-3- | MAPQPIDDDHVVYEHHVAALAFPFSTHASPTLALVRR | 71 |
| O-glycotransferase | LAAASPNTLFSFFSTSQSNNSLFSNTITNLPRNIKVFDV | |
| (3GT) | ADGVPDGYVFAGKPQEDIELFMKAAPHNFTTSLDTCV | |
| Prunus cerasifera | AHTGKRLTCLITDAFLWFGAHLAHDLGVPWLPLWLS | |
| Accession: | GLNSLSLHVHTDLLRHTIGTQSIAGRENELITKNVNIPG | |
| AKV89253.1 | MSKVRIKDLPEGVIFGNLDSVFSRMLHQMGQLLPRAN | |
| | AVLVNSFEELDITVINDLKSKFNKLLNVGPFNLAAAA | |
| | SPPLPEAPTAADDVTGCLSWLDKQKAASSVVYVSFGS | |
| | VARPPEKELLAMAQALEASGVPFLWSLKDSFKTPLLN | |
| | ELLIKASNGMVVPWAPQPRVLAHASVGAFVTHCGWN | |
| | SLLETIAGGVPMICRPFFGDQRVNARLVEDVLEIGVTV | |
| | EDGVFTKHGLIKYFDQVLSQQRGKKMRDNINTVKLL | |
| | AQQPVEPKGSSAQNFKLLLDVISGSTKV | |
| Anthocyanidin-3- | MVFQSHIGVLAFPFGTHAAPLLTVVQRLATSSPHTLFS | 72 |
| O-glycotransferase | FFNSAVSNSTLFNNGVLDSYDNIRVYHVWDGTPQGQ | |
| (3GT) | AFTGSHFEAVGLFLKASPGNFDKVIDEAEVETGLKISC | |
| Scutellaria | LITDAFLWFGYDLAEKRGVPWLAFWTSAQCALSAHM | |
| baicalensis | YTHEILKAVGSNGVGETAEEELIQSLIPGLEMAHLSDL | |
| Accession: | PPEIFFDKNPNPLAITINKMVLKLPKSTAVILNSFEEIDP | |
| A0A482AQV3 | IITTDLKSKFHHFLNIGPSILSSPTPPPPDDKTGCLAWLD | |
| | SQTRPKSVVYISFGTVITPPENELAALSEALETCNYPFL | |
| | WSLNDRAKKSLPTGFLDRTKELGMIVPWAPQPRVLA | |
| | HRSVGVFVTHCGWNSILESICSGVPLICRPFFGDQKLN | |
| | SRMVEDSWKIGVRLEGGVLSKTATVEALGRVMMSEE | |
| | GEIIRENVNEMNEKAKIAVEPKGSSFKNFNKLLEIINAP | |
| | QSS | |
| Anthocyanidin-3- | MSQTTTNPHVAVLAFPFSTHAAPLLAVVRRLAAAAPH | 73 |
| O-glycotransferase | AVFSFFSTSQSNASIFHDSMHTMQCNIKSYDISDGVPE | |
| (3GT) | GYVFAGRPQEDIELFTRAAPESFRQGMVMAVAETGRP | |
| Vitis 109hikimat | VSCLVADAFIWFAADMAAEMGLAWLPFWTAGPNSLS | |
| Accession: | THVYIDEIREKIGVSGIQGREDELLNFIPGMSKVRFRDL | |
| P51094 | QEGIVFGNLNSLFSRMLHRMGQVLPKATAVFINSFEEL | |
| | DDSLTNDLKSKLKTYLNIGPFNLITPPPVVPNTTGCLQ | |
| | WLKERKPTSVVYISFGTVTTPPPAEVVALSEALEASRV | |
| | PFIWSLRDKARVHLPEGFLEKTRGYGMVVPWAPQAE | |
| | VLAHEAVGAFVTHCGWNSLWESVAGGVPLICRPFFG | |
| | DQRLNGRMVEDVLEIGVRIEGGVFTKSGLMSCFDQIL | |
| | SQEKGKKLRENLRALRETADRAVGPKGSSTENFITLV | |
| | DLVSKPKDV | |
| Acetyl-CoA | MPPPDHKAVSQFIGGNPLETAPASPVADFIRKQGGHS | 74 |
| carboxylase (ACC) | VITKVLICNNGIAAVKEIRSIRKWAYETFGDERAIEFTV | |
| Ustilago maydis | MATPEDLKVNADYIRMADQYVEVPGGSNNNNYANV | |
| 521 | DLIVDVAERAGVHAVWAGWGHASENPRLPESLAASK | |
| Accession: | HKIIFIGPPGSAMRSLGDKISSTIVAQHADVPCMPWSG | |
| XP_011390921.1 | TGIKETMMSDQGFLTVSDDVYQQACIHTAEEGLEKAE | |
| | KIGYPVMIKASEGGGGKGIRKCTNGEEFKQLYNAVLG | |
| | EVPGSPVFVMKLAGQARHLEVQLLADQYGNAISIFGR | |
| | DCSVQRRHQKIIEEAPVTIAPEDARESMEKAAVRLAK | |
| | LVGYVSAGTVEWLYSPESGEFAFLELNPRLQVEHPTT | |
| | EMVSGVNIPAAQLQVAMGIPLYSIRDIRTLYGMDPRG | |
| | NEVIDFDFSSPESFKTQRKPQPQGHVVACRITAENPDT | |
| | GFKPGMGALTELNFRSSTSTWGYFSVGTSGALHEYAD | |
| | SQFGHIFAYGADRSEARKQMVISLKELSIRGDFRTTVE | |
| | YLIKLLETDAFESNKITTGWLDGLIQDRLTAERPPADL | |
| | AVICGAAVKAHLLARECEDEYKRILNRGQVPPRDTIK | |
| | TVFSIDFIYENVKYNFTATRSSVSGWVLYLNGGRTLV | |
| | QLRPLTDGGLLIGLSGKSHPVYWREEVGMTRLMIDSK | |
| | TCLIEQENDPTQIRSPSPGKLVRFLVDSGDHVKANQAI | |
| | AEIEVMKMYLPLVAAEDGVVSFVKTAGVALSPGDIIG | |
| | ILSLDDPSRVQHAKPFAGQLPDFGMPVIVGNKPHQRY | |
| | TALVEVLNDILDGYDQSFRMQAVIKELIETLRNPELPY | |
| | GQASQILSSLGGRIPARLEDVVRNTIEMGHSKNIEFPA | |
| | ARLRKLTENFLRDSVDPAIRGQVQITIAPLYQLFETYA | |
| | GGLKAHEGNVLASFLQKYYEVESQFTGEADVVLELR | |
| | LQADGDLDKVVALQTSRNGINRKNALLLTLLDKHIKG | |
| | TSLVSRTSGATMIEALRKLASLQGKSTAPIALKAREVS | |
| | LDADMPSLADRSAQMQAILRGSVTSSKYGGDDEYHA | |
| | PSLEVLRELSDSQYSVYDVLHSFFGHREHHVAFAALC | |
| | TYVVRAYRAYEIVNFDYAVEDFDVEERAVLTWQFQL | |
| | PRSASSLKERERQVSISDLSMMDNNRRARPIRELRTGA | |
| | MTSCADVADIPELLPKVLKFFKSSAGASGAPINVLNV | |
| | AVVDQTDFVDAEVRSQLALYTNACSKEFSAARVRRV | |
| | TYLLCQPGLYPFFATFRPNEQGIWSEEKAIRNIEPALA | |
| | YQLELDRVSKNFELTPVPVSSSTIHLYFARGIQNSADT | |
| | RFFVRSLVRPGRVQGDMAAYLISESDRIVNDILNVIEV | |
| | ALGQPEYRTADASHIFMSFIYQLDVSLVDVQKAIAGFL | |
| | ERHGTRFFRLRITGAEIRMILNGPNGEPRPIRAFVTNET | |
| | GLVVRYETYEETVADDGSVILRGIEPQGKDATLNAQS | |
| | AHFPYTTKVALQSRRSRAHALQTTFVYDFIDVLGQAV | |
| | RASWRKVAASKIPGDVIKSAVELVFDEQENLREVKRA | |
| | PGMNNIGMVAWLVEVLTPEYPAGRKLVVIGNDVTIQ | |
| | AGSFGPVEDRFFAAASKLARELGVPRLYISANSGARIG | |
| | LATEALDLFKVKFVGDDPAKGFEYIYLDDESLQAVQA | |
| | KAPNSVMTKPVQAADGSVHNIITDIIGKPQGGLGVEC | |
| | LSGSGLIAGETSRAKDQIFTATIITGRSVGIGAYLARLG | |
| | ERVIQVEGSPLILTGYQALNKLLGREVYTSNLQLGGPQ | |
| | IMYKNGVSHLTAQDDLDAVRSFVNWISYVPAQRGGP | |
| | LPIMPTTDSWDRAVTYQPPRGPYDPRWLINGTKAEDG | |
| | TKLTGLFDEGSFVETLGGWATSVVTGRARLGGIPVGV | |
| | LAVETRTLERVVPADPANPNSTEQRIMEAGQVWYPNS | |
| | AYKTAQAIWDFDKEGLPLVILANWRGFSGGQQDMYD | |
| | EILKQGSKIVDGLSSYKQPVFVHIPPMGELRGGSWVV | |
| | VDSAINDNGMIEMSADVNSARGGVLEASGLVEIKYRA | |
| | DKQRATMERLDSVYAKLSKEAAEATDFTAQTTARKA | |
| | LAEREKQLAPIFTAIATEYADAHDRAGRMLATGVLRS | |
| | ALPWENARRYFYWRLRRRLTEVAAERTVGEANPTLK | |
| | HVERLAVLRQFVGAAASDDDKAVAEHLEASADQLLA | |
| | ASKQLKAQYILAQISTLDPELRAQLAASLK | |
| Acetyl-CoA | MVDHKSLPGHFLGGNSVDTAPQDPVCEFVKSHQGHT | 75 |
| carboxylase (ACC) | VISKVLIANNGMAAMKEIRSVRKWAYETFGNERAIEF | |
| Hesseltinella | TVMATPEDLKANAEYIRMADNYIEVPGGTNNNNYAN | |
| vesiculosa | VELIVDVAERTGVHAVWAGWGHASENPRLPEMLAKS | |
| Accession: | KNKCVFIGPPASAMRSLGDKISSTIVAQSADVPTMGW | |
| ORX57605.1 | SGDGVSETTTDHNGHVLVNDDVYNSACVKTAEAGLA | |
| | SAEKIGFPVMIKASEGGGGKGIRKVEDPSTFKQAFAQ | |
| | VQGEIPGSPIFIMKLAGNARHLEVQLLADQYGNAISLF | |
| | GRDCSVQRRHQKIIEEAPVTIAKPDIFEQMEKAAVRLG | |
| | KLVGYVSAGTVEYLYSHHDEKFYFLELNPRLQVEHPT | |
| | TEMVSGVNLPAAQLQIAMGIPMHRIRDIRVLYGVQPN | |
| | SASEIDFDLEHPTALQSQRRPMPKGHVIAVRITAENPD | |
| | AGFKPSGGVMQELNFRSSTNVWGYFSVVSSGAMHEY | |
| | ADSQFGHIFAYGENRQQARKNMVIALKELSIRGDFRT | |
| | TVEYIIRLLETPDFTDNTINTGWLDMLISKKLTAERPDT | |
| | MLAVFCGAVTKAHLASVECWQQYKNSLERGQIPSKE | |
| | SLKTVFTVDFIYENIRYNFTVTRSAPGIYTLYLNGTKT | |
| | QVGVRDLSDGGLLISLNGRSHTTYNREEVQATRLMID | |
| | GKTCLLEKESDPTQLRSPSPGKLVSLLLENGDHIRTGQ | |
| | AYAEIEVMKMYMPLVASEDGHVQFIKQVGATLEAGD | |
| | IIGILSLDDPSRVKHALPFTGQVPKYGLPHLTGDKPHQ | |
| | RFTHLKQTLEYVLQGYDNQGLIQTIVKELSEVLNNPEL | |
| | PYSELSASMSVLSGRIPGRLEQQLHDLINQAHAQNKG | |
| | FPAVDIQQAIDTFARDHLTTQAEVNAYKTAVAPIMTIA | |
| | ASYSNGLKQHEHSVYVDLMEQYYNVEVLFNSNQSRD | |
| | EEVILALRDQHKDDLEKVINIILSHAKVNIKNNLILMLL | |
| | DIIYPATSSEALDRCFLPILKHLSEIDSRGTQKVTLKAR | |
| | EYLILCQLPSLEERQSQMYNILKSSVTESVYGGGTEYR | |
| | TPSYDAFKDLIDTKFNVFDVLPNFFYHPDSYVSLAALE | |
| | VYCRRSYHAYKILDVAYNLEHQPYIVAWKFLLQSSA | |
| | GGGFNNQRIASYSDLTFLLNKTEEEPIRTGAMVALKTL | |
| | EELEAELPRIMTAFEEEPLPPMLMKQPPPDKTEERMEN | |
| | ILNISIQGQDMEDDTLRKNMTTLIQAHSDAFRKAALR | |
| | RITLVVCRDNQTPDYYTFRERNGYEEDETIRHIEPALA | |
| | YQLELARLSNFDIKPCFIENRQMHVYYAVAKENPSDC | |
| | RFFIRALVRPGRVKSSMRTADYLISESDRLLTDILDTLE | |
| | IVSHDYKNSDCNHLFINFIPTFAIEADEVETALKDFVDR | |
| | HGKRLWKLRVTGAEIRFNIQSKRPDAPVIPLRFTVDNV | |
| | SGYILKVDVYQEVKTDKNGWILKSVGKIPGAMHMQP | |
| | LSTPYPTKEWLQPRRYKAHLMGTTYVYDFPELFRQAI | |
| | HNLWAQACKADAAVKIPSQVIEAKELVLDDDNQLQA | |
| | IDRAPGTNTVGMVAWLLTLRTPDYPRGRRVIAIANDI | |
| | TFKIGSFGVQEDLVFYKASEYARELGVPRVYLSANSG | |
| | ARIGLADELISRFHVAWKDEDQPGSGFEYLYLLPEEY | |
| | DALIQQGDAQSVLVQEVQDKGERRFRITDIIGHTDGL | |
| | GVENLRGSGLIAGATSRAYDDIFTITLVTCRSVGIGAY | |
| | LVRLGQRTVQNEGQPIILTGAPALNKVLGREVYTSNL | |
| | QLGGTQIMYKNGVSHLTAENDLEGINKIMQWLSFVPE | |
| | CRGAPLPMRAGADPIDREIEYLPPKGPSDPRFFLAGKQ | |
| | ENGKWLSGFFDHGSFVETLSGWARTVVVGRARLGGI | |
| | PMGVVAVETRTVENIVPADPANADSQEQVVMEAGGV | |
| | WFPNSAYKTAQAINDFNKGEQLPLMIFANWRGFSGG | |
| | QRDMYNEVLKYGAQIVDALSNYKQPVFVYVVPNGEL | |
| | RGGAWVVVDSTINEDMMEMYADTQARGGVLEPEGI | |
| | VEIKYRRPQLLATMERLDPVYSDLKRRLAALDDSQKE | |
| | QADELIAQVEAREQALLPVYQQVAIQFADLHDRSGR | |
| | MEAKGVIRKTLEWRTARHYFYWRVRRRLLEEYAIRK | |
| | MDESRDQAKTLLQQWFQADTNLDDFDKNDQAVVA | |
| | WFDAKNLLLDQRIAKLKSEKLKDHVVQLASVDQDAV | |
| | VEGFSKLMESLSVDQRKEVLHKLATRF | |
| Acetyl-CoA | MASTTPHDSRVVSVSSGKKLYIEVDDGAGKDAPAIVF | 76 |
| carboxylase (ACC) | MHGLGSSTSFWEAPFSRSNLSSRFRLIRYDFDGHGLSP | |
| Rhodotorula | VSLLDAADDGAMIPLVDLVEDLAAVMEWTGVDKVA | |
| toruloides | GIVGHSMSGLVASTFAAKYPQKVEKLVLLGAMRSLN | |
| NBRC10032 | PTVQTNMLKRADTVLESGLSAIVAQVVSAALSDKSKQ | |
| Accession: | DSPLAPAMVRTLVLGTDPLGYAAACRALAGAKDPDY | |
| GEM08739.1 | STIKAKTLVVSGESDYLSNKETTEALVNDIPGAKEVQ | |
| | MDGVGHWHAVEDPAGLAKILDGFFLQGKFSGEAKA | |
| | VNGSHAVDETPKKPKYDHGRVVKYLGGNSLESAPPS | |
| | NVADWVRERGGHTVITKILIANNGIAAVKEIRSVRKW | |
| | AYETFGSERAIEFTVMATPEDLKVNADYIRMADQYVE | |
| | VPGGTNNNNYANVDVIVDVAERAGVHAVWAGWGH | |
| | ASENPRLPESLAASKHKIVFIGPPGSAMRSLGDKISSTI | |
| | VAQHAEVPCMDWSGQGVDQVTQSLEGYVTVADDVY | |
| | QQACVHDADEGLARASRIGYPVMIKASEGGGGKGIR | |
| | KVEREQDFKQAFQAVLTEVPGSPVFIMKLAGAARHLE | |
| | VQVLADQYGNAISLFGRDCSVQRRHQKIIEEAPVTIAK | |
| | PDTFEQMEKSAVRLAKLVGYVSAGTVEFLYSAADDK | |
| | FAFLELNPRLQVEHPTTEMVSGVNLPAAQLQVAMGV | |
| | PLHRIRDIRTLYGKAPNGSSEIDFEFENPESAKTQRKPS | |
| | PKGHVVAVRITAENPDAGFKPSMGTLQELNFRSSTNV | |
| | WGYFSVGSAGGLHEFADSQFGHIFAYGSDRSESRKN | |
| | MVVALKELSIRGDFRTTVEYLIKLLETDAFEQNTITTA | |
| | WLDSLISARLTAERPDTTLAIICGAVTKAHLASEANIA | |
| | EYKRILEKGQSPPKELLATVVPLEFVLEDVKYRATASR | |
| | SSPSSWSIYVNGSNVSVGIRPLADGGLLILLDGRSYTC | |
| | YAKEEVGALRLSIDSRTVLVAQENDPTQLRSPSPGKL | |
| | VRYFIESGEHISKGEAYAEIEVMKMIMPLIAAEDGIAQ | |
| | FIKQPGATLEAGDILGILSLDDPSRVHHAKPFDGQLPA | |
| | LGLPSIIGTKPHQRFAYLKDVLSNILMGYDNQAIMQSS | |
| | IKELISVLRNPELPYGEANAVLSTLSGRIPAKLEQTLRQ | |
| | YIDSAHESGAEFPSAKCRKAIDTTLEQLRPAEAQTVRN | |
| | FLVAFDDIVYRYRSGLKHHEWSTLAGIFAAYAETEKP | |
| | FSGKDSDVVLELRDAHRDSLDSVVKIVLSHYKAASKN | |
| | SLVLALLDVVKDSDSVPLIEQVVSPALKDLADLDSKA | |
| | TTKVALKAREVLIHIQLPSLDERLGQLEQILKASVTPT | |
| | VYGEPGHDRTPRGEVLKDVIDSRFTVFDVLPSFFQHQ | |
| | DQWVSLAALDTYVRRAYRSYNLLNIEHIEADAAEDEP | |
| | ATVAWSFRMRKAASESEPPTPTTGLTSQRTASYSDLT | |
| | FLLNNAQSEPIRYGAMFSVRSLDGFRQELGTVLRHFP | |
| | DSNKGKLQQQPAASSSQEQWNVINVALTVPASAQVD | |
| | EDALRADFAAHVNAMSAEIDARGMRRLTLLICREGQ | |
| | YPSYYTVRKQDGTWKELETIRDIEPALAFQLELGRLSN | |
| | FHLEPCPVENRQVHIYYATAKGNSSDCRFFVRALVRP | |
| | GRLRGNMKTADYLVSEADRLVTDVLDSLEVASSQRR | |
| | AADGNHISLNFLYSLRLDFDEVQAALAGFIDRHGKRF | |
| | WRLRVTGAEIRIVLEDAQGNIQPIRAIIENVSGFVVKYE | |
| | AYREVTTDKGQVILKSIGPQGALHLQPVNFPYPTKEW | |
| | LQPKRYKAHVVGTTYVYDFPDLFRQAIRKQWKAVGK | |
| | TAPAELLVAKELVLDEFGKPQEVARPPGTNNIGMVG | |
| | WIYTIFTPEYPSGRRVVVIANDITFKIGSFGPEEDRYFY | |
| | AVTQLARQLGLPRVYLSANSGARLGIAEELVDLFSVA | |
| | WADSSRPEKGFKYLYLTAEKLGELKNKGEKSVITKRI | |
| | EDEGETRYQITDIIGLQEGLGVESLKGSGLIAGETSRAY | |
| | DDIFTITLVTARSVGIGAYLVRLGQRAVQVEGQPIILTG | |
| | AGALNKVLGREVYSSNLQLGGTQIMYKNGVSHLTAA | |
| | NDLEGVLSIVQWLAFVPEHRGAPLPVLPSPVDPWDRSI | |
| | DYTPIKGAYDPRWFLAGKTDEADGRWLSGFFDKGSF | |
| | QETLSGWAQTVVVGRARLGGIPMGAIAVETRTIERIIP | |
| | ADPANPLSNEQKIMEAGQVWYPNSSFKTGQAIFDFNR | |
| | EGLPLIIFANWRGFSGGQQDMFDEVLKRGSLIVDGLS | |
| | AYKQPVFVYIVPNGELRGGAWVVLDPSINAEGMMEM | |
| | YVDETARAGVLEPEGIVEIKLRKDKLLALMDRLDPTY | |
| | HALRVKSTDASLSPTDAAQAKTELAAREKQLMPIYQQ | |
| | VALQFADSHDKAGRILSKGCAREALEWSNARRYFYA | |
| | RLRRRLAEEAAVKRLGEADPTLSRDERLAIVHDAVGQ | |
| | GVDLNNDLAAAAAFEQGAAAITERVKLARATTVAST | |
| | LAQLAQDDKEAFAASLQQVLGDKLTAADLARILA | |
| Malonyl-CoA | MNANLFSRLFDGLVEADKLAIETLEGERISYGDLVAR | 77 |
| synthase (matB) | SGRMANVLVARGVKPGDRVAAQAEKSVAALVLYLA | |
| Rhodopseudomonas | TVRAGAVYLPLNTAYTLHELDYFIGDAEPKLVVCDPA | |
| palustris | KREGIAALAQKVGAGVETLDAKGQGSLSEAAAQASV | |
| Accession: | DFATVPREGDDLAAILYTSGTTGRSKGAMLSHDNLAS | |
| WP_011661926.1 | NSLTLVEFWRFTPDDVLIHALPIYHTHGLFVASNVTLF | |
| | ARASMIFLPKFDPDAIIQLMSRASVLMGVPTFYTRLLQ | |
| | SDGLTKEAARHMRLFISGSAPLLADTHREWASRTGHA | |
| | VLERYGMTETNMNTSNPYDGARVPGAVGPALPGVSL | |
| | RVVDPETGAELSPGEIGMIEVKGPNVFQGYWRMPEKT | |
| | KAEFRDDGFFITGDLGKIDADGYVFIVGRGKDLVITGG | |
| | FNVYPKEVESEIDAISGVVESAVIGVPHADLGEGVTAV | |
| | VVRDKGASVDEAAVLGALQGQLAKFKMPKRVLFVD | |
| | DLPRNTMGKVQKNVLREAYAKLYAK | |
| Malonyl-CoA | MVNHLFDAIRLSITSPESTFIELEDGKVWTYGAMFNCS | 78 |
| synthase (matB) | ARITHVLVKLGVSPGDRVAVQVEKSAQALMLYLGCL | |
| Rhizobium | RAGAVYLPLNTAYTPAELEYFLGDATPKLVVVSPCAA | |
| sp. BUS003 | EQLEPLARRVGTRLLTLGVNGDGSLMDMASLEPVEF | |
| Accession: | ADIERKADDLAAILYTSGTTGRSKGAMLTHDNLLSNA | |
| NKF42351.1 | QTLREHWRFTSADRLIHALPIFHTHGLFVATNVTLLAG | |
| | GAIYLLSKFDPDQIFALMTRATVMMGVPTFYTRLLQD | |
| | ERLNKANTRHMRLFISGSAPLLAETHRLFEEYTGHAIL | |
| | ERYGMTETNMITSNPCDGARVPGTVGYALPGVSVRIT | |
| | DPVSGEPLAAGEPGMIEVKGPNVFQGYWNMPDKTKE | |
| | EFRSDGYFTTGDIGVMETDGRISIVGRGKDLIISGGYNI | |
| | YPKEIENEIDAIEGVVESAVIGVPHPDLGEGVTAIVVG | |
| | QPKAHLDLTTITNNLQGRLARFKQPKNVIFVDELPRNT | |
| | MGKVQKNVLRDRYRDLYLK | |
| Malonyl-CoA | MANHLFDLVRANATDLTKTFIETETGLKLTYDDLMT | 79 |
| synthase (matB) | GTARYANVLVGLGVKPGDRVAVQVEKSAGAIFLYLA | |
| Ochrobactrum sp. | CVRAGAVFLPLNTAYTLTEIEYFLGDAEPALVVCDPA | |
| 3-3 | RRDGITEVAKKTGVPAVETLGKGQDGSLFDKAAAAP | |
| Accession: | ETFADVARGPGDLAAILYTSGTTGRSKGAMLSHDNLA | |
| WP_114216069.1 | SNALTLKDYWRFGADDVLLHALPIFHTHGLFVATNTI | |
| | LVAGASMLFLPKFDADKVFELMPRATTMMGVPTFYV | |
| | RLVQDARLTREATKHMRLFISGSAPLLAETHKLFREK | |
| | TGVSILERYGMTETNMNTSNPYDGDRVAGTVGFPLPG | |
| | VALRVADPETGAAIPQGEIGVIEVKGPNVFSGYWRMP | |
| | EKTAAEFRQDGFFITGDLGKIDDQGYVHIVGRGKDLV | |
| | ISGGYNVYPKEVETEIDGMAGVVESAVIGVPHPDFGE | |
| | GVTAVVVAEKGASLDEATIIKTLEQRLARYKLPKRVI | |
| | VVDDLPRNTMGKVQKNLLRDAYKGLYGG | |
| Malonate | MSPELISILVLVVVFVIATTRSVNMGALAFAAAFGVGT | 80 |
| transporter (matC) | LVADLDADGIFAGFPGDLFVVLVGVTYLFAIARANGT | |
| Rhizobiales | TDWLVHAAVRLVRGRVALIPWVMFALTGALTAIGAV | |
| bacterium | SPAAVAIVAPVALSFATRYSISPLLMGTMVVHGAQAG | |
| Accession: | GFSPISIYGSIVNGIVEREKLPGSEIGLFLASLVANLLIA | |
| MBN8942514.1 | AVLFAVLGGRKLWARGAVTPEGDGAPGKAGTGTTGS | |
| | GSDTGTGTGTGTGTSAGTGGTAPTAVAVRSDRETGG | |
| | AEGTGVRLTPARVATLVALVALVVAVLGFDLDAGLT | |
| | AVTLAVVLSTAWPDDSRRAVGEIAWSTVLLICGVLTY | |
| | VGVLEEMGTITWAGEGVGGIGVPLLAAVLLCYIGAIV | |
| | SAFASSVGIMGALIPLAVPFLAQGEIGAVGMVAALAV | |
| | SATVVDVSPFSTNGALVLAAAPDVDRDRFFRQLMVY | |
| | GGIVVAAVPALAWLVLVVPGFG | |
| Malonate | MGIELLSIGLLIAMFIIATIQPINMGALAFAGAFVLGSMI | 81 |
| transporter (matC) | IGMKTNEIFAGFPSDLFLTLVAVTYLFAIAQINGTIDWL | |
| Rhizobium | VECAVRLVRGRIGLIPWVMFLVAAIITGFGALGPAAV | |
| leguminosarum | AILAPVALSFAVQYRIHPVMMGLMVIHGAQAGGFSPI | |
| Accession: | SIYGGITNQIVAKAGLPFAPTSLFLSSFFFNLAIAVLVFF | |
| AAC83457.1 | VFGGARVMKHDPASLGPLPELHPEGVSASIRGHGGTP | |
| | AKPIREHAYGTAADTATTLRLNNERITTLIGLTALGIG | |
| | ALVFKFNVGLVAMTVAVVLALLSPKTQKAAIDKVSW | |
| | STVLLIAGIITYVGVMEKAGTVDYVANGISSLGMPLLV | |
| | ALLLCFTGAIVSAFASSTALLGAIIPLAVPFLLQGHISAI | |
| | GVVAAIAISTTIVDTSPFSTNGALVVANAPDDSREQVL | |
| | RQLLIYSALIAIIGPIVAWLVFVVPGLV | |
| Malonate | MNIEILSIGLLVAIFIIATIQPINMGVLAFGCTFVLGSLII | 82 |
| transporter (matC) | GMKPADIFAGFPADLFLTLVAVTYLFAIAQINGTIDWL | |
| Agrobacterium vitis | VERSVRMVRGRVGWIPWVMFLVAAIITGFGALGPAA | |
| Accession: | VAILAPVALSFAVQYRIHPVLMGLMVIHGAQAGGFSPI | |
| WP_180575084.1 | SIYGGITNQIVAKAGLPFAPTSLFLSSFFFNLAIAVLIFFI | |
| | FGGLSILKQRSSVKGPLPELHPEGISASIKGHGGTPAKP | |
| | FREHAYGTAADTQSKVRLTTEKVTTLIGLTALGVGAL | |
| | VFKFNVGLVAITVAVLLALLSPTTQKAAIDKVSWSTV | |
| | LLISGIITYVGVMEKAGTIDYVAHGISSLGMPLLVALL | |
| | LCFTGAIVSAFASSTALLGAIIPLAVPFLLQGHISAVGV | |
| | VAAIAISTTIVDTSPFSTNGALVVANAPDDQRDKVMR | |
| | QMLIYSALIALIGPVIAWLVFVVPGII | |
| Malonate | MSIEILSILLLVAMFVIATIQPINMGALAFACTFVLGSLI | 83 |
| transporter (matC) | IGMKTSDIFAGFPSDLFLTLVAVTYLFAIAQINGTIDWL | |
| Neorhizobium sp. | VECAVRMVRGHVAWIPWVMFVVAAIITGFGALGPAA | |
| Accession: | VAILAPVALSFAVQYRIHPVMMGLMVIHGAQAGGFSP | |
| WP_105370917.1 | ISVYGGITNQIVAKAGLPFAPTSLFLSSFFFNLAIAVLVF | |
| | FVFGGARIMKQAAGPTGPLPELHPEGVSAAIRGHGGT | |
| | PAKPIREHAYGTAADTLQTLRLTPEKVFTLIGLTALGI | |
| | GALVFKFNVGLVAITVAVALALISPKTQKAAVDKVS | |
| | WSTVLLIAGIITYVGVLEKAGTVNYVANGISSLGMPLL | |
| | VALLLCFTGAIVSAFASSTALLGAIIPLAVPFLLQGHIS | |
| | AVGVVAAIAISTTIVDTSPFSTNGALVVANAPDETREQ | |
| | VLRQLLIYSALIAIIGPVVAWLVFVVPGLV | |
| Malonate CoA- | MTTWNQKQQRKAQKLAKACDSGFDKYVPHERIIALL | 84 |
| transferase (MdcA) | ETVIDRGDRVCLEGNNQKQADFLSKSLSSCNPDIVNG | |
| Moraxella | LHIVQSVLALPSHIDVFERGIASKVDFSFAGPQSLRLAQ | |
| catarrhalis | LVQAQKITIGAIHTYLELYGRYFIDLTPNVALITAHAA | |
| Accession: | DKRGNLYTGANTEDTPAIVEATTFKSGIVIAQVNEIVD | |
| WP_064617969.1 | ELPRVDIPSDWVDYYTQSPKHNYIEPLFTRDPAQITEIQ | |
| | ILMAMMAIKGIYAPYKINRLNHGIGFDTAAIELLLPTY | |
| | AESLGLKGEICTHWALNPHPTLIPAIESGFIHSVHSFGS | |
| | EVGMENYVKARSDVFFTGADGSMRSNRAFSQTAGLY | |
| | ACDLFIGSTLQIDLQGNSSTATADRIAGFGGAPNMGSD | |
| | PHGRRHASYAYMKAGREAVDGSPIKGRKLVVQMVE | |
| | TYREHMQSVFVNELDAFKLQQKMGADLPPIMIYGDD | |
| | VTHIVTEEGIANLLLCRTPDEREQAIRGVAGYTPIGLG | |
| | RDDTMVARLRERKVIQRPEDLGINPMHATRDLLAAKS | |
| | VKDLVRWSDRLYEPPSRFRNW | |
| Malonate CoA- | MNAPQPRQWDSLRQNRARRLERAASLGLAGQNGKEI | 85 |
| transferase (MdcA) | PVDRIIDLLEAVIQPGDRVCLEGNNQKQADFLSESLAD | |
| Dechloromonas | CDPARINHLSMVQSVLALPSHVDLFERGLATRLDFSFS | |
| aromatica | GPQGARLAKLVQEQRIEIGAIHTYLELFGRYFMDLTPN | |
| Accession: | VALIAAQAADAEGNLYLGPNTEDTPAIVEATAFKGGI | |
| WP_011289741.1 | VIAQVNERLDKLPRVDVPADWVDFTVLAPKPNYIEPL | |
| | FTRDPAQITEVQVLMAMMAIKGIYAEYGVTRINHGIG | |
| | FDTAAIELLLPTYAADLGLKGKICTHWALNPHPTLIPA | |
| | IEAGFVESVHCFGSEVGMDDYISARSDIFFTGADGSMR | |
| | SNRAFSQTAGLYACDMFIGSTLQMDLAGNSSTATLGR | |
| | ITGFGGAPNMGSDPHGRRHASPAWLKAGREAYGPQA | |
| | IRGRKLVVQMVETFREHMAPVFVDDLDAWKLQASM | |
| | GSDLPPIMIYGDDVSHIVTEEGIANLLLCRTPAEREQAI | |
| | RGVAGFTPVGMARDKGTVENLRDRGIIRRPEDLGIDP | |
| | RQASRDLLAARSIKDLVRCSGGLYAPPSRFRNW | |
| Malonate CoA- | MSRQWDTQADSRRQRLQRAAALAPQGRVVAADDVV | 86 |
| transferase (MdcA) | ALLEAVIEPGDRVCLEGNNQKQADFLARCLTEVDPAR | |
| Pseudomonas | VHDLHMVQSVLSLAAHLDVFERGIAKRLDFSFSGPQA | |
| cissicola | ARLAGLVSEGRIEIGAIHTYLELFGRYFIDLTPRIALVT | |
| Accession: | AQAADRHGNLYTGPNTEDTPVIVEATAFKGGIVIAQV | |
| WP_078590875.1 | NEILDTLPRVDIPADWVDFVTQAPKPNYIEPLFTRDPA | |
| | QISEIQVLMAMMAIKGIYAEYGVDRLNHGIGFDTAAIE | |
| | LLLPTYAQSLGLKGKICRHWALNPHPALIPAIESGFVQ | |
| | SVHSFGSELGMENYIAARPDIFFTGADGSMRSNRALS | |
| | QTAGLYACDMFIGSTLQIDLQGNSSTATRDRIAGFGG | |
| | APNMGSDARGRRHASAAWLKAGREAATPGEMPRGR | |
| | KLVVQMVETFREHMAPAFVDRLDAWELAERANMPL | |
| | PPVMIYGDDVSHVLTEEGIANLLLCRTPEEREQAIRGV | |
| | SGYTAVGLGRDKRMVENLRDRGVIKRPDDLGIRPRD | |
| | ATRDLLAARTVKDLVRWSGGLYDPPKRFRNW | |
| Malonate CoA- | MNKIYREKRSWRTRRDRKAKRIEHMKQIAKGKIIPTE | 87 |
| transferase (MdcA) | KIVEALTALIFPGDRVVIEGNNQKQASFLSKALSQVNP | |
| Geobacillus | EKVNGLHIIMSSVSRPEHLDLFEKGIARKIDFSYAGPQS | |
| subterraneus | LRMSQMLEDGKLVIGEIHTYLELYGRLFIDLTPSVALV | |
| Accession: | AADKADASGNLYTGPNTEETPTLVEATAFRDGIVIAQ | |
| WP_184319829.1 | VNELADELPRVDIPGSWIDFVVAADHPYELEPLFTRDP | |
| | RLITEIQILMAMMVIKGIYERHNIQSLNHGIGFNTAAIE | |
| | LLLPTYGESLGLKGKICKHWALNPHPTLIPAIETGWVE | |
| | SIHCFGGEVGMEKYIAARPDIFFTGKDGNLRSNRTLSQ | |
| | VAGQYAVDLFIGSTLQIDRDGNSSTVTNGRLAGFGGA | |
| | PNMGHDPRGRRHSSPAWLDMITSDHPAAKGRKLVVQ | |
| | MVETFQKGNRPVFVESLDAIEVGRSARLATTPIMIYGE | |
| | DVTHIVTEEGIAYLYKASSLEERRQAIAAIAGVTPIGLE | |
| | RDPRKTEQLRRDGVVAFPEDLGIRRTDAKRSLLAAKSI | |
| | EELVEWSEGLYEPPARFRSW | |
| Pantothenate kinase | MLLTIDVGNTHTVLGLFDGEEIVEHWRISTDSRRTADE | 88 |
| (CoaX) | LAVLLQGLMGTHPLLGMELGEGIDGIAICSTVPAVLH | |
| Streptomyces sp. | ELREVSRRYYGDVPAILVEPGVKTGVPILMDNPKEVG | |
| CLI2509 | TDRIINAVAAQHLYGGPAIVVDFGTATTFDAVSARGE | |
| Accession: | YTGGVIAPGIEISVEALGLRGAQLRKIELARPRSVIGKS | |
| WP_095682415.1 | TVEAMQSGILYGFAGQVDGVVQRMACELAPDPADVT | |
| | VIATGGLAPMVLGEAAVIDHHEPWLTLIGLRLVYERN | |
| | AGRR | |
| Pantothenate kinase | MTKLWLDLGNTRLKYWLTDDSGQVLDHAAEQHLQA | 89 |
| (CoaX) | PAELLKGLTFRLERLNPDFIGVSSVLGQAVNNHVAESL | |
| Streptomyces | ERLQKPFEFAQVHAKHALMSSDYNPAQLGVDRWLQ | |
| cinereus | MLGIIEPSKKQCVIGCGTAVTIDLVDQGHHLGGYIFPSI | |
| Accession: | YLQRESLFSGTRQISIIDGTFDSIDSGTNTQDAVHHGIM | |
| WP_188874884.1 | LSIVGAINETIHRYPQFEITMTGGDAHTFEPHLSASVEI | |
| | RQDLVLAGLQRFFAAKNNTKNQN | |
| Pantothenate kinase | MLLTIDVGNTQTTLGLFDGEEVVDHWRISTDPRRTAD | 90 |
| (CoaX) | ELAVLMQGLMGRQPGGAGRERVDGLAICSSVPAVLH | |
| Kitasatospora | ELREVTRRYYGDLPAVLVAPGVKTGVHVLMDNPKEV | |
| kifunensis | GADRIVNALAANHLYGGPCIVVDFGTATTFDAINERG | |
| Accession: | DYVGGAIAPGIEISVEALGVRGAQLRKIELAKPRNVIG | |
| WP_184936930.1 | KNTVEGMQSGVLYGFAGQVDGLVTRMAKELSPTDPE | |
| | DVQVIATGGLAPLVLDEASSIDVHEPWLTLIGLRLVYE | |
| | RNTAS | |
| glutamyl-tRNA | MTLLALGINHKTAPVSLRERVTFSPDTLDQALDSLQA | 91 |
| reductase (hemA) | LPMVQGGVVLSTCNRTEIYLSVEEQDNLREALIRWLC | |
| Citrobacter | EYHNLNEEDLRNSLYWHQDNDAVSHLMRVASGLDS | |
| freundii | LVLGEPQILGQVKKAFADSQKGHQNASALERMFQKS | |
| Accession: | FSVAKRVRTETDIGSSAVSVAFAACTLARQIFESLSTV | |
| NTY05430.1 | TVLLVGAGETIELVARHLREHKVKKMIIANRTRERAQ | |
| | VLADEVGAEVISLSDIDARLQDADIIISSTASPLPIIGKG | |
| | MVERALKNRRNQPMLLVDIAVPRDVEPEVGKLSNAY | |
| | LYSVDDLQSIISHNLAQRKAAAVEAETIVEQEASEFMA | |
| | WLRAQGASDTIREYRSQSEQIRDELTAKALAALQQGG | |
| | DAQAIMQDLAWKLTNRLIHAPTKSLQQAARDGDSER | |
| | LNILRDSLGLE | |
| glutamyl-tRNA | MTLLALGINHKTAPVSLRERVTFSPETIEQALSSLLQQP | 92 |
| reductase (hemA) | LVQGGVVLSTCNRTELYLSVEQQENLQEQLVKWLCD | |
| Pseudomonas | YHHLSADEVRKSLYWHQDNAAVSHLMRVASGLDSL | |
| reactans | VVGEPQILGQVKKAFAESQHGQAVSGELERLFQKSFS | |
| Accession: | VAKRVRTETDIGASAVSVAFAACTLARQIFESLSDVSV | |
| NWA43040.1 | LLVGAGETIELVARHLREHKVRHMMIANRTRERAQV | |
| | LASEVGAEVITLQDIDARLADADIIISSTASPLPIIGKGM | |
| | VERALKARRNQPMLMVDIAVPRDIEPEVGKLANAYL | |
| | YSVDDLHSIIQNNMAQRKAAAVQAESIVEQESSNFMA | |
| | WLRSQGAVEIIRDYRSRADLVRAEAEAKALAAIAQGA | |
| | DVSAVIHELAHKLTNRLIHAPTRSLQQAASDGDVERL | |
| | QILRDSLGLDQQ | |
| glutamyl-tRNA | MTLLALGINHKTAPVALREKVSFSPDTMGDALNNLLQ | 93 |
| reductase (hemA) | QPAVRGGVVLSTCNRTELYLSMEDKENSHEQLIRWLC | |
| Gammaproteobacteria | QYHQIEPNELQSSIYWHQDNQAVSHLMRVASGLDSL | |
| Accession: | VLGEPQILGQVKKAFADSQNYDSLSSELERLFQKSFSV | |
| WP_193016510.1 | AKRVRTETQIGANAVSVAFAACTLARQIFESLSSLTILL | |
| | VGAGETIELVARHLREHQVKKIIIANRTKERAQRLASE | |
| | VDAEVITLSEIDECLAQADIVISSTASPLPIIGKGMVER | |
| | ALKKRRNQPMLLVDIAVPRDIEQDVEKLNNVYLYSV | |
| | DDLEAIIQHNREQRQAAAVQAEHIVQQESGQFMDWL | |
| | RAQGAVGAIREYRDSAETLRAEMTEKAITLIQNGADA | |
| | EKVIQQLSHQLMNRLIHTPTKSLQQAASDGDIERLNLL | |
| | RESLGITHN | |
| 5-aminolevulinic | MGPALDVRGKQLAAGYASVAGQADVEKIHQDQGITI | 94 |
| acid synthase | PPNATVEMCPHAKAARDAARIAEDLAAAAASKQQPA | |
| (ALAS) | KKAGGCPFHAAQAQAQAKPAAAPKETVATADKKGK | |
| Schizophyllum | SPRAAGGFDYEKFYEEELDKKHQDKSYRYFNNINRLA | |
| commune H4-8 | ARFPTAHTAKVTDEVEVWCSNDYLGMGGNPVVLET | |
| Accession: | MHRVLDKYGHGAGGTRNIAGNGALHLSLEQELARLH | |
| XP_003036856.1 | RKEGALVFTSCYVANDATLSTLGSKMPGCVIFSDRMN | |
| | HASMIQGIRHSGTKKVIFEHNDLADLEKKLAEYPKETP | |
| | KIIAFESVYSMCGSIGPIKEICDLAEKYGAITFLDEVHA | |
| | VGLYGPRGAGVAEHLDYDLHKAAGDSPDAIPGTVMD | |
| | RVDIITGTLGKSYGAIGGYIAGSARFVDMIRSYAPGFIF | |
| | TTSLPPATVAGAQASVVYQKEYLGDRQLKQVNVREV | |
| | KRRFAELDIPVVPGPSHIVPVLVGDAALAKQASDKLL | |
| | AEHDIYVQAINYPTVARGEERLRITVTQRHTLEQMDH | |
| | LIGAVDQVFNELNINRVQDWKRLGGRASVGVPGGQD | |
| | FVEPIWTDEQVGLADGSAPLTLRNGQPNEVSHDAVV | |
| | AARSRFDWLLGPIPSHIQAKRLGQSLEGTPIAPLAPKQ | |
| | SSGLKLPVEEMTMGQTIAVAA | |
| 5-aminolevulinic | MDKIARFKQTCPFLGRTKNSTLRNLSTSSSPRFPSLTAL | 95 |
| acid synthase | TERATKCPVMGPALNVRSKEIVAGYASVAANSDVALI | |
| (ALAS) | HKEKGVFPPPGATVEMCPHASAARAAARMADDLAA | |
| Crassisporium | AAEKKKGHFTSAAPRDEAAQAAAAGCPFHVKAAAD | |
| funariophilum | AAAARKAAAAPAPVKAKEDGGFNYESFYVNELDKK | |
| Accession: | HQDKSYRYFNNINRLAAKFPVAHTSNVKDEVEVWCA | |
| KAF8165006.1 | NDYLGMGNNPVVLETMHRTLDKYGHGAGGTRNIAG | |
| | NGAMHLSLEQELATLHRKPAALVFSSCYVANDATLST | |
| | LGAKLPGCIFFSDTMNHASMIQGMRHSGAKRVLFKH | |
| | NDLEDLENKLKQYPKDTPKVIAFESVYSMCGSIGPIKE | |
| | ICDLAEQYGALTFLDEVHAVGLYGPRGAGVAEHLDY | |
| | DAHVAAGESPHPIKGSVMDRVDIITGTLGKAYGAVGG | |
| | YIAGSDDFVDMIRSYAPGFIFTTSLPPATVAGARASVV | |
| | YQKHYVGDRQLKQVNVREVKRRFAELDVPVVPGPSH | |
| | IVPVLVGDAALAKAASDKLLAEHNIYVQSINYPTVAR | |
| | GEERLRITVTPRHTLEQMDKLVRAVDKIFAELKINRLA | |
| | DWKALGGRAGVGLTAGAEEAHVDPMWTEEQLGLLD | |
| | GTSPRTLRNGEAAVVDAMAVGQARAVFDNLLGPISG | |
| | KLQSERSVLASSTPAAANPARPAARKVVKMKTGGVP | |
| | MSEDIPLPPPDVSASA | |
| 5-aminolevulinic | MDKLSSLSRFKASCPFLGRTKTSTLRTLCTSSSPRFPSIS | 96 |
| acid synthase | ILTERATKCPVMGPALNVRSKEITAGYASVAGSSEVD | |
| (ALAS) | QIHKQQGVTVPVNATVEMCPHASAARAAARMADDL | |
| Dendrothele | AAAAAQKKVGSGASSAKAAAAGCPFHKSVAAGASA | |
| bispora CBS | STASKPSAPIHKASVPGGFDYDNFYNNELEKKHKDKS | |
| 962.96 | YRYFNNINRLASKFPVAHTGDVKDEVQVWCSNDYLG | |
| Accession: | MGNNPVVLETMHRTLDKYGHGAGGTRNIAGNGALH | |
| THV05492.1 | LGLEQELAALHRKEAALVFSSCYVANDATLSTLGSKL | |
| | PGCILFSDKMNHASMIQGMRHSGAKKVIFNHNDLEDL | |
| | ENKLKQYPKETPKIIAFESVYSMCGSIGPIKEICDLAEK | |
| | YGALTFLDEVHAVGLYGPHGAGVAEHLDYNAQKAA | |
| | GKSPEPIPGSVMDRVDIITGTLGKAYGAVGGYIAGSM | |
| | DFVDTIRSYAPGFIFTTSLPPATVSGAQASVAYQKEYL | |
| | GDRQLKQVNVREVKRRFAELDIPVIPGPSHILPVLVGD | |
| | AALAKAASDKLLTDHDIYVQSINYPTVAVGEERLRIT | |
| | VTPRHTLEQMDKLVRAVNQVFTELNINRISDWKVAG | |
| | GRAGVGMGVESVEPIWTDEQLGITDGTTPKTLRDGQR | |
| | FLVDAQGVTAARGRFDTLLGPMSGSLQANPTLPLVD | |
| | DELKVPLPTLVAAAA | |
| 5-aminolevulinic | MDYAQFFNTALDRLHTERRYRVFADLERIAGRFPHAL | 97 |
| acid synthase | WHSPKGKRDVVIWCSNDYLGMGQHPKVVGAMVETA | |
| (ALAS) | TRVGTGAGGTRNIAGTHHPLVQLEAELADLHGKEASL | |
| Bradyrhizobium | LFTSGYVSNQTGIATIAKLIPNCLILSDELNHNSMIEGIR | |
| japonicum | QSGCERVVFRHNDLADLEEKLKAAGPNRPKLIACESL | |
| Accession: | YSMDGDVAPLAKICDLAEKYGAMTYVDEVHAVGMY | |
| A0A0A3YXD2 | GPRGGGIAERDGVMHRIDILEGTLAKAFGCLGGYIAA | |
| | NGQIIDAVRSYAPGFIFTTALPPAICSAATAAIRHLKTS | |
| | NWERERHQDRAARVKAILNAAGLPVMSSDTHIVPLFI | |
| | GDAEKCKQASDLLLEQHGIYIQPINYPTVAKGTERLRI | |
| | TPSPYHDDGLIDQLAEALLQVWDRLGLPLKQKSLAAE | |
| Cytochrome b5 | MDKQRVFTLSQVAEHKSKQDCWIIINGRVVDVTKFLE | 98 |
| Petunia x hybrida, | EHPGGEEVLIESAGKDATKEFQDIGHSKAAKNLLFKY | |
| Accession: | QIGYLQGYKASDDSELELNLVTDSIKEPNKAKEMKAY | |
| AAD10774.1 | VIKEDPKPKYLTFVEYLLPFLAAAFYLYYRYLTGALQ | |
| | F | |
| Flavonol Synthase | MEVERVQAISKMSRCMDTIPSEYIRSESEQPAVTTMQ | 99 |
| AAP57395.1 | GVVLQVPVIDLGSSNNTEENLVELIAEASREWGIFQVV | |
| | NHGIPDDAIAKLQKVGKEFFELPQQEKEVIAKPEGYQ | |
| | GVEGYGTKLQKELGGKKGWVDHLFHIIWPKSAVNYN | |
| | FWPNNPPLYREANEEYAVALRGVVDKLFEALSLGIGL | |
| | EKHELKKASGGDDLIYMLKINYYPPCPRPDLALGVVA | |
| | HTDMSAITILVPNEVQGLQVHKDDHWYDVKYIPNALI | |
| | IHIGDQIEIMSNGKYKSVYHRTTVNKDKTRMSWPVFL | |
| | EPPPELLTGPISKLITDENPAKFKTKKYKDYVYCKLNK | |
| | LPQ | |
| Flavonol Synthase | MGVERVQDIASTISEDTIPAEYIRSENEQPGITTVPNTV | 100 |
| AAZ78661.1 | LECPTIDFSDPDEEKLLKQIFEASIDWGMYQIVNHDISN | |
| | EAISKLQAVGKEFFELPQEEKEVYAKDPNSKSVEGYG | |
| | TFLQKELEGKKGWVDHLFHKIWPPSTINYRFWPKTPA | |
| | SYREANEEYAKNLHKVVEKLFKLLSLGLGLEAQELK | |
| | KAIGGDDLVYLLKINYYPPCPRPDLALGVVAHTDMSA | |
| | LTILVPNEVQGLQACRDGQWYDVKYIPNALVIHIGDQ | |
| | MEIMSNGKYRAVLHRTTVSKDKTRISWPVFLEPPADQ | |
| | VIGPHPKLVNDKENPPEYKTKKYSEYVYNKLNKMPQ | |
| Flavonol Synthase | MEVERVQALSHVTLHELPAKFIRPVHEQPENSKAIEG | 101 |
| ABM88786.1 | VTVPVISLSQPHDVVVDALSKACSEWGFFLITDHGVEP | |
| | SLIGRLKEVGEEFFNLPQKEKESYANDPSSGSFEGYGT | |
| | KMTKNFDEKVEWIDYYFHVMHPPKKLNLDMWPKNP | |
| | SSYRGVTEEYNVEIMRTTNKLFELLSEGLGLDGKVLSS | |
| | SLGGDEIEFEMKINMYPPCPQPQLALGVEPHTDMSAL | |
| | TLLVPNDVPGLQVWKDGNWVAVNYLPNALFVHVGD | |
| | QLEVLSNGKYKSVLHRSLVNKERTRMSWAVFVVPPH | |
| | EAVIGPLPELIDEKNPAKYSTKTYAEYRHRKFNKIPQ | |
| Flavonol Synthase | MKTIQGQSATTALTMEVARVQAISSITKCMDTIPSEYI | 102 |
| I81826.1 | RSENEQPAATTLQGVVLEVPVIDISNVDDDEEKLVKEI | |
| | VEASKEWGIFQVINHGIPDEVIENLQKVGKEFFEEVPQ | |
| | EEKELIAKKPGAQSLEGYGTSLQKEIEGKKGWVDHLF | |
| | HKIWPPSAINYRYWPKNPPSYREANEEYAKWLRKVA | |
| | DGIFRSLSLGLGLEGHEMMEAAGSEDIVYMLKINYYP | |
| | PCPRPDLALGVVAHTDMSYITLLVPNEVQGLQVFKDG | |
| | HWYDVNYIPNAIIVHIGDQVEILSNGKYKSVYHRTTV | |
| | NKYKTRMSWPVFLEPSSEHEVGPIPKLINEANPPKFKT | |
| | KKYKDYVYCKLNKLPQ | |
| Flavonol Synthase | MAPTRVQYVAESRPQTIPLEFVRPVEERPINTTFNDDI | 103 |
| ACY00393.1 | GLGRQIPVIDMCSLEAPELREKTFKEIARASKEWGIFQ | |
| | VINHAISPSLFESLETVGKQFFQLPQEEKEAYACTGED | |
| | GSFTGYGTKLACTTDGRQGWSDFFFHMLWPPSLRDFS | |
| | KWPQKPSSYIEVTEEYSNRILGVLNKLLSALSISLELQE | |
| | SALKDALGGENLEMELKINYYPTCPQPEVAFGVVPHT | |
| | DMSALTILKPNDVPGLQVWKDDKWITAHYVPNALIIH | |
| | IGDQIQILSNGKFKSVLHRSLVNKEKVRMSWPVFCSPP | |
| | LDTVIGPLKELIDDSNPPLYNAKTYREYKHRKINKLGQ | |
| Flavonol Synthase | MEVERVQAIATLTANLGTIPSEFIRSDHERPDLTTYHG | 104 |
| AQR58516.1 | PVPELPIIDLANSSQENVVKQISEAAQEYGIFQLVNHGI | |
| | PNEVINELQRVGKEFFQLPQEEKEVYATVPDSGSFEGY | |
| | GTKLQKDLEGKKAWVDYLFHNVWPKHKINYKFWPR | |
| | NPPAYRKANEEYTKHLQVVVDKMHSYLSLGLGLESH | |
| | VLKEAVGGDDLEYLLKINYYPPCPRPDLALGVVAHTD | |
| | MSSLTILVPNEVPGLQVFKDDHWFDAKYIPNALICHIG | |
| | DQLEILSNGKYKSVLHRTTVNKEKSRMSWPVFCSPPG | |
| | DTMIGPLPQLVNDENPPKFKTKKYKDYAYCKINKLPQ | |
| Flavonol Synthase | MELERVQVIASLSKCIDTIPAEYIRSENEQPAITTIQGK | 105 |
| ASU87437.1 | VLEVPVIDLSNDSDDQNIVQSVAEACRDWGIFQVVNH | |
| | GIPDEVIGNLQRVGKDFFELPQEEKEVYAKPPESKSVE | |
| | GYGTKLQKEVEGKKGWVDHLFHKIWPPSAINYKFWP | |
| | KNPPSYREANEVYAERLRAVADKLFRWLSLGLGLEG | |
| | HEVKEAVGGDDLIYLMKINYYPPCPRPDLALGVVAHT | |
| | DMSTLTILVPNEVPGLQVFKDDHWYEVKYIPNALIVHI | |
| | GDQIEIVSNGKYKAVYHRSTVNKETTRMSWPVFIEPSP | |
| | EVEVGPHPMLIDDENPPKYKTKKYEDYMYCKLNKLP | |
| | Q | |
| Flavonol Synthase | MEVERVQALSDVTLHELPANFIRPAHEQPENSKAIDG | 106 |
| AUM57432.1 | VTVPVISLSQPHDVVVDELSMACSEWGFFLITDHGIEP | |
| | SLIGRLNEVGEEFFKLPQKEKESYANDPSSGSFEGYGT | |
| | KMTKNLDEKVEWVDYYFHIMHPPKKLNHDMWPKNP | |
| | SSYRGVTEEYNVEILRTTNQILELLSEGLGLDGKVLNS | |
| | SLGGDEIEFEMKINMYPPCAQPQLALGVEPHTDMSAL | |
| | TILVPNDVPGLQVWKDGNWVAVNYLPNALFVHVGD | |
| | QLEVLSNGKYKSVLHRSLVNKERRRMSWAVFVVPPH | |
| | EAVIGPLPELIDEKIPAKYSTKTYAEYRYRKFNKIPQ | |
| Flavonol Synthase | MEVERVQAIASLSHSNGTIPAEFIRPEKEQPASTTYHGP | 107 |
| BAA36554.1 | APEIPTIDLDDPVQDRLVRSIAEASREWGIFQVINHGIP | |
| | SDLICKLQAVGKEFFELPQEEKEVYSRPADAKDVQGY | |
| | GTKLQKEVEGKKSWVDHLFHRVWPPSSINYRFWPKN | |
| | PPSYRAVNEEYAKYMREVVDKLFTYLSLGLGVEGGV | |
| | LKEAAGGDDIEYMLKINYYPPCPRPDLALGVVAHTDL | |
| | SALTVLVPNEVPGLQVFKDDRWIDAKYIPNALVIHIGD | |
| | QIEILSNGKYKAVLHRTTVNKDKTRMSWPVFLEPPAD | |
| | TVVGPLPQLVDDENPPKYKAKKFKDYSYCKLNKLPQ | |
| Flavonol Synthase | MELKTVQAIAFSSMSAGIIPSEFIRSEKEQPAITTFHGYI | 108 |
| BAE75808.1 | PQVPTIDLSDPDEEKLTRLIVEASSEWGMFQIVNHGIPS | |
| | DVISNLQKVGKEFFELPQEEKELYAKSPDSKSIQGYGS | |
| | KLQKEVEGKKAWVDHLFHNIWPPPAIDYQFWPKKPP | |
| | TYRAANEEYAKWLQGVADKLFGRLSLGLGLGEGTLK | |
| | ESVGGDELLYLLKINYYPPCPRPDLALGVVAHTDMSA | |
| | ITILIPNEVQGLQVFRDDHWFDVKYISNALVIHVGDQL | |
| | EILSNGKYKAVLHRTTVNKEKTRMSWPVFLEPPSDQV | |
| | VGPLPQLVNEENPARYKTKKYSDYVYCKLNKIPQ | |
| Flavonol Synthase | MKTAQGVSATLTMEVARVQAIASLSKCMDTIPSEYIR | 109 |
| CAA80264.1 | SENEQPAATTLHGVVLQVPVIDLRDPDENKMVKLIAD | |
| | ASKEWGIFQLINHGIPDEAIADLQKVGKEFFEHVPQEE | |
| | KELIAKTPGSNDIEGYGTSLQKEVEGKKGWVDHLFHK | |
| | IWPPSAVNYRYWPKNPPSYREANEEYGKRMREVVDR | |
| | IFKSLSLGLGLEGHEMIEAAGGDEIVYLLKINYYPPCPR | |
| | PDLALGVVAHTDMSYITILVPNEVQGLQVFKDGHWY | |
| | DVKYIPNALIVHIGDQVEILSNGKYKSVYHRTTVNKD | |
| | KTRMSWPVFLEPPSEHEVGPIPKLLSEANPPKFKTKKY | |
| | KDYVYCKLNKLPQ | |
| Flavonol Synthase | RYPPLLPATQEITKKPMEIERVQDISSSSLLTEAIPLEFIR | 110 |
| JAU56058.1 | SEKDQPAISTFRGPTPAIPVVDLSDPDKESVVRAVVKA | |
| | SEEWGIFQVVNHGIPTKLIQRLQEVGRKFFELPSTEKES | |
| | VAKPEDSKDIEGYGTKLQKDLEGKKAWVDHLFHRIW | |
| | PQSCVNYRFWPKNPPEYREVNEEYALQVNKLSETLLG | |
| | ILSEGLGLRREALKEGLGGDLAEYMMKINYYPPCPRP | |
| | DLVLGVPAHTDLSGITLLVPNEVPGLQVFKDDHWIDA | |
| | EYIPSAVIVHIGDQILRLSNGRYKNVLHRTTVDKEKTR | |
| | MSWPVLVDPKRGLVVGPLSELIGDENPPKFESLTFED | |
| | YIYRKINMLLRDG | |
| Flavonol Synthase | MGRCHCHLWVRVFLQIQQSKKSKFHNKRKSLKKMEF | 111 |
| KAF1863632.1 | LRVQTVASNSKDVAIPDMFVRPETEQPGITTVHGVKL | |
| | EVPIIDFNNPDEGKVLNEIMEASKKWGMFQIVNHEIPS | |
| | HVIKKLQSVGKEFFELPQEEKELYAKDPESKSIEGYGT | |
| | KLQKELNGKKGWVDHLFHIIWPLSSINYRFWPKNPPS | |
| | YREANEEYLKYLHGVVDKLFKSMSIGLGFEENELKEA | |
| | AGGDSMIHLLKINYYPPCPFPDLVVGVPPHTDMSYITI | |
| | LVPNDVQGLQAFRDGHWYDVKYVPNALVIHIGDQM | |
| | EIMSNGKYKAVFHRTTVSKEETRISWPVFIEPQPEYEV | |
| | GPHPKLVNEENPPKYKTKKYKDYAYCKLNKIPQ | |
| Flavonol Synthase | MEVERVQAVASLSMLMDTIPSEFIRSEHERPGTTTFHG | 112 |
| KAF8404124.1 | PVPELPAIDLSDQDQDNLVRLITKASEEFGIFQVVNHGI | |
| | PVDIINKLQKVGREFFELPQEEKEAYATPPGAGHLEGE | |
| | ANEEFAKCLRGVVDKLLMNLSLGLGLEGNELKEAVG | |
| | GDGLEYLLKINYYPTCPRPDLALGVVAHTDMPAITVL | |
| | VPNDVPGLQVFKDEHCFEAKYIPNALIIHIGDQIEILSN | |
| | GKYKSVLHRRTVNKEKVRMSWPVFCSPPGEQVIGPLP | |
| | QLVSDDNPAKYKTKKYKDYMYCKINKLPQ | |
| Flavonol Synthase | MEVGRVQSIANKTASKDTIPLDFIWPENVQPAITTVHG | 113 |
| KMS96524.1 | ADIEIPLIELDDDDEERLIKLIAKASSEVGLFQIVNHGIP | |
| | SDVIERFQKVGKEFFELSQEEKERYGKLPGSVEGYGSS | |
| | LQKEDDGKKGMKKGWVDHIFHRVWPPSAINLRFWPT | |
| | ILTSYREVNEEYAKYLRRVGDKLFTYLSLGLGLERHE | |
| | LREGAGGDDIEYLLKINYYPPCPRPDLALGVPAHSDM | |
| | SAITLLVPNDVPGLQICKDDRWYNVNYVPNAIVIHIGD | |
| | QIEIMSNGKYKAVMHRTTVSKDKTRFSWPVFLEPPLE | |
| | QIVGPISKLINKQNPARFTSQKYRDYSSYKYNNISQQKI | |
| Flavonol Synthase | MEVERVQDISSSSLLTEAIPLEFIRSEKEQPAITTFRGPT | 114 |
| NP_196481.1 | PAIPVVDLSDPDEESVRRAVVKASEEWGLFQVVNHGI | |
| | PTELIRRLQDVGRKFFELPSSEKESVAKPEDSKDIEGY | |
| | GTKLQKDPEGKKAWVDHLFHRIWPPSCVNYRFWPKN | |
| | PPEYREVNEEYAVHVKKLSETLLGILSDGLGLKRDAL | |
| | KEGLGGEMAEYMMKINYYPPCPRPDLALGVPAHTDL | |
| | SGITLLVPNEVPGLQVFKDDHWFDAEYIPSAVIVHIGD | |
| | QILRLSNGRYKNVLHRTTVDKEKTRMSWPVFLEPPRE | |
| | KIVGPLPELTGDDNPPKFKPFAFKDYSYRKLNKLPLD | |
| Flavonol Synthase | MDMERVQAIASLAGDLGTIPAEFIRPEHERPAMTTHH | 115 |
| QBO54037.1 | GPSPEIPVVDMTSVDRVKAIADAAKEWGIFQLVNHGI | |
| | PVAAIEELKRVGTEFFALPQEEKESYAAAPGSLEGYGT | |
| | RLQRESEGKKAWADYLFHNVWPDSSINYKFWPNNPP | |
| | EYRKASEEYARRLQGVVDETLGSLSLWLGLERHVLK | |
| | EAVGGERLEYLLKINYYPPCPRPDLALGVVAHTDMSA | |
| | ITVLVPNEVPGLQVFKDGRWLDAEYVPNALVVHVGD | |
| | QVEIVSNGRYKSVMHRTVVSRDRVRMSWPVFCSPPG | |
| | EEVVGPLPQLVGDGSPARFKAKKYKDYSYCKINKLPQ | |
| Flavonol Synthase | MEVERVQAIASMTASLGTIPAEFIRSEHERPDITTYHGP | 116 |
| QBQ58058.1 | VPDIPVVDLSATDRAGVTKAIAEAAQEWGIFQVVNHR | |
| | VPIEAIKELQRVGKEFFELPQEEKEAYATVPDSGSFEG | |
| | YGTRLQKDLEGKKAWVDYLFHNVWPQSKITYRFWP | |
| | NNPPEYRKANEEYAKYLQGVVDEMLTSLSLGLGLES | |
| | HVLKEAVGGDELEYLLKINYYPPCPRPDLALGVVAHT | |
| | DMSAITILVPNEVPGLQVFKDDHWFDAKYIPNALIVHI | |
| | GDQIEILSNGKYKSVLHRTTVNKEKARMSWPVFCSPP | |
| | GEMIVGPLPQLVSDENSAKFKTKKYKDYSYCKINKLP | |
| | Q | |
| Flavonol Synthase | MEVERVQTIAFSSILVDTIPPEFIRSEKEQPALTTCRGCI | 117 |
| RVW17775.1 | PQVPTIDFSDPDEGNLTRLIAEASSEWGMFQIVNHGIPS | |
| | EVITNLQKAGKEFFELPQEEKELYAKPPDSKSIEGYGS | |
| | KLQKEVEGKKAWVDHLFHNIWPPSAINYQYWPKNPP | |
| | SYRAVNEEYCKWVQRVGHRLLSLLSLGLGLEKNELK | |
| | ENVGGDELKYLLKINYYPPCPRPDLALGVVAHTDMSS | |
| | ITILVPNEVQGLQVFRDDHWFDVKYIPDALVIHIGDQL | |
| | EVHLSNGKYKSVLHRTTVTKEITRMSWPVFLEPPSEL | |
| | AISPLPKLINEKNPPKYQKKKYCDYVYCKLNKIAQ | |
| Flavonol Synthase | MEFDRVQAIASLSFDKETIPEEFIWPEKEQPATTTFDGR | 118 |
| XP_002325697.1 | VPEIPTIDLNDPNPENLVRLIADASKEWGIFQVVNHGIP | |
| | SDLIAKLQDVGKKFFELPQEEKEVYAKPHDSKSIEGY | |
| | GSKLQNNPQVKKSWVDHLFHIIWPPSSINYQFWPNNP | |
| | PSYREVNEEYAKYMREVTDKLFTALSLGLGLEGHAL | |
| | KEGAGGEEIEYMLKINYYPPCPRPDLTLGVAAHTDLS | |
| | ALTILVPNEVPGLQIFKDGNWFEAKYIPNALIIHIGDQI | |
| | EILSNGKYKAVLHRTTVAKDKARMSWPVFLEPPGEL | |
| | VVGPLPHLIKKDNPPKFKAKKFEDYMYCKLNRLPQ | |
| Flavonol Synthase | MEFDRVQTIASLSFDKEAIPLEFIRPEKEQPAITTFRGL | 119 |
| XP_006384398.2 | APEIPAIDLSDPDQEKLVGLIADASKEWGIFQVINHGIP | |
| | SGVIAELQGAGKKFFELPQEEKEVCARPPDSKSIEGYD | |
| | SKLQKDPQEKKSWVDHLFHRIWPPPSINYQFWPENPP | |
| | SYREVTKEYAKYMRDVVDKLFTTLSLGLGLEGHVLK | |
| | EAAGGEQIEYMLKINYYPPCPRPDLTLGVVAHTDLSAI | |
| | TILVPNEVPGLQIFKDDHWIEAKYIPDALIIHIGDQIEIL | |
| | SNGKYKAVLHRTTVAKNRTRMSWPVFLEPPGELVVG | |
| | PLPQFINKDNPPKFKTKKFKDYTYCKLNKLPQ | |
| Flavonol Synthase | MEVERVQAIASLSVATNDIPPEFVRSEHEQPGITTYRG | 120 |
| XP_009391364.1 | PVPEIPVIDLEDGDEGRVTRAIAEASQEWGIFQLVNHG | |
| | IPGEVIRALQRVGREFFELPPEEKEKYAAAPGSLQGYG | |
| | TKLQKDLEGKKAWVDFLFHNIWPPTHVDHRAWPENP | |
| | VDYRKANEEYAKHLVGLVEKMLVSLSKGLGLEADVL | |
| | KHAVGGDDLEFLLKINYYPPCPRPDLALGVVAHTDM | |
| | SAITILIPNDVPGLQVFKDDHWFDAKYVPDAIIVHIGD | |
| | QIEKLSNGRYKSVLHRTTVNKEKARMSWPVFCSPPGE | |
| | TVIGPLPQLVSDEQPAQYKTKKYKDYAFCKLNKLPQ | |
| Flavonol Synthase | MAEVQSVQALASSLAALPPEFVRSEHERPGATTFRGG | 121 |
| XP_015624815.1 | DAPEIPVIDMAAPESAARVAEAAAEWGLFQVVNHGV | |
| | PAAAVAELQRVGREFFALPQEEKARYAMDASSGKME | |
| | GYGSKLQKDLEGKKAWADFFFHNVAPPAMVNHDIW | |
| | PSHPAGYREANEEYCKHMQRLARKLFEHLSTALGLD | |
| | GGAMWEAFGGDELVFLHKINFYPPCPEPELTLGVAPH | |
| | TDMSTFTVLVPNDVQGLQVFKDGHWYDVKYVPDALI | |
| | IHIGDQIEILSNGRYKAVLHRTTVDKDRTRMSWPVFV | |
| | EPPPEHVVGPHPQLVTDGSPAKYKAKKFKDYRHCKIN | |
| | KLPM | |
| Flavonol Synthase | MEIERVQDISSSSLHTEVIPLEFIRSEKEQPAITTFRGPV | 122 |
| XP_048623106.1 | PAIPVVDLSDPDEESVARAVVKASEEWGIFQVVNHGIP | |
| | TELIKRLQEVGRTFFELPSSEKESVAKPADAKDIEGYG | |
| | TKLQKEVEGKKAWVDHLFHRIWPPSCVNYSFWPKNP | |
| | PEYREVNEEYALHVKKLSETLLGILSEGLGLRREALRE | |
| | GLGGDLVEYMMKINYYPPCPRPDLALGVPAHTDLSGI | |
| | TLLVPNEVPGLQVFKDDHWFDAEYIPSAVIVHIGDQIL | |
| | RLSNGRYKNVLHRTTVDKDRTRMSWPVFLEPHREMI | |
| | VGPLPELISDGNPPKYKPFAFKDYSYRKLNKLPLD | |

TABLE 12
Glossary of abbreviations

| Abbreviation | Full Name |
|---|---|
| 3GT | anthocyanidin-3-O-glycotransferase |
| 4CL | 4-coumarate-CoA ligase |
| ACC | acetyl-CoA carboxylase |
| ACOT | acyl-CoA thioesterase |
| acpP | acyl carrier protein |
| ACS | acetyl-CoA synthase |
| adhE | aldehyde-alcohol dehydrogenase |
| ADP | adenosine diphosphate |
| ALA | 5-aminolevulinic acid |
| ALAS | ALA synthase |
| ANS | anthocyanin dioxygenase |
| aroG | DAHP synthase |
| aroK | shikimate kinase |
| aroL | shikimate kinase |
| ATP | adenosine triphosphate |
| C3G | cyanidin-3-O-glycoside |
| C4H | hikimate-4-hydroxylase |
| CHI | chalcone isomerase |
| CHS | chalcone synthase |
| CoA | coenzyme A |
| CPR | cytochrome P450 Reductase |
| DAD | diode array detector |
| DAHP | deoxy-d-arabino-heptulosonate-7-phosphate |
| DctPQM | a malonate transporter |
| DFR | dihydroflavonol 4-reductase |
| DHK | dihydrokaempferol |
| DHM | dihydromyricein |
| DHQ | dihydroquercetin |
| DMSO | dimethyl sulfoxide |
| E4P | erythrose-4-phosphate |
| F3′H | flavonoid 3′ hydroxylase |
| F3H | flavanone 3-hydroxylase |
| fabB | beta-ketoacyl-ACP synthase I |
| fabD | malonyl-coA-ACP transacylase |
| fabF | beta-ketoacyl-ACP synthase II |
| FadA | 3-ketoacyl-CoA thiolase |
| FadB | fatty acid oxidation complex subunit alpha |
| FadE | acyl-CoA dehydrogenase |
| FLS | Flavonol synthase |
| GltX | glutamyl-tRNA synthetase |
| hemA | glutamyl-tRNA reductase |
| hemL | glutamate-1-semialdehyde aminotransferase |
| HPLC | high performance liquid chromatography |
| ldhA | lactate dehydrogenase |
| LAR | leucoanthocyanidin reductase |
| matB | malonyl-CoA synthase |
| matC | malonate transporter |
| mdcA | malonate coA-transferase |
| mdcC | acyl-carrier protein, subunit of mdc |
| mdcD | malonyl-CoA decarboxylase, subunit of mdc |
| mdcE | co-decarboxylase, subunit of mdc |
| pABA | para-aminobenzoic acid |
| PAL | phenylalanine ammonia-lyase |
| PanK | pantothenase kinase |
| Pdh | pyruvate dehydrogenase |
| PEP | phosphoenolpyruvate |
| pHBA | para-hydroxybenzoic acid |
| PHE | phenylalanine |
| pheA | hikimate mutase/prephenate dehydrogenase |
| poxB | pyruvate dehydrogenase |
| ppsA | phosphoenolpyruvate synthase |
| TAL | tyrosine ammonia-lyase |
| TCA | tricarboxylic acid cycle |
| tesA | thioesterase I |
| tesB | thioesterase II |
| tktA | Transketolase |
| TRP | Tryptophan |
| TYR | Tyrosine |
| TyrA | hikimate mutase |
| tyrR | transcriptional regulator |
| ybgC | a thioesterase |
| yciA | a thioesterase |
| ydiB | QUIN hikimate dehydrogenase |
| ackA-pta | Acetate kinase-phosphate acetyltransferase |

### INCORPORATION BY REFERENCE

References and citations to other documents, such as patents, patent applications, patent publications, journals, books, papers, web contents, publicly accessible databases, have been made throughout this disclosure. All such documents are hereby incorporated herein by reference in their entirety for all purposes.

### EQUIVALENTS

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

1. An engineered host cell, wherein the engineered host cell comprises one or more genetic modifications to increase the production of flavonols through transformation by one or more enzymes of a carbon source by the engineered host cell.

2. The engineered host cell of claim 1, wherein the flavonol is selected from a group consisting of kaempferol, myricetin, and quercetin.

3. The engineered host cell of claim 1, wherein the carbon source is selected from a group consisting of naringenin, dihydrokaempferol (DHK), dihydromyricetin (DHM), dihydroquercetin (DHQ), and eriodictyol (EDL).

4. The engineered host cell of claim 1, wherein the one or more genetic modifications are at least one genetic modification selected from the group consisting of: (i) one or more modifications for over-expressing one or more endogenous genes in the engineered host cells; (ii) one or more modifications for under-expressing one or more endogenous genes in the engineered host cells; (iii) one or more genetic modification is expressing one or more non-native genes in the engineered host cells; and (iv) a combination thereof.

5. The engineered host cell of claim 1, wherein the engineered host cell comprises at least one or more nucleic acid sequences selected from the group consisting of: (i) nucleic acid sequences encoding native or modified flavanone-3-hydroxylase (F3H) or a homolog thereof, (ii) nucleic acid sequences encoding native or modified flavanone-3′-hydroxylase (F3′H) or a homolog thereof, (iii) nucleic acid sequences encoding native or modified flavonoid 3′,5′-hydroxylase (F3′5′H) or a homolog thereof, (iv) nucleic acid sequences encoding native or modified flavonol synthase (FLS) or a homolog thereof, and (v) any combinations thereof.

6. The engineered host cell of claim 1, wherein the engineered host cell is E. coli.

7. The engineered host cell of claim 1, wherein the production of flavonols through enzymatic transformation of a carbon source includes one or more chemical intermediates.

8. The engineered host cell of claim 1, wherein the flavonol is myricetin, the carbon source is dihydromyricetin (DHM), and the one or more enzyme is flavonol synthase (FLS).

9. The engineered host cell of claim 1, wherein the flavonol is myricetin, the carbon source is dihydrokaempferol (DHK), and the one or more enzymes are selected from a group consisting of (i) flavonoid 3′,5′-hydroxylase (F3′5′H), (ii) flavonol synthase (FLS), and (iii) any combination thereof.

10. The engineered host cell of claim 9, wherein flavonoid 3′,5′-hydroxylase (F3′5′H) has an amino acid sequence at least 80% identical to the polypeptides selected from a group consisting of: (i) SEQ ID NO. 10, (ii) SEQ ID NO. 56, and (iii) SEQ ID NO. 57.

11. The engineered host cell of claim 1, wherein the flavonol is kaempferol, the carbon source is dihydrokaempferol (DHK), and the one or more enzyme is flavonol synthase (FLS).

12. The engineered host cell of claim 11, wherein flavonol synthase has an amino acid sequence at least 80% identical to any one of the polypeptides of SEQ ID NOS. 99-122.

13. The engineered host cell of claim 1, wherein the flavonol is quercetin, the carbon source is eriodictoyl (EDL), and the one or more enzyme is selected from a group consisting of (i) flavanone-3-hydroxylase (F3H), (ii) flavonol synthase (FLS), and (iii) any combination thereof.

14. The engineered host cell of claim 13, wherein the flavanone-3-hydroxylase (F3H) has an amino acid sequence at least 80% identical to the polypeptides selected from a group consisting of: (i) SEQ ID NO. 7, (ii) SEQ ID NO. 45, (iii) SEQ ID NO. 46, (iv) SEQ ID NO. 47, and (v) SEQ ID NO. 48.

15. The engineered host cell of claim 13, wherein production of quercetin leads to formation of dihydroquercetin (DHQ) as an intermediate.

16. The engineered host cell of claim 1, wherein the flavonol is quercetin, the carbon source is dihydroquercetin (DHQ) and the one or more enzyme is flavonol synthase (FLS).

17. The engineered host cell of claim 1, wherein the flavonol is quercetin, the carbon source is kaempferol, and the enzyme is flavanone-3′-hydroxylase (F3′H).

18. The engineered host cell of claim 17, wherein the flavanone-3′-hydroxylase (F3′H) has an amino acid sequence at least 80% identical to the polypeptide set forth in (i) SEQ ID NO: 8 (ii) SEQ ID NO. 49, (iii) SEQ ID NO. 50, (iv) SEQ ID NO. 51, and (v) SEQ ID NO. 52.

19. The engineered host cell of claim 1, wherein the flavonol is kaempferol; the carbon source is selected from a group consisting of: (i) dihydrokaempferol (DHK), (ii) naringenin, and (iii) any combination thereof, and the one or more enzyme is selected from a group consisting of (i) flavanone-3-hydroxylase (F3H), (ii) flavonol synthase (FLS), and (iii) any combination thereof.

20. The engineered host cell of claim 1, wherein the flavonol is quercetin; the carbon source is selected from a group consisting of: (i) dihydroquerctin (DHQ), (ii) dihydrokaempferol (DHK), (iii) eriodictyol (EDL), (iv) naringenin, (v) kaempferol, and (vi) any combination thereof; and the one or more enzyme is selected from a group consisting of: (i) flavonol synthase (FLS), (ii) flavanone-3′-hydroxylase (F3′H), (iii) flavanone-3-hydroxylase (F3H), and (iv) any combination thereof.

21. The engineered host cell of claim 1, wherein the flavonol is myricetin, the carbon source is selected from a group consisting of: (i) dihydromyricetin (DHM), (ii) dihydroquerctin (DHQ), (iii) dihydrokaempferol (DHK), (iv) eriodictyol (EDL), (v) naringenin, (vi) quercetin, (vii) kaempferol, and (viii) any combination thereof, and the one or more enzyme is selected from a group consisting of: (i) flavonol synthase (FLS), (ii) flavanone-3′-hydroxylase (F3′H), (iii) flavanone-3-hydroxylase (F3H), (iv) flavonoid-3′-5′-hydroxylase (F3′5′H), and (v) any combination thereof.

22. A method of increasing production of flavonols, comprising an engineered host cell, wherein the engineered host cell comprises one or more genetic modifications to increase the production of flavonols through transformation by one or more enzymes of a carbon source by the engineered host cell.

23. The method of claim 22, wherein the flavonol is selected from a group consisting of kaempferol, myricetin, and quercetin.

24. The method of claim 22, wherein the carbon source is selected from a group consisting of naringenin, dihydrokaempferol (DHK), dihydromyricetin (DHM), dihydroquercetin (DHQ), and eriodictyol (EDL).

25. The method of claim 22, wherein the one or more genetic modifications are at least one genetic modification selected from the group consisting of: (i) one or more modifications for over-expressing one or more endogenous genes in the engineered host cells; (ii) one or more modifications for under-expressing one or more endogenous genes in the engineered host cells; (iii) one or more genetic modification is expressing one or more non-native genes in the engineered host cells; and (iv) a combination thereof.

26. The method of claim 22, wherein the engineered host cell comprises at least one or more nucleic acid sequences selected from the group consisting of: (i) nucleic acid sequences encoding native or modified flavanone-3-hydroxylase (F3H) or a homolog thereof, (ii) nucleic acid sequences encoding native or modified flavanone-3′-hydroxylase (F3′H) or a homolog thereof, (iii) nucleic acid sequences encoding native or modified flavonoid 3′,5′-hydroxylase (F3′5′H) or a homolog thereof, (iv) nucleic acid sequences encoding native or modified flavonol synthase (FLS) or a homolog thereof, and (v) any combinations thereof.

27. The method of claim 22, wherein the engineered host cell is E. coli.

28. The method of claim 22, wherein the production of flavonols through enzymatic transformation of a carbon source includes one or more chemical intermediates.

29. The method of claim 22, wherein the flavonol is myricetin, the carbon source is dihydromyricetin (DHM), and the one or more enzyme is flavonol synthase (FLS).

30. The method of claim 22, wherein the flavonol is myricetin, the carbon source is dihydrokaempferol (DHK), and the one or more enzymes are selected from a group consisting of (i) flavonoid 3′,5′-hydroxylase (F3′5′H), (ii) flavonol synthase (FLS), and (iii) any combination thereof.

31. The method of claim 30, wherein flavonoid 3′,5′-hydroxylase (F3′5′H) has an amino acid sequence at least 80% identical to the polypeptides selected from a group consisting of: (i) SEQ ID NO. 10, (ii) SEQ ID NO. 56, and (iii) SEQ ID NO. 57.

32. The method of claim 22, wherein the flavonol is kaempferol, the carbon source is dihydrokaempferol (DHK), and the one or more enzyme is flavonol synthase (FLS).

33. The method of claim 32, wherein flavonol synthase (FLS) has an amino acid sequence at least 80% identical to any one of the polypeptides of SEQ ID NOS. 99-122.

34. The method of claim 22, wherein the flavonol is quercetin, the carbon source is eriodictoyl (EDL), and the one or more enzyme is selected from a group consisting of (i) flavanone-3-hydroxylase (F3H), (ii) flavonol synthase (FLS), and (iii) any combination thereof.

35. The method of claim 34, wherein the flavanone-3-hydroxylase (F3H) has an amino acid sequence at least 80% identical to the polypeptides selected from a group consisting of: (i) SEQ ID NO. 7, (ii) SEQ ID NO. 45, (iii) SEQ ID NO. 46, (iv) SEQ ID NO. 47, and (v) SEQ ID NO. 48.

36. The method of claim 34, wherein the production of quercetin leads to formation of dihydroquercetin (DHQ) as an intermediate.

37. The method of claim 22, wherein the flavonol is quercetin, the carbon source is dihydroquercetin (DHQ) and the one or more enzyme is flavonol synthase (FLS).

38. The method of claim 22, wherein the flavonol is quercetin, the carbon source is kaempferol, and the enzyme is flavanone-3′-hydroxylase (F3′H).

39. The method of claim 38, wherein the flavanone-3′-hydroxylase (F3′H) has an amino acid sequence at least 80% identical to the polypeptide set forth in (i) SEQ ID NO: 8 (ii) SEQ ID NO. 49, (iii) SEQ ID NO. 50, (iv) SEQ ID NO. 51, and (v) SEQ ID NO. 52.

40. The method of claim 22, wherein the flavonol is kaempferol; the carbon source is selected from a group consisting of: (i) dihydrokaempferol (DHK), (ii) naringenin, and (iii) any combination thereof, and the one or more enzyme is selected from a group consisting of (i) flavanone-3-hydroxylase (F3H), (ii) flavonol synthase (FLS), and (iii) any combination thereof.

41. The method of claim 22, wherein the flavonol is quercetin; the carbon source is selected from a group consisting of: (i) dihydroquerctin (DHQ), (ii) dihydrokaempferol (DHK), (iii) eriodictyol (EDL), (iv) naringenin, (v) kaempferol, and (vi) any combination thereof, and the one or more enzyme is selected from a group consisting of: (i) flavonol synthase (FLS), (ii) flavanone-3′-hydroxylase (F3′H), (iii) flavanone-3-hydroxylase (F3H), and (iv) any combination thereof.

42. The method of claim 22, wherein the flavonol is myricetin, the carbon source is selected from a group consisting of: (i) dihydromyricetin (DHM), (ii) dihydroquerctin (DHQ), (iii) dihydrokaempferol (DHK), (iv) eriodictyol (EDL), (v) naringenin, (vi) quercetin, (vii) kaempferol, and (viii) any combination thereof, and the one or more enzyme is selected from a group consisting of: (i) flavonol synthase (FLS), (ii) flavanone-3′-hydroxylase (F3′H), (iii) flavanone-3-hydroxylase (F3H), (iv) flavonoid-3′-5′-hydroxylase (F3′5′H), and (v) any combination thereof.


SEQUENCE LISTING (122 sequences)

SEQ ID NO: 1 - protein, 510 aa, synthetic construct
MTQVVERQADRLSSREYLARVVRSAGWDAGLTSCTDEEIVRMGASARTIEEYLKSDKPIYGLTQGFGPLVLFDADSELEQGGSLISHLGTGQGAPLAPEVSRLILWLRIQNMRKGYSAVSPVFWQKLADLWNKGFTPAIPRHGTVSASGDLQPLAHAALAFTGVGEAWTRDADGRWSTVPAVDALAALGAEPFDWPVREALAFVNGTGASLAVAVLNHRSALRLVRACAVLSARLATLLGANPEHYDVGHGVARGQVGQLTAAEWIRQGLPRGMVRDGSRPLQEPYSLRCAPQVLGAVLDQLDGAGDVLAREVDGCQDNPITYEGELLHGGNFHAMPVGFASDQIGLAMHMAAYLAERQLGLLVSPVTNGDLPPMLTPRAGRGAGLAGVQISATSFVSRIRQLVFPASLTTLPTNGWNQDHVPMALNGANSVFEALELGWLTVGSLAVGVAQLAAMTGHAAEGVWAELAGICPPLDADRPLGAEVRAARDLLSAHADQLLVDEADGKDFG

SEQ ID NO: 2 - protein, 550 aa, synthetic construct
MSQVALFEQELMLHGKHTLLLNGNDLTITDVAQMAKGTFEAFTFHISEEANKRIEECNELKHEIMNQHNPIYGVTTGFGDSVHRQISGEKAWDLQRNLIRFLSCGVGPVADEAVARATMLIRTNCLVKGNSAVRLEVIHQLIAYMERGITPIIPERGSVGASGDLVPLSYLASILVGEGKVLYKGEEREVAEALGAEGLEPLTLEAKEGLALVNGTSFMSAFACLAYADAEEIAFIADICTAMASEALLGNRGHFYSFIHEQKPHLGQMASAKNIYTLLEGSQLSKEYSQIVGNNEKLDSKAYLELTQSIQDRYSIRCAPHVTGVLYDTLDWVKKWLEVEINSTNDNPIFDVETRDVYNGGNFYGGHVVQAMDSLKVAVANIADLLDRQLQLVVDEKFNKDLTPNLIPRFNNDNYEIGLHHGFKGMQIASSALTAEALKMSGPVSVFSRSTEAHNQDKVSMGTISSRDARTIVELTQHVAAIHLIALCQALDLRDSKKMSPQTTKIYNMIRKQVPFVERDRALDGDIEKVVQLIRSGNLKKEIHDQNVND

SEQ ID NO: 3 - protein, 505 aa, synthetic construct
MDLLLIEKTLLALFAAIIGAIVISKLRGKRFKLPPGPLPVPIFGNWLQVGDDLNHRNLTDLAKKFGEIFLLRMGQRNLVVVSSPDLAKEVLHTQGVEFGSRTRNVVFDIFTGKGQDMVFTVYGEHWRKMRRIMTVPFFTNKVVQQYRYGWEAEAAAVVEDVKKNPAAATEGVVIRRRLQLMMYNNMFRIMFDRRFESEDDPLFVKLKALNGERSRLAQSFEYNYGDFIPILRPFLKGYLKLCKEVKEKRFQLFKDYFVDERKKLESTKSVDNNQLKCAIDHILDAKEKGEINEDNVLYIVENINVAAIETTLWSIEWGIAELVNHPEIQAKLRNELDTKLGPGVQVTEPDLHKLPYLQAVIKETLRLRMAIPLLVPHMNLHDAKLGGYDIPAESKILVNAWWLANNPEQWKKPEEFRPERFFEEESKVEANGNDFRYLPFGVGRRSCPGIILALPILGITIGRLVQNFELLPPPGQSKVDTTEKGGQFSLHILKHSTIVAKPRAL

SEQ ID NO: 4 - protein, 544 aa, synthetic construct
MGDCVAPKEDLIFRSKLPDIYIPKHLPLHTYCFENISKVGDKSCLINGATGETFTYSQVELLSRKVASGLNKLGIQQGDTIMLLLPNSPEYFFAFLGASYRGAISTMANPFFTSAEVIKQLKASQAKLIITQACYVDKVKDYAAEKNIQIICIDDAPQDCLHFSKLMEADESEMPEVVINSDDVVALPYSSGTTGLPKGVMLTHKGLVTSVAQQVDGDNPNLYMHSEDVMICILPLFHIYSLNAVLCCGLRAGVTILIMQKFDIVPFLELIQKYKVTIGPFVPPIVLAIAKSPVVDKYDLSSVRTVMSGAAPLGKELEDAVRAKFPNAKLGQGYGMTEAGPVLAMCLAFAKEPYEIKSGACGTVVRNAEMKIVDPETNASLPRNQRGEICIRGDQIMKGYLNDPESTRTTIDEEGWLHTGDIGFIDDDDELFIVDRLKEIIKYKGFQVAPAELEALLLTHPTISDAAVVPMIDEKAGEVPVAFVVRTNGFTTTEEEIKQFVSKQVVFYKRIFRVFFVDAIPKSPSGKILRKDLRARIASGDLPK

SEQ ID NO: 5 - protein, 389 aa, synthetic construct
MVTVEEYRKAQRAEGPATVMAIGTATPTNCVDQSTYPDYYFRITNSEHKTDLKEKFKRMCEKSMIKKRYMHLTEEILKENPSMCEYMAPSLDARQDIVVVEVPKLGKEAAQKAIKEWGQPKSKITHLVFCTTSGVDMPGCDYQLTKLLGLRPSVKRLMMYQQGCFAGGTVLRLAKDLAENNKGARVLVVCSEITAVTFRGPNDTHLDSLVGQALFGDGAGAIIIGSDPIPGVERPLFELVSAAQTLLPDSHGAIDGHLREVGLTFHLLKDVPGLISKNIEKSLEEAFRPLSISDWNSLFWIAHPGGPAILDQVEIKLGLKPEKLKATRNVLSNYGNMSSACVLFILDEMRKASAKEGLGTTGEGLEWGVLFGFGPGLTVETVVLHSVAT

SEQ ID NO: 6 - protein, 222 aa, synthetic construct
MAASITAITVENLEYPAVVTSPVTGKSYFLGGAGERGLTIEGNFIKFTAIGVYLEDIAVASLAAKWKGKSSEELLETLDFYRDIISGPFEKLIRGSKIRELSGPEYSRKVMENCVAHLKSVGTYGDAEAEAMQKFAEAFKPVNFPPGASVFYRQSPDGILGLSFSPDTSIPEKEAALIENKAVSSAVLETMIGEHAVSPDLKRCLAARLPALLNEGAFKIGN

SEQ ID NO: 7 - protein, 365 aa, synthetic construct
MAPTPTTLTAIAGEKTLQQSFVRDEDERPKVAYNQFSNEIPIISLSGIDEVEGRRAEICNKIVEACEDWGVFQIVDHGVDAKLISEMTRLARDFFALPPEEKLRFDMSGGKKGGFIVSSHLQGEAVQDWREIVTYFSYPVRHRDYSRWPDKPEGWRAVTQQYSDELMGLACKLLEVLSEAMGLEKEALTKACVDMDQKVVVNFYPKCPQPDLTLGLKRHTDPGTITLLLQDQVGGLQATRDGGKTWITVQPVEGAFVVNLGDHGHFLSNGRFKNADHQAVVNSNHSRLSIATFQNPAQEAIVYPLKVREGEKPILEEPITYTEMYKKKMSKDLELARLKKLAKEQQPEDSEKAKLEVKQVDDIFA

SEQ ID NO: 8 - protein, 511 aa, synthetic construct
MTNLYLTILLPTFIFLIVLVLSRRRNNRLPPGPNPWPIIGNLPHMGPKPHQTLAAMVTTYGPILHLRLGFADVVVAASKSVAEQFLKVHDANFASRPPNSGAKHMAYNYQDLVFAPYGQRWRMLRKISSVHLFSAKALEDFKHVRQEEVGTLMRELARANTKPVNLGQLVNMCVLNALGREMIGRRLFGADADHKAEEFRSMVTEMMALAGVFNIGDFVPALDCLDLQGVAGKMKRLHKRFDAFLSSILEEHEAMKNGQDQKHTDMLSTLISLKGTDFDGEGGTLTDTEIKALLLNMFTAGTDTSASTVDWAIAELIRHPEIMRKAQEELDSVVGRGRPINESDLSQLPYLQAVIKENFRLHPPTPLSLPHIASESCEINGYHIPKGSTLLTNIWAIARDPDQWSDPLTFRPERFLPGGEKAGVDVKGNDFELIPFGAGRRICAGLSLGLRTIQLLTATLVHGFEWELAGGVTPEKLNMEETYGITLQRAVPLVVHPKLRLDMSAYGLGSA

SEQ ID NO: 9 - protein, 714 aa, synthetic construct
MDSSSEKLSPFELMSAILKGAKLDGSNSSDSGVAVSPAVMAMLLENKELVMILTTSVAVLIGCVVVLIWRRSSGSGKKVVEPPKLIVPKSVVEPEEIDEGKKKFTIFFGTQTGTAEGFAKALAEEAKARYEKAVIKVIDIDDYAADDEEYEEKFRKETLAFFILATYGDGEPTDNAARFYKWFVEGNDRGDWLKNLQYGVFGLGNRQYEHFNKIAKVVDEKVAEQGGKRIVPLVLGDDDQCIEDDFAAWRENVWPELDNLLRDEDDTTVSTTYTAAIPEYRVVFPDKSDSLISEANGHANGYANGNTVYDAQHPCRSNVAVRKELHTPASDRSCTHLDFDIAGTGLSYGTGDHVGVYCDNLSETVEEAERLLNLPPETYFSLHADKEDGTPLAGSSLPPPFPPCTLRTALTRYADLLNTPKKSALLALAAYASDPNEADRLKYLASPAGKDEYAQSLVANQRSLLEVMAEFPSAKPPLGVFFAAIAPRLQPRFYSISSSPRMAPSRIHVTCALVYEKTPGGRIHKGVCSTWMKNAIPLEESRDCSWAPIFVRQSNFKLPADPKVPVIMIGPGTGLAPFRGFLQERLALKEEGAELGTAVFFFGCRNRKMDYIYEDELNHFLEIGALSELLVAFSREGPTKQYVQHKMAEKASDIWRMISDGAYVYVCGDAKGMARDVHRTLHTIAQEQGSMDSTQAEGFVKNLQMTGRYLRDVW

SEQ ID NO: 10 - protein, 506 aa, synthetic construct
MSTSLLLAAAAILFFITHLFLRFLLSPRRTRKLPPGPKGWPVVGALPMLGNMPHAALADLSRRYGPIVYLKLGSRGMVVASTPDSARAFLKTQDLNFSNRPTDAGATHIAYNSQDMVFADYGPRWKLLRKLSSLHMLGGKAVEDWAVVRRDEVGYMVKAIYESSCAGEAVHVPDMLVFAMANMLGQVILSRRVFVTKGVESNEFKEMVIELMTSAGLFNVGDFIPSIAWMDLQGIVRGMKRLHKKFDALLDKILREHTATRRERKEKPDLVDVLMDNRDNKSEQERLTDTNIKALLLNLFSAGTDTSSSTIEWALTEMIKNPSIFGRAHAEMDQVIGRNRRLEESDIPKLPYLQAICKETFRKHPSTPLNLPRVAIEPCEVEGYHIPKGTRLSVNIWAIGRDPNVWENPLEFNPDRFLTGKMAKIDPRGNNFELIPFGAGRRICAGTRMGIVLVEYILGSLVHAFEWKLRDGETLNMEETFGIALQKAVPLAAVVTPRLPPSAYVV

SEQ ID NO: 11 - protein, 347 aa, synthetic construct
MMHKGTVCVTGAAGFVGSWLIMRLLEQGYSVKATVRDPSNMKKVKHLLDLPGAANRLTLWKADLVDEGSFDEPIQGCTGVFHVATPMDFESKDPESEMIKPTIEGMLNVLRSCARASSTVRRVVFTSSAGTVSIHEGRRHLYDETSWSDVDFCRAKKMTGWMYFVSKTLAEKAAWDFAEKNNIDFISIIPTLVNGPFVMPTMPPSMLSALALITRNEPHYSILNPVQFVHLDDLCNAHIFLFECPDAKGRYICSSHDVTIAGLAQILRQRYPEFDVPTEFGEMEVFDIISYSSKKLTDLGFEFKYSLEDMFDGAIQSCREKGLLPPATKEPSYATEQLIATGQDNGH

SEQ ID NO: 12 - protein, 382 aa, synthetic construct
MTVSGAIPSMTKNRTLVVGGTGFIGQFITKASLGFGYPTFLLVRPGPVSPSKAVIIKTFQDKGAKVIYGVINDKECMEKILKEYEIDVVISLVGGARLLDQLTLLEAIKSVKTIKRFLPSEFGHDVDRTDPVEPGLTMYKEKRLVRRAVEEYGIPFTNICCNSIASWPYYDNCHPSQVPPPMDQFQIYGDGNTKAYFIDGNDIGKFTMKTIDDIRTLNKNVHFRPSSNCYSINELASLWEKKIGRTLPRFTVTADKLLAHAAENIIPESIVSSFTHDIFINGCQVNFSIDEHSDVEIDTLYPDEKFRSLDDCYEDFVPMVHDKIHAGKSGEIKIKDGKPLVQTGTIEEINKDIKTLVETQPNEEIKKDMKALVEAVPISAMG

SEQ ID NO: 13 - protein, 356 aa, synthetic construct
MFSSVAVPRVEILASSGIESIPKEYVRPQEELTTIGNIFDEEKKDEGPQVPTIDLRDIDSDDQQVRQRCRDELKKAAVDWGVMHLVNHGIPDHLIDRVKKAGQAFFELPVEVKEKYANDQASGNIQGYGSKLANNASGQLEWEDYYFHLIFPEEKRDLAIWPNNPADYIEVTSEYARQLRRLVSKILGVLSLGLGLEEGRLEKEVGGLDELLLQMKINYYPTCPQPELALGVEAHTDISALTFILHNMVPGLQLFYEGKWVTAKCVPNSIVMHVGDTIEILSNGKYKSILHRGLVNKEKVRISWAVFCEPPKEKIILKPLPETVSENEPPLFPPRTFAQHIQHKLFRKNQENLEAK

SEQ ID NO: 14 - protein, 456 aa, synthetic construct
MSQTTTNPHVAVLAFPFSTHAAPLLAVVRRLAVAAPHAVFSFFSTSESNASIFHDSMHTMQCNIKSYDVSDGVPEGYVFTGRPQEGIDLFMRAAPESFRQGMVMAVAETGRPVSCLVADAFIWFAADMAAEMGVAWLPFWTAGPNSLSTHVYIDEIREKIGVSGIQGREDELLNFIPGMSKVRFRDLQEGIVFGNLNSLFSRLLHRMGQVLPKATAVFINSFEELDDSLTNDLKSKLKTYLNIGPFNLITPPPVVPNTTGCLQWLKERKPTSVVYISFGTVTTPPPAELVALAEALEASRVPFIWSLRDKARMHLPEGFLEKTRGHGMVVPWAPQAEVLAHEAVGAFVTHCGWNSLWESVAGGVPLICRPFFGDQRLNGRMVEDVLEIGVRIEGGVFTKSGLMSCFDQILSQEKGKKLRENLRALRETADRAVGPKGSSTENFKTLVDLVSKPKDV

SEQ ID NO: 15 - protein, 2216 aa, synthetic construct
MVEHRSLPGHFLGGNSLESAPQGPVKDFVQAHEGHTVISKVLIANNGMAAMKEIRSVRKWAYETFGNERAIEFTVMATPEDLKANAEYIRMADNFVEVPGGSNNNNYANVELIVDVAERTAVHAVWAGWGHASENPRLPEMLAKSKHKCLFIGPPASAMRSLGDKISSTIVAQSAQVPTMGWSGDGITETEFDAAGHVIVPDNAYNEACVKTAEQGLKAAEKIGFPVMIKASEGGGGKGIRMVKDGSNFAQLFAQVQGEIPGSPIFIMKLAGNARHLEVQLLADQYGNAISLFGRDCSVQRRHQKIIEEAPVTIAKPDVFEQMEKAAVRLGKLVGYVSAGTVEYLYSHHDDQFYFLELNPRLQVEHPTTEMVSGVNLPAAQLQIAMGIPLHRIRDIRVLYGVQPNSASEIDFGFEHPTSLTSHRRPTPKGHVIACRITAENPDAGFKPSSGIMQELNFRSSTNVWGYFSVVSAGGLHEYADSQFGHIFAYGENRQQARKNMVIALKELSIRADFRSTVEYIIRLLETPDFEENTINTGWLDMLISKKLTAERPDTMLAVFCGAVTKAHMASLDCFQQYKQSLEKGQVPSKGSLKTVFTVDFIYEEVRYNFTVTQSAPGIYTLYLNGTKTQVGIRDLSDGGLLISIDGKSHTTYSRDEVQATRMMVDGKTCLLEKESDPTQLRSPSPGKLVNLLVENGDHLNAGDAYAEIEVMKMYMPLIATEDGHVQFIKQAGATLEAGDIIGILSLDDPSRVKHALPFNGTVPAFGAPHITGDKPVQRFNATKLTLQHILQGYDNQALVQTVVKDFADILNNPDLPYSELNSVLSALSGRIPQRLEASIHKLADESKAANQEFPAAQFEKLVEDFAREHITLQSEATAYKNSVAPLSSIFARYRNGLTEHAYSNYVELMEAYYDVEILFNQQREEEVILSLRDQHKDDLDKVLAVTLSHAKVNIKNNVILMLLDLINPVSTGSALDKYFTPILKRLSEIESRATQKVTLKARELLILCQLPSYEERQAQMYQILKNSVTESVYGGGSEYRTPSYDAFKDLIDTKFNVFDVLPHFFYHADPYIALAAIEVYCRRSYHAYKILDVAYNLEHKPYVVAWKFLLQTAANGIDSNKRIASYSDLTFLLNKTEEEPIRTGAMTACNSLADLQAELPRILTAFEEEPLPPMLQRNAAPKEERMENILNIAVRADEDMDDTAFRTKICEMITANADVFRQAHLRRLSVVVCRDNQWPDYYTFRERENYQEDETIRHIEPAMAYQLELARLSNFDIKPCFIENRQMHVYYAVAKENPSDCRFFIRALVRPGRVKSSMRTADYLISESDRLLTDILDTLEIVSHEYKNSDCNHLFINFIPTFAIEADDVEHALKDFVDRHGKRLWKLRVTGAEIRFNVQSKKPDAPIIPMRFTVDNVSGFILKVEVYQEVKTEKSGWILKSVNKIPGAMHMQPLSTPYPTKEWLQPRRYKAHLMGTTYVYDFPELFRQSVQNQWTQAIKRNPLLKQPSHLVEAKELVLDEDDVLQEIDRAPGTNTVGMVAWIMTIRTPEYPSGRRIIAIANDITFKIGSFGVAEDQVFYKASELARALGIPRIYLSANSGARIGLADELISQFRAAWKDASNPTAGFKYLYLTPAEYDVLAQQGDAKSVLVEEIQDEGETRLRITDVIGHTDGLGVENLKGSGLIAGATSRAYDDIFTITLVTCRSVGIGAYLVRLGQRTIQNEGQPIILTGAPALNKVLGREVYTSNLQLGGTQIMYKNGVSHLTAENDLEGIAKIVQWLSFVPDVRNAPVSMRLGADPIDRDIEYTPPKGPSDPRFFLAGKSENGKWLSGFFDQDSFVETLSGWARTVVVGRARLGGIPMGVVSVETRTVENIVPADPANSDSTEQVFMEAGGVWFPNSAYKTAQAINDFNKGEQLPLMIFANWRGFSGGQRDMYNEVLKYGAQIVDALSNYKQPVFVYIIPNGELRGGAWVVVDPTINKDMMEMYADNNARGGVLEPEGIVEIKYRKPALLATMERLDATYASLKKQLAEEGKTDEEKAALKVQVEAREQELLPVYQQISIQFADLHDRAGRMKAKGVIRKALDWRRARHYFYWRVRRRLCEEYTFRKIVTATSAAPMPREQMLDLVKQWFTNDNETVNFEDADELVSEWFEKRASVIDQRISKLKSDATKEQIVSLGNADQEAVIEGFSQLIENLSEDARAEILRKLNSRF

SEQ ID NO: 16 - protein, 652 aa, synthetic construct
MSQTHKHAIPANIADRCLINPEQYETKYKQSINDPDTFWGEQGKILDWITPYQKVKNTSFAPGNVSIKWYEDGTLNLAANCLDRHLQENGDRTAIIWEGDDTSQSKHISYRELHRDVCRFANTLLDLGIKKGDVVAIYMPMVPEAAVAMLACARIGAVHSVIFGGFSPEAVAGRIIDSSSRLVITADEGVRAGRSIPLKKNVDDALKNPNVTSVEHVIVLKRTGSDIDWQEGRDLWWRDLIEKASPEHQPEAMNAEDPLFILYTSGSTGKPKGVLHTTGGYLVYAATTFKYVFDYHPGDIYWCTADVGWVTGHSYLLYGPLACGATTLMFEGVPNWPTPARMCQVVDKHQVNILYTAPTAIRALMAEGDKAIEGTDRSSLRILGSVGEPINPEAWEWYWKKIGKEKCPVVDTWWQTETGGFMITPLPGAIELKAGSATRPFFGVQPALVDNEGHPQEGATEGNLVITDSWPGQARTLFGDHERFEQTYFSTFKNMYFSGDGARRDEDGYYWITGRVDDVLNVSGHRLGTAEIESALVAHPKIAEAAVVGIPHAIKGQAIYAYVTLNHGEEPSPELYAEVRNWVRKEIGPLATPDVLHWTDSLPKTRSGKIMRRILRKIAAGDTSNLGDTSTLADPGVVEKLLEEKQAIAMPS

SEQ ID NO: 17 - protein, 485 aa, synthetic construct
MSSLFPALSPAPTGAPADRPALRFGERSLTYAELAAAAGATAGRIGGAGRVAVWATPAMETGVAVVAALLAGVAAVPLNPKSGDKELAHILSDSAPSLVLAPPDAELPPALGALERVDVDVRARGAVPEDGADDGDPALVVYTSGTTGPPKGAVIPRRALATTLDALADAWQWTGEDVLVQGLPLFHVHGLVLGILGPLRRGGSVRHLGRFSTEGAARELNDGATMLFGVPTMYHRIAETLPADPELAKALAGARLLVSGSAALPVHDHERIAAATGRRVIERYGMTETLMNTSVRADGEPRAGTVGVPLPGVELRLVEEDGTPIAALDGESVGEIQVRGPNLFTEYLNRPDATAAAFTEDGFFRTGDMAVRDPDGYVRIVGRKATDLIKSGGYKIGAGEIENALLEHPEVREAAVTGEPDPDLGERIVAWIVPADPAAPPALGTLADHVAARLAPHKRPRVVRYLDAVPRNDMGKIMKRALNRD

SEQ ID NO: 18 - protein, 465 aa, synthetic construct
MSPELISILVLVVVFVIATTRSVNMGALAFAAAFGVGTLVADLDADGIFAGFPGDLFVVLVGVTYLFAIARANGTTDWLVHAAVRLVRGRVALIPWVMFALTGALTAIGAVSPAAVAIVAPVALSFATRYSISPLLMGTMVVHGAQAGGFSPISIYGSIVNGIVEREKLPGSEIGLFLASLVANLLIAAVLFAVLGGRKLWARGAVTPEGDGAPGKAGTGTTGSGSDTGTGTGTGTGTSAGTGGTAPTAVAVRSDRETGGAEGTGVRLTPARVATLVALVALVVAVLGFDLDAGLTAVTLAVVLSTAWPDDSRRAVGEIAWSTVLLICGVLTYVGVLEEMGTITWAGEGVGGIGVPLLAAVLLCYIGAIVSAFASSVGIMGALIPLAVPFLAQGEIGAVGMVAALAVSATVVDVSPFSTNGALVLAAAPDVDRDRFFRQLMVYGGIVVAAVPALAWLVLVVPGFG

SEQ ID NO: 19 - protein, 548 aa, synthetic construct
MVKKRLWDKQRTRRQEKLNLAQQKGFAKQVEHARAIELLETVIASGDRVCLEGNNQKQADFLSKCLSQCNPDAVNDLHIVQSVLALPSHIDVFEKGIASKVDFSFAGPQSLRLAQLVQQQKISIGSIHTYLELYGRYFIDLTPNICLITAHAADREGNLYTGPNTEDTPAIVEATAFKSGIVIAQVNEIVDKLPRVDVPADWVDFYIESPKHNYIEPLFTRDPAQITEVQILMAMMVIKGIYAPYQVQRLNHGIGFDTAAIELLLPTYAASLGLKGQICTNWALNPHPTLIPAIESGFVDSVHSFGSEVGMEDYIKERPDVFFTGSDGSMRSNRAFSQTAGLYACDSFIGSTLQIELQGNSSTATVDRISGFGGAPNMGSDPHGRRHASYAYTKAGREATDGKLIKGRKLVVQTVETYREHMHPVFVEELDAWQLQDKMDSELPPIMIYGEDVTHIVTEEGIANLLLCRTDEEREQAIRGVAGYTPVGLKRDAAKVEELRQRGIIQRPEDLGIDPTQVSRDLLAAKSVKDLVKWSGGLYSPPSRFRNW

SEQ ID NO: 20 - protein, 248 aa, synthetic construct
MILELDCGNSLIKWRVIEGAARSVAGGLAESDDALVEQLTSQQALPVRACRLVSVRSEQETSQLVARLEQLFPVSALVASSGKQLAGVRNGYLDYQRLGLDRWLALVAAHHLAKKACLVIDLGTAVTSDLVAADGVHLGGYICPGMTLMRSQLRTHTRRIRYDDAEARRALASLQPGQATAEAVERGCLLMLRGFVREQYAMACELLGPDCEIFLTGGDAELVRDELAGARIMPDLVFVGLALACPIE

SEQ ID NO: 21 - protein, 420 aa, synthetic construct
MTKKLLALGINHKTAPVSLRERVTFSPDTLDQALDSLLAQPMVQGGVVLSTCNRTELYLSVEEQDNLQEALIRWLCDYHNLNEDDLRNSLYWHQDNDAVSHLMRVASGLDSLVLGEPQILGQVKKAFADSQKGHLNASALRRMFQKSFSVAKRVRTETDIGASAVSVAFAACTLARQIFESLSTVTVLLVGAGETIELVARHLREHKVQKMIIANRTRERAQALADEVGAEVISLSDIDARLQDADIIISSTASPLPIIGKGMVERALKSRRNQPMLLVDIAVPRDVEPEVGKLANAYLYSVDDLQSIISHNLAQRQAAAVEAETIVEQEASEFMAWLRAQGASETIREYRSQSEQIRDELTTKALSALQQGGDAQAILQDLAWKLTNRLIHAPTKSLQQAARDGDDERLNILRDSLGLE

SEQ ID NO: 22 - protein, 401 aa, synthetic construct
MDYNLALDKAIQKLHDEGRYRTFIDIEREKGAFPKAQWNRPDGGKQDITVWCGNDYLGMGQHPVVLAAMHEALEAVGAGSGGTRNISGTTAYHRRLEAEIADLHGKEAALVFSSAYIANDATLSTLRLLFPGLIIYSDSLNHASMIEGIKRNAGPKRIFRHNDVAHLRELIAADDPAAPKLIAFESVYSMDGDFGPIKEICDIADEFGALTYIDEVHAVGMYGPRGAGVAERDGLMHRIDIFNGTLAKAYGVFGGYIAASAKMVDAVRSYAPGFIFSTSLPPAIAAGAQASIAFLKTAEGQKLRDAQQMHAKVLKMRLKALGMPIIDHGSHIVPVVIGDPVHTKAVSDMLLSDYGVYVQPINFPTVPRGTERLRFTPSPVHDLKQIDGLVHAMDLLWARCA

SEQ ID NO: 23 - protein, 531 aa, synthetic construct
MTLQSQTAKDCLALDGALTLVQCEAIATHRSRISVTPALRERCARAHARLEHAIAEQRHIYGITTGFGPLANRLIGADQGAELQQNLIYHLATGVGPKLSWAEARALMLARLNSILQGASGASPETIDRIVAVLNAGFAPEVPAQGTVGASGDLTPLAHMVLALQGRGRMIDPSGRVQEAGAVMDRLCGGPLTLAARDGLALVNGTSAMTAIAALTGVEAARAIDAALRHSAVLMEVLSGHAEAWHPAFAELRPHPGQLRATERLAQALDGAGRVCRTLTAARRLTAADLRPEDHPAQDAYSLRVVPQLVGAVWDTLDWHDRVVTCELNSVTDNPIFPEGCAVPALHGGNFMGVHVALASDALNAALVTLAGLVERQIARLTDEKLNKGLPAFLHGGQAGLQSGFMGAQVTATALLAEMRANATPVSVQSLSTNGANQDVVSMGTIAARRARAQLLPLSQIQAILALALAQAMDLLDDPEGQAGWSLTARDLRDRIRAVSPGLRADRPLAGHIEAVAQGLRHPSAAADPPA

SEQ ID NO: 24 - protein, 690 aa, synthetic construct
MITETNVAKPASTKVMNGDAAKAAPVEPFATYAHSQATKTVVIDGHNMKVGDVVAVARHGAKVELAASVAGPVQASVDFKESKKHTSIYGVTTGFGGSADTRTSDTEALQISLLEHQLCGYLPTDPTYEGMLLAAMPIPIVRGAMAVRVNSCVRGHSGVRLEVLQSFADFINIGLVPCVPLRGTISASGDLSPLSYIAGAICGHPDVKVFDTAASPPTVLTAPEAIAKYKLKTVRLASKEGLGLVNGTAVSAAAGALALYDAECLAMMSQTNTALTVEALDGHVGSFAPFIQEIRPHVGQIEAAKNIRHMLSNSKLAVHEEPELLADQDAGILRQDRYALRTSAQWIGPQLEMLGLARQQIETELNSTTDNPLIDVEGGMFHHGGNFQAMAVTSAMDSTRIVLQNLGKLSFAQVTELINCEMNHGLPSNLAGSEPSTNYHCKGLDIHCGAYCAELGFLANPMSNHVQSTEMHNQSVNSMAFASARKTMEANEVLSLLLGSQMYCATQALDLRVMEVKFKMAIVKLLNDTLTKHFSTFLTPEQLAKLNTTAAITLYKRLNQTPSWDSAPRFEDAAKHLVGCIMDALMVNDDITDLTNLPKWKKEFAKDAGDLYRSILTATTADGRNDLEPAEYLGQTRAVYEAIRSDLGVKVRRGDVAEGKSGKSIGSNVARIVEAMRDGRLMGAVSKMFF

SEQ ID NO: 25 - protein, 506 aa, synthetic construct
MNTINEYLSLEEFEAIIFGNQKVTISDVVVNRVNESFNFLKEFSGNKVIYGVNTGFGPMAQYRIKESDQIQLQYNLIRSHSSGTGKPLSPVCAKAAILARLNTLSLGNSGVHPSVINLMSELINKDITPLIFEHGGVGASGDLVQLSHLALVLIGEGEVFYKGERRPTPEVFEIEGLKPIQVEIREGLALINGTSVMTGIGVVNVYHAKKLLDWSLKSSCAINELVQAYDDHFSAELNQTKRHKGQQEIALKMRQNLSDSTLIRKREDHLYSGENTEEIFKEKVQEYYSLRCVPQILGPVLETINNVASILEDEFNSANDNPIIDVKNQHVYHGGNFHGDYISLEMDKLKIVITKLTMLAERQLNYLLNSKINELLPPFVNLGTLGFNFGMQGVQFTATSTTAESQMLSNPMYVHSIPNNNDNQDIVSMGTNSAVITSKVIENAFEVLAIEMITIVQAIDYLGQKDKISSVSKKWYDEIRNIIPTFKEDQVMYPFVQKVKDHLINN

SEQ ID NO: 26 - protein, 552 aa, synthetic construct
MSTTLILTGEGLGIDDVVRVARHQDRVELTTDPAILAQIEASCAYINQAVKEHQPVYGVTTGFGGMANVIISPEEAAELQNNAIWYHKTGAGKLLPFTDVRAAMLLRANSHMRGASGIRLEIIQRMVTFLNANVTPHVREFGSIGASGDLVPLISITGALLGTDQAFMVDFNGETLDCISALERLGLPRLRLQPKEGLAMMNGTSVMTGIAANCVHDARILLALALEAHALMIQGLQGTNQSFHPFIHRHKPHTGQVWAADHMLELLQGSQLSRNELDGSHDYRDGDLIQDRYSLRCLPQFLGPIIDGMAFISHHLRVEINSANDNPLIDTASAASYHGGNFLGQYIGVGMDQLRYYMGLMAKHLDVQIALLVSPQFNNGLPASLVGNIQRKVNMGLKGLQLTANSIMPILTFLGNSLADRFPTHAEQFNQNINSQGFGSANLARQTIQTLQQYIAITLMFGVQAVDLRTHKLAGHYNAAELLSPLTAKIYHAVRSIVKHPPSPERPYIWNDDEQVLEAHISALAHDIANDGSLVSAVEQTLSGLRSIILFR

SEQ ID NO: 27 - protein, 714 aa, synthetic construct
MHDDNTSPYCIGQLGNGAVHGADPLNWAKTAKAMECSHLEEIKRMVDTYQNATQVMIEGATLTVPQVAAIARRPEVHVVLDAANARSRVDESSNWVLDRIMGGGDIYGVTTGFGATSHRRTQQGVELQRELIRFLNAGVLSKGNSLPSETARAAMLVRTNTLMQGYSGIRWEILHAMEKLLNAHVTPKLPLRGTITASGDLVPLSYIAGLLTGRPNSKAVTEDGREVSALEALRIAGVEKPFELAPKEGLALVNGTAVGSALASTVCYDANIMVLLAEVLSALFCEVMQGKPEFADPLTHKLKHHPGQMEAAAVMEWVLDGSSFMKAAAKFNETDPLRKPKQDRYALRTSPQWLGPQVEVIRNATHAIEREINSVNDNPIIDAARGIALHGGNFQGTPIGVSMDNMRLSLAAIAKLMFAQFSELVNDYYNNGLPSNLSGGPNPSLDYGMKGAEIAMASYLSEINYLANPVTTHVQSAEQHNQDVNSLGLVSARKTEEAMEILKLMSATFLVGLCQAIDLRHVEETMQSAVKQVVTQVAKKTLFMGSDGSLLPSRFCEKELLMVVDRQPVFSYIDDSTSDSYPLMEKLRGVLVSRALKSADKETSNAVFRQIPVFEAELKLQLSRVVPAVREAYDTKGLSLVPNRIQDCRTYPLYKLVRGDLKTQLLSGQRTVSPGQEIEKVFNAISAGQLVAPLLECVQGWTGTPGPFSARASC

SEQ ID NO: 28 - protein, 529 aa, synthetic construct
MIETNHKDNFLIDGENKNLEINDIISISKGEKNIIFTNELLEFLQKGRDQLENKLKENVAIYGINTGFGGNGDLIIPFDKLDYHQSNLLDFLTCGTGDFFNDQYVRGIQFIIIIALSRGWSGVRPMVIQTLAKHLNKGIIPQVPMHGSVGASGDLVPLSYIANVLCGKGMVKYNEKLMNASDALKITSIEPLVLKSKEGLALVNGTRVMSSVSCISINKFETIFKAAIGSIALAVEGLLASKDHYDMRIHNLKNHPGQILIAQILNKYFNTSDNNTKSSNITFNQSENVQKLDKSVQEVYSLRCAPQILGIISENISNAKIVIKREILSVNDNPLIDPYYGDVLSGGNFMGNHIARIMDGIKLDISLVANHLHSLVALMMHSEFSKGLPNSLSPNPGIYQGYKGMQISQTSLVVWLRQEAAPACIHSLTTEQFNQDIVSLGLHSANGAASMLIKLCDIVSMTLIIAFQAISLRMKSIENFKLPNKVQKLYSSIIKIIPILENDRRTDIDVREITNAILQDKLDFINLNL

SEQ ID NO: 29 - protein, 550 aa, synthetic construct; identical to SEQ ID NO: 2

SEQ ID NO: 30 - protein, 504 aa, synthetic construct
MDLLLMEKTLLGLFVAVVVAITVSKLRGKKFKLPPGPIPVPVFGNWLQVGDDLNHRNLTEMAKKFGEVFMLRMGQRNLVVVSSPDLAKEVLHTQGVEFGSRTRNVVFDIFTGKGQDMVFTVYGEHWRKMRRIMTVPFFTNKVVQQYRYGWESEAAAVVEDVKKHPEAATNGMVLRRRLQLMMYNNMYRIMFDRRFESEDDPLFVKLKGLNGERSRLAQSFEYNYGDFIPVLRPFLRGYLKICKEVKEKRIQLFKDYFVDERKKLSSTQATTNEGLKCAIDHILDAQQKGEINEDNVLYIVENINVAAIETTLWSIEWGIAELVNHPEIQKKLRDELDTVLGRGVQITEPEIQKLPYLQAVVKETLRLRMAIPLLVPHMNLHDAKLGGFDIPAESKILVNAWWLANNPAHWKKPEEFRPERFLEEESKVEANGNDFRYLPFGVGRRSCPGIILALPILGITLGRLVQNFELLPPPGQTQLDTTEKGGQFSLHILKHSPIVMKPRT

SEQ ID NO: 31 - protein, 504 aa, synthetic construct
MDLLLLEKTLIGLFIAIVVAIIVSKLRGKKFKLPPGPIPVPVFGNWLQVGDDLNHRNLTDMAKKFGDVFMLRMGQRNLVVVSSPDLAKEVLHTQGVEFGSRTRNVVFDIFTGKGQDMVFTVYGEHWRKMRRIMTVPFFTNKVVQQYRHGWEAEAAAVVEDVKKHPEAATSGMVLRRRLQLMMYNNMYRIMFDRRFESEEDPLFVKLKGLNGERSRLAQSFEYNYGDFIPVLRPFLRGYLKICKEVKEKRIQLFKDYFVDERKKLASTQVTTNEGLKCAIDHILDAQQKGEINEDNVLYIVENINVAAIETTLWSIEWGIAELVNHPEIQKKLRDELDTVLGHGVQVTEPELHKLPYLQAVVKETLRLRMAIPLLVPHMNLHDAKLGGFDIPAESKILVNAWWLANNPAHWKKPEEFRPERFLEEESKVEANGNDFRYLPFGVGRRSCPGIILALPILGVTLGRLVQNFEMLPPPGQTQLDTTEKGGQFSLHILKHSTIVMKPRA

SEQ ID NO: 32 - protein, 505 aa, synthetic construct
MDLLLLEKTLIGLFFAILIAIIVSKLRSKRFKLPPGPIPVPVFGNWLQVGDDLNHRNLTEYAKKFGDVFLLRMGQRNLVVVSSPELAKEVLHTQGVEFGSRTRNVVFDIFTGKGQDMVFTVYGEHWRKMRRIMTVPFFTNKVVQQYRGGWESEAASVVEDVKKNPESATNGIVLRKRLQLMMYNNMFRIMFDRRFESEDDPLFVKLRALNGERSRLAQSFEYNYGDFIPILRPFLRGYLKICKEVKEKRLKLFKDYFVDERKKLANTKSMDSNALKCAIDHILEAQQKGEINEDNVLYIVENINVAAIETTLWSIEWGIAELVNHPHIQKKLRDEIDTVLGPGMQVTEPDMPKLPYLQAVIKETLRLRMAIPLLVPHMNLHDAKLAGYDIPAESKILVNAWWLANNPAHWKKPEEFRPERFFEEEKHVEANGNDFRFLPFGVGRRSCPGIILALPILGITLGRLVQNFEMLPPPGQSKLDTSEKGGQFSLHILKHSTIVMKPRSF

SEQ ID NO: 33 - protein, 544 aa, synthetic construct
MGDCAAPKQEIIFRSKLPDIYIPKHLPLHSYCFENISKVSDRACLINGATGETFSYAQVELISRRVASGLNKLGIHQGDTMMILLPNTPEYFFAFLGASYRGAVSTMANPFFTSPEVIKQLKASQAKLIITQACYVEKVKEYAAENNITVVCIDEAPRDCLHFTTLMEADEAEMPEVAIDSDDVVALPYSSGTTGLPKGVMLTHKGLVTSVAQRVDGENPNLYIHSEDVMICILPLFHIYSLNAVLCCGLRAGATILIMQKFDIVPFLELIQKYKVTIGPFVPPIVLAIAKSPVVDNYDLSSVRTVMSGAAPLGKELEDAVRAKFPNAKLGQGYGMTEAGPVLAMCLAFAKEPYEIKSGACGTVVRNAEMKIVDPETHASLPRNQSGEICIRGDQIMKGYLNDPESTKTTIDEEGWLHTGDIGFIDEDDELFIVDRLKEIIKYKGFQVAPAEIEALLLTHPTISDAAVVPMIDEKAGEVPVAFVVRLNGSTTTEEEIKQFVSKQVVFYKRVFRVFFVDAIPKSPSGKILRKELRARIASGDLPK

SEQ ID NO: 34 - protein, 543 aa, synthetic construct
MEPTTKSKDIIFRSKLPDIYIPKHLPLHTYCFENISRFGSRPCLINGSTGEILTYDQVELASRRVGSGLHRLGIRQGDTIMLLLPNSPEFVLAFLGASHIGAVSTMANPFFTPAEVVKQAAASRAKLIVTQACHVDKVRDYAAEHGVKVVCVDGAPPEECLPFSEVASGDEAELPAVKISPDDVVALPYSSGTTGLPKGVMLTHKGLVTSVAQQVDGENPNLYIHSDDVIMCVLPLFHIYSLNSIMLCGLRVGAAILIMQKFEIVPFLELIQRYRVTIGPFVPPIVLAIEKSPVVEKYDLSSVRTVMSGAAPLGRELEDAVRLKFPNAKLGQGYGMTEAGPVLAMCLAFAKEPFEIKSGACGTVVRNAEMKIVDTETGASLGRNQPGEICIRGDQIMKGYLNDPESTERTIDKEGWLHTGDIGFIDDDDELFIVDRLKEIIKYKGFQVAPAELEALLLNHPNISDAAVVSMKDEQAGEVPVAYVVKSNGSTITEDEIKQFVSKQVIFYKRINRVFFIDAIPKSPSGKILRKDLRARLAAGVPN

SEQ ID NO: 35 - protein, 545 aa, synthetic construct
MPMENEAKQGDIIFRSKLPDIYIPNHLSLHSYCFENISEFSSRPCLINGANNQIYTYADVELNSRKVAAGLHKQFGIQQKDTIMILLPNSPEFVFAFLGASYLGAISTMANPLFTPAEVVKQVKASNAEIIVTQACHVNKVKDYALENDVKIVCIDSAPEGCVHFSELIQADEHDIPEVQIKPDDVVALPYSSGTTGLPKGVMLTHKGLVTSVAQQVDGENPNLYIHSEDVMLCVLPLFHIYSLNSVLLCGLRVGAAILIMQKFDIVPFLELIQNYKVTIGPFVPPIVLAIAKSPMVDNYDLSSVRTVMSGAAPLGKELEDTVRAKFPNAKLGQGYGMTEAGPVLAMCLAFAKEPFEIKSGACGTVVRNAEMKIVDPDTGNSLHRNQSGEICIRGDQIMKGYLNDPEATAGTIDKEGWLHTGDIGYIDNDDELFIVDRLKELIKYKGFQVAPAELEALLLNHPNISDAAVVPMKDEQAGEVPVAFVVRSNGSTITEDEVKEFISKQVIFYKRIKRVFFVDAVPKSPSGKILRKDLRAKLAAGFPN

SEQ ID NO: 36 - protein, 542 aa, synthetic construct
MDTKTTQQEIIFRSKLPDIYIPKQLPLHSYCFENISQFSSKPCLINGSTGKVYTYSDVELTSRKVAAGFHNLGIQQRDTIMLLLPNCPEFVFAFLGASYLGAIITMANPFFTPAETIKQAKASNSKLIITQSSYTSKVLDYSSENNVKIICIDSPPDGCLHFSELIQSNETQLPEVEIDSNEVVALPYSSGTTGLPKGVMLTHKGLVTSVAQQVDGENPNLYIHSEDMMMCVLPLFHIYSLNSVLLCGLRVGAAILIMQKFEIGSFLKLIQRYKVTIGPFVPPIVLAIAKSEVVDDYDLSTIRTMMSGAAPLGKELEDAVRAKFPHAKLGQGYGMTEAGPVLAMCLAFAKKPFEIKSGACGTVVRNAEMKIVDPDAGFSLPRNQPGEICIRGDQIMKGYLNDPEATERTIDKQGWLHTGDIGYIDDDDELFIVDRLKELIKYKGFQVAPAELEALLLNHPTISDAAVVPMKDESAGEVPVAFVVRTNGFEVTENEIKKYISEQVVFYKKINRVYFVDAIPKAPSGKILRKDLRARLAAGIPS

SEQ ID NO: 37 - protein, 389 aa, synthetic construct
MVTVEEYRKAQRAEGPATVMAIGTATPSNCVDQSTYPDYYFRITNSEHKTELKEKFKRMCEKSMIKTRYMHLTEEILKENPNMCAYMAPSLDARQDIVVVEVPKLGKEAAQKAIKEWGQPKSKITHLVFCTTSGVDMPGCDYQLAKLLGLRPSVKRLMMYQQGCFAGGTVLRLAKDLAENNKGARVLVVCSEITAVTFRGPSESHLDSLVGQALFGDGAAAIIMGSDPIPGVERPLFQLVSAAQTLLPDSEGAIDGHLREVGLTFHLLKDVPGLISKNIEKSLVEAFQPLGISDWNSLFWIAHPGGPAILDQVELKLGLKPEKLKATREVLSNYGNMSSACVLFILDEMRKASTKEGLGTSGEGLEWGVLFGFGPGLTVETVVLHSVAI

SEQ ID NO: 38 - protein, 389 aa, synthetic construct
MVTVEEVRKAQRAEGPATVLAIGTATPPNCIDQSTYPDYYFRITKSEHKAELKEKFQRMCDKSMIKKRYMYLTEEILKENPSMCEYMAPSLDARQDMVVVEIPKLGKEAATKAIKEWGQPKSKITHLVFCTTSGVDMPGADYQLTKLLGLRPSVKRLMMYQQGCFAGGTVLRLAKDLAENNKGARVLVVCSEITAVTFRGPSDTHLDSLVGQALFGDGAAAIIVGSDPLPEVEKPLFELVSAAQTILPDSDGAIDGHLREVGLTFHLLKDVPGLISKNIEKSLNEAFKPLNITDWNSLFWIAHPGGPAILDQVEAKLGLKPEKLEATRHILSEYGNMSSACVLFILDEVRRKSAANGHKTTGEGLEWGVLFGFGPGLTVETVVLHSVAA

SEQ ID NO: 39 - protein, 393 aa, synthetic construct
MSMTPSVHEIRKAQRSEGPATVLSIGTATPTNFVPQADYPDYYFRITNSDHMTDLKDKFKRMCEKSMITKRHMYLTEEILKENPKMCEYMAPSLDARQDIVVVEVPKLGKEAAAKAIKEWGQPKSKITHLIFCTTSGVDMPGADYQLTKLLGLRPSVKRFMMYQQGCFAGGTVLRLAKDLAENNKGARVLVVCSEITAVTFRGPSHTHLDSLVGQALFGDGAAAVILGADPDTSVERPIFELVSAAQTILPDSEGAIDGHLREVGLTFHLLKDVPGLISKNIEKSLVEAFTPIGISDWNSIFWIAHPGGPAILDQVEAKLGLKQEKLSATRHVLSEYGNMSSACVLFILDEVRKKSVEEGKATTGEGLEWGVLFGFGPGLTVETIVLHSLPAV

SEQ ID NO: 40 - protein, 395 aa, synthetic construct
MAPPAMEEIRRAQRAEGPATVLAIGASTPPNALYQADYPDYYFRITKSEHLTELKEKFKQMCDKSMIRKRYMYLTEEILKENPNICAFMAPSLDARQDIVVTEVPKLAKEASARAIKEWGQPKSRITHLIFCTTSGVDMPGADYQLTRLLGLRPSVNRIMLYQQGCFAGGTVLRLAKDLAENNAGARVLVVCSEITAVTFRGPSESHLDSLVGQALFGDGAAAIIVGSDPDLTTERPLFQLVSASQTILPESEGAIDGHLREMGLTFHLLKDVPGLISKNIQKSLVETFKPLGIHDWNSIFWIAHPGGPAILDQVEIKLGLKEEKLASSRNVLAEYGNMSSACVLFILDEMRRRSAEAGQATTGEGLEWGVLFGFGPGLTVETVVLRSVPIAGAV

SEQ ID NO: 41 - protein, 169 aa, synthetic construct
MSAITAIHVENIEFPAVITSPVTGKSYFLGGAGERGLTIEGNFIKFTAIGVYLEDVAVASLATKWKGKSSEELLETLDFYRDIISGPFEKLIRGSKIRELSGPEYSRKVTENCVAHLKSVGTYGDAEVEAMEKFVEAFKPINFPPGASVFYRQSPDGILGVSISIHFFP

SEQ ID NO: 42 - protein, 223 aa, synthetic construct
MAAASLTAVQVENLEFPAVVTSPATGKTYFLGGAGVRGLTIEGNFIKFTGIGVYLEDQAVASLATKWKGKSSEELVESLDFFRDIISGPFEKLIRGSKIRQLSGPEYSKKVMENCVAHMKSVGTYGDAEAAGIEEFAQAFKPVNFPPGASVFYRQSPDGVLGLSFSQDATIPEEEAAVIKNKPVSAAVLETMIGEHAVSPDLKRSLAARLPAVLSHGVFKIGN

SEQ ID NO: 43 - protein, 227 aa, synthetic construct
MAAEPSITAIQFENLVFPAVVTPPGSSKSYFLAGAGERGLTIDGKFIKFTGIGVYLEDKAVPSLAGKWKDKSSQQLLQTLHFYRDIISGPFEKLIRGSKILALSGVEYSRKVMENCVAHMKSVGTYGDAEAEAIQQFAEAFKNVNFKPGASVFYRQSPLGHLGLSFSQDGNIPEKEAAVIENKPLSSAVLETMIGEHAVSPDLKCSLAARLPAVLQQGIIVTPPQHN

SEQ ID NO: 44 - protein, 215 aa, synthetic construct
MGPSPSVTELQVENVTFPPSVKPPGSTKTLFLGGAGERGLEIQGKFIKFTAIGVYLEGDAVASLAVKWKGKSKEELTDSVEFFRDIVTGPFEKFTQVTTILPLTGQQYSEKVSENCVAFWKSVGIYTDAEAKAIEKFIEVFKEETFPPGSSILFTQSPNGALTIAFSKDGVIPEVGKAVIENKLLAEGLLESIIGKHGVSPVAKQCLATRLSELL

SEQ ID NO: 45 - protein, 337 aa, synthetic construct
MGSASETVCVTGAAGFIGSWLVMRLIQNGYKVRATVRDPANMKKVKHLLELPNAKTNLSLWKADLAEEGSFDEAIKGCTGVFHVATPMDFESKDPENEVIKPTINGLIDIMKACMKAKTVRRLVFTSSAGTVDVTEHPKPLFDESCWSDVQFCRRVRMTGWMYFVSKTLAEQEAWKFAKENNIDFISVIPPLVVGPFLVPTMPPSLITALSLITGNESHYAIIKQGQFVHLDDLCLAHIFLFQHPKAQGRYICCSHEATIHDIASLLNQKYPEFNVPTKFKNIPDQLEIIRFSSKKITDLGFKFKYSLEDMFTGAVETCKEKRLLSETAEISGTTQK

SEQ ID NO: 46 - protein, 347 aa, synthetic construct
MKDSVASATASAPGTVCVTGAAGFIGSWLVMRLLERGYIVRATVRDPANLKKVKHLLDLPKADTNLTLWKADLNEEGSFDEAIEGCSGVFHVATPMDFESKDPENEVIKPTINGVLSIIRSCTKAKTVKRLVFTSSAGTVNVQEHQQPVFDENNWSDLHFINKKKMTGWMYFVSKTLAEKAAWEAAKENNIDFISIIPTLVGGPFIMPTFPPSLITALSPITRNEGHYSIIKQGQFVHLDDLCESHIFLYERPQAEGRYICSSHDATIHDLAKLMREKWPEYNVPTEFKGIDKDLPVVSFSSKKLIGMGFEFKYSLEDMFRGAIDTCREKGLLPHSFAENPVNGNKV

SEQ ID NO: 47 - protein, 359 aa, synthetic construct
MVDMKDDDSPATVCVTGAAGFIGSWLIMRLLQQGYIVRATVRDPANMKKVKHLQELEKADKNLTLWKADLTEEGSFDEAIKGCSGVFHVATPMDFESKDPENEVIKPTINGVLSIVRSCVKAKTVKRLVFTSSAGTVNLQEHQQLVYDENNWSDLDLIYAKKMTGWMYFVSKILAEKAAWEATKENNIDFISIIPTLVVGPFITPTFPPSLITALSLITGNEAHYSIIKQGQFVHLDDLCEAHIFLYEQPKAEGRYICSSHDATIYDLAKMIREKWPEYNVPTELKGIEKDLQTVSFSSKKLIGMGFEFKYSLEDMYKGAIDTCREKGLLPYSTHETPANANANANANVKKNQNENTEI

SEQ ID NO: 48 - protein, 349 aa, synthetic construct
MASESESVCVTGASGFVGSWLVMRLLDRGYTVRATVRDPANKKKVKHLLDLPKAATHLTLWKADLAEEGSFDEAIKGCTGVFHVATPMDFESKDPENEVIKPTINGVLDIMKACLKAKTVRRLVFTASAGSVNVEETQKPVYDESNWSDVEFCRRVKMTGWMYFASKTLAEQEAWKFAKENNIDFITIIPTLVIGPFLMPAMPPSLITGLSPLTGNESHYSIIKQGQFIHLDDLCQSHIYLYEHPKAEGRYICSSHDATIHEIAKLLREKYPEYNVPTTFKGIEENLPKVHFSSKKLLETGFEFKYSLEDMFVGAVDACKAKGLLPPPTERVEKQEVDESSVVGVKVTG

SEQ ID NO: 49 - protein, 511 aa, synthetic construct
MSPLILYSIALAIFLYCLRTLLKRHPHRLPPGPRPWPIIGNLPHMGQMPHHSLAAMARTYGPLMHLRLGFVDVIVAASASVASQLLKTHDANFSSRPHNSGAKYIAYNYQDLVFAPYGPRWRMLRKISSVHLFSGKALDDYRHVRQEEVAVLIRALARAESKQAVNLGQLLNVCTANALGRVMLGRRVFGDGSGVSDPMAEEFKSMVVEVMALAGVFNIGDFIPALDWLDLQGVAAKMKNLHKRFDTFLTGLLEEHKKMLVGDGGSEKHKDLLSTLISLKDSADDEGLKLTDTEIKALLLNMFTAGTDTSSSTVEWAIAELIRHPKILAQVLKELDTVVGRDRLVTDLDLPQLTYLQAVIKETFRLHPSTPLSLPRVAAESCEIMGYHIPKGSTLLVNVWAIARDPKEWAEPLEFRPERFLPGGEKPNVDIKGNDFEVIPFGAGRRICAGMSLGLRMVQLLTATLVHAFDWDLTSGLMPEDLSMEEAYGLTLQRAEPLMVHPRPRLSPNVY

SEQ ID NO: 50 - protein, 507 aa, synthetic construct
MASFLLYSILSAVFLYFIFATLRKRHRLPLPPGPKPWPIIGNLPHMGPVPHHSLAALAKVYGPLMHLRLGFVDVVVAASASVAAQFLKVHDANFSSRPPNSGAKYVAYNYQDLVFAPYGPRWRMLRKISSVHLFSGKALDDFRHVRQDEVGVLVRALADAKTKVNLGQLLNVCTVNALGRVMLGKRVFGDGSGKADPEADEFKSMVVELMVLAGVVNIGDFIPALEWLDLQGVQAKMKKLHKRFDRFLSAILEEHKIKARDGSGQHKDLLSTFISLEDADGEGGKLTDTEIKALLLNMFTAGTDTSSSTVEWAIAELIRHPKILAQVRKELDSVVGRDRLVSDLDLPNLTYFQAVIKETFRLHPSTPLSLPRMASESCEINGYHIPKGATLLVNVWAIARDPDEWKDPLEFRPERFLPGGERPNADVRGNDFEVIPFGAGRRICAGMSLGLRMVQLLAATLVHAFDWELADGLMPEKLNMEEAFGLTLQRAAPLMVHPRPRLSPRAY

SEQ ID NO: 51 - protein, 512 aa, synthetic construct
MTPLTLLIGTCVTGLFLYVLLNRCTRNPNRLPPGPTPWPVVGNLPHLGTIPHHSLAAMAKKYGPLMHLRLGFVDVVVAASASVAAQFLKTHDANFADRPPNSGAKHIAYNYQDLVFAPYGPRWRMLRKICSVHLFSTKALDDFRHVRQEEVAILARALVGAGKSPVKLGQLLNVCTTNALARVMLGRRVFDSGDAQADEFKDMVVELMVLAGEFNIGDFIPVLDWLDLQGVTKKMKKLHAKFDSFLNTILEEHKTGAGDGVASGKVDLLSTLISLKDDADGEGGKLSDIEIKALLLNLFTAGTDTSSSTIEWAIAELIRNPQLLNQARKEMDTIVGQDRLVTESDLGQLTFLQAIIKETFRLHPSTPLSLPRMALESCEVGGYYIPKGSTLLVNVWAISRDPKIWADPLEFQPTRFLPGGEKPNTDIKGNDFEVIPFGAGRRICVGMSLGLRMVQLLTATLIHAFDWELADGLNPKKLNMEEAYGLTLQRAAPLVVHPRPRLAPHVYETTKV

SEQ ID NO: 52 - protein, 517 aa, synthetic construct
MAPLLLLFFTLLLSYLLYYYFFSKERTKGSRAPLPPGPRGWPVLGNLPQLGPKPHHTLHALSRAHGPLFRLRLGSVDVVVAASAAVAAQFLRAHDANFSNRPPNSGAEHIAYNYQDLVFAPYGPGWRARRKLLNVHLFSGKALEDLRPVREGELALLVRALRDRAGANELVDLGRAANKCATNALARAMVGRRVFQEEEDEKAAEFENMVVELMRLAGVFNVGDFVPGIGWLDLQGVVRRMKELHRRYDGFLDGLIAAHRRAAEGGGGGGKDLLSVLLGLKDEDLDFDGEGAKLTDTDIKALLLNLFTAGTDTTSSTVEWALSELVKHPDILRKAQLELDSVVGGDRLVSESDLPNLPFMQAIIKETFRLHPSTPLSLPRMAAEECEVAGYCIPKGATLLVNVWAIARDPAVWRDPLEFRPARFLPDGGCEGMDVKGNDFGIIPFGAGRRICAGMSLGIRMVQFMTATLAHAFHWDLPEGQMPEKLDMEEAYGLTLQRATPLMVHPVPRLAPTAYQS

SEQ ID NO: 53 - protein, 1007 aa, synthetic construct
MASNSNLIRAIESALGVSFGSELVSDTAIVVVTTSVAVIIGLLFFLLKRSSDRSKESKPVVISKPLLVEEEEEEDEVEAGSGKTKVTMFYGTQTGTAEGFAKSLAKEIKARYEKAIVKVVDLDDYAADDDQYEQKLKKETLVFFMLATYGDGEPTDDAARFYKWFTEENERGAWLQQLTYGVFSLGNRQYEHFNKIGKVVDEQLSKQGAKRLIPVGLGDDDQCIEDDFAAWRETLWPELDQLLRDEDDANTVSTPYAAAIPEYRVVIHDPLSGRGEAPSFSIDSHLTICEIWSTSREGSNQQISEYFWTSNSLKTMASNSNLIRSIESALGVSFGSESVSDTAIVVVTTSVAVIIGLLFFLLKRSSDRSKESKPVVISKPLLVEEEEDEVEAGSGKTKVTLFYGTQTGTAEGFAKSLAEEIKARYEKAIVKVVDLDDYAADDDQYEQKLKKETLVFFMLATYGDGEPTDNAARFYKWFTEENERGAWLQQLTYGVFSLGNRQYEHFNKIGKVVDEQLSKQGAKRLIPVGLGDDDQCIEDDFAAWRETLWPELDQLLRDEDDANTVSTPYTAAIPEYRVVIHDPTTTSYEDKNLNMANGNASYDIHHPCRVNVAVQRELHKPESDRSCIHLEFDISGTGIIYETGDHVGVYADNFDEVVEEAANLLGQPLELLFSVHADKDDGTSLGGSLPPPFPGPCTLRDALAHYADLLNPPRKAALSALAAHAVEPSEAERLKFLSSPQGKEDYSQWVVASQRSLLEIMAEFPSAKPPLGVFFAAVAPRLQPRYYSISSSPRFVPNRVHVTCALVYGPSPTGRIHKGVCSTWMKNAVPLEKSHDCSSAPIFTRTSNFKLPTDPSIPIIMVGPGTGLAPFRGFLQERLALKEDGVQLGHAMLFFGCRNRRMDFIYEDELNNFVDQGAVSELVVAFSREGPEKEYVQHKLNAKAAQVWGLISQGGYLYVCGDAKGMARDVHRMLHTIVEQQENVDSRKAEVIVKKLQMEGRYLRDVW

SEQ ID NO: 54 - protein, 1007 aa, synthetic construct; identical to SEQ ID NO: 53

SEQ ID NO: 55 - protein, 708 aa, synthetic construct
MSSSSSSPFDLMSAIIKGEPVVVSDPANASAYESVAAELSSMLIENRQFAMIISTSIAVLIGCIVMLLWRRSGGSGSSKRAETLKPLVLKPPREDEVDDGRKKVTIFFGTQTGTAEGFAKALGEEARARYEKTRFKIVDLDDYAADDDEYEEKLKKEDVAFFFLATYGDGEPTDNAARFYKWFTEGDDRGEWLKNLKYGVFGLGNRQYEHFNKVAKVVDDILVEQGAQRLVHVGLGDDDQCIEDDFTAWREALWPELDTILREEGDTAVTPYTAAVLEYRVSIHNSADALNEKNLANGNGHAVFDAQHPYRANVAVRRELHTPESDRSCTHLEFDIAGSGLTYETGDHVGVLSDNLNETVEEALRLLDMSPDTYFSLHSDKEDGTPISSSLPPTFPPCSLRTALTRYACLLSSPKKSALLALAAHASDPTEAERLKHLASPAGKDEYSKWVVESQRSLLEVMAEFPSAKPPLGVFFAAVAPRLQPRFYSISSSPKIAETRIHVTCALVYEKMPTGRIHKGVCSTWMKSAVPYEKSENCCSAPIFVRQSNFKLPSDSKVPIIMIGPGTGLAPFRGFLQERLALVESGVELGPSVLFFGCRNRRMDFIYEEELQRFLESGALSELSVAFSREGPTKEYVQHKMMDKASDIWNMISQGAYVYVCGDAKGMARDVHRSLHTIAQEQGSMDSTKAESFVKNLQMSGRYLRDVW

SEQ ID NO: 56 - protein, 508 aa, synthetic construct
MALDTFLLRELAAAAVLFLISHYLIHSLLKKSTPPLPPGPKGWPFVGALPLLGTMPHVALAQMAKKYGPVMYLKMGTCGMVVASTPDAARAFLKTLDLNFSNRPPNAGATHLAYNAQDMVFADYGPRWKLLRKLSNLHMLGGKALEDWTQVRTVELGHMIQAMCEASRAKEPVVVPEMLTYAMANMIGKVILGHRVFVTQGSESNEFKDMVVELMTSAGYFNIGDFIPSIAWMDLQGIERGMKKLHKRFDALLTKMFEEHMATAHERKGNPDLLDIVMANRDNSEGERLTTTNIKALLLNLFSAGTDTSSSIIEWSLAEMLKNPSILKRAHEEMDQVIGRNRRLEESDIKKLPYLQAICKESFRKHPSTPLNLPRVSSQACQVNGYYIPKDTRLSVNIWAIGRDPEVWENPLDFTPERFLSGKNAKIDPRGNDFELIPFGAGRRICAGTRMGIVLVEYILGTLVHSFDWSLPHGVKLNMDEAFGLALQKAVPLAAIVSPRLAPTAYVV

SEQ ID NO: 57 - protein, 504 aa, synthetic construct
MSIFLITSLLLCLSLHLLLRRRHISRLPLPPGPPNLPIIGALPFIGPMPHSGLALLARRYGPIMFLKMGIRRVVVASSSTAARTFLKTFDSHFSDRPSGVISKEISYNGQNMVFADYGPKWKLLRKVSSLHLLGSKAMSRWAGVRRDEALSMIQFLKKHSDSEKPVLLPNLLVCAMANVIGRIAMSKRVFHEDGEEAKEFKEMIKELLVGQGASNMEDLVPAIGWLDPMGVRKKMLGLNRRFDRMVSKLLVEHAETAGERQGNPDLLDLVVASEVKGEDGEGLCEDNIKGFISDLFVAGTDTSAIVIEWAMAEMLKNPSILRRAQEETDRVIGRHRLLDESDIPNLPYLQAICKEALRKHPPTPLSIPHYASEPCEVEGYHIPGETWLLVNIWAIGRDPDVWENPLVFDPERFLQGEMARIDPMGNDFELIPFGAGRRICAGKLAGMVMVQYYLGTLVHAFDWSLPEGVGELDMEEGPGLVLPKAVPLAVMATPRLPAAAYGLL

SEQ ID NO: 58 - protein, 360 aa, synthetic construct
MGSEAETVCVTGASGFIGSWLIMRLLERGYTVRATVRDPDNEKKVKHLVELPKAKTHLTLWKADLSDEGSFDEAIHGCTGVFHVATPMDFESKDPENEVIKPTINGVLGIMKACKKAKTVKRLVFTSSAGTVDVEEHKKPVYDENSWSDLDFVQSVKMTGWMYFVSKTLAEKAAWKFAEENSIDFISVIPPLVVGPFLMPSMPPSLITALSPITRNEGHYAIIKQGNYVHLDDLCMGHIFLYEHAESKGRYFCSSHSATILELSKFLRERYPEYDLPTEYKGVDDSLENVVFCSKKILDLGFQFKYSLEDMFTGAVETCREKGLIPLTNIDKKHVAAKGLIPNNSDEIHVAAAEKTTATA

SEQ ID NO: 59 - protein, 337 aa, synthetic construct; identical to SEQ ID NO: 45

SEQ ID NO: 60 - protein, 351 aa, synthetic construct
MENEKKGPVVVTGASGYVGSWLVMKLLQKGYEVRATVRDPTNLKKVKPLLDLPRSNELLSIWKADLDGIEGSFDEVIRGSIGVFHVATPMNFQSKDPENEVIQPAINGLLGILRSCKNAGSVQRVIFTSSAGTVNVEEHQAAAYDETCWSDLDFVNRVKMTGWMYFLSKTLAEKAAWEFVKDNHIHLITIIPTLVVGSFITSEMPPSMITALSLITGNDAHYSILKQIQFVHLDDLCDAHIFLFEHPKANGRYICSSYDSTIYGLAEMLKNRYPTYAIPHKFKEIDPDIKCVSFSSKKLMELGFKYKYTMEEMFDDAIKTCREKKLIPLNTEEIVLAAEKFEEVKEQIAVK

SEQ ID NO: 61 - protein, 349 aa, synthetic construct; identical to SEQ ID NO: 48

SEQ ID NO: 62 - protein, 326 aa, synthetic construct
MTVSSPCVGEGQGRVLIIGASGFIGEFIAQASLDSGRTTFLLVRSLDKGAIPSKSKTINSLHDKGAILIHGVIEDQEFVEGILKDHKIDIVISAVGGANILNQLTIVKAIKAVGTIKRFLPSEFGHDVDRANPVEPGLAMYKEKRMVRRLIEESGVPYTYICCNSIASWPYYDNTHPSEVIPPLDRFQIYGDGTVKAYFVDGSDIGKFTMKVVDDIRTLNKSVHFRPSCNFLNMNELSSLWEKKIGYMLPRLTVTEDDLLAAAAENIIPQSIVASFTHDIFIKGCQVNFSIDGPNEVEVSNLYPDETFRTMDECFDDFVMKMDRWN

SEQ ID NO: 63 - protein, 352 aa, synthetic construct
MTRSPSPNGQAEKGSRILIIGATGFIGHFIAQASLASGKSTYILSRAAARCPSKARAIKALEDQGAISIHGSVNDQEFMEKTLKEHEIDIVISAVGGGNLLEQVILIRAMKAVGTIKRFLPSEFGHDVDRAEPVEPGLTMYNEKRRVRRLIEESGVPYTYICCNSIASWPYYDNTHPSEVSPPLDQFQIYGDGSVKAYFVAGADIGKFTVKATEDVRTLNKIVHFRPSCNFLNINELATLWEKKIGRTLPRVVVSEDDLLAAAEENIIPQSVVASFTHDIFIKGCQVNFPVDGPNEIEVSSLYPDEPFQTMDECFNEFAGKIEEDKKHVVGTKGKNIAHRLVDVLTAPKLCA

SEQ ID NO: 64 - protein, 359 aa, synthetic construct
MKSTNMNGSSPNVSEETGRTLVVGSGGFMGRFVTEASLDSGRPTYILARSSSNSPSKASTIKFLQDRGATVIYGSITDKEFMEKVLKEHKIEVVISAVGGGSILDQFNLIEAIRNVDTVKRFLPSEFGHDTDRADPVEPGLTMYEQKRQIRRQIEKSGIPYTYICCNSIAAWPYHDNTHPADVLPPLDRFKIYGDGTVKAYFVAGTDIGKFTIMSIEDDRTLNKTVHFQPPSNLLNINEMASLWEEKIGRTLPRVTITEEDLLQMAKEMRIPQSVVAALTHDIFINGCQINFSLDKPTDVEVCSLYPDTPFRTINECFEDFAKKIIDNAKAVSKPAASNNAIFVPTAKPGALPITAICT

SEQ ID NO: 65 - protein, 350 aa, synthetic construct
MTVSPSIASAAKSGRVLIIGATGFIGKFVAEASLDSGLPTYVLVRPGPSRPSKSDTIKSLKDRGAIILHGVMSDKPLMEKLLKEHEIEIVISAVGGATILDQITLVEAITSVGTVKRFLPSEFGHDVDRADPVEPGLTMYLEKRKVRRAIEKSGVPYTYICCNSIASWPYYDNKHPSEVVPPLDQFQIYGDGTVKAYFVDGPDIGKFTMKTVDDIRTMNKNVHFRPSSNLYDINGLASLWEKKIGRTLPKVTITENDLLTMAAENRIPESIVASFTHDIFIKGCQTNFPIEGPNDVDIGTLYPEESFRTLDECFNDFLVKVGGKLETDKLAAKNKAAVGVEPMAITATCA

SEQ ID NO: 66 - protein, 369 aa, synthetic construct
MTQNKEPVNQGKSEHDEQRVESLASSGIESIPKEYVRLNEELTSMGNVFEEEKKEEGSQVPTIDIKDIASEDPEVRGKAIQELKRAAMEWGVMHLVNHGISDELIDRVKVAGQTFFELPVEEKEKYANDQASGNVQGYGSKLANSASGRLEWEDYYFHLSYPEDKRDLSIWPETPADYIPAVSEYSKELRYLATKILSALSLALGLEEGRLEKEVGGLEELLLQFKINYYPKCPQPELALGVEAHTDVSALTFILHNMVPGLQLFYEGKWVTAKCVPNSIIMHIGDTIEILSNGKYKSILHRGLVNKEKVRISWAVFCEPPKEKIILKPLPDLVSDEEPARYPPRTFAQHVQYKLFRKTQGPQTTITKN

SEQ ID NO: 67 - protein, 370 aa, synthetic construct
MASSKVMPAPARVESLASSGLASIPTEYVRPEWERDDSLGDALEEIKKTEEGPQIPIVDLRGFDSGDEKERLHCMEEVKEAAVEWGVMHIVNHGIAPELIERVRAAGKGFFDLPVEAKERYANNQSEGKIQGYGSKLANNASGQLEWEDYFFHLIFPSDKVDLSIWPKEPADYTEVMMEFAKQLRVVVTKMLSILSLGLGFEEEKLEKKLGGMEELLMQMKINYYPKCPQPELALGVEAHTDVSSLSFILHNGVPGLQVFHGGRWVNARLVPGSLVVHVGDTLEILSNGRYKSVLHRGLVNKEKVRISWAVFCEPPKEKIVLEPLAELVDKRSPAKYPPRTFAQHIQHKLFKKAQEQLAGGVHIPEAIQN

SEQ ID NO: 68 - protein, 360 aa, synthetic construct
MATQVASIPRVEMLASAGIQAIPTEYVRPEAERNSIGDVFEEEKKLEGPQIPVVDLMGLEWENEEVFKKVEEDMKKAASEWGVMHIINHGISMELMDRVRIAGKAFFDLPIEEKEMYANDQASGKIAGYGSKLANNASGQLEWEDYFFHLIFPEDKRDMSIWPKQPSDYVEATEEFAKQLRGLVTKVLVLLSRGLGVEEDRLEKEFGGMEELLLQMKINYYPKCPQPDLALGVEAHTDVSALTFILHNMVPGLQVFFDDKWVTAKCIPGALVVHIGDSLEILSNGKYRSILHRGLVNKEKVRISWAIFCEPPKEKVVLQPLPELVSEAEPARFTPRTFSQHVRQKLFKKQQDALENLKSE

SEQ ID NO: 69 - protein, 356 aa, synthetic construct
MVSSAAVVATRVERLATSGIKSIPKEYVRPQEELTNIGNVFEEEKKEGPEVPTIDLTEIESEDEVVRARCHETLKKAAQEWGVMNLVNHGIPEELLNQLRKAGETFFSLPIEEKEKYANDQASGKIQGYGSKLANNASGQLEWEDYFFHLVFPEDKCDLSIWPRTPSDYIEVTSEYARQLRGLATKILGALSLGLGLEKGRLEEEVGGMEELLLQMKINYYPICPQPELALGVEAHTDVSSLTFLLHNMVPGLQLFYNGQWITAKCVPNSIFMHIGDTVEILSNGRYKSILHRGLVNKEKVRISWAVFCEPPKEKIILKPLPELVTDDEPARFPPRTFAQHIQHKLFRKCQEGLSK

SEQ ID NO: 70 - protein, 455 aa, synthetic construct
MPQFTTNEPHVAVLAFPFGTHAAPLITIIHRLAVASPNTHFSFLNTSQSNNSIFSSDVYNRQPNLKAHNVWDGVPEGYVFVGKPQESIELFVKAAPETFRKGVEAAVAETGRKVSCLVTDAFFWFAAEIAGELGVPWVPFWTAGPCSLSTHVYTDLIRKTIGVGGIEGREDESLEFIPGMSQVVIRDLQEGIVFGNLESVFSDMVHRMGIVLPQAAAIFINSFEELDLTITNDLKSKFKQFLSIGPLNLASPPPRVPDTNGCLPWLDQQKVASVAYISFGTVMAPSPPELVALAEALEASKIPFIWSLGEKLKVHLPKGFLDKTRTHGIVVPWAPQSDVLENGAVGVFITHCGWNSLLESIAGGVPMICRPFFGDQRLNGRMVQDVWEIGVTATGGPFTTEGVMGDLDLILSQARGKKMKDNISVLKTLAQTAVGPEGSSAKNYEALLNLVRLSI

SEQ ID NO: 71 - protein, 474 aa, synthetic construct
MAPQPIDDDHVVYEHHVAALAFPFSTHASPTLALVRRLAAASPNTLFSFFSTSQSNNSLFSNTITNLPRNIKVFDVADGVPDGYVFAGKPQEDIELFMKAAPHNFTTSLDTCVAHTGKRLTCLITDAFLWFGAHLAHDLGVPWLPLWLSGLNSLSLHVHTDLLRHTIGTQSIAGRENELITKNVNIPGMSKVRIKDLPEGVIFGNLDSVFSRMLHQMGQLLPRANAVLVNSFEELDITVTNDLKSKFNKLLNVGPFNLAAAASPPLPEAPTAADDVTGCLSWLDKQKAASSVVYVSFGSVARPPEKELLAMAQALEASGVPFLWSLKDSFKTPLLNELLIKASNGMVVPWAPQPRVLAHASVGAFVTHCGWNSLLETIAGGVPMICRPFFGDQRVNARLVEDVLEIGVTVEDGVFTKHGLIKYFDQVLSQQRGKKMRDNINTVKLLAQQPVEPKGSSAQNFKLLLDVISGSTKV

SEQ ID NO: 72 - protein, 457 aa, synthetic construct
MVFQSHIGVLAFPFGTHAAPLLTVVQRLATSSPHTLFSFFNSAVSNSTLFNNGVLDSYDNIRVYHVWDGTPQGQAFTGSHFEAVGLFLKASPGNFDKVIDEAEVETGLKISCLITDAFLWFGYDLAEKRGVPWLAFWTSAQCALSAHMYTHEILKAVGSNGVGETAEEELIQSLIPGLEMAHLSDLPPEIFFDKNPNPLAITINKMVLKLPKSTAVILNSFEEIDPIITTDLKSKFHHFLNIGPSILSSPTPPPPDDKTGCLAWLDSQTRPKSVVYISFGTVITPPENELAALSEALETCNYPFLWSLNDRAKKSLPTGFLDRTKELGMIVPWAPQPRVLAHRSVGVFVTHCGWNSILESICSGVPLICRPFFGDQKLNSRMVEDSWKIGVRLEGGVLSKTATVEALGRVMMSEEGEIIRENVNEMNEKAKIAVEPKGSSFKNFNKLLEIINAPQSS

SEQ ID NO: 73 - protein, 456 aa, synthetic construct
MSQTTTNPHVAVLAFPFSTHAAPLLAVVRRLAAAAPHAVFSFFSTSQSNASIFHDSMHTMQCNIKSYDISDGVPEGYVFAGRPQEDIELFTRAAPESFRQGMVMAVAETGRPVSCLVADAFIWFAADMAAEMGLAWLPFWTAGPNSLSTHVYIDEIREKIGVSGIQGREDELLNFIPGMSKVRFRDLQEGIVFGNLNSLFSRMLHRMGQVLPKATAVFINSFEELDDSLTNDLKSKLKTYLNIGPFNLITPPPVVPNTTGCLQWLKERKPTSVVYISFGTVTTPPPAEVVALSEALEASRVPFIWSLRDKARVHLPEGFLEKTRGYGMVVPWAPQAEVLAHEAVGAFVTHCGWNSLWESVAGGVPLICRPFFGDQRLNGRMVEDVLEIGVRIEGGVFTKSGLMSCFDQILSQEKGKKLRENLRALRETADRAVGPKGSSTENFITLVDLVSKPKDV

SEQ ID NO: 74 - protein, 2185 aa, synthetic construct
MPPPDHKAVSQFIGGNPLETAPASPVADFIRKQGGHSVITKVLICNNGIAAVKEIRSIRKWAYETFGDERAIEFTVMATPEDLKVNADYIRMADQYVEVPGGSNNNNYANVDLIVDVAERAGVHAVWAGWGHASENPRLPESLAASKHKIIFIGPPGSAMRSLGDKISSTIVAQHADVPCMPWSGTGIKETMMSDQGFLTVSDDVYQQACIHTAEEGLEKAEKIGYPVMIKASEGGGGKGIRKCTNGEEFKQLYNAVLGEVPGSPVFVMKLAGQARHLEVQLLADQYGNAISIFGRDCSVQRRHQKIIEEAPVTIAPEDARESMEKAAVRLAKLVGYVSAGTVEWLYSPESGEFAFLELNPRLQVEHPTTEMVSGVNIPAAQLQVAMGIPLYSIRDIRTLYGMDPRGNEVIDFDFSSPESFKTQRKPQPQGHVVACRITAENPDTGFKPGMGALTELNFRSSTSTWGYFSVGTSGALHEYADSQFGHIFAYGADRSEARKQMVISLKELSIRGDFRTTVEYLIKLLETDAFESNKITTGWLDGLIQDRLTAERPPADLAVICGAAVKAHLLARECEDEYKRILNRGQVPPRDTIKTVFSIDFIYENVKYNFTATRSSVSGWVLYLNGGRTLVQLRPLTDGGLLIGLSGKSHPVYWREEVGMTRLMIDSKTCLIEQENDPTQIRSPSPGKLVRFLVDSGDHVKANQAIAEIEVMKMYLPLVAAEDGVVSFVKTAGVALSPGDIIGILSLDDPSRVQHAKPFAGQLPDFGMPVIVGNKPHQRYTALVEVLNDILDGYDQSFRMQAVIKELIETLRNPELPYGQASQILSSLGGRIPARLEDVVRNTIEMGHSKNIEFPAARLRKLTENFLRDSVDPAIRGQVQITIAPLYQLFETYAGGLKAHEGNVLASFLQKYYEVESQFTGEADVVLELRLQADGDLDKVVALQTSRNGINRKNALLLTLLDKHIKGTSLVSRTSGATMIEALRKLASLQGKSTAPIALKAREVSLDADMPSLADRSAQMQAILRGSVTSSKYGGDDEYHAPSLEVLRELSDSQYSVYDVLHSFFGHREHHVAFAALCTYVVRAYRAYEIVNFDYAVEDFDVEERAVLTWQFQLPRSASSLKERERQVSISDLSMMDNNRRARPIRELRTGAMTSCADVADIPELLPKVLKFFKSSAGASGAPINVLNVAVVDQTDFVDAEVRSQLALYTNACSKEFSAARVRRVTYLLCQPGLYPFFATFRPNEQGIWSEEKAIRNIEPALAYQLELDRVSKNFELTPVPVSSSTIHLYFARGIQNSADTRFFVRSLVRPGRVQGDMAAYLISESDRIVNDILNVIEVALGQPEYRTADASHIFMSFIYQLDVSLVDVQKAIAGFLERHGTRFFRLRITGAEIRMILNGPNGEPRPIRAFVTNETGLVVRYETYEETVADDGSVILRGIEPQGKDATLNAQSAHFPYTTKVALQSRRSRAHALQTTFVYDFIDVLGQAVRASWRKVAASKIPGDVIKSAVELVFDEQENLREVKRAPGMNNIGMVAWLVEVLTPEYPAGRKLVVIGNDVTIQAGSFGPVEDRFFAAASKLARELGVPRLYISANSGARIGLATEALDLFKVKFVGDDPAKGFEYIYLDDESLQAVQAKAPNSVMTKPVQAADGSVHNIITDIIGKPQGGLGVECLSGSGLIAGETSRAKDQIFTATIITGRSVGIGAYLARLGERVIQVEGSPLILTGYQALNKLLGREVYTSNLQLGGPQIMYKNGVSHLTAQDDLDAVRSFVNWISYVPAQRGGPLPIMPTTDSWDRAVTYQPPRGPYDPRWLINGTKAEDGTKLTGLFDEGSFVETLGGWATSVVTGRARLGGIPVGVIAVETRTLERVVPADPANPNSTEQRIMEAGQVWYPNSAYKTAQAIWDFDKEGLPLVILANWRGFSGGQQDMYDEILKQGSKIVDGLSSYKQPVFVHIPPMGELRGGSWVVVDSAINDNGMIEMSADVNSARGGVLEASGLVEIKYRADKQRATMERLDSVYAKLSKEAAEATDFTAQTTARKALAEREKQLAPIFTAIATEYADAHDRAGRMLATGVLRSALPWENARRYFYWRLRRRLTEVAAERTVGEANPTLKHVERLAVLRQFVGAAASDDDKAVAEHLEASADQLLAASKQLKAQYILAQISTLDPELRAQLAASLK

SEQ ID NO: 75 - protein, 2213 aa, synthetic construct
MVDHKSLPGHFLGGNSVDTAPQDPVCEFVKSHQGHTVISKVLIANNGMAAMKEIRSVRKWAYETFGNERAIEFTVMATPEDLKANAEYIRMADNYIEVPGGTNNNNYANVELIVDVAERTGVHAVWAGWGHASENPRLPEMLAKSKNKCVFIGPPASAMRSLGDKISSTIVAQSADVPTMGWSGDGVSETTTDHNGHVLVNDDVYNSACVKTAEAGLASAEKIGFPVMIKASEGGGGKGIRKVEDPSTFKQAFAQVQGEIPGSPIFIMKLAGNARHLEVQLLADQYGNAISLFGRDCSVQRRHQKIIEEAPVTIAKPDIFEQMEKAAVRLGKLVGYVSAGTVEYLYSHHDEKFYFLELNPRLQVEHPTTEMVSGVNLPAAQLQIAMGIPMHRIRDIRVLYGVQPNSASEIDFDLEHPTALQSQRRPMPKGHVIAVRITAENPDAGFKPSGGVMQELNFRSSTNVWGYFSVVSSGAMHEYADSQFGHIFAYGENRQQARKNMVIALKELSIRGDFRTTVEYIIRLLETPDFTDNTINTGWLDMLISKKLTAERPDTMLAVFCGAVTKAHLASVECWQQYKNSLERGQIPSKESLKTVFTVDFIYENIRYNFTVTRSAPGIYTLYLNGTKTQVGVRDLSDGGLLISLNGRSHTTYNREEVQATRLMIDGKTCLLEKESDPTQLRSPSPGKLVSLLLENGDHIRTGQAYAEIEVMKMYMPLVASEDGHVQFIKQVGATLEAGDIIGILSLDDPSRVKHALPFTGQVPKYGLPHLTGDKPHQRFTHLKQTLEYVLQGYDNQGLIQTIVKELSEVLNNPELPYSELSASMSVLSGRIPGRLEQQLHDLINQAHAQNKGFPAVDIQQAIDTFARDHLTTQAEVNAYKTAVAPIMTIAASYSNGLKQHEHSVYVDLMEQYYNVEVLFNSNQSRDEEVILALRDQHKDDLEKVINIILSHAKVNIKNNLILMLLDIIYPATSSEALDRCFLPILKHLSEIDSRGTQKVTLKAREYLILCQLPSLEERQSQMYNILKSSVTESVYGGGTEYRTPSYDAFKDLIDTKFNVFDVLPNFFYHPDSYVSLAALEVYCRRSYHAYKILDVAYNLEHQPYIVAWKFLLQSSAGGGFNNQRIASYSDLTFLLNKTEEEPIRTGAMVALKTLEELEAELPRIMTAFEEEPLPPMLMKQPPPDKTEERMENILNISIQGQDMEDDTLRKNMTTLIQAHSDAFRKAALRRITLVVCRDNQTPDYYTFRERNGYEEDETIRHIEPALAYQLELARLSNFDIKPCFIENRQMHVYYAVAKENPSDCRFFIRALVRPGRVKSSMRTADYLISESDRLLTDILDTLEIVSHDYKNSDCNHLFINFIPTFAIEADEVETALKDFVDRHGKRLWKLRVTGAEIRFNIQSKRPDAPVIPLRFTVDNVSGYILKVDVYQEVKTDKNGWILKSVGKIPGAMHMQPLSTPYPTKEWLQPRRYKAHLMGTTYVYDFPELFRQAIHNLWAQACKADAAVKIPSQVIEAKELVLDDDNQLQAIDRAPGTNTVGMVAWLLTLRTPDYPRGRRVIAIANDITFKIGSFGVQEDLVFYKASEYARELGVPRVYLSANSGARIGLADELISRFHVAWKDEDQPGSGFEYLYLLPEEYDALIQQGDAQSVLVQEVQDKGERRFRITDIIGHTDGLGVENLRGSGLIAGATSRAYDDIFTITLVTCRSVGIGAYLVRLGQRTVQNEGQPIILTGAPALNKVLGREVYTSNLQLGGTQIMYKNGVSHLTAENDLEGINKIMQWLSFVPECRGAPLPMRAGADPIDREIEYLPPKGPSDPRFFLAGKQENGKWLSGFFDHGSFVETLSGWARTVVVGRARLGGIPMGVVAVETRTVENIVPADPANADSQEQVVMEAGGVWFPNSAYKTAQAINDFNKGEQLPLMIFANWRGFSGGQRDMYNEVLKYGAQIVDALSNYKQPVFVYVVPNGELRGGAWVVVDSTINEDMMEMYADTQARGGVLEPEGIVEIKYRRPQLLATMERLDPVYSDLKRRLAALDDSQKEQADELIAQVEAREQALLPVYQQVAIQFADLHDRSGRMEAKGVIRKTLEWRTARHYFYWRVRRRLLEEYAIRKMDESRDQAKTLLQQWFQADTNLDDFDKNDQAVVAWFDAKNLLLDQRIAKLKSEKLKDHVVQLASVDQDAVVEGFSKLMESLSVDQRKEVLHKLATRF

SEQ ID NO: 76 - protein, 2515 aa, synthetic construct
MASTTPHDSRVVSVSSGKKLYIEVDDGAGKDAPAIVFMHGLGSSTSFWEAPFSRSNLSSRFRLIRYDFDGHGLSPVSLLDAADDGAMIPLVDLVEDLAAVMEWTGVDKVAGIVGHSMSGLVASTFAAKYPQKVEKLVLLGAMRSLNPTVQTNMLKRADTVLESGLSAIVAQVVSAALSDKSKQDSPLAPAMVRTLVLGTDPLGYAAACRALAGAKDPDYSTIKAKTLVVSGESDYLSNKETTEALVNDIPGAKEVQMDGVGHWHAVEDPAGLAKILDGFFLQGKFSGEAKAVNGSHAVDETPKKPKYDHGRVVKYLGGNSLESAPPSNVADWVRERGGHTVITKILIANNGIAAVKEIRSVRKWAYETFGSERAIEFTVMATPEDLKVNADYIRMADQYVEVPGGTNNNNYANVDVIVDVAERAGVHAVWAGWGHASENPRLPESLAASKHKIVFIGPPGSAMRSLGDKISSTIVAQHAEVPCMDWSGQGVDQVTQSLEGYVTVADDVYQQACVHDADEGLARASRIGYPVMIKASEGGGGKGIRKVEREQDFKQAFQAVLTEVPGSPVFIMKLAGAARHLEVQVLADQYGNAISLFGRDCSVQRRHQKIIEEAPVTIAKPDTFEQMEKSAVRLAKLVGYVSAGTVEFLYSAADDKFAFLELNPRLQVEHPTTEMVSGVNLPAAQLQVAMGVPLHRIRDIRTLYGKAPNGSSEIDFEFENPESAKTQRKPSPKGHVVAVRITAENPDAGFKPSMGTLQELNFRSSTNVWGYFSVGSAGGLHEFADSQFGHIFAYGSDRSESRKNMVVALKELSIRGDFRTTVEYLIKLLETDAFEQNTITTAWLDSLISARLTAERPDTTLAIICGAVTKAHLASEANIAEYKRILEKGQSPPKELLATVVPLEFVLEDVKYRATASRSSPSSWSIYVNGSNVSVGIRPLADGGLLILLDGRSYTCYAKEEVGALRLSIDSRTVLVAQENDPTQLRSPSPGKLVRYFIESGEHISKGEAYAEIEVMKMIMPLIAAEDGIAQFIKQPGATLEAGDILGILSLDDPSRVHHAKPFDGQLPALGLPSIIGTKPHQRFAYLKDVLSNILMGYDNQAIMQSSIKELISVLRNPELPYGEANAVLSTLSGRIPAKLEQTLRQYIDSAHESGAEFPSAKCRKAIDTTLEQLRPAEAQTVRNFLVAFDDIVYRYRSGLKHHEWSTLAGIFAAYAETEKPFSGKDSDVVLELRDAHRDSLDSVVKIVLSHYKAASKNSLVLALLDVVKDSDSVPLIEQVVSPALKDLADLDSKATTKVALKAREVLIHIQLPSLDERLGQLEQILKASVTPTVYGEPGHDRTPRGEVLKDVIDSRFTVFDVLPSFFQHQDQWVSLAALDTYVRRAYRSYNLLNIEHIEADAAEDEPATVAWSFRMRKAASESEPPTPTTGLTSQRTASYSDLTFLLNNAQSEPIRYGAMFSVRSLDGFRQELGTVLRHFPDSNKGKLQQQPAASSSQEQWNVINVALTVPASAQVDEDALRADFAAHVNAMSAEIDARGMRRLTLLICREGQYPSYYTVRKQDGTWKELETIRDIEPALAFQLELGRLSNFHLEPCPVENRQVHIYYATAKGNSSDCRFFVRALVRPGRLRGNMKTADYLVSEADRLVTDVLDSLEVASSQRRAADGNHISLNFLYSLRLDFDEVQAALAGFIDRHGKRFWRLRVTGAEIRIVLEDAQGNIQPIRAIIENVSGFVVKYEAYREVTTDKGQVILKSIGPQGALHLQPVNFPYPTKEWLQPKRYKAHVVGTTYVYDFPDLFRQAIRKQWKAVGKTAPAELLVAKELVLDEFGKPQEVARPPGTNNIGMVGWIYTIFTPEYPSGRRVVVIANDITFKIGSFGPEEDRYFYAVTQLARQLGLPRVYLSANSGARLGIAEELVDLFSVAWADSSRPEKGFKYLYLTAEKLGELKNKGEKSVITKRIEDEGETRYQITDIIGLQEGLGVESLKGSGLIAGETSRAYDDIFTITLVTARSVGIGAYLVRLGQRAVQVEGQPIILTGAGALNKVLGREVYSSNLQLGGTQIMYKNGVSHLTAANDLEGVLSIVQWLAFVPEHRGAPLPVLPSPVDPWDRSIDYTPIKGAYDPRWFLAGKTDEADGRWLSGFFDKGSFQETLSGWAQTVVVGRARLGGIPMGAIAVETRTIERIIPADPANPLSNEQKIMEAGQVWYPNSSFKTGQAIFDFNREGLPLIIFANWRGFSGGQQDMFDEVLKRGSLIVDGLSAYKQPVFVYIVPNGELRGGAWVVLDPSINAEGMMEMYVDETARAGVLEPEGIVEIKLRKDKLLALMDRLDPTYHALRVKSTDASLSPTDAAQAKTELAAREKQLMPIYQQVALQFADSHDKAGRILSKGCAREALEWSNARRYFYARLRRRLAEEAAVKRLGEADPTLSRDERLAIVHDAVGQGVDLNNDLAAAAAFEQGAAAITERVKLARATTVASTLAQLAQDDKEAFAASLQQVLGDKLTAADLARILA

SEQ ID NO: 77 - protein, 504 aa, synthetic construct
MNANLFSRLFDGLVEADKLAIETLEGERISYGDLVARSGRMANVLVARGVKPGDRVAAQAEKSVAALVLYLATVRAGAVYLPLNTAYTLHELDYFIGDAEPKLVVCDPAKREGIAALAQKVGAGVETLDAKGQGSLSEAAAQASVDFATVPREGDDLAAILYTSGTTGRSKGAMLSHDNLASNSLTLVEFWRFTPDDVLIHALPIYHTHGLFVASNVTLFARASMIFLPKFDPDAIIQLMSRASVLMGVPTFYTRLLQSDGLTKEAARHMRLFISGSAPLLADTHREWASRTGHAVLERYGMTETNMNTSNPYDGARVPGAVGPALPGVSLRVVDPETGAELSPGEIGMIEVKGPNVFQGYWRMPEKTKAEFRDDGFFITGDLGKIDADGYVFIVGRGKDLVITGGFNVYPKEVESEIDAISGVVESAVIGVPHADLGEGVTAVVVRDKGASVDEAAVLGALQGQLAKFKMPKRVLFVDDLPRNTMGKVQKNVLREAYAKLYAK

SEQ ID NO: 78 - protein, 504 aa, synthetic construct
MVNHLFDAIRLSITSPESTFIELEDGKVWTYGAMFNCSARITHVLVKLGVSPGDRVAVQVEKSAQALMLYLGCLRAGAVYLPLNTAYTPAELEYFLGDATPKLVVVSPCAAEQLEPLARRVGTRLLTLGVNGDGSLMDMASLEPVEFADIERKADDLAAILYTSGTTGRSKGAMLTHDNLLSNAQTLREHWRFTSADRLIHALPIFHTHGLFVATNVTLLAGGAIYLLSKFDPDQIFALMTRATVMMGVPTFYTRLLQDERLNKANTRHMRLFISGSAPLLAETHRLFEEYTGHAILERYGMTETNMITSNPCDGARVPGTVGYALPGVSVRITDPVSGEPLAAGEPGMIEVKGPNVFQGYWNMPDKTKEEFRSDGYFTTGDIGVMETDGRISIVGRGKDLIISGGYNIYPKEIENEIDAIEGVVESAVIGVPHPDLGEGVTAIVVGQPKAHLDLTTITNNLQGRLARFKQPKNVIFVDELPRNTMGKVQKNVLRDRYRDLYLK

SEQ ID NO: 79 - protein, 505 aa, synthetic construct
MANHLFDLVRANATDLTKTFIETETGLKLTYDDLMTGTARYANVLVGLGVKPGDRVAVQVEKSAGAIFLYLACVRAGAVFLPLNTAYTLTEIEYFLGDAEPALVVCDPARRDGITEVAKKTGVPAVETLGKGQDGSLFDKAAAAPETFADVARGPGDLAAILYTSGTTGRSKGAMLSHDNLASNALTLKDYWRFGADDVLLHALPIFHTHGLFVATNTILVAGASMLFLPKFDADKVFELMPRATTMMGVPTFYVRLVQDARLTREATKHMRLFISGSAPLLAETHKLFREKTGVSILERYGMTETNMNTSNPYDGDRVAGTVGFPLPGVALRVADPETGAAIPQGEIGVIEVKGPNVFSGYWRMPEKTAAEFRQDGFFITGDLGKIDDQGYVHIVGRGKDLVISGGYNVYPKEVETEIDGMAGVVESAVIGVPHPDFGEGVTAVVVAEKGASLDEATIIKTLEQRLARYKLPKRVIVVDDLPRNTMGKVQKNLLRDAYKGLYGG

SEQ ID NO: 80 - protein, 465 aa, synthetic construct; identical to SEQ ID NO: 18

SEQ ID NO: 81 - protein, 448 aa, synthetic construct
MGIELLSIGLLIAMFIIATIQPINMGALAFAGAFVLGSMIIGMKTNEIFAGFPSDLFLTLVAVTYLFAIAQINGTIDWLVECAVRLVRGRIGLIPWVMFLVAAIITGFGALGPAAVAILAPVALSFAVQYRIHPVMMGLMVIHGAQAGGFSPISIYGGITNQIVAKAGLPFAPTSLFLSSFFFNLAIAVLVFFVFGGARVMKHDPASLGPLPELHPEGVSASIRGHGGTPAKPIREHAYGTAADTATTLRLNNERITTLIGLTALGIGALVFKFNVGLVAMTVAVVLALLSPKTQKAAIDKVSWSTVLLIAGIITYVGVMEKAGTVDYVANGISSLGMPLLVALLLCFTGAIVSAFASSTALLGAIIPLAVPFLLQGHISAIGVVAAIAISTTIVDTSPFSTNGALVVANAPDDSREQVLRQLLIYSALIAIIGPIVAWLVFVVPGLV

SEQ ID NO: 82 - protein, 448 aa, synthetic construct
MNIEILSIGLLVAIFIIATIQPINMGVLAFGCTFVLGSLIIGMKPADIFAGFPADLFLTLVAVTYLFAIAQINGTIDWLVERSVRMVRGRVGWIPWVMFLVAAIITGFGALGPAAVAILAPVALSFAVQYRIHPVLMGLMVIHGAQAGGFSPISIYGGITNQIVAKAGLPFAPTSLFLSSFFFNLAIAVLIFFIFGGLSILKQRSSVKGPLPELHPEGISASIKGHGGTPAKPFREHAYGTAADTQSKVRLTTEKVTTLIGLTALGVGALVFKFNVGLVAITVAVLLALLSPTTQKAAIDKVSWSTVLLISGIITYVGVMEKAGTIDYVAHGISSLGMPLLVALLLCFTGAIVSAFASSTALLGAIIPLAVPFLLQGHISAVGVVAAIAISTTIVDTSPFSTNGALVVANAPDDQRDKVMRQMLIYSALIALIGPVIAWLVFVVPGII

SEQ ID NO: 83 - protein, 448 aa, synthetic construct
MSIEILSILLLVAMFVIATIQPINMGALAFACTFVLGSLIIGMKTSDIFAGFPSDLFLTLVAVTYLFAIAQINGTIDWLVECAVRMVRGHVAWIPWVMFVVAAIITGFGALGPAAVAILAPVALSFAVQYRIHPVMMGLMVIHGAQAGGFSPISVYGGITNQIVAKAGLPFAPTSLFLSSFFFNLAIAVLVFFVFGGARIMKQAAGPTGPLPELHPEGVSAAIRGHGGTPAKPIREHAYGTAADTLQTLRLTPEKVFTLIGLTALGIGALVFKFNVGLVAITVAVALALISPKTQKAAVDKVSWSTVLLIAGIITYVGVLEKAGTVNYVANGISSLGMPLLVALLLCFTGAIVSAFASSTALLGAIIPLAVPFLLQGHISAVGVVAAIAISTTIVDTSPFSTNGALVVANAPDETREQVLRQLLIYSALIAIIGPVVAWLVFVVPGLV

SEQ ID NO: 84 - protein, 545 aa, synthetic construct
MTTWNQKQQRKAQKLAKACDSGFDKYVPHERIIALLETVIDRGDRVCLEGNNQKQADFLSKSLSSCNPDIVNGLHIVQSVLALPSHIDVFERGIASKVDFSFAGPQSLRLAQLVQAQKITIGAIHTYLELYGRYFIDLTPNVALITAHAADKRGNLYTGANTEDTPAIVEATTFKSGIVIAQVNEIVDELPRVDIPSDWVDYYTQSPKHNYIEPLFTRDPAQITEIQILMAMMAIKGIYAPYKINRLNHGIGFDTAAIELLLPTYAESLGLKGEICTHWALNPHPTLIPAIESGFIHSVHSFGSEVGMENYVKARSDVFFTGADGSMRSNRAFSQTAGLYACDLFIGSTLQIDLQGNSSTATADRIAGFGGAPNMGSDPHGRRHASYAYMKAGREAVDGSPIKGRKLVVQMVETYREHMQSVFVNELDAFKLQQKMGADLPPIMIYGDDVTHIVTEEGIANLLLCRTPDEREQAIRGVAGYTPIGLGRDDTMVARLRERKVIQRPEDLGINPMHATRDLLAAKSVKDLVRWSDRLYEPPSRFRNW

SEQ ID NO: 85 - protein, 554 aa, synthetic construct
MNAPQPRQWDSLRQNRARRLERAASLGLAGQNGKEIPVDRIIDLLEAVIQPGDRVCLEGNNQKQADFLSESLADCDPARINHLSMVQSVLALPSHVDLFERGLATRLDFSFSGPQGARLAKLVQEQRIEIGAIHTYLELFGRYFMDLTPNVALIAAQAADAEGNLYLGPNTEDTPAIVEATAFKGGIVIAQVNERLDKLPRVDVPADWVDFTVLAPKPNYIEPLFTRDPAQITEVQVLMAMMAIKGIYAEYGVTRLNHGIGFDTAAIELLLPTYAADLGLKGKICTHWALNPHPTLIPAIEAGFVESVHCFGSEVGMDDYISARSDIFFTGADGSMRSNRAFSQTAGLYACDMFIGSTLQMDLAGNSSTATLGRITGFGGAPNMGSDPHGRRHASPAWLKAGREAYGPQAIRGRKLVVQMVETFREHMAPVFVDDLDAWKLQASMGSDLPPIMIYGDDVSHIVTEEGIANLLLCRTPAEREQAIRGVAGFTPVGMARDKGTVENLRDRGIIRRPEDLGIDPRQASRDLLAARSIKDLVRCSGGLYAPPSRFRNW

SEQ ID NO: 86 - protein, 548 aa, synthetic construct
MSRQWDTQADSRRQRLQRAAALAPQGRVVAADDVVALLEAVIEPGDRVCLEGNNQKQADFLARCLTEVDPARVHDLHMVQSVLSLAAHLDVFERGIAKRLDFSFSGPQAARLAGLVSEGRIEIGAIHTYLELFGRYFIDLTPRIALVTAQAADRHGNLYTGPNTEDTPVIVEATAFKGGIVIAQVNEILDTLPRVDIPADWVDFVTQAPKPNYIEPLFTRDPAQISEIQVLMAMMAIKGIYAEYGVDRLNHGIGFDTAAIELLLPTYAQSLGLKGKICRHWALNPHPALIPAIESGFVQSVHSFGSELGMENYIAARPDIFFTGADGSMRSNRALSQTAGLYACDMFIGSTLQIDLQGNSSTATRDRIAGFGGAPNMGSDARGRRHASAAWLKAGREAATPGEMPRGRKLVVQMVETFREHMAPAFVDRLDAWELAERANMPLPPVMIYGDDVSHVLTEEGIANLLLCRTPEEREQAIRGVSGYTAVGLGRDKRMVENLRDRGVIKRPDDLGIRPRDATRDLLAARTVKDLVRWSGGLYDPPKRFRNW

SEQ ID NO: 87 - protein, 550 aa, synthetic construct
MNKIYREKRSWRTRRDRKAKRIEHMKQIAKGKIIPTEKIVEALTALIFPGDRVVIEGNNQKQASFLSKALSQVNPEKVNGLHIIMSSVSRPEHLDLFEKGIARKIDFSYAGPQSLRMSQMLEDGKLVIGEIHTYLELYGRLFIDLTPSVALVAADKADASGNLYTGPNTEETPTLVEATAFRDGIVIAQVNELADELPRVDIPGSWIDFVVAADHPYELEPLFTRDPRLITEIQILMAMMVIKGIYERHNIQSLNHGIGFNTAAIELLLPTYGESLGLKGKICKHWALNPHPTLIPAIETGWVESIHCFGGEVGMEKYIAARPDIFFTGKDGNLRSNRTLSQVAGQYAVDLFIGSTLQIDRDGNSSTVTNGRLAGFGGAPNMGHDPRGRRHSSPAWLDMITSDHPAAKGRKLVVQMVETFQKGNRPVFVESLDAIEVGRSARLATTPIMIYGEDVTHIVTEEGIAYLYKASSLEERRQAIAAIAGVTPIGLERDPRKTEQLRRDGVVAFPEDLGIRRTDAKRSLLAAKSIEELVEWSEGLYEPPARFRSW

SEQ ID NO: 88 - protein, 265 aa, synthetic construct
MLLTIDVGNTHTVLGLFDGEEIVEHWRISTDSRRTADELAVLLQGLMGTHPLLGMELGEGIDGIAICSTVPAVLHELREVSRRYYGDVPAILVEPGVKTGVPILMDNPKEVGTDRIINAVAAQHLYGGPAIVVDFGTATTFDAVSARGEYTGGVIAPGIEISVEALGLRGAQLRKIELARPRSVIGKSTVEAMQSGILYGFAGQVDGVVQRMACELAPDPADVTVIATGGLAPMVLGEAAVIDHHEPWLTLIGLRLVYERNAGRR

SEQ ID NO: 89 - protein, 248 aa, synthetic construct
MTKLWLDLGNTRLKYWLTDDSGQVLDHAAEQHLQAPAELLKGLTFRLERLNPDFIGVSSVLGQAVNNHVAESLERLQKPFEFAQVHAKHALMSSDYNPAQLGVDRWLQMLGIIEPSKKQCVIGCGTAVTIDLVDQGHHLGGYIFPSIYLQRESLFSGTRQISIIDGTFDSIDSGTNTQDAVHHGIMLSIVGAINETIHRYPQFEITMTGGDAHTFEPHLSASVEIRQDLVLAGLQRFFAAKNNTKNQN

SEQ ID NO: 90 - protein, 263 aa, synthetic construct
MLLTIDVGNTQTTLGLFDGEEVVDHWRISTDPRRTADELAVLMQGLMGRQPGGAGRERVDGLAICSSVPAVLHELREVTRRYYGDLPAVLVAPGVKTGVHVLMDNPKEVGADRIVNALAANHLYGGPCIVVDFGTATTFDAINERGDYVGGAIAPGIEISVEALGVRGAQLRKIELAKPRNVIGKNTVEGMQSGVLYGFAGQVDGLVTRMAKELSPTDPEDVQVIATGGLAPLVLDEASSIDVHEPWLTLIGLRLVYERNTAS

SEQ ID NO: 91 - protein, 418 aa, synthetic construct
MTLLALGINHKTAPVSLRERVTFSPDTLDQALDSLQALPMVQGGVVLSTCNRTEIYLSVEEQDNLREALIRWLCEYHNLNEEDLRNSLYWHQDNDAVSHLMRVASGLDSLVLGEPQILGQVKKAFADSQKGHQNASALERMFQKSFSVAKRVRTETDIGSSAVSVAFAACTLARQIFESLSTVTVLLVGAGETIELVARHLREHKVKKMIIANRTRERAQVLADEVGAEVISLSDIDARLQDADIIISSTASPLPIIGKGMVERALKNRRNQPMLLVDIAVPRDVEPEVGKLSNAYLYSVDDLQSIISHNLAQRKAAAVEAETIVEQEASEFMAWLRAQGASDTIREYRSQSEQIRDELTAKALAALQQGGDAQAIMQDLAWKLTNRLIHAPTKSLQQAARDGDSERLNILRDSLGLE

SEQ ID NO: 92 - protein, 420 aa, synthetic construct
MTLLALGINHKTAPVSLRERVTFSPETIEQALSSLLQQPLVQGGVVLSTCNRTELYLSVEQQENLQEQLVKWLCDYHHLSADEVRKSLYWHQDNAAVSHLMRVASGLDSLVVGEPQILGQVKKAFAESQHGQAVSGELERLFQKSFSVAKRVRTETDIGASAVSVAFAACTLARQIFESLSDVSVLLVGAGETIELVARHLREHKVRHMMIANRTRERAQVLASEVGAEVITLQDIDARLADADIIISSTASPLPIIGKGMVERALKARRNQPMLMVDIAVPRDIEPEVGKLANAYLYSVDDLHSIIQNNMAQRKAAAVQAESIVEQESSNFMAWLRSQGAVEIIRDYRSRADLVRAEAEAKALAAIAQGADVSAVIHELAHKLTNRLIHAPTRSLQQAASDGDVERLQILRDSLGLDQQ

SEQ ID NO: 93 - protein, 420 aa, synthetic construct
MTLLALGINHKTAPVALREKVSFSPDTMGDALNNLLQQPAVRGGVVLSTCNRTELYLSMEDKENSHEQLIRWLCQYHQIEPNELQSSIYWHQDNQAVSHLMRVASGLDSLVLGEPQILGQVKKAFADSQNYDSLSSELERLFQKSFSVAKRVRTETQIGANAVSVAFAACTLARQIFESLSSLTILLVGAGETIELVARHLREHQVKKIIIANRTKERAQRLASEVDAEVITLSEIDECLAQADIVISSTASPLPIIGKGMVERALKKRRNQPMLLVDIAVPRDIEQDVEKLNNVYLYSVDDLEAIIQHNREQRQAAAVQAEHIVQQESGQFMDWLRAQGAVGAIREYRDSAETLRAEMTEKAITLIQNGADAEKVIQQLSHQLMNRLIHTPTKSLQQAASDGDIERLNLLRESLGITHN

SEQ ID NO: 94 - protein, 645 aa, synthetic construct
MGPALDVRGKQLAAGYASVAGQADVEKIHQDQGITIPPNATVEMCPHAKAARDAARIAEDLAAAAASKQQPAKKAGGCPFHAAQAQAQAKPAAAPKETVATADKKGKSPRAAGGFDYEKFYEEELDKKHQDKSYRYFNNINRLAARFPTAHTAKVTDEVEVWCSNDYLGMGGNPVVLETMHRVLDKYGHGAGGTRNIAGNGALHLSLEQELARLHRKEGALVFTSCYVANDATLSTLGSKMPGCVIFSDRMNHASMIQGIRHSGTKKVIFEHNDLADLEKKLAEYPKETPKIIAFESVYSMCGSIGPIKEICDLAEKYGAITFLDEVHAVGLYGPRGAGVAEHLDYDLHKAAGDSPDAIPGTVMDRVDIITGTLGKSYGAIGGYIAGSARFVDMIRSYAPGFIFTTSLPPATVAGAQASVVYQKEYLGDRQLKQVNVREVKRRFAELDIPVVPGPSHIVPVLVGDAALAKQASDKLLAEHDIYVQAINYPTVARGEERLRITVTQRHTLEQMDHLIGAVDQVFNELNINRVQDWKRLGGRASVGVPGGQDFVEPIWTDEQVGLADGSAPLTLRNGQPNEVSHDAVVAARSRFDWLLGPIPSHIQAKRLGQSLEGTPIAPLAPKQSSGLKLPVEEMTMGQTIAVAA

SEQ ID NO: 95 - protein, 708 aa, synthetic construct
MDKIARFKQTCPFLGRTKNSTLRNLSTSSSPRFPSLTALTERATKCPVMGPALNVRSKEIVAGYASVAANSDVALIHKEKGVFPPPGATVEMCPHASAARAAARMADDLAAAAEKKKGHFTSAAPRDEAAQAAAAGCPFHVKAAADAAAARKAAAAPAPVKAKEDGGFNYESFYVNELDKKHQDKSYRYFNNINRLAAKFPVAHTSNVKDEVEVWCANDYLGMGNNPVVLETMHRTLDKYGHGAGGTRNIAGNGAMHLSLEQELATLHRKPAALVFSSCYVANDATLSTLGAKLPGCIFFSDTMNHASMIQGMRHSGAKRVLFKHNDLEDLENKLKQYPKDTPKVIAFESVYSMCGSIGPIKEICDLAEQYGALTFLDEVHAVGLYGPRGAGVAEHLDYDAHVAAGESPHPIKGSVMDRVDIITGTLGKAYGAVGGYIAGSDDFVDMIRSYAPGFIFTTSLPPATVAGARASVVYQKHYVGDRQLKQVNVREVKRRFAELDVPVVPGPSHIVPVLVGDAALAKAASDKLLAEHNIYVQSINYPTVARGEERLRITVTPRHTLEQMDKLVRAVDKIFAELKINRLADWKALGGRAGVGLTAGAEEAHVDPMWTEEQLGLLDGTSPRTLRNGEAAVVDAMAVGQARAVFDNLLGPISGKLQSERSVLASSTPAAANPARPAARKVVKMKTGGVPMSEDIPLPPPDVSASA

SEQ ID NO: 96 - protein, 677 aa, synthetic construct
MDKLSSLSRFKASCPFLGRTKTSTLRTLCTSSSPRFPSISILTERATKCPVMGPALNVRSKEITAGYASVAGSSEVDQIHKQQGVTVPVNATVEMCPHASAARAAARMADDLAAAAAQKKVGSGASSAKAAAAGCPFHKSVAAGASASTASKPSAPIHKASVPGGFDYDNFYNNELEKKHKDKSYRYFNNINRLASKFPVAHTGDVKDEVQVWCSNDYLGMGNNPVVLETMHRTLDKYGHGAGGTRNIAGNGALHLGLEQELAALHRKEAALVFSSCYVANDATLSTLGSKLPGCILFSDKMNHASMIQGMRHSGAKKVIFNHNDLEDLENKLKQYPKETPKIIAFESVYSMCGSIGPIKEICDLAEKYGALTFLDEVHAVGLYGPHGAGVAEHLDYNAQKAAGKSPEPIPGSVMDRVDIITGTLGKAYGAVGGYIAGSMDFVDTIRSYAPGFIFTTSLPPATVSGAQASVAYQKEYLGDRQLKQVNVREVKRRFAELDIPVIPGPSHILPVLVGDAALAKAASDKLLTDHDIYVQSINYPTVAVGEERLRITVTPRHTLEQMDKLVRAVNQVFTELNINRISDWKVAGGRAGVGMGVESVEPIWTDEQLGITDGTTPKTLRDGQRFLVDAQGVTAARGRFDTLLGPMSGSLQANPTLPLVDDELKVPLPTLVAAAA

SEQ ID NO: 97 - protein, 409 aa, synthetic construct
MDYAQFFNTALDRLHTERRYRVFADLERIAGRFPHALWHSPKGKRDVVIWCSNDYLGMGQHPKVVGAMVETATRVGTGAGGTRNIAGTHHPLVQLEAELADLHGKEASLLFTSGYVSNQTGIATIAKLIPNCLILSDELNHNSMIEGIRQSGCERVVFRHNDLADLEEKLKAAGPNRPKLIACESLYSMDGDVAPLAKICDLAEKYGAMTYVDEVHAVGMYGPRGGGIAERDGVMHRIDILEGTLAKAFGCLGGYIAANGQIIDAVRSYAPGFIFTTALPPAICSAATAAIRHLKTSNWERERHQDRAARVKAILNAAGLPVMSSDTHIVPLFIGDAEKCKQASDLLLEQHGIYIQPINYPTVAKGTERLRITPSPYHDDGLIDQLAEALLQVWDRLGLPLKQKSLAAE

SEQ ID NO: 98 - protein, 149 aa, synthetic construct
MDKQRVFTLSQVAEHKSKQDCWIIINGRVVDVTKFLEEHPGGEEVLIESAGKDATKEFQDIGHSKAAKNLLFKYQIGYLQGYKASDDSELELNLVTDSIKEPNKAKEMKAYVIKEDPKPKYLTFVEYLLPFLAAAFYLYYRYLTGALQF

SEQ ID NO: 99 - protein, 337 aa, synthetic construct
MEVERVQAISKMSRCMDTIPSEYIRSESEQPAVTTMQGVVLQVPVIDLGSSNNTEENLVELIAEASREWGIFQVVNHGIPDDAIAKLQKVGKEFFELPQQEKEVIAKPEGYQGVEGYGTKLQKELGGKKGWVDHLFHIIWPKSAVNYNFWPNNPPLYREANEEYAVALRGVVDKLFEALSLGIGLEKHELKKASGGDDLIYMLKINYYPPCPRPDLALGVVAHTDMSAITILVPNEVQGLQVHKDDHWYDVKYIPNALIIHIGDQIEIMSNGKYKSVYHRTTVNKDKTRMSWPVFLEPPPELLTGPISKLITDENPAKFKTKKYKDYVYCKLNKLPQ

SEQ ID NO: 100 - protein, 335 aa, synthetic construct
MGVERVQDIASTISEDTIPAEYIRSENEQPGITTVPNTVLECPTIDFSDPDEEKLLKQIFEASIDWGMYQIVNHDISNEAISKLQAVGKEFFELPQEEKEVYAKDPNSKSVEGYGTFLQKELEGKKGWVDHLFHKIWPPSTINYRFWPKTPASYREANEEYAKNLHKVVEKLFKLLSLGLGLEAQELKKAIGGDDLVYLLKINYYPPCPRPDLALGVVAHTDMSALTILVPNEVQGLQACRDGQWYDVKYIPNALVIHIGDQMEIMSNGKYRAVLHRTTVSKDKTRISWPVFLEPPADQVIGPHPKLVNDKENPPEYKTKKYSEYVYNKLNKMPQ

SEQ ID NO: 101 - protein, 331 aa, synthetic construct
MEVERVQALSHVTLHELPAKFIRPVHEQPENSKAIEGVTVPVISLSQPHDVVVDALSKACSEWGFFLITDHGVEPSLIGRLKEVGEEFFNLPQKEKESYANDPSSGSFEGYGTKMTKNFDEKVEWIDYYFHVMHPPKKLNLDMWPKNPSSYRGVTEEYNVEIMRTTNKLFELLSEGLGLDGKVLSSSLGGDEIEFEMKINMYPPCPQPQLALGVEPHTDMSALTLLVPNDVPGLQVWKDGNWVAVNYLPNALFVHVGDQLEVLSNGKYKSVLHRSLVNKERTRMSWAVFVVPPHEAVIGPLPELIDEKNPAKYSTKTYAEYRHRKFNKIPQ

SEQ ID NO: 102 - protein, 352 aa, synthetic construct
MKTIQGQSATTALTMEVARVQAISSITKCMDTIPSEYIRSENEQPAATTLQGVVLEVPVIDISNVDDDEEKLVKEIVEASKEWGIFQVINHGIPDEVIENLQKVGKEFFEEVPQEEKELIAKKPGAQSLEGYGTSLQKEIEGKKGWVDHLFHKIWPPSAINYRYWPKNPPSYREANEEYAKWLRKVADGIFRSLSLGLGLEGHEMMEAAGSEDIVYMLKINYYPPCPRPDLALGVVAHTDMSYITLLVPNEVQGLQVFKDGHWYDVNYIPNAIIVHIGDQVEILSNGKYKSVYHRTTVNKYKTRMSWPVFLEPSSEHEVGPIPKLINEANPPKFKTKKYKDYVYCKLNKLPQ

SEQ ID NO: 103 - protein, 340 aa, synthetic construct
MAPTRVQYVAESRPQTIPLEFVRPVEERPINTTFNDDIGLGRQIPVIDMCSLEAPELREKTFKEIARASKEWGIFQVINHAISPSLFESLETVGKQFFQLPQEEKEAYACTGEDGSFTGYGTKLACTTDGRQGWSDFFFHMLWPPSLRDFSKWPQKPSSYIEVTEEYSNRILGVLNKLLSALSISLELQESALKDALGGENLEMELKINYYPTCPQPEVAFGVVPHTDMSALTILKPNDVPGLQVWKDDKWITAHYVPNALIIHIGDQIQILSNGKFKSVLHRSLVNKEKVRMSWPVFCSPPLDTVIGPLKELIDDSNPPLYNAKTYREYKHRKINKLGQ

SEQ ID NO: 104 - protein, 335 aa, synthetic construct
MEVERVQAIATLTANLGTIPSEFIRSDHERPDLTTYHGPVPELPIIDLANSSQENVVKQISEAAQEYGIFQLVNHGIPNEVINELQRVGKEFFQLPQEEKEVYATVPDSGSFEGYGTKLQKDLEGKKAWVDYLFHNVWPKHKINYKFWPRNPPAYRKANEEYTKHLQVVVDKMHSYLSLGLGLESHVLKEAVGGDDLEYLLKINYYPPCPRPDLALGVVAHTDMSSLTILVPNEVPGLQVFKDDHWFDAKYIPNALICHIGDQLEILSNGKYKSVLHRTTVNKEKSRMSWPVFCSPPGDTMIGPLPQLVNDENPPKFKTKKYKDYAYCKINKLPQ

SEQ ID NO: 105 - protein, 336 aa, synthetic construct
MELERVQVIASLSKCIDTIPAEYIRSENEQPAITTIQGKVLEVPVIDLSNDSDDQNIVQSVAEACRDWGIFQVVNHGIPDEVIGNLQRVGKDFFELPQEEKEVYAKPPESKSVEGYGTKLQKEVEGKKGWVDHLFHKIWPPSAINYKFWPKNPPSYREANEVYAERLRAVADKLFRWLSLGLGLEGHEVKEAVGGDDLIYLMKINYYPPCPRPDLALGVVAHTDMSTLTILVPNEVPGLQVFKDDHWYEVKYIPNALIVHIGDQIEIVSNGKYKAVYHRSTVNKETTRMSWPVFIEPSPEVEVGPHPMLIDDENPPKYKTKKYEDYMYCKLNKLPQ

SEQ ID NO: 106 - protein, 331 aa, synthetic construct
MEVERVQALSDVTLHELPANFIRPAHEQPENSKAIDGVTVPVISLSQPHDVVVDELSMACSEWGFFLITDHGIEPSLIGRLNEVGEEFFKLPQKEKESYANDPSSGSFEGYGTKMTKNLDEKVEWVDYYFHIMHPPKKLNHDMWPKNPSSYRGVTEEYNVEILRTTNQILELLSEGLGLDGKVLNSSLGGDEIEFEMKINMYPPCAQPQLALGVEPHTDMSALTILVPNDVPGLQVWKDGNWVAVNYLPNALFVHVGDQLEVLSNGKYKSVLHRSLVNKERRRMSWAVFVVPPHEAVIGPLPELIDEKIPAKYSTKTYAEYRYRKFNKIPQ

SEQ ID NO: 107 - protein, 335 aa, synthetic construct
MEVERVQAIASLSHSNGTIPAEFIRPEKEQPASTTYHGPAPEIPTIDLDDPVQDRLVRSIAEASREWGIFQVTNHGIPSDLICKLQAVGKEFFELPQEEKEVYSRPADAKDVQGYGTKLQKEVEGKKSWVDHLFHRVWPPSSINYRFWPKNPPSYRAVNEEYAKYMREVVDKLFTYLSLGLGVEGGVLKEAAGGDDIEYMLKINYYPPCPRPDLALGVVAHTDLSALTVLVPNEVPGLQVFKDDRWIDAKYIPNALVIHIGDQIEILSNGKYKAVLHRTTVNKDKTRMSWPVFLEPPADTVVGPLPQLVDDENPPKYKAKKFKDYSYCKLNKLPQ

SEQ ID NO: 108 - protein, 335 aa, synthetic construct
MELKTVQAIAFSSMSAGIIPSEFIRSEKEQPAITTFHGYIPQVPTIDLSDPDEEKLTRLIVEASSEWGMFQIVNHGIPSDVISNLQKVGKEFFELPQEEKELYAKSPDSKSIQGYGSKLQKEVEGKKAWVDHLFHNIWPPPAIDYQFWPKKPPTYRAANEEYAKWLQGVADKLFGRLSLGLGLGEGTLKESVGGDELLYLLKINYYPPCPRPDLALGVVAHTDMSAITILIPNEVQGLQVFRDDHWFDVKYISNALVIHVGDQLEILSNGKYKAVLHRTTVNKEKTRMSWPVFLEPPSDQVVGPLPQLVNEENPARYKTKKYSDYVYCKLNKIPQ

SEQ ID NO: 109 - protein, 348 aa, synthetic construct
MKTAQGVSATLTMEVARVQAIASLSKCMDTIPSEYIRSENEQPAATTLHGVVLQVPVIDLRDPDENKMVKLIADASKEWGIFQLINHGIPDEAIADLQKVGKEFFEHVPQEEKELIAKTPGSNDIEGYGTSLQKEVEGKKGWVDHLFHKIWPPSAVNYRYWPKNPPSYREANEEYGKRMREVVDRIFKSLSLGLGLEGHEMIEAAGGDEIVYLLKINYYPPCPRPDLALGVVAHTDMSYITILVPNEVQGLQVFKDGHWYDVKYIPNALIVHIGDQVEILSNGKYKSVYHRTTVNKDKTRMSWPVFLEPPSEHEVGPIPKLLSEANPPKFKTKKYKDYVYCKLNKLPQ

SEQ ID NO: 110 - protein, 353 aa, synthetic construct
RYPPLLPATQEITKKPMEIERVQDISSSSLLTEAIPLEFIRSEKDQPAISTFRGPTPAIPVVDLSDPDKESVVRAVVKASEEWGIFQVVNHGIPTKLIQRLQEVGRKFFELPSTEKESVAKPEDSKDIEGYGTKLQKDLEGKKAWVDHLFHRIWPQSCVNYRFWPKNPPEYREVNEEYALQVNKLSETLLGILSEGLGLRREALKEGLGGDLAEYMMKINYYPPCPRPDLVLGVPAHTDLSGITLLVPNEVPGLQVFKDDHWIDAEYIPSAVIVHIGDQILRLSNGRYKNVLHRTTVDKEKTRMSWPVLVDPKRGLVVGPLSELIGDENPPKFESLTFEDYIYRKINMLLRDG

SEQ ID NO: 111 - protein, 367 aa, synthetic construct
MGRCHCHLWVRVFLQIQQSKKSKFHNKRKSLKKMEFLRVQTVASNSKDVAIPDMFVRPETEQPGITTVHGVKLEVPIIDFNNPDEGKVLNEIMEASKKWGMFQIVNHEIPSHVIKKLQSVGKEFFELPQEEKELYAKDPESKSIEGYGTKLQKELNGKKGWVDHLFHIIWPLSSINYRFWPKNPPSYREANEEYLKYLHGVVDKLFKSMSIGLGFEENELKEAAGGDSMIHLLKINYYPPCPFPDLVVGVPPHTDMSYITILVPNDVQGLQAFRDGHWYDVKYVPNALVIHIGDQMEIMSNGKYKAVFHRTTVSKEETRISWPVFIEPQPEYEVGPHPKLVNEENPPKYKTKKYKDYAYCKLNKIPQ

SEQ ID NO: 112 - protein, 293 aa, synthetic construct
MEVERVQAVASLSMLMDTIPSEFIRSEHERPGTTTFHGPVPELPAIDLSDQDQDNLVRLITKASEEFGIFQVVNHGIPVDIINKLQKVGREFFELPQEEKEAYATPPGAGHLEGEANEEFAKCLRGVVDKLLMNLSLGLGLEGNELKEAVGGDGLEYLLKINYYPTCPRPDLALGVVAHTDMPAITVLVPNDVPGLQVFKDEHCFEAKYIPNALIIHIGDQIEILSNGKYKSVLHRRTVNKEKVRMSWPVFCSPPGEQVIGPLPQLVSDDNPAKYKTKKYKDYMYCKINKLPQ

SEQ ID NO: 113 - protein, 340 aa, synthetic construct
MEVGRVQSIANKTASKDTIPLDFIWPENVQPAITTVHGADIEIPLIELDDDDEERLIKLIAKASSEVGLFQIVNHGIPSDVIERFQKVGKEFFELSQEEKERYGKLPGSVEGYGSSLQKEDDGKKGMKKGWVDHIFHRVWPPSAINLRFWPTILTSYREVNEEYAKYLRRVGDKLFTYLSLGLGLERHELREGAGGDDIEYLLKINYYPPCPRPDLALGVPAHSDMSAITLLVPNDVPGLQICKDDRWYNVNYVPNAIVIHIGDQIEIMSNGKYKAVMHRTTVSKDKTRFSWPVFLEPPLEQIVGPISKLINKQNPARFTSQKYRDYSSYKYNNISQQKI

SEQ ID NO: 114 - protein, 336 aa, synthetic construct
MEVERVQDISSSSLLTEAIPLEFIRSEKEQPAITTFRGPTPAIPVVDLSDPDEESVRRAVVKASEEWGLFQVVNHGIPTELIRRLQDVGRKFFELPSSEKESVAKPEDSKDIEGYGTKLQKDPEGKKAWVDHLFHRIWPPSCVNYRFWPKNPPEYREVNEEYAVHVKKLSETLLGILSDGLGLKRDALKEGLGGEMAEYMMKINYYPPCPRPDLALGVPAHTDLSGITLLVPNEVPGLQVFKDDHWFDAEYIPSAVIVHIGDQILRLSNGRYKNVLHRTTVDKEKTRMSWPVFLEPPREKIVGPLPELTGDDNPPKFKPFAFKDYSYRKLNKLPLD

SEQ ID NO: 115 - protein, 330 aa, synthetic construct
MDMERVQAIASLAGDLGTIPAEFIRPEHERPAMTTHHGPSPEIPVVDMTSVDRVKAIADAAKEWGIFQLVNHGIPVAAIEELKRVGTEFFALPQEEKESYAAAPGSLEGYGTRLQRESEGKKAWADYLFHNVWPDSSINYKFWPNNPPEYRKASEEYARRLQGVVDETLGSLSLWLGLERHVLKEAVGGERLEYLLKINYYPPCPRPDLALGVVAHTDMSAITVLVPNEVPGLQVFKDGRWLDAEYVPNALVVHVGDQVEIVSNGRYKSVMHRTVVSRDRVRMSWPVFCSPPGEEVVGPLPQLVGDGSPARFKAKKYKDYSYCKINKLPQ

SEQ ID NO: 116 - protein, 335 aa, synthetic construct
MEVERVQAIASMTASLGTIPAEFIRSEHERPDITTYHGPVPDIPVVDLSATDRAGVTKAIAEAAQEWGIFQVVNHRVPIEAIKELQRVGKEFFELPQEEKEAYATVPDSGSFEGYGTRLQKDLEGKKAWVDYLFHNVWPQSKITYRFWPNNPPEYRKANEEYAKYLQGVVDEMLTSLSLGLGLESHVLKEAVGGDELEYLLKINYYPPCPRPDLALGVVAHTDMSAITILVPNEVPGLQVFKDDHWFDAKYIPNALIVHIGDQIEILSNGKYKSVLHRTTVNKEKARMSWPVFCSPPGEMIVGPLPQLVSDENSAKFKTKKYKDYSYCKINKLPQ

SEQ ID NO: 117 - protein, 336 aa, synthetic construct
MEVERVQTIAFSSILVDTIPPEFIRSEKEQPALTTCRGCIPQVPTIDFSDPDEGNLTRLIAEASSEWGMFQIVNHGIPSEVITNLQKAGKEFFELPQEEKELYAKPPDSKSIEGYGSKLQKEVEGKKAWVDHLFHNIWPPSAINYQYWPKNPPSYRAVNEEYCKWVQRVGHRLLSLLSLGLGLEKNELKENVGGDELKYLLKINYYPPCPRPDLALGVVAHTDMSSITILVPNEVQGLQVFRDDHWFDVKYIPDALVIHIGDQLEVHLSNGKYKSVLHRTTVTKEITRMSWPVFLEPPSELAISPLPKLINEKNPPKYQKKKYCDYVYCKLNKIAQ

SEQ ID NO: 118 - protein, 335 aa, synthetic construct
MEFDRVQAIASLSFDKETIPEEFIWPEKEQPATTTFDGRVPEIPTIDLNDPNPENLVRLIADASKEWGIFQVVNHGIPSDLIAKLQDVGKKFFELPQEEKEVYAKPHDSKSIEGYGSKLQNNPQVKKSWVDHLFHIIWPPSSINYQFWPNNPPSYREVNEEYAKYMREVTDKLFTALSLGLGLEGHALKEGAGGEEIEYMLKINYYPPCPRPDLTLGVAAHTDLSALTILVPNEVPGLQIFKDGNWFEAKYIPNALIIHIGDQIEILSNGKYKAVLHRTTVAKDKARMSWPVFLEPPGELVVGPLPHLIKKDNPPKFKAKKFEDYMYCKLNRLPQ

SEQ ID NO: 119 - protein, 335 aa, synthetic construct
MEFDRVQTIASLSFDKEAIPLEFIRPEKEQPAITTFRGLAPEIPAIDLSDPDQEKLVGLIADASKEWGIFQVINHGIPSGVIAELQGAGKKFFELPQEEKEVCARPPDSKSIEGYDSKLQKDPQEKKSWVDHLFHRIWPPPSINYQFWPENPPSYREVTKEYAKYMRDVVDKLFTTLSLGLGLEGHVLKEAAGGEQIEYMLKINYYPPCPRPDLTLGVVAHTDLSAITILVPNEVPGLQIFKDDHWIEAKYIPDALIIHIGDQIEILSNGKYKAVLHRTTVAKNRTRMSWPVFLEPPGELVVGPLPQFINKDNPPKFKTKKFKDYTYCKLNKLPQ

SEQ ID NO: 120 - protein, 333 aa, synthetic construct
MEVERVQAIASLSVATNDIPPEFVRSEHEQPGITTYRGPVPEIPVIDLEDGDEGRVTRAIAEASQEWGIFQLVNHGIPGEVIRALQRVGREFFELPPEEKEKYAAAPGSLQGYGTKLQKDLEGKKAWVDFLFHNIWPPTHVDHRAWPENPVDYRKANEEYAKHLVGLVEKMLVSLSKGLGLEADVLKHAVGGDDLEFLLKINYYPPCPRPDLALGVVAHTDMSAITILIPNDVPGLQVFKDDHWFDAKYVPDAIIVHIGDQIEKLSNGRYKSVLHRTTVNKEKARMSWPVFCSPPGETVIGPLPQLVSDEQPAQYKTKKYKDYAFCKLNKLPQ

SEQ ID NO: 121 - protein, 331 aa, synthetic construct
MAEVQSVQALASSLAALPPEFVRSEHERPGATTFRGGDAPEIPVIDMAAPESAARVAEAAAEWGLFQVVNHGVPAAAVAELQRVGREFFALPQEEKARYAMDASSGKMEGYGSKLQKDLEGKKAWADFFFHNVAPPAMVNHDIWPSHPAGYREANEEYCKHMQRLARKLFEHLSTALGLDGGAMWEAFGGDELVFLHKINFYPPCPEPELTLGVAPHTDMSTFTVLVPNDVQGLQVFKDGHWYDVKYVPDALIIHIGDQIEILSNGRYKAVLHRTTVDKDRTRMSWPVFVEPPPEHVVGPHPQLVTDGSPAKYKAKKFKDYRHCKINKLPM

SEQ ID NO: 122 - protein, 336 aa, synthetic construct
MEIERVQDISSSSLHTEVIPLEFIRSEKEQPAITTFRGPVPAIPVVDLSDPDEESVARAVVKASEEWGIFQVVNHGIPTELIKRLQEVGRTFFELPSSEKESVAKPADAKDIEGYGTKLQKEVEGKKAWVDHLFHRIWPPSCVNYSFWPKNPPEYREVNEEYALHVKKLSETLLGILSEGLGLRREALREGLGGDLVEYMMKINYYPPCPRPDLALGVPAHTDLSGITLLVPNEVPGLQVFKDDHWFDAEYIPSAVIVHIGDQILRLSNGRYKNVLHRTTVDKDRTRMSWPVFLEPHREMIVGPLPELISDGNPPKYKPFAFKDYSYRKLNKLPLD